(19) United States
(12) Patent Application Publication
(10) Pub. No.: US 2015/0243892 A1
(43) Pub. Date: Aug. 27, 2015

(54) Organic Compound, Light-Emitting Element, Display Module, Lighting Module, Light-Emitting Device, Display Device, Electronic Device, and Lighting Device

(71) Applicant: Semiconductor Energy Laboratory Co., Ltd., Kanagawa-ken (JP)

(72) Inventors: Kaori Ogita, Isehara (JP); Tsunenori Suzuki, Yokohama (JP); Naoaki Hashimoto, Atsugi (JP); Toshiki Hamada, Atsugi (JP); Kunihiko Suzuki, Ishehara (JP); Shunsuke Hosoumi, Atsugi (JP); Satoshi Seo, Sagamihara (JP)

(73) Assignee: Semiconductor Energy Laboratory Co., Ltd., Kanagawa-ken (JP)

(21) Appl. No.: 14/625,834

(22) Filed: Feb. 19, 2015

(30) Foreign Application Priority Data
Feb. 21, 2014 (JP) 2014-031853
Feb. 21, 2014 (JP) 2014-032002

(51) Int. Cl. H01L 51/00; C07D 307/91; C07C 211/61

(52) U.S. Cl. CPC H01L 51/006; C07C 211/61; H01L 51/0058; C07D 307/91; H01L 51/0061; H01L 51/0073; H01L 51/5012

(57) ABSTRACT

A light-emitting element with high emission efficiency. The light-emitting element includes a pair of electrodes and an EL layer between the pair of electrodes. In the light-emitting element, the EL layer contains at least a light-emitting material, the light-emitting material is a 1,6-bis(diphenylamino)pyrene derivative, and a structural change between an excited state and a ground state in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

### BACKGROUND OF THE INVENTION

1. Field of the Invention

One embodiment of the present invention relates to an organic compound, and a light-emitting element, a display module, a lighting module, a display device, a light-emitting device, an electronic device, and a lighting device each including the organic compound. Note that one embodiment of the present invention is not limited to the above technical field. The technical field of one embodiment of the invention disclosed in this specification and the like relates to an object, a method, or a manufacturing method. In addition, one embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting device, a lighting device, a power storage device, a storage device, a method of driving any of them, and a method of manufacturing any of them.

2. Description of the Related Art

As next generation lighting devices or display devices, display devices using light-emitting elements (organic EL elements) in which organic compounds are used as light-emitting substances have been developed and reported because of their potential for thinness, lightness, high speed response to input signals, low power consumption, and the like.

In an organic EL element, voltage application between electrodes, between which a light-emitting layer is interposed, causes recombination of electrons and holes injected from the electrodes, which brings a light-emitting substance (an organic compound) into an excited state, and the return from the excited state to the ground state is accompanied by light emission. Since the spectrum of light emitted from a light-emitting substance depends on the light-emitting substance, use of different types of organic compounds as light-emitting substances makes it possible to obtain light-emitting elements which exhibit various colors.

In the case of display devices which are used to display images, such as displays, at least three-color light, i.e., red light, green light, and blue light is necessary for reproduction of full-color images. For higher color reproducibility and higher quality of the display images, the color purity of emitted light is increased with the use of a microcavity structure or a color filter.

A microcavity structure is designed so that light with a desired wavelength is amplified and light with the other wavelengths is diminished. A color filter intercepts light except light with a desired wavelength. Therefore, in a light-emitting element having a microcavity structure or a color filter, light with relatively high spectral intensity with respect to a non-desired wavelength is mostly diminished or intercepted and thus cannot be extracted.

To make maximum use of given energy, an organic compound that has high internal quantum efficiency, high spectral intensity with respect to a desired wavelength, and a small half width of an emission spectrum is needed.

Patent Document 1 discloses an organic compound that emits excellent blue light.

### REFERENCE

### Patent Document

### [Patent Document 1] Japanese Published Patent Application No. 2012-46478

### SUMMARY OF THE INVENTION

An object of one embodiment of the present invention is to provide a novel organic compound. An object of another embodiment of the present invention is to provide an organic compound with a small half width of an emission spectrum. An object of another embodiment of the present invention is to provide an organic compound having high color purity.

An object of another embodiment of the present invention is to provide a novel light-emitting element. An object of another embodiment of the present invention is to provide a light emitting element with high emission efficiency. An object of another embodiment of the present invention is to provide a display module, a lighting module, a light-emitting device, a display device, an electronic device, and a lighting device each having low power consumption.

It is only necessary that at least one of the above-described objects be achieved in one embodiment of the present invention.

One embodiment of the present invention is a light-emitting element including a pair of electrodes and an EL layer between the pair of electrodes. In the light-emitting element, the EL layer contains at least a light-emitting material; the light-emitting material is a 1,6-bis(diphenylamino)pyrene derivative; and a structural change between an excited state and a ground state in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

Another embodiment of the present invention is a light-emitting element including a pair of electrodes and an EL layer between the pair of electrodes. In the light-emitting element, the EL layer contains at least a light-emitting material; the light-emitting material is a 1,6-bis(diphenylamino)pyrene derivative; and a Stokes shift in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

Another embodiment of the present invention is a light-emitting element including a pair of electrodes and an EL layer between the pair of electrodes. In the light-emitting element, the EL layer contains at least a light-emitting material; the light-emitting material is a 1,6-bis(diphenylamino)pyrene derivative; and a half width of an emission spectrum in the 1,6-bis(diphenylamino)pyrene derivative is narrower than that in a 1,6-bis(diphenylamino)pyrene derivative in which hydrogen is bonded to ortho positions of two phenyl groups of each of two diphenylamino groups.

Another embodiment of the present invention is a light-emitting element including a pair of electrodes and an EL layer between the pair of electrodes. In the light-emitting element, the EL layer contains at least a light-emitting material; the light-emitting material is a 1,6-bis(diphenylamino)pyrene derivative; the 1,6-bis(diphenylamino)pyrene derivative includes two diphenylamino groups; each of the two diphenylamino groups includes two phenyl groups; and an alkyl group is bonded to each of two ortho positions of at least one of the two phenyl groups.

Another embodiment of the present invention is a light-emitting element including a pair of electrodes and an EL layer between the pair of electrodes. In the light-emitting element, the EL layer contains at least a light-emitting material; the light-emitting material is a 1,6-bis(diphenylamino)pyrene derivative; the 1,6-bis(diphenylamino)pyrene derivative includes two diphenylamino groups; each of the two diphenylamino groups includes two phenyl groups; an alkyl group is bonded to each of two ortho positions of at least one of the two phenyl groups; and a structural change between an excited state and a ground state in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

Another embodiment of the present invention is a light-emitting element including a pair of electrodes and an EL layer between the pair of electrodes. In the light-emitting element, the EL layer contains at least a light-emitting material; the light-emitting material is a 1,6-bis(diphenylamino)pyrene derivative; the 1,6-bis(diphenylamino)pyrene derivative includes two diphenylamino groups; each of the two diphenylamino groups includes two phenyl groups; an alkyl group is bonded to each of two ortho positions of at least one of the two phenyl groups; and a Stokes shift in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

Another embodiment of the present invention is a light-emitting element including a pair of electrodes and an EL layer between the pair of electrodes. In the light-emitting element, the EL layer contains at least a light-emitting material; the light-emitting material is a 1,6-bis(diphenylamino)pyrene derivative; the 1,6-bis(diphenylamino)pyrene derivative includes two diphenylamino groups; each of the two diphenylamino groups includes two phenyl groups, wherein an alkyl group is bonded to each of two ortho positions of at least one of the two phenyl groups; and a half width of an emission spectrum in the 1,6-bis(diphenylamino)pyrene derivative is narrower than that in a 1,6-bis(diphenylamino)pyrene derivative in which hydrogen is bonded to ortho positions of two phenyl groups of each of two diphenylamino groups.

Another embodiment of the present invention is a light-emitting element with any of the above structures, in which the 1,6-bis(diphenylamino)pyrene derivative contained as a light-emitting material in the EL layer includes two diphenylamino groups; each of the two diphenylamino groups includes two phenyl groups; an alkyl group is bonded to each of two ortho positions of one of the two phenyl groups; and hydrogen is bonded to each of two ortho positions of the other of the two phenyl groups.

Another embodiment of the present invention is a light-emitting element with any of the above structures, in which a Stokes shift of the light-emitting material is less than or equal to 0.18 eV.

Another embodiment of the present invention is a light-emitting element with any of the above structures, in which a Stokes shift of the light-emitting material is less than or equal to 0.15 eV.

Another embodiment of the present invention is a light-emitting element with any of the above structures, in which the EL layer further contains a host material; the light-emitting material is dispersed in the host material; and an absorption spectrum peak of the light-emitting material on the longest wavelength side overlaps with an emission spectrum of the host material.

Another embodiment of the present invention is a light-emitting element with the above structure, in which the half width of an emission spectrum of the light-emitting material is less than or equal to 40 nm.

Another embodiment of the present invention is a light-emitting element with the above structure, in which the half width of an emission spectrum of the light-emitting material is less than or equal to 35 nm.

Another embodiment of the present invention is a light-emitting element with the above structure, in which the y-coordinate of the CIE chromaticity of the light-emitting element is less than or equal to 0.15.

Another embodiment of the present invention is a light-emitting element with the above structure, in which the peak wavelength of light emitted from the light-emitting element is less than or equal to 465 nm.

Another embodiment of the present invention is a 1,6-bis(diphenylamino)pyrene derivative including two diphenylamino groups, in which each of the two diphenylamino groups includes two phenyl groups; an alkyl group is bonded to each of two ortho positions of at least one of the two phenyl groups; and a structural change between an excited state and a ground state in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

Another embodiment of the present invention is a 1,6-bis(diphenylamino)pyrene derivative including two diphenylamino groups, in which each of the two diphenylamino groups includes two phenyl groups; an alkyl group is bonded to each of two ortho positions of at least one of the two phenyl groups; and a Stokes shift in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

Another embodiment of the present invention is a 1,6-bis(diphenylamino)pyrene derivative including two diphenylamino groups, in which each of the two diphenylamino groups includes two phenyl groups; an alkyl group is bonded to each of two ortho positions of at least one of the two phenyl groups; and a half width of an emission spectrum in the 1,6-bis(diphenylamino)pyrene derivative is narrower than that in a 1,6-bis(diphenylamino)pyrene derivative in which hydrogen is bonded to ortho positions of two phenyl groups of each of two diphenylamino groups.

Another embodiment of the present invention is a 1,6-bis(diphenylamino)pyrene derivative with any of the above structures, in which in each of the two diphenylamino groups, an alkyl group is bonded to two ortho positions of one phenyl group, and hydrogen is bonded to two ortho positions of the other phenyl group.

Another embodiment of the present invention is a 1,6-bis(diphenylamino)pyrene derivative with any of the above structures, in which a Stokes shift is less than or equal to 0.18 eV.

Another embodiment of the present invention is a 1,6-bis(diphenylamino)pyrene derivative with any of the above structures, in which a Stokes shift is less than or equal to 0.15 eV.

Another embodiment of the present invention is a 1,6-bis(diphenylamino)pyrene derivative with any of the above structures, in which a half width of an emission spectrum is less than or equal to 40 nm.

Another embodiment of the present invention is a 1,6-bis(diphenylamino)pyrene derivative with any of the above structures, in which a half width of an emission spectrum is less than or equal to 35 nm.

Another embodiment of the present invention is a 1,6-bis(diphenylamino)pyrene derivative with any of the above structures, in which an emission peak wavelength is less than or equal to 465 nm.

Another embodiment of the present invention is an organic compound represented by General Formula (G1-1).

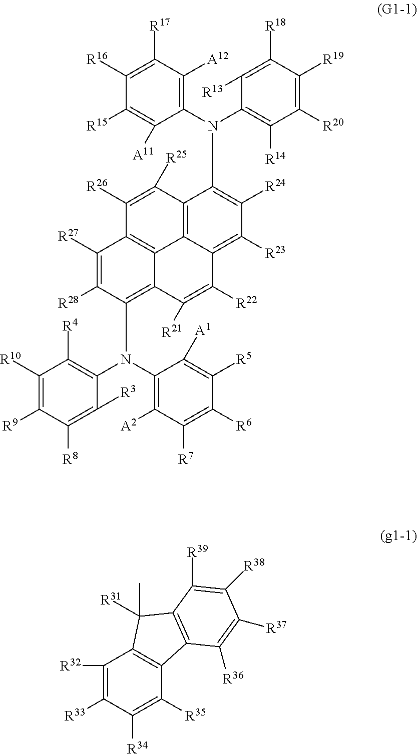

In General Formula (G1-1), A¹, A², A¹¹, and A¹² each represent an alkyl group having 1 to 6 carbon atoms; at least one of R³ and R⁴ represents hydrogen and the other represents hydrogen or an alkyl group having 1 to 6 carbon atoms; at least one of R¹³ and R¹⁴ represents hydrogen and the other represents hydrogen or an alkyl group having 1 to 6 carbon atoms; and R⁵ to R¹⁰, R¹⁵ to R²⁰, and R²¹ to R²⁸ each independently represent hydrogen, an alkyl group having 1 to 6 carbon atoms, or an aryl group having 6 to 25 carbon atoms. Note that any one of R⁵ to R¹⁰ and any one of R¹⁵ to R²⁰ are substituents represented by General Formula (g1-1). In General Formula (g1-1), R³¹ to R³⁹ each independently represent hydrogen, an alkyl group having 1 to 6 carbon atoms, or an aryl group having 6 to 25 carbon atoms.

Another embodiment of the present invention is an organic compound represented by General Formula (G2-1).

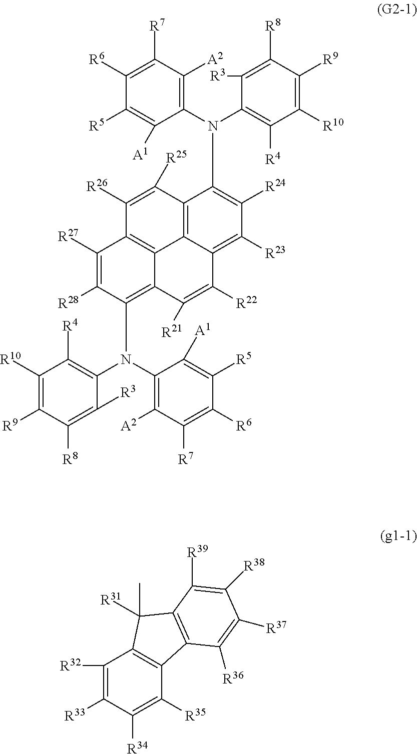

In General Formula (G2-1), A¹ and A² each represent an alkyl group having 1 to 6 carbon atoms; at least one of R³ and R⁴ represents hydrogen and the other represents hydrogen or an alkyl group having 1 to 6 carbon atoms; and R⁵ to R¹⁰ and R²¹ to R²⁸ each independently represent hydrogen, an alkyl group having 1 to 6 carbon atoms, or an aryl group having 6 to 25 carbon atoms. Note that any one of R⁵ to R¹⁰ is a substituent represented by General Formula (g1-1). In General Formula (g1-1), R³¹ to R³⁹ each independently represent hydrogen, an alkyl group having 1 to 6 carbon atoms, or an aryl group having 6 to 25 carbon atoms.

Another embodiment of the present invention is an organic compound represented by General Formula (G3-1).

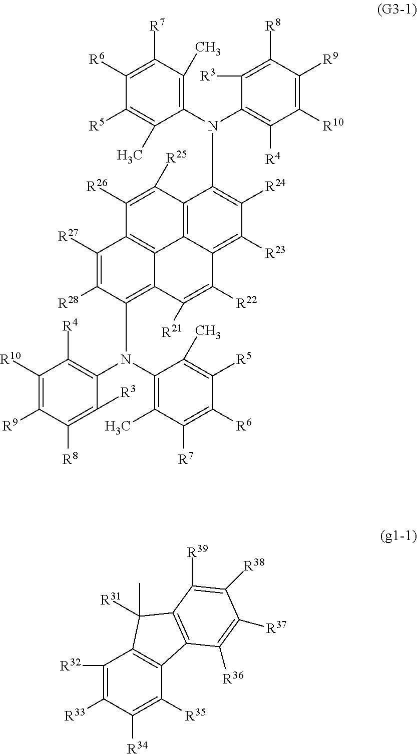

In General Formula (G3-1), at least one of R³ and R⁴ represents hydrogen and the other represents hydrogen or an alkyl group having 1 to 6 carbon atoms; and R⁵ to R¹⁰ and R²¹ to R²⁸ each independently represent hydrogen, an alkyl group having 1 to 6 carbon atoms, or an aryl group having 6 to 25 carbon atoms. Note that any one of R⁵ to R¹⁰ is a substituent represented by General Formula (g1-1). In General Formula (g1-1), R³¹ to R³⁹ each independently represent hydrogen, an alkyl group having 1 to 6 carbon atoms, or an aryl group having 6 to 25 carbon atoms.

Another embodiment of the present invention is an organic compound represented by General Formula (G4-1).

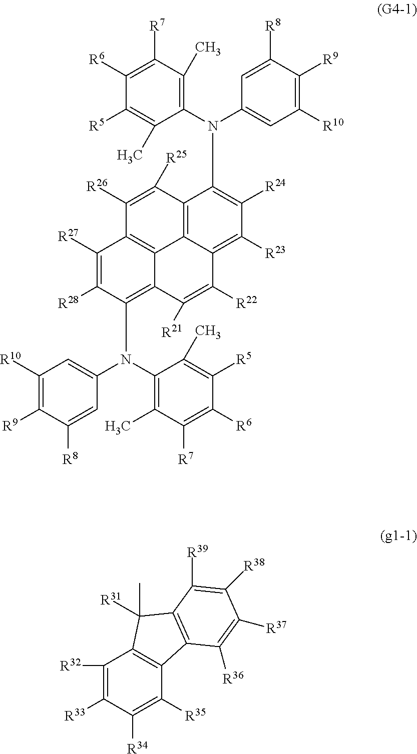

In General Formula (G4-1), R⁵ to R¹⁰ and R²¹ to R²⁸ each independently represent hydrogen, an alkyl group having 1 to 6 carbon atoms, or an aryl group having 6 to 25 carbon atoms. Note that any one of R⁵ to R¹⁰ is a substituent represented by General Formula (g1-1). In General Formula (g1-1), R³¹ to R³⁹ each independently represent hydrogen, an alkyl group having 1 to 6 carbon atoms, or an aryl group having 6 to 25 carbon atoms.

Another embodiment of the present invention is an organic compound with the above structure, in which R⁷ or R⁸ in General Formula (G2-1) is a substituent represented by General Formula (g1-1).

Another embodiment of the present invention is an organic compound with the above structure, in which R⁸ in General Formula (G2-1) is a substituent represented by General Formula (g1-1).

Another embodiment of the present invention is an organic compound with the above structure, in which R⁷ in General Formula (G2-1) is a substituent represented by General Formula (g1-1).

Another embodiment of the present invention is an organic compound represented by General Formula (G5-1).

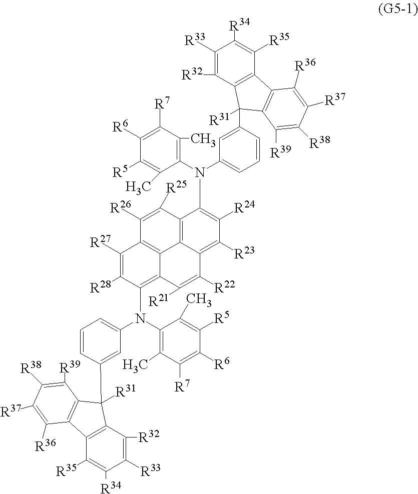

In General Formula (G5-1), R⁵ to R⁷, R¹⁰, R²¹ to R²⁸, and R³¹ to R³⁹ each independently represent hydrogen, an alkyl group having 1 to 6 carbon atoms, or an aryl group having 6 to 25 carbon atoms.

Another embodiment of the present invention is an organic compound represented by Structural formula (1200).

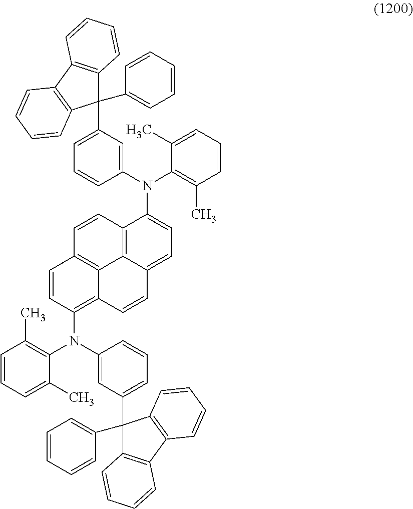 [SMILES: Cc1cccc(C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C)cccc4C)ccc4ccc1c2c43]

Another embodiment of the present invention is a light-emitting element including the above organic compound.

Another embodiment of the present invention is a light-emitting element including the above organic compound in a light-emitting layer.

Another embodiment of the present invention is a light-emitting element with any of the above structures, in which the y-coordinate of the CIE chromaticity of the light-emitting element is less than or equal to 0.15.

Another embodiment of the present invention is a light-emitting element with any of the above structures, in which the peak wavelength of light emitted from the light-emitting element is less than or equal to 465 nm.

Another embodiment of the present invention is a light-emitting element with any of the above structures, in which the half width of an emission spectrum of the light-emitting element is less than or equal to 40 nm.

Another embodiment of the present invention is an organic compound represented by General Formula (G1-2).

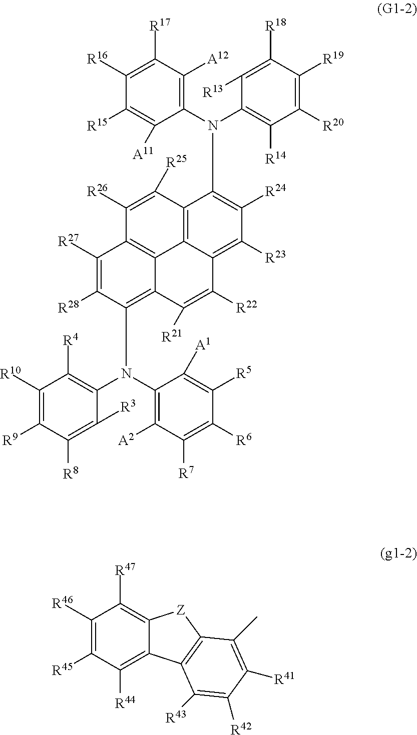

In General Formula (G1-2), A¹, A², A¹¹, and A¹² each represent an alkyl group having 1 to 6 carbon atoms; at least one of R³ and R⁴ represents hydrogen and the other represents hydrogen or an alkyl group having 1 to 6 carbon atoms; at least one of R¹³ and R¹⁴ represents hydrogen and the other represents hydrogen or an alkyl group having 1 to 6 carbon atoms; and R⁵ to R¹⁰, R¹⁵ to R²⁰, and R²¹ to R²⁸ each independently represent hydrogen, an alkyl group having 1 to 6 carbon atoms, or an aryl group having 6 to 25 carbon atoms. Note that any one of R⁵ to R¹⁰ and any one of R¹⁵ to R²⁰ are substituents represented by General Formula (g1-2). In General Formula (g1-2), R⁴¹ to R⁴⁷ each independently represent hydrogen, an alkyl group having 1 to 6 carbon atoms, or an aryl group having 6 to 25 carbon atoms; and Z represents an oxygen atom or a sulfur atom.

Another embodiment of the present invention is an organic compound represented by General Formula (G2-2).

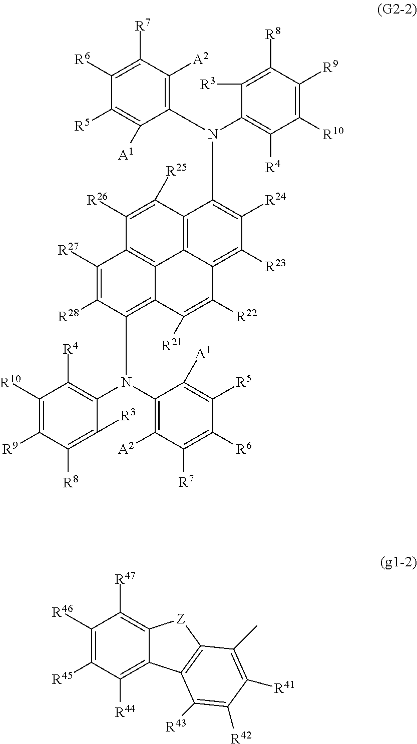

In General Formula (G2-2), A¹ and A² each represent an alkyl group having 1 to 6 carbon atoms; at least one of R³ and R⁴ represents hydrogen and the other represents hydrogen or an alkyl group having 1 to 6 carbon atoms; R⁵ to R¹⁰ and R²¹ to R²⁸ each independently represent hydrogen; an alkyl group having 1 to 6 carbon atoms; or an aryl group having 6 to 25 carbon atoms; any one of R⁵ to R¹⁰ is a substituent represented by General Formula (g1-2). In General Formula (g1-2), R⁴¹ to R⁴⁷ each independently represent hydrogen, an alkyl group having 1 to 6 carbon atoms, or an aryl group having 6 to 25 carbon atoms; and Z represents an oxygen atom and a sulfur atom.

Another embodiment of the present invention is an organic compound represented by General Formula (G3-2).

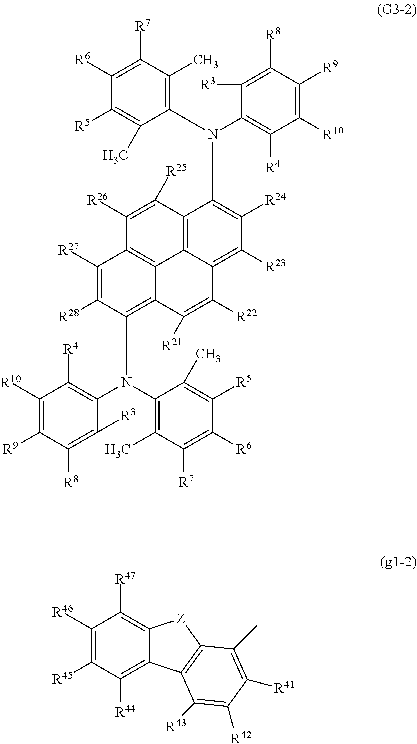

In General Formula (G3-2), at least one of R³ and R⁴ represents hydrogen and the other represents hydrogen or an alkyl group having 1 to 6 carbon atoms; R⁵ to R¹⁰ and R²¹ to R²⁸ each independently represent hydrogen; an alkyl group having 1 to 6 carbon atoms; or an aryl group having 6 to 25 carbon atoms; any one of R⁵ to R¹⁰ is a substituent represented by General Formula (g1-2). In General Formula (g1-2), R⁴¹ to R⁴⁷ each independently represent hydrogen, an alkyl group having 1 to 6 carbon atoms, or an aryl group having 6 to 25 carbon atoms; and Z represents an oxygen atom and a sulfur atom.

Another embodiment of the present invention is an organic compound represented by General Formula (G4-2).

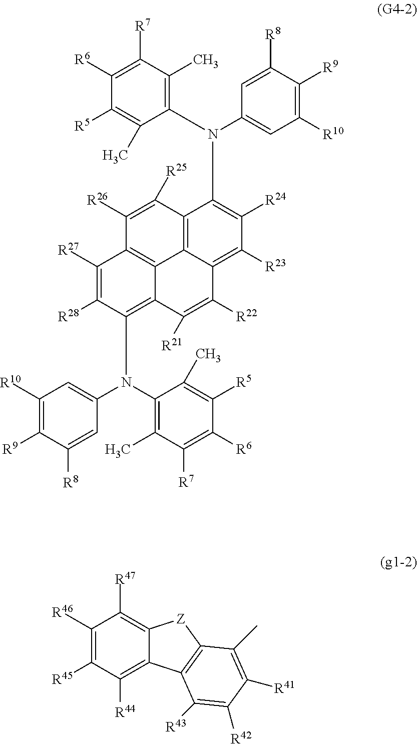

In General Formula (G4-2), R⁵ to R¹⁰ and R²¹ to R²⁸ each independently represent hydrogen; an alkyl group having 1 to 6 carbon atoms; or an aryl group having 6 to 25 carbon atoms; any one of R⁵ to R¹⁰ is a substituent represented by General Formula (g1-2). In General Formula (g1-2), R⁴¹ to R⁴⁷ each independently represent hydrogen, an alkyl group having 1 to 6 carbon atoms, or an aryl group having 6 to 25 carbon atoms; and Z represents an oxygen atom and a sulfur atom.

Another embodiment of the present invention is an organic compound with the above structure, in which R⁷ or R⁸ in General Formula (G2-2) is a substituent represented by General Formula (g1-2).

Another embodiment of the present invention is an organic compound with the above structure, in which R⁸ in General Formula (G2-2) is a substituent represented by General Formula (g1-2).

Another embodiment of the present invention is an organic compound with the above structure, in which R⁷ in General Formula (G2-2) is a substituent represented by General Formula (g1-2).

Another embodiment of the present invention is an organic compound represented by General Formula (G5-2).

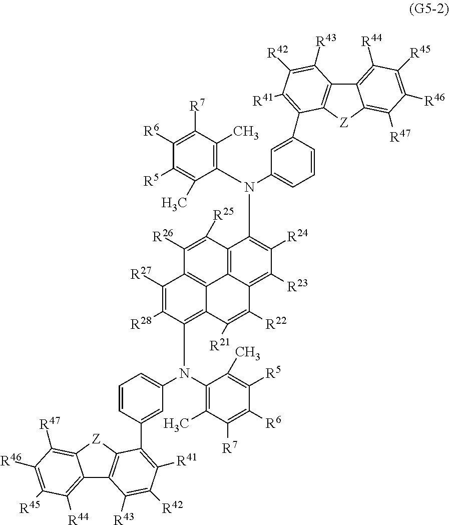

In General Formula (G5-2), R⁵ to R⁷, R²¹ to R²⁸, and R⁴¹ to R⁴⁷ each independently represent hydrogen, an alkyl group having 1 to 6 carbon atoms, or an aryl group having 6 to 25 carbon atoms; and Z represents an oxygen atom or a sulfur atom.

Another embodiment of the present invention is an organic compound represented by General Formula (G6-2).

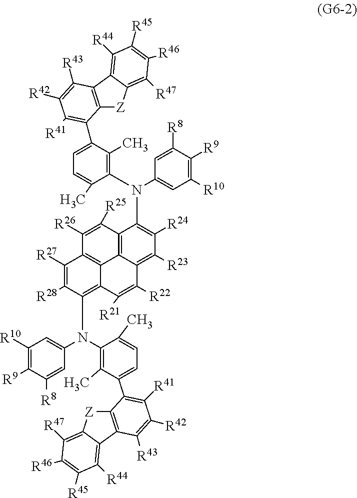

In General Formula (G6-2), R⁸ to R¹⁰, R²¹ to R²⁸, and R⁴¹ to R⁴⁷ each independently represent hydrogen, an alkyl group having 1 to 6 carbon atoms, or an aryl group having 6 to 25 carbon atoms; and Z represents an oxygen atom or a sulfur atom.

Another embodiment of the present invention is any of the above-described organic compounds in which Z represents an oxygen atom.

Another embodiment of the present invention is an organic compound represented by Structural formula (2100).

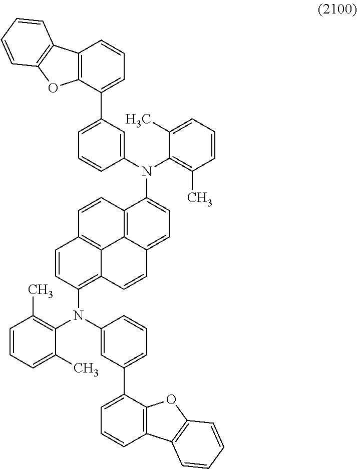 [SMILES: Cc1cccc(C)c1N(c1cccc(-c2cccc3c2oc2ccccc23)c1)c1ccc2ccc3c(N(c4cccc(-c5cccc6c5oc5ccccc56)c4)c4c(C)cccc4C)ccc4ccc1c2c43]

Another embodiment of the present invention is an organic compound represented by Structural formula (2200).

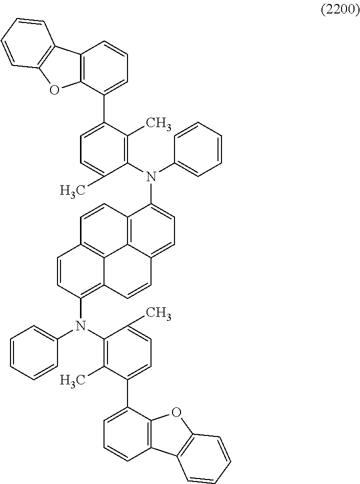 [SMILES: Cc1ccc(-c2cccc3c2oc2ccccc23)c(C)c1N(c1ccccc1)c1ccc2ccc3c(N(c4ccccc4)c4c(C)ccc(-c5cccc6c5oc5ccccc56)c4C)ccc4ccc1c2c43]

Another embodiment of the present invention is a light-emitting element with any of the above structures, including a pair of electrodes and an EL layer between the pair of electrodes. The EL layer contains any of the organic compounds described above.

Another embodiment of the present invention is a light-emitting element including a pair of electrodes and an EL layer between the pair of electrodes. The EL layer contains any of the organic compounds described above as a light-emitting material.

Another embodiment of the present invention is a light-emitting element with any of the above structures, in which the light-emitting element has a tandem structure.

Another embodiment of the present invention is a light-emitting element with any of the above structures, in which the light-emitting element has a microcavity structure that increases the intensity of light in a blue region.

Another embodiment of the present invention is a light-emitting element including any of the above organic compounds.

Another embodiment of the present invention is a light-emitting element including any of the above organic compounds in a light-emitting layer.

Another embodiment of the present invention is a display module that includes any of the above-described light-emitting elements.

Another embodiment of the present invention is a lighting module that includes any of the above-described light-emitting elements.

Another embodiment of the present invention is a light-emitting device that includes any of the above-described light-emitting elements and a unit for controlling the light-emitting element.

Another embodiment of the present invention is a display device that includes any of the above-described light-emitting elements in a display portion and a unit for controlling the light-emitting element.

Another embodiment of the present invention is a lighting device that includes any of the above-described light-emitting elements in a lighting portion and a unit for controlling the light-emitting element.

Another embodiment of the present invention is an electronic device that includes any of the above-described light-emitting elements.

Note that the light-emitting device in this specification includes an image display device using a light-emitting element. The light-emitting device may be included in a module in which a light-emitting element is provided with a connector such as an anisotropic conductive film or a tape carrier package (TCP), a module in which a printed wiring board is provided at the end of a TCP, and a module in which an integrated circuit (IC) is directly mounted on a light-emitting element by a chip on glass (COG) method. The light-emitting device may be included in lighting equipment.

One embodiment of the present invention makes it possible to provide a novel organic compound. Another embodiment of the present invention makes it possible to provide an organic compound that can be used in a light-emitting element. Another embodiment of the present invention makes it possible to provide an organic compound with high triplet excitation level. Another embodiment of the present invention makes it possible to provide an organic compound with high heat resistance.

Another embodiment of the present invention makes it possible to provide a novel light-emitting element, a novel display module, a novel lighting module, a novel light-emitting device, a novel display device, a novel electronic device, and a novel lighting device. Another embodiment of the present invention makes it possible to provide a light-emitting element having high emission efficiency. Another embodiment of the present invention makes it possible to provide a display module, a lighting module, a light-emitting device, a display device, an electronic device, and a lighting device each having low power consumption.

It is only necessary that at least one of the above effects be achieved in one embodiment of the present invention.

### BRIEF DESCRIPTION OF THE DRAWINGS

FIGS. 1A and 1B are conceptual diagrams of light-emitting elements.

FIGS. 2A and 2B are conceptual diagrams of an active matrix light-emitting device.

FIGS. 3A and 3B are conceptual diagrams of an active matrix light-emitting device.

FIG. 4 is a conceptual diagram of an active matrix light-emitting device.

FIGS. 5A and 5B are conceptual diagrams of a passive matrix light-emitting device.

FIGS. 6A and 6B illustrate a lighting device.

FIGS. 7A, 7B1, 7B2, 7C, 7D1, and 7D2 illustrate electronic devices.

FIG. 8 illustrates a light source device.

FIG. 9 illustrates a lighting device.

FIG. 10 illustrates a lighting device.

FIG. 11 illustrates in-vehicle display devices and lighting devices.

FIGS. 12A to 12C illustrate an electronic device.

FIG. 13 shows calculation results.

FIGS. 14A to 14C show calculation results.

FIGS. 15A and 15B are NMR charts of 1,6oDMemFLPAPrn.

FIG. 16 shows an MS spectrum of 1,6oDMemFLPAPrn.

FIGS. 17A and 17B show an absorption spectrum and an emission spectrum of 1,6oDMemFLPAPrn.

FIG. 18 shows luminance-current efficiency characteristics of Light-emitting element 1, Light-emitting element 2, and Comparative light-emitting element 1.

FIG. 19 shows voltage-luminance characteristics of Light-emitting element 1, Light-emitting element 2, and Comparative light-emitting element 1.

FIG. 20 shows voltage-current characteristics of Light-emitting element 1, Light-emitting element 2, and Comparative light-emitting element 1.

FIG. 21 shows luminance-power efficiency characteristics of Light-emitting element 1, Light-emitting element 2, and Comparative light-emitting element 1.

FIG. 22 shows luminance-external quantum efficiency characteristics of Light-emitting element 1, Light-emitting element 2, and Comparative light-emitting element 1.

FIGS. 23A and 23B show emission spectra of Light-emitting element 1, Light-emitting element 2, and Comparative light-emitting element 1.

FIG. 24 shows time dependences of normalized luminances of Light-emitting element 1, Light-emitting element 2, and Comparative light-emitting element 1.

FIGS. 25A to 25C show comparison of emission spectra.

FIGS. 26A and 26B are NMR charts of oDMemFLPA.

FIGS. 27A and 27B are NMR charts of mFrBA-04.

FIGS. 28A and 28B are NMR charts of 1,6mFrBAPrn-04.

FIG. 29 shows an MS spectrum of 1,6mFrBAPrn-04.

FIGS. 30A and 30B show an absorption spectrum and an emission spectrum of 1,6mFrBAPrn-04.

FIGS. 31A and 31B are NMR charts of oDMemFrBA.

FIG. 32 shows an MS spectrum of 1,6oDMemFrBAPrn.

FIGS. 33A and 33B show an absorption spectrum and an emission spectrum of 1,6oDMemFrBAPrn.

FIG. 34 shows luminance-current efficiency characteristics of Light-emitting element 3 and Comparative light-emitting element 2.

FIG. 35 shows voltage-luminance characteristics of Light-emitting element 3 and Comparative light-emitting element 2.

FIG. 36 shows voltage-current characteristics of Light-emitting element 3 and Comparative light-emitting element 2.

FIG. 37 shows luminance-power efficiency characteristics of Light-emitting element 3 and Comparative light-emitting element 2.

FIG. 38 shows luminance-external quantum efficiency characteristics of Light-emitting element 3 and Comparative light-emitting element 2.

FIGS. 39A and 39B show emission spectra of Light-emitting element 3 and Comparative light-emitting element 2.

FIG. 40 shows luminance-current efficiency characteristics of Light-emitting element 4.

FIG. 41 shows voltage-luminance characteristics of Light-emitting element 4.

FIG. 42 shows voltage-current characteristics of Light-emitting element 4.

FIG. 43 shows luminance-power efficiency characteristics of Light-emitting element 4.

FIG. 44 shows luminance-external quantum efficiency characteristics of Light-emitting element 4.

FIGS. 45A and 45B show emission spectra of Light-emitting element 4 and Comparative light-emitting element 2.

### DETAILED DESCRIPTION OF THE INVENTION

Embodiments of the present invention will be explained below with reference to the drawings. Note that the present invention is not limited to the description below, and it is easily understood by those skilled in the art that modes and details can be modified in various ways without departing from the spirit and scope of the present invention. Accordingly, the present invention should not be interpreted as being limited to the content of the embodiments below.

Full-color displays use light of the three primary colors (i.e., red, green, and blue), or four or more colors, (i.e., the three primary colors and white and/or yellow) for displaying images. The color reproducibility of the images greatly depends on the tone of the three primary colors.

A separate coloring method and a white color filter method are mainly used as a full-color display method of an organic EL display. A color filter is essential for the white color filter method, and is used for the separate coloring method in some cases to achieve excellent color reproducibility. For the same reason, a microcavity structure is employed in the both full-color display methods.

An organic compound or an organometallic complex is used as a light-emitting substance of such an organic EL element. An emission spectrum obtained from the organic compound or the organometallic complex is expressed by a band spectrum with high intensity with respect to a particular wavelength of the substance. Since a color filter intercepts light except light with a desired wavelength and a microcavity structure amplifies light with a desired wavelength and diminishes light with the other wavelengths, light with a broad spectrum results in a significant energy loss.

The present inventors found that a light-emitting material with a small Stokes shift can reduce the energy loss described above because its half width is narrow, and with the use of the light-emitting material, a light-emitting element with high emission efficiency can be obtained.

A light-emitting element formed with a light-emitting material with a small Stokes shift and a narrow half width of an emission spectrum can emit light having high color purity.

Moreover, a light-emitting material with a small Stokes shift has a peak on a short wavelength side as compared with a light-emitting material having a similar structure. Therefore, the light-emitting material with a small Stokes shift has an advantage in color purity particularly when used as a light-emitting material emitting light in a blue region.

In general, light emitted from a plurality of light-emitting materials is mixed to produce white light. A large amount of blue light is needed for white light emission with a high color temperature, and a necessary and sufficient amount of blue light consumes a large amount of power. By improving the color purity of blue light, the luminance of blue light needed for white light emission can be decreased, reducing power consumption. A light-emitting material with a small Stokes shift is likely to reduce driving voltage as compared with other materials with the same emission wavelength, which further reduces power consumption.

Comparing materials having substantially the same peak wavelength, the excitation energy of a light-emitting material with a small Stokes shift is smaller than the excitation energy of a light-emitting material with a large Stokes shift. For this reason, a light-emitting element using a light-emitting material with a small Stokes shift can emit light efficiently even when it uses a host material with a relatively small band gap, and can reduce driving voltage. In addition, a molecular structure of a material with a large band gap is limited, which narrows the range of choice for materials. The use of a light-emitting material with a small Stokes shift widens the range of choice for the host material, so that an inexpensive light-emitting element with favorable characteristics can be provided.

Thus, various effects described above can be obtained with the use of a light-emitting material with a small Stokes shift. One embodiment of the present invention is a light-emitting element using a light-emitting material with a small Stokes shift. For the above reasons, the light-emitting element has high color purity, high emission efficiency, has low driving voltage, and/or is inexpensive.

The present inventors found that in the 1,6-bis(diphenylamino)pyrene derivative in which an alkyl group is bonded to each of the two ortho positions of at least one of the two phenyl groups in each of the two diphenylamino groups, a structural change between an excited state and a ground state is smaller, i.e., Stokes shift, is smaller than in a 1,6-bis(diphenylamino)pyrene derivative without the above-mentioned structure; consequently, the 1,6-bis(diphenylamino)pyrene derivative with the above-mentioned structure has a narrower half width of an emission spectrum peak.

One embodiment of the present invention is the 1,6-bis(diphenylamino)pyrene derivative or a light-emitting element that contains the 1,6-bis(diphenylamino)pyrene derivative as a light-emitting material.

The 1,6-bis(diphenylamino)pyrene derivative emits light in a blue region. The 1,6-bis(diphenylamino)pyrene derivative with a small Stokes shift has a narrow half width of an emission spectrum and a peak wavelength on a short wavelength side; thus, light with excellent color purity is emitted. The derivative that emits light with high color purity decreases luminance needed for a blue light-emitting element which consumes a large amount of power; therefore, power consumed for white light emission can be reduced. The 1,6-bis(diphenylamino)pyrene derivative with a small Stokes shift needs a lower excitation energy than other substances that emit light with a similar color purity. For this reason, a material with a relatively narrow band gap can be used as a host material, i.e., the range of choices of host materials can be widened; accordingly, the 1,6-bis(diphenylamino)pyrene derivative with a small Stokes shift has an advantage in cost and enables fabrication of a blue light-emitting element with favorable characteristics. In addition, the use of a material relatively narrow band gap as a host material can reduce driving voltage.

The Stokes shift of the light-emitting material or the 1,6-bis(diphenylamino)pyrene derivative is greater than 0 eV and less than or equal to 0.18 eV, preferably less than or equal to 0.15 eV. The half width of an emission spectrum of the light-emitting material or the 1,6-bis(diphenylamino)pyrene derivative is less than or equal to 40 nm, ideally less than or equal to 35 nm. In addition, the light emitted from the light-emitting material or the 1,6-bis(diphenylamino)pyrene derivative is particularly effective when having a peak wavelength of 465 nm or less. Consequently, the y-coordinate of the CIE chromaticity of light emitted from a light-emitting element including the light-emitting material or the 1,6-bis(diphenylamino)pyrene derivative can be easily less than or equal to 0.15.

The present inventors found that organic compounds having a structure represented by the following general formulae have a narrow half width of an emission spectrum and emit excellent light in a blue region.

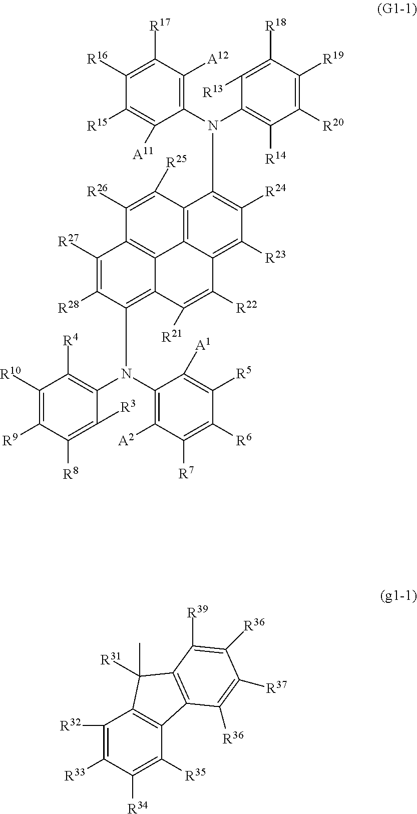

In General Formula (G1-1), A¹, A², A¹¹, and A¹² each represent an alkyl group having 1 to 6 carbon atoms; at least one of R³ and R⁴ represents hydrogen and the other represents hydrogen or an alkyl group having 1 to 6 carbon atoms; at least one of R¹³ and R¹⁴ represents hydrogen and the other represents hydrogen or an alkyl group having 1 to 6 carbon atoms; and R⁵ to R¹⁰, R¹⁵ to R²⁰, and R²¹ to R²⁸ each independently represent hydrogen, an alkyl group having 1 to 6 carbon atoms, or an aryl group having 6 to 25 carbon atoms. Note that any one of R⁵ to R¹⁰ and any one of R¹⁵ to R²⁰ are substituents represented by General Formula (g1-1). In General Formula (g1-1), R³¹ to R³⁹ each independently represent hydrogen, an alkyl group having 1 to 6 carbon atoms, or an aryl group having 6 to 25 carbon atoms.

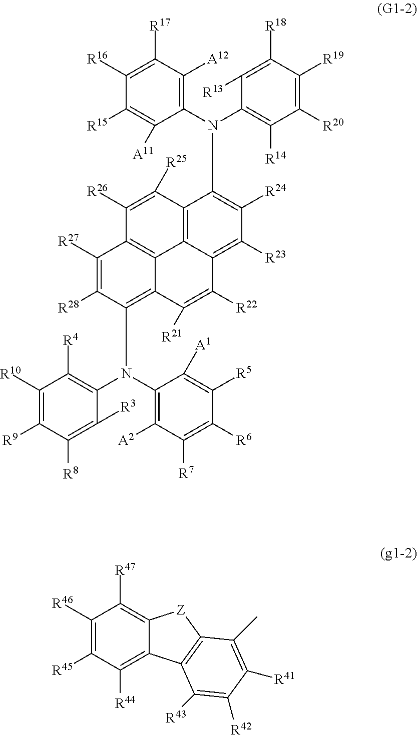

In General Formula (G1-2), A¹, A², A¹¹, and A¹² each represent an alkyl group having 1 to 6 carbon atoms; at least one of R³ and R⁴ represents hydrogen and the other represents hydrogen or an alkyl group having 1 to 6 carbon atoms; at least one of R¹³ and R¹⁴ represents hydrogen and the other represents hydrogen or an alkyl group having 1 to 6 carbon atoms; and R⁵ to R¹⁰, R¹⁵ to R²⁰, and R²¹ to R²⁸ each independently represent hydrogen, an alkyl group having 1 to 6 carbon atoms, or an aryl group having 6 to 25 carbon atoms. Note that any one of R⁵ to R¹⁰ and any one of R¹⁵ to R²⁰ are substituents represented by General Formula (g1-2). In General Formula (g1-2), R⁴¹ to R⁴⁷ each independently represent hydrogen, an alkyl group having 1 to 6 carbon atoms, or an aryl group having 6 to 25 carbon atoms; and Z represents an oxygen atom or a sulfur atom.

For easy synthesis, two acylamino groups in an organic compound represented by General Formula (G1-1) preferably have the same structure. Another embodiment of the present invention is an organic compound represented by General Formula (G2-1) or (G2-2).

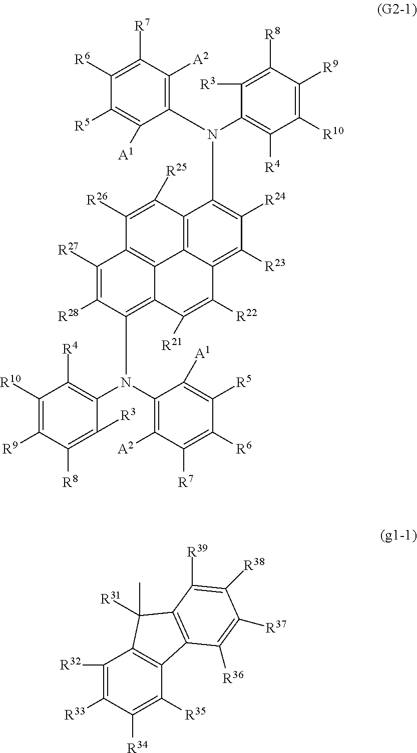

In General Formula (G2-1), A¹ and A² each represent an alkyl group having 1 to 6 carbon atoms; at least one of R³ and R⁴ represents hydrogen and the other represents hydrogen or an alkyl group having 1 to 6 carbon atoms; and R⁵ to R¹⁰ and R²¹ to R²⁸ each independently represent hydrogen, an alkyl group having 1 to 6 carbon atoms, or an aryl group having 6 to 25 carbon atoms. Note that any one of R⁵ to R¹⁰ is a substituent represented by General Formula (g1-1). In General Formula (g1-1), R³¹ to R³⁹ each independently represent hydrogen, an alkyl group having 1 to 6 carbon atoms, or an aryl group having 6 to 25 carbon atoms.

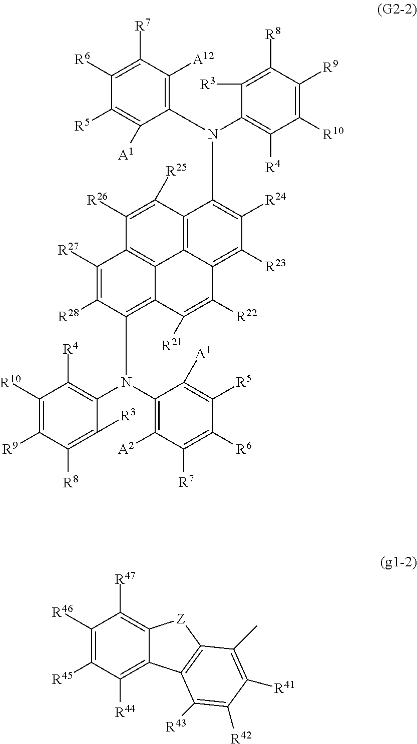

In General Formula (G2-2), A¹ and A² each represent an alkyl group having 1 to 6 carbon atoms; at least one of R³ and R⁴ represents hydrogen and the other represents hydrogen or an alkyl group having 1 to 6 carbon atoms; and R⁵ to R¹⁰ and R²¹ to R²⁸ each independently represent hydrogen, an alkyl group having 1 to 6 carbon atoms, or an aryl group having 6 to 25 carbon atoms. Note that any one of R⁵ to R¹⁰ is a substituent represented by General Formula (g1-2). In General Formula (g1-2), R⁴¹ to R⁴⁷ each independently represent hydrogen, an alkyl group having 1 to 6 carbon atoms, or an aryl group having 6 to 25 carbon atoms; and Z represents an oxygen atom or a sulfur atom.

The organic compound includes two arylamino groups one of which is bonded to the 1-position and the other is bonded to the 6-position of the pyrene skeleton. Each arylamino group has two phenyl groups. One of the two phenyl groups has two ortho positions to each of which an alkyl group is bonded. The other phenyl group included in the arylamino group has an ortho position to which hydrogen is bonded and an ortho position to which hydrogen or an alkyl group is bonded.

A phenyl group to which a substituent represented by General Formula (g1-1) or (g1-2) is bonded may be either the phenyl group in which an alkyl group is bonded to each of the two ortho positions or the phenyl group in which hydrogen is bonded to at least one of the two ortho positions. An organic compound with either structure can emit excellent blue light with a narrow half width of an emission spectrum. Note that when an organic compound has a structure in which the substituent is bonded to the phenyl group in which an alkyl group is bonded to each of the two ortho positions, a light-emitting element including the organic compound can suppress a reduction in luminance relative to driving time, and thus has high durability.

When an organic compound has a structure in which the substituent is bonded to the phenyl group in which hydrogen is bonded to at least one of the two ortho positions, hydrogen is preferably bonded to each of the ortho positions, in which case synthesis can be performed in a high yield.

The position of a phenyl group to which the substituent represented by General Formula (g1-1) or (g1-2) is bonded is preferably a meta position in either phenyl group in the arylamino group, in which case an emission peak is in a short wavelength side and deep blue light can be obtained. In addition, the organic compound with this structure is easily dissolved in a solvent.

In the substituent represented by General Formula (g1-2) which is bonded to the organic compound represented by the General Formula (G2-2), Z preferably represents an oxygen atom, in which case element characteristics and reliability can be favorable.

The substituent represented by General Formula (g1-1) or (g1-2) may have a substituted or unsubstituted benzene ring.

In organic compounds represented by General Formulae (G1-1), (G1-2), (G2-1), and (G2-2), specific examples of alkyl groups having 1 to 6 carbon atoms are a methyl group, an ethyl group, a propyl group, an isopropyl group, an n-butyl group, a sec-butyl group, an isobutyl group, a tert-butyl group, an n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, and a branched or non-branched hexyl group.

In the organic compounds represented by General Formulae (G1-1), (G1-2), (G2-1), and (G2-2), specific examples of aryl groups having 6 to 25 carbon atoms are a phenyl group, a naphthyl group, a biphenylyl group, a fluorenyl group, and an anthryl group. Note that each of these aryl groups may have a substituent. When such an aryl group has a substituent, the substituent of the aryl group is preferably an alkyl group having 1 to 4 carbon atoms or a phenyl group having 1 to 4 carbon atoms. In addition to the phenyl group, specifically, a methyl group, an ethyl group, an n-propyl group, an isopropyl group, an n-butyl group, an isobutyl group, an s-butyl group, a t-butyl group and the like are given. Among them, the methyl group and the t-butyl group are preferable. The fluorenyl group may be a 9,9-dimethylfluoren-2-yl group, a 9,9-diphenylfluoren-2-yl group, or a spiro-9,9′-bifluoren-2-yl group.

It is preferable that each of A¹, A², A¹¹, and A¹² in an organic compound represented by General Formula (G1-1) or (G1-2) and A¹ and A² in an organic compound represented by General Formula (G2-1) or (G2-2) represent a methyl group. In addition, in an organic compound represented by General Formula (G1-1), (G1-2), (G2-1), or (G2-2), when one of R³ and R⁴ represents an alkyl group, the alkyl group is preferably a methyl group.

It is preferable that each of R²¹ to R²⁸ in General Formulae (G1-1), (G1-2), (G2-1), and (G2-2), R³¹ to R³⁹ in General Formula (g1-1), and R⁴¹ to R⁴⁷ in General Formula (g1-2) represent hydrogen because a source material can be obtained easily and synthesis can be performed easily at low cost. For the same reason, R⁵ to R¹⁰ and R¹⁵ to R²⁰ other than a substituent represented by General Formula (g1-1) or (g1-2) preferably represent hydrogen.

In addition, R³¹ is preferably a phenyl group.

Some specific examples of the organic compounds of embodiments of the present invention with the above-described structure are shown below.

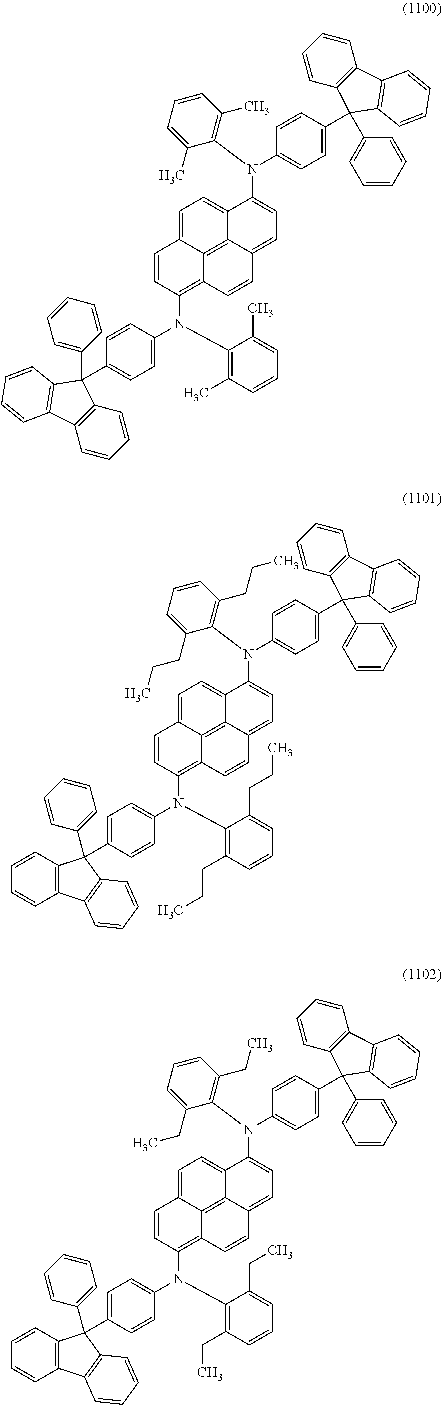 [SMILES: Cc1cccc(C)c1N(c1ccc(C2(c3ccccc3)c3ccccc3-c3ccccc32)cc1)c1ccc2ccc3c(N(c4ccc(C5(c6ccccc6)c6ccccc6-c6ccccc65)cc4)c4c(C)cccc4C)ccc4ccc1c2c43 ; CCCc1cccc(CCC)c1N(c1ccc(C2(c3ccccc3)c3ccccc3-c3ccccc32)cc1)c1ccc2ccc3c(N(c4ccc(C5(c6ccccc6)c6ccccc6-c6ccccc65)cc4)c4c(CCC)cccc4CCC)ccc4ccc1c2c43 ; CCc1cccc(CC)c1N(c1ccc(C2(c3ccccc3)c3ccccc3-c3ccccc32)cc1)c1ccc2ccc3c(N(c4ccc(C5(c6ccccc6)c6ccccc6-c6ccccc65)cc4)c4c(CC)cccc4CC)ccc4ccc1c2c43]

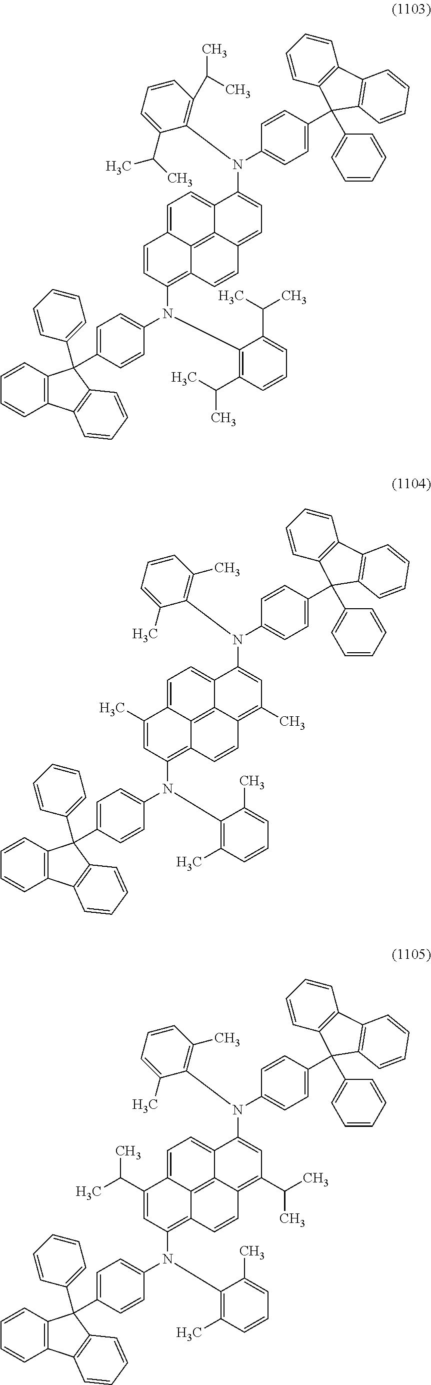 [SMILES: CC(C)c1cccc(C(C)C)c1N(c1ccc(C2(c3ccccc3)c3ccccc3-c3ccccc32)cc1)c1ccc2ccc3c(N(c4ccc(C5(c6ccccc6)c6ccccc6-c6ccccc65)cc4)c4c(C(C)C)cccc4C(C)C)ccc4ccc1c2c43 ; Cc1cccc(C)c1N(c1ccc(C2(c3ccccc3)c3ccccc3-c3ccccc32)cc1)c1cc(C)c2ccc3c(N(c4ccc(C5(c6ccccc6)c6ccccc6-c6ccccc65)cc4)c4c(C)cccc4C)cc(C)c4ccc1c2c43 ; Cc1cccc(C)c1N(c1ccc(C2(c3ccccc3)c3ccccc3-c3ccccc32)cc1)c1cc(C(C)C)c2ccc3c(N(c4ccc(C5(c6ccccc6)c6ccccc6-c6ccccc65)cc4)c4c(C)cccc4C)cc(C(C)C)c4ccc1c2c43]

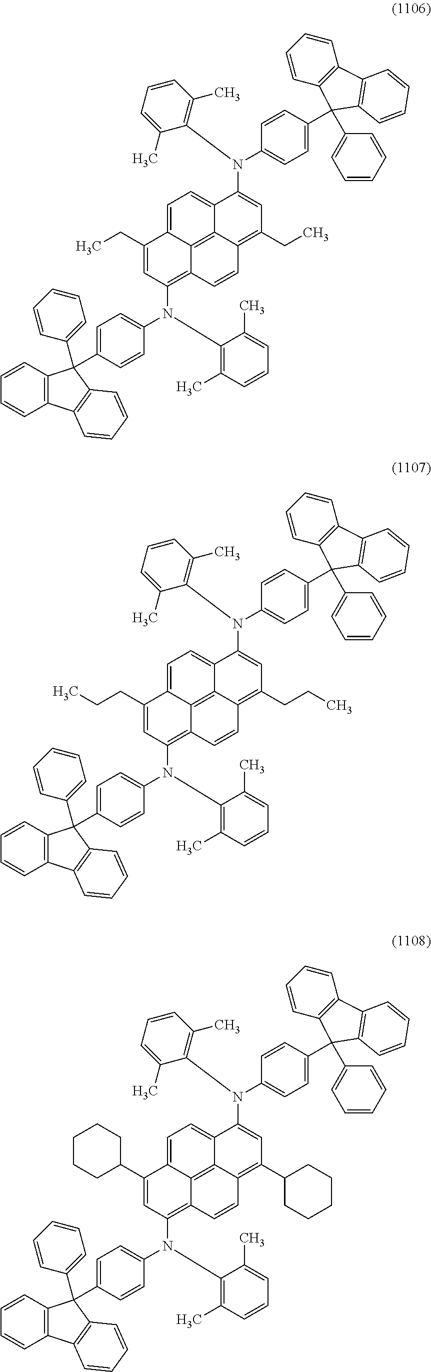 [SMILES: CCc1cc(N(c2ccc(C3(c4ccccc4)c4ccccc4-c4ccccc43)cc2)c2c(C)cccc2C)c2ccc3c(CC)cc(N(c4ccc(C5(c6ccccc6)c6ccccc6-c6ccccc65)cc4)c4c(C)cccc4C)c4ccc1c2c34 ; CCCc1cc(N(c2ccc(C3(c4ccccc4)c4ccccc4-c4ccccc43)cc2)c2c(C)cccc2C)c2ccc3c(CCC)cc(N(c4ccc(C5(c6ccccc6)c6ccccc6-c6ccccc65)cc4)c4c(C)cccc4C)c4ccc1c2c34 ; Cc1cccc(C)c1N(c1ccc(C2(c3ccccc3)c3ccccc3-c3ccccc32)cc1)c1cc(C2CCCCC2)c2ccc3c(N(c4ccc(C5(c6ccccc6)c6ccccc6-c6ccccc65)cc4)c4c(C)cccc4C)cc(C4CCCCC4)c4ccc1c2c43]

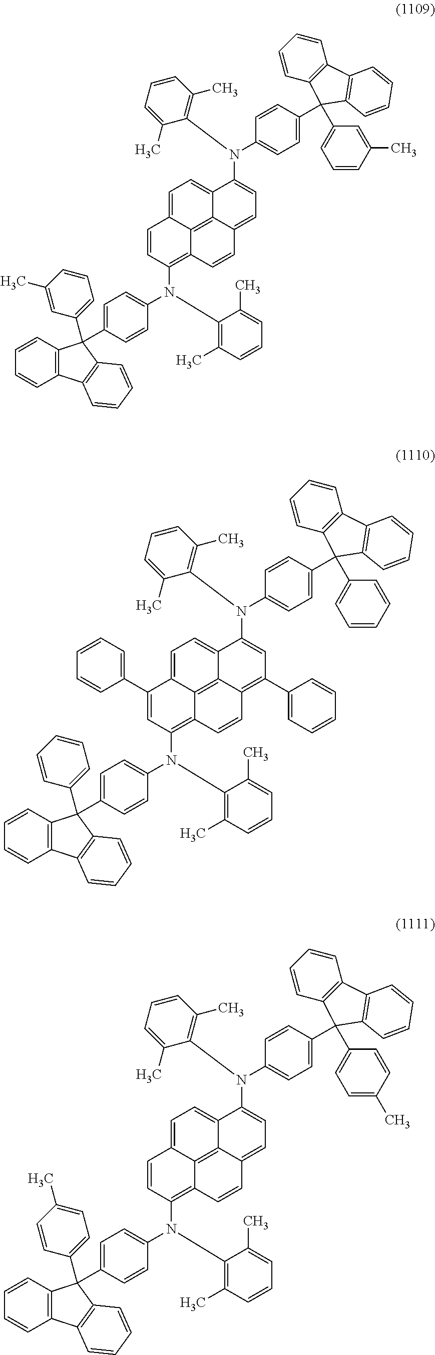 [SMILES: Cc1cccc(C2(c3ccc(N(c4c(C)cccc4C)c4ccc5ccc6c(N(c7ccc(C8(c9cccc(C)c9)c9ccccc9-c9ccccc98)cc7)c7c(C)cccc7C)ccc7ccc4c5c76)cc3)c3ccccc3-c3ccccc32)c1 ; Cc1cccc(C)c1N(c1ccc(C2(c3ccccc3)c3ccccc3-c3ccccc32)cc1)c1cc(-c2ccccc2)c2ccc3c(N(c4ccc(C5(c6ccccc6)c6ccccc6-c6ccccc65)cc4)c4c(C)cccc4C)cc(-c4ccccc4)c4ccc1c2c43 ; Cc1ccc(C2(c3ccc(N(c4c(C)cccc4C)c4ccc5ccc6c(N(c7ccc(C8(c9ccc(C)cc9)c9ccccc9-c9ccccc98)cc7)c7c(C)cccc7C)ccc7ccc4c5c76)cc3)c3ccccc3-c3ccccc32)cc1]

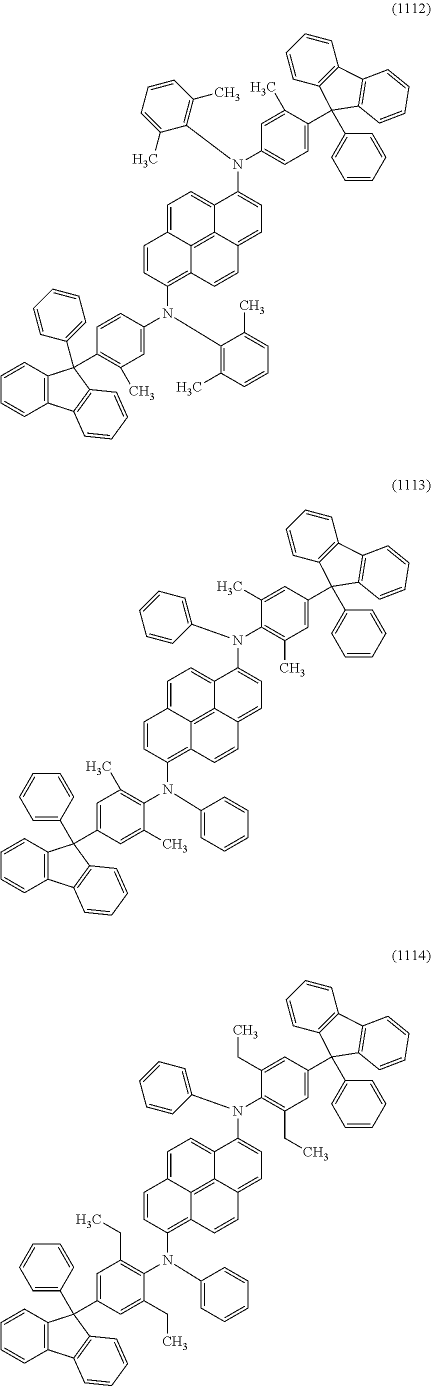 [SMILES: Cc1cc(N(c2c(C)cccc2C)c2ccc3ccc4c(N(c5ccc(C6(c7ccccc7)c7ccccc7-c7ccccc76)c(C)c5)c5c(C)cccc5C)ccc5ccc2c3c54)ccc1C1(c2ccccc2)c2ccccc2-c2ccccc21 ; Cc1cc(C2(c3ccccc3)c3ccccc3-c3ccccc32)cc(C)c1N(c1ccccc1)c1ccc2ccc3c(N(c4ccccc4)c4c(C)cc(C5(c6ccccc6)c6ccccc6-c6ccccc65)cc4C)ccc4ccc1c2c43 ; CCc1cc(C2(c3ccccc3)c3ccccc3-c3ccccc32)cc(CC)c1N(c1ccccc1)c1ccc2ccc3c(N(c4ccccc4)c4c(CC)cc(C5(c6ccccc6)c6ccccc6-c6ccccc65)cc4CC)ccc4ccc1c2c43]

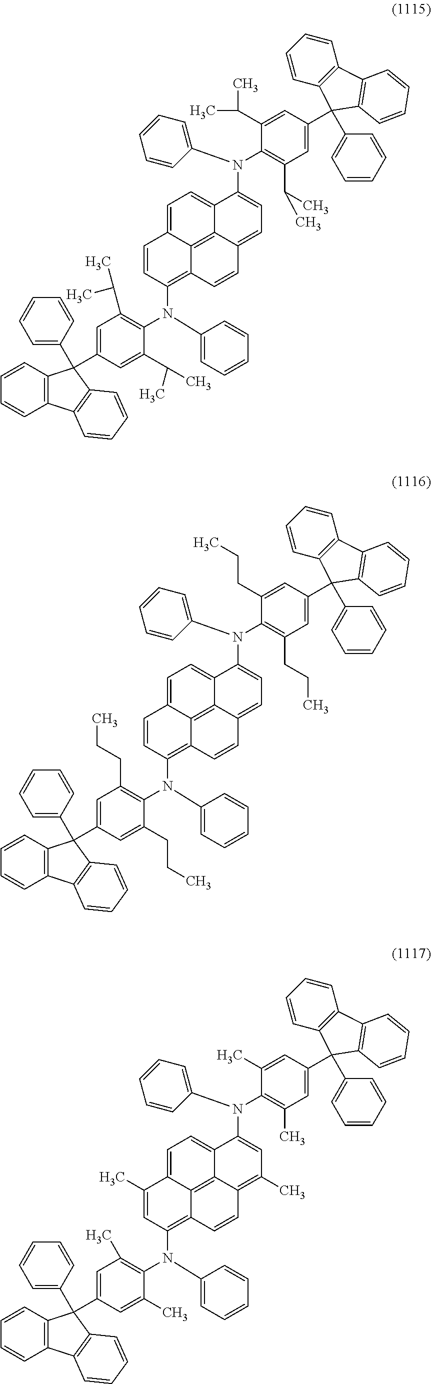 [SMILES: CC(C)c1cc(C2(c3ccccc3)c3ccccc3-c3ccccc32)cc(C(C)C)c1N(c1ccccc1)c1ccc2ccc3c(N(c4ccccc4)c4c(C(C)C)cc(C5(c6ccccc6)c6ccccc6-c6ccccc65)cc4C(C)C)ccc4ccc1c2c43 ; CCCc1cc(C2(c3ccccc3)c3ccccc3-c3ccccc32)cc(CCC)c1N(c1ccccc1)c1ccc2ccc3c(N(c4ccccc4)c4c(CCC)cc(C5(c6ccccc6)c6ccccc6-c6ccccc65)cc4CCC)ccc4ccc1c2c43 ; Cc1cc(C2(c3ccccc3)c3ccccc3-c3ccccc32)cc(C)c1N(c1ccccc1)c1cc(C)c2ccc3c(N(c4ccccc4)c4c(C)cc(C5(c6ccccc6)c6ccccc6-c6ccccc65)cc4C)cc(C)c4ccc1c2c43]

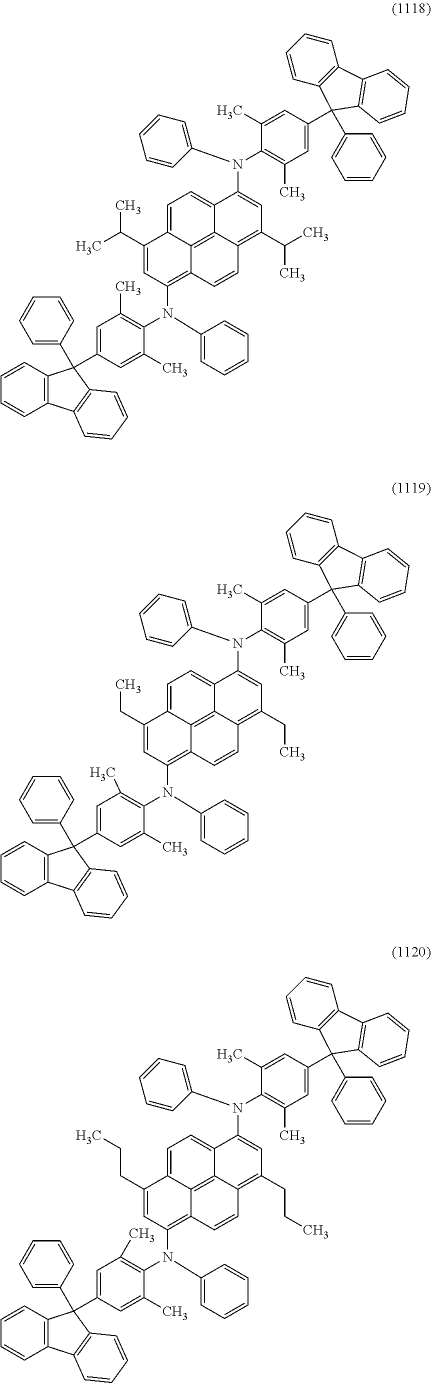 [SMILES: Cc1cc(C2(c3ccccc3)c3ccccc3-c3ccccc32)cc(C)c1N(c1ccccc1)c1cc(C(C)C)c2ccc3c(N(c4ccccc4)c4c(C)cc(C5(c6ccccc6)c6ccccc6-c6ccccc65)cc4C)cc(C(C)C)c4ccc1c2c43 ; CCc1cc(N(c2ccccc2)c2c(C)cc(C3(c4ccccc4)c4ccccc4-c4ccccc43)cc2C)c2ccc3c(CC)cc(N(c4ccccc4)c4c(C)cc(C5(c6ccccc6)c6ccccc6-c6ccccc65)cc4C)c4ccc1c2c34 ; CCCc1cc(N(c2ccccc2)c2c(C)cc(C3(c4ccccc4)c4ccccc4-c4ccccc43)cc2C)c2ccc3c(CCC)cc(N(c4ccccc4)c4c(C)cc(C5(c6ccccc6)c6ccccc6-c6ccccc65)cc4C)c4ccc1c2c34]

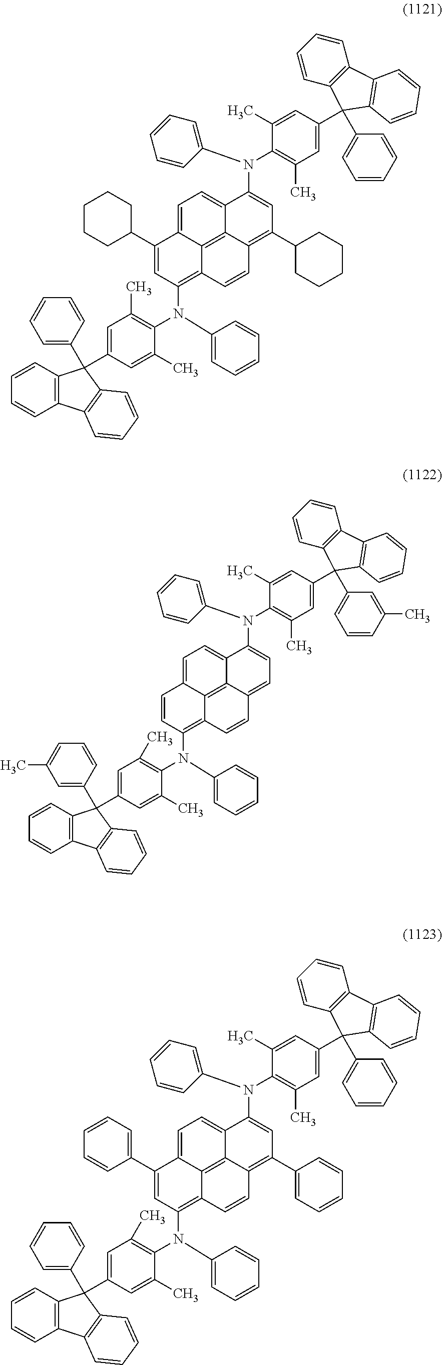

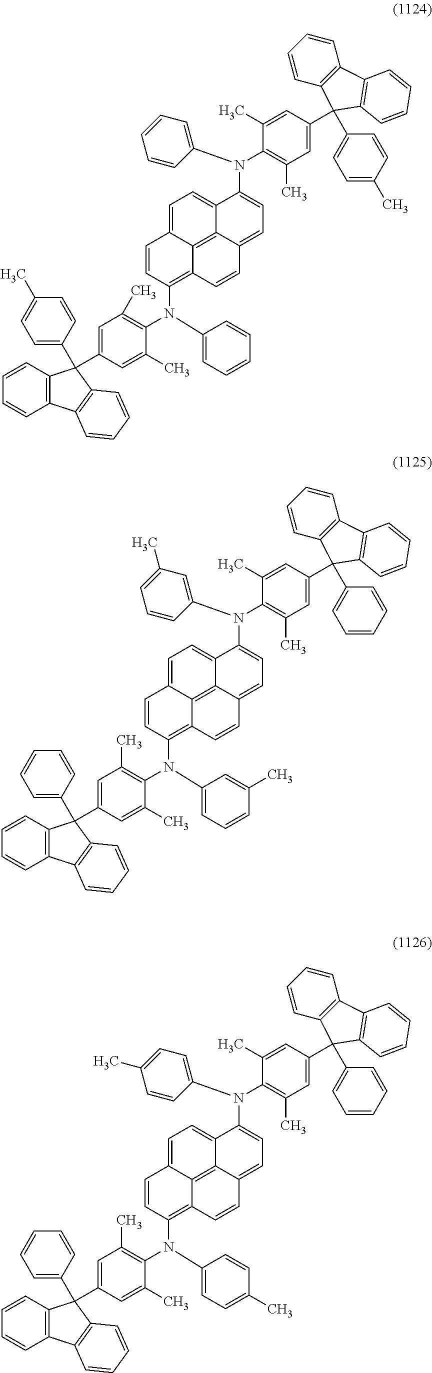 [SMILES: Cc1ccc(C2(c3cc(C)c(N(c4ccccc4)c4ccc5ccc6c(N(c7ccccc7)c7c(C)cc(C8(c9ccc(C)cc9)c9ccccc9-c9ccccc98)cc7C)ccc7ccc4c5c76)c(C)c3)c3ccccc3-c3ccccc32)cc1 ; Cc1cccc(N(c2c(C)cc(C3(c4ccccc4)c4ccccc4-c4ccccc43)cc2C)c2ccc3ccc4c(N(c5cccc(C)c5)c5c(C)cc(C6(c7ccccc7)c7ccccc7-c7ccccc76)cc5C)ccc5ccc2c3c54)c1 ; Cc1ccc(N(c2c(C)cc(C3(c4ccccc4)c4ccccc4-c4ccccc43)cc2C)c2ccc3ccc4c(N(c5ccc(C)cc5)c5c(C)cc(C6(c7ccccc7)c7ccccc7-c7ccccc76)cc5C)ccc5ccc2c3c54)cc1]

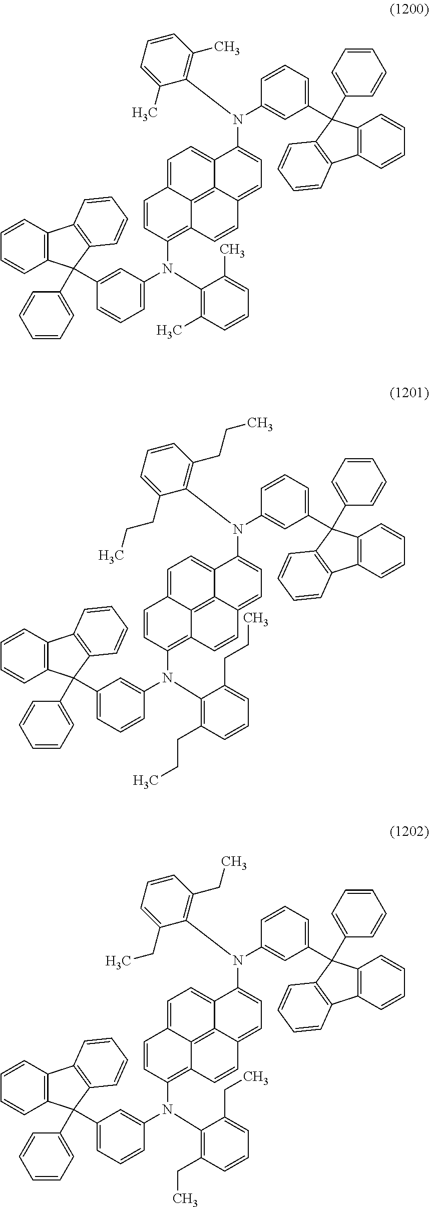 [SMILES: Cc1cccc(C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C)cccc4C)ccc4ccc1c2c43 ; CCCc1cccc(CCC)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(CCC)cccc4CCC)ccc4ccc1c2c43 ; CCc1cccc(CC)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(CC)cccc4CC)ccc4ccc1c2c43]

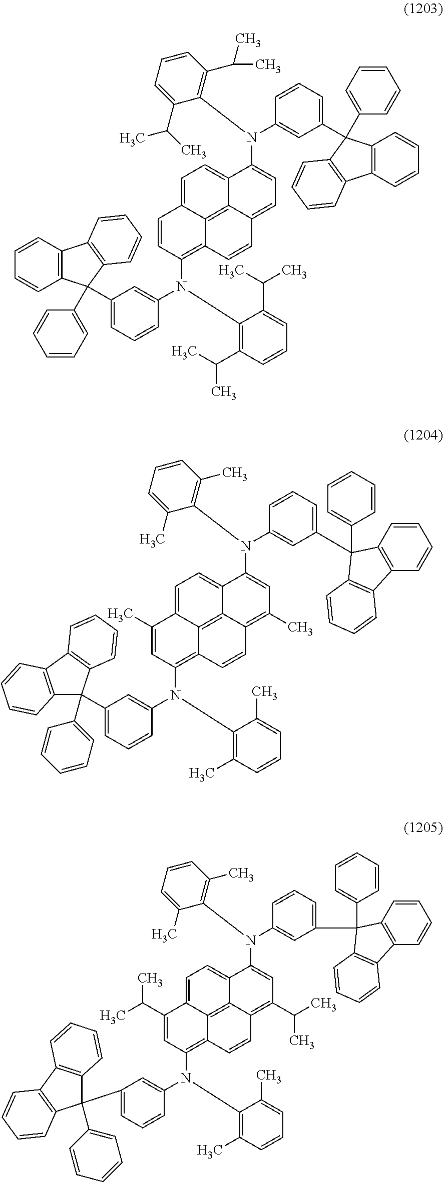 [SMILES: CC(C)c1cccc(C(C)C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C(C)C)cccc4C(C)C)ccc4ccc1c2c43 ; Cc1cccc(C)c1N(c1cccc(C2(c3ccccc3)c3ccccc3-c3ccccc32)c1)c1cc(C)c2ccc3c(N(c4cccc(C5(c6ccccc6)c6ccccc6-c6ccccc65)c4)c4c(C)cccc4C)cc(C)c4ccc1c2c43 ; Cc1cccc(C)c1N(c1cccc(C2(c3ccccc3)c3ccccc3-c3ccccc32)c1)c1cc(C(C)C)c2ccc3c(N(c4cccc(C5(c6ccccc6)c6ccccc6-c6ccccc65)c4)c4c(C)cccc4C)cc(C(C)C)c4ccc1c2c43]

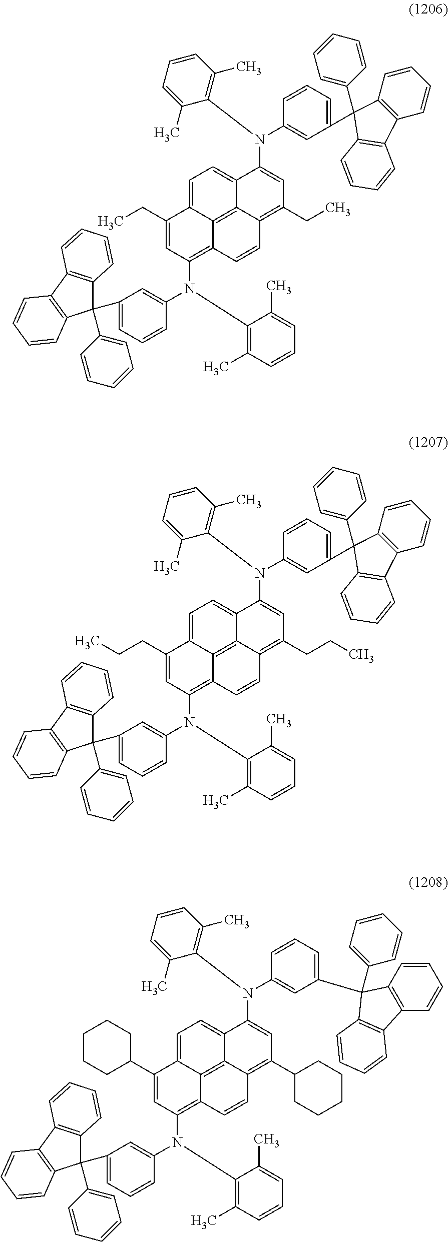 [SMILES: CCc1cc(N(c2cccc(C3(c4ccccc4)c4ccccc4-c4ccccc43)c2)c2c(C)cccc2C)c2ccc3c(CC)cc(N(c4cccc(C5(c6ccccc6)c6ccccc6-c6ccccc65)c4)c4c(C)cccc4C)c4ccc1c2c34 ; CCCc1cc(N(c2cccc(C3(c4ccccc4)c4ccccc4-c4ccccc43)c2)c2c(C)cccc2C)c2ccc3c(CCC)cc(N(c4cccc(C5(c6ccccc6)c6ccccc6-c6ccccc65)c4)c4c(C)cccc4C)c4ccc1c2c34 ; Cc1cccc(C)c1N(c1cccc(C2(c3ccccc3)c3ccccc3-c3ccccc32)c1)c1cc(C2CCCCC2)c2ccc3c(N(c4cccc(C5(c6ccccc6)c6ccccc6-c6ccccc65)c4)c4c(C)cccc4C)cc(C4CCCCC4)c4ccc1c2c43]

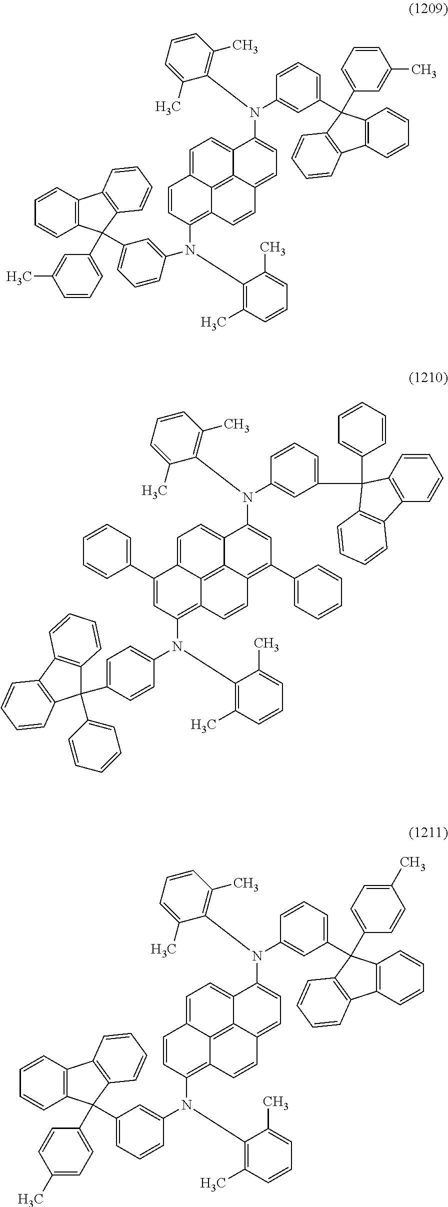

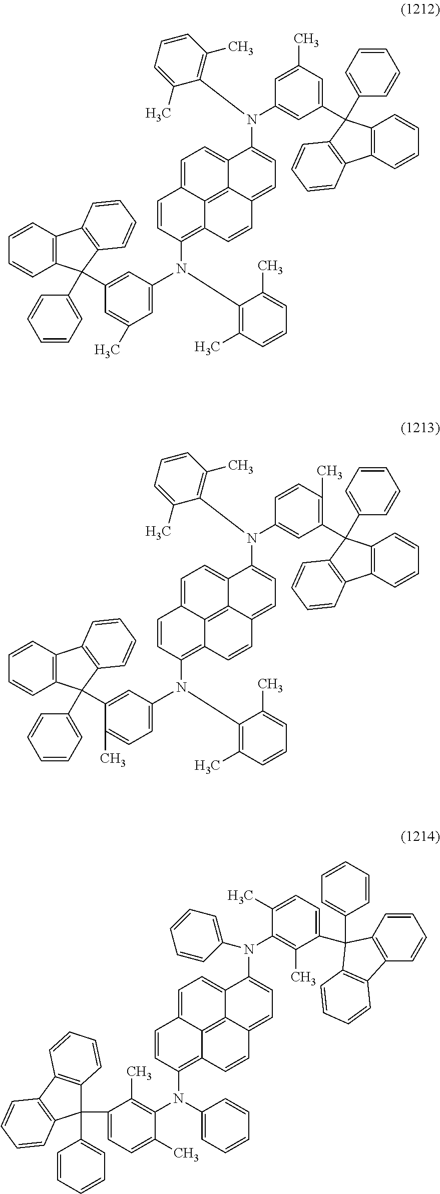 [SMILES: Cc1cc(N(c2c(C)cccc2C)c2ccc3ccc4c(N(c5cc(C)cc(C6(c7ccccc7)c7ccccc7-c7ccccc76)c5)c5c(C)cccc5C)ccc5ccc2c3c54)cc(C2(c3ccccc3)c3ccccc3-c3ccccc32)c1 ; Cc1ccc(N(c2c(C)cccc2C)c2ccc3ccc4c(N(c5ccc(C)c(C6(c7ccccc7)c7ccccc7-c7ccccc76)c5)c5c(C)cccc5C)ccc5ccc2c3c54)cc1C1(c2ccccc2)c2ccccc2-c2ccccc21 ; Cc1ccc(C2(c3ccccc3)c3ccccc3-c3ccccc32)c(C)c1N(c1ccccc1)c1ccc2ccc3c(N(c4ccccc4)c4c(C)ccc(C5(c6ccccc6)c6ccccc6-c6ccccc65)c4C)ccc4ccc1c2c43]

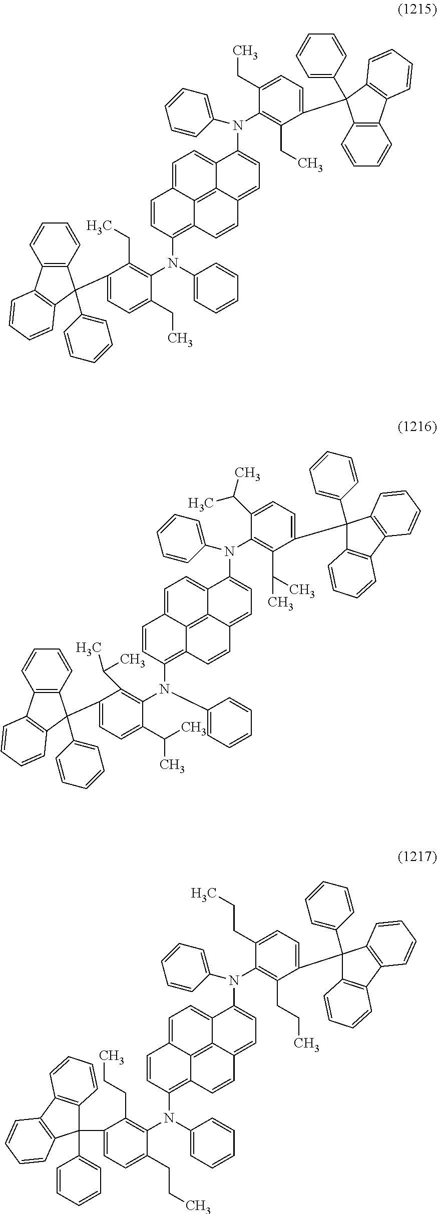 [SMILES: CCc1ccc(C2(c3ccccc3)c3ccccc3-c3ccccc32)c(CC)c1N(c1ccccc1)c1ccc2ccc3c(N(c4ccccc4)c4c(CC)ccc(C5(c6ccccc6)c6ccccc6-c6ccccc65)c4CC)ccc4ccc1c2c43 ; CC(C)c1ccc(C2(c3ccccc3)c3ccccc3-c3ccccc32)c(C(C)C)c1N(c1ccccc1)c1ccc2ccc3c(N(c4ccccc4)c4c(C(C)C)ccc(C5(c6ccccc6)c6ccccc6-c6ccccc65)c4C(C)C)ccc4ccc1c2c43 ; CCCc1ccc(C2(c3ccccc3)c3ccccc3-c3ccccc32)c(CCC)c1N(c1ccccc1)c1ccc2ccc3c(N(c4ccccc4)c4c(CCC)ccc(C5(c6ccccc6)c6ccccc6-c6ccccc65)c4CCC)ccc4ccc1c2c43]

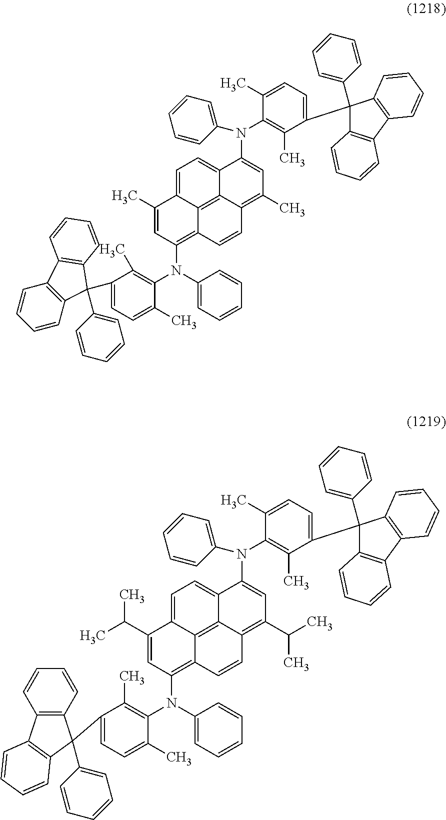 [SMILES: Cc1ccc(C2(c3ccccc3)c3ccccc3-c3ccccc32)c(C)c1N(c1ccccc1)c1cc(C)c2ccc3c(N(c4ccccc4)c4c(C)ccc(C5(c6ccccc6)c6ccccc6-c6ccccc65)c4C)cc(C)c4ccc1c2c43 ; Cc1ccc(C2(c3ccccc3)c3ccccc3-c3ccccc32)c(C)c1N(c1ccccc1)c1cc(C(C)C)c2ccc3c(N(c4ccccc4)c4c(C)ccc(C5(c6ccccc6)c6ccccc6-c6ccccc65)c4C)cc(C(C)C)c4ccc1c2c43]

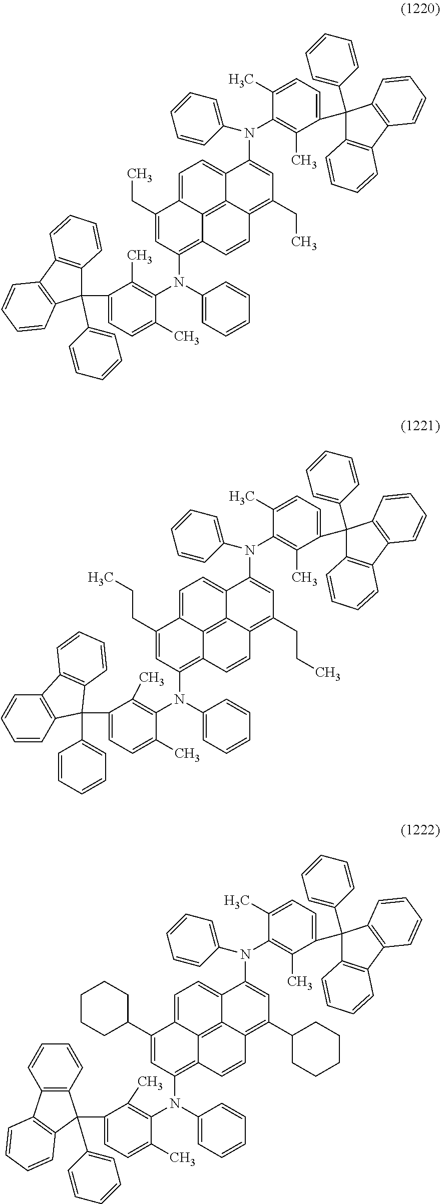 [SMILES: CCc1cc(N(c2ccccc2)c2c(C)ccc(C3(c4ccccc4)c4ccccc4-c4ccccc43)c2C)c2ccc3c(CC)cc(N(c4ccccc4)c4c(C)ccc(C5(c6ccccc6)c6ccccc6-c6ccccc65)c4C)c4ccc1c2c34 ; CCCc1cc(N(c2ccccc2)c2c(C)ccc(C3(c4ccccc4)c4ccccc4-c4ccccc43)c2C)c2ccc3c(CCC)cc(N(c4ccccc4)c4c(C)ccc(C5(c6ccccc6)c6ccccc6-c6ccccc65)c4C)c4ccc1c2c34 ; Cc1ccc(C2(c3ccccc3)c3ccccc3-c3ccccc32)c(C)c1N(c1ccccc1)c1cc(C2CCCCC2)c2ccc3c(N(c4ccccc4)c4c(C)ccc(C5(c6ccccc6)c6ccccc6-c6ccccc65)c4C)cc(C4CCCCC4)c4ccc1c2c43]

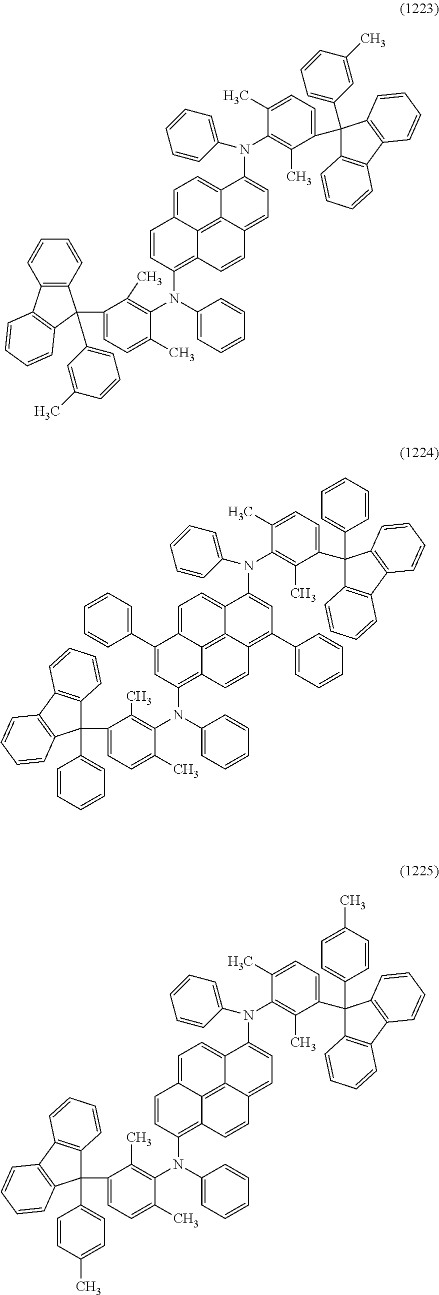 [SMILES: Cc1cccc(C2(c3ccc(C)c(N(c4ccccc4)c4ccc5ccc6c(N(c7ccccc7)c7c(C)ccc(C8(c9cccc(C)c9)c9ccccc9-c9ccccc98)c7C)ccc7ccc4c5c76)c3C)c3ccccc3-c3ccccc32)c1 ; Cc1ccc(C2(c3ccccc3)c3ccccc3-c3ccccc32)c(C)c1N(c1ccccc1)c1cc(-c2ccccc2)c2ccc3c(N(c4ccccc4)c4c(C)ccc(C5(c6ccccc6)c6ccccc6-c6ccccc65)c4C)cc(-c4ccccc4)c4ccc1c2c43 ; Cc1ccc(C2(c3ccc(C)c(N(c4ccccc4)c4ccc5ccc6c(N(c7ccccc7)c7c(C)ccc(C8(c9ccc(C)cc9)c9ccccc9-c9ccccc98)c7C)ccc7ccc4c5c76)c3C)c3ccccc3-c3ccccc32)cc1]

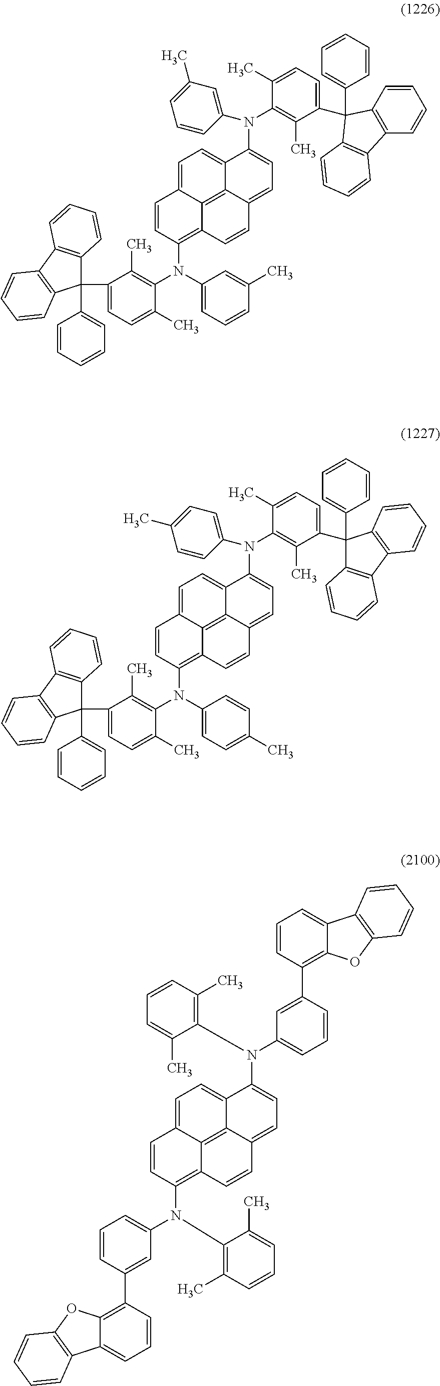 [SMILES: Cc1cccc(N(c2c(C)ccc(C3(c4ccccc4)c4ccccc4-c4ccccc43)c2C)c2ccc3ccc4c(N(c5cccc(C)c5)c5c(C)ccc(C6(c7ccccc7)c7ccccc7-c7ccccc76)c5C)ccc5ccc2c3c54)c1 ; Cc1ccc(N(c2c(C)ccc(C3(c4ccccc4)c4ccccc4-c4ccccc43)c2C)c2ccc3ccc4c(N(c5ccc(C)cc5)c5c(C)ccc(C6(c7ccccc7)c7ccccc7-c7ccccc76)c5C)ccc5ccc2c3c54)cc1 ; Cc1cccc(C)c1N(c1cccc(-c2cccc3c2oc2ccccc23)c1)c1ccc2ccc3c(N(c4cccc(-c5cccc6c5oc5ccccc56)c4)c4c(C)cccc4C)ccc4ccc1c2c43]

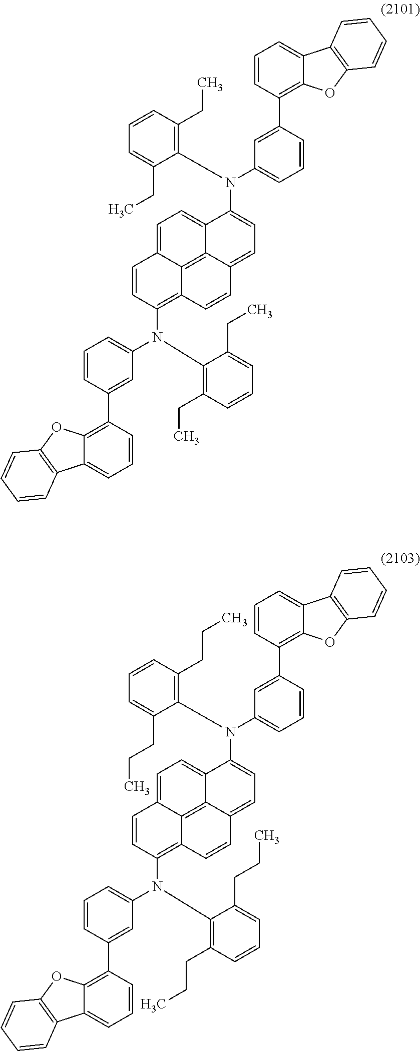 [SMILES: CCc1cccc(CC)c1N(c1cccc(-c2cccc3c2oc2ccccc23)c1)c1ccc2ccc3c(N(c4cccc(-c5cccc6c5oc5ccccc56)c4)c4c(CC)cccc4CC)ccc4ccc1c2c43 ; CCCc1cccc(CCC)c1N(c1cccc(-c2cccc3c2oc2ccccc23)c1)c1ccc2ccc3c(N(c4cccc(-c5cccc6c5oc5ccccc56)c4)c4c(CCC)cccc4CCC)ccc4ccc1c2c43]

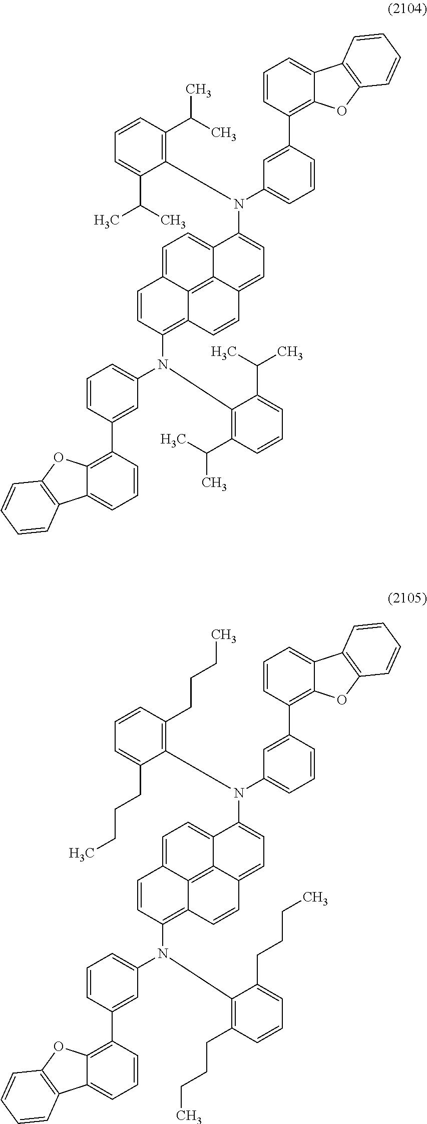 [SMILES: CC(C)c1cccc(C(C)C)c1N(c1cccc(-c2cccc3c2oc2ccccc23)c1)c1ccc2ccc3c(N(c4cccc(-c5cccc6c5oc5ccccc56)c4)c4c(C(C)C)cccc4C(C)C)ccc4ccc1c2c43 ; CCCCc1cccc(CCCC)c1N(c1cccc(-c2cccc3c2oc2ccccc23)c1)c1ccc2ccc3c(N(c4cccc(-c5cccc6c5oc5ccccc56)c4)c4c(CCCC)cccc4CCCC)ccc4ccc1c2c43]

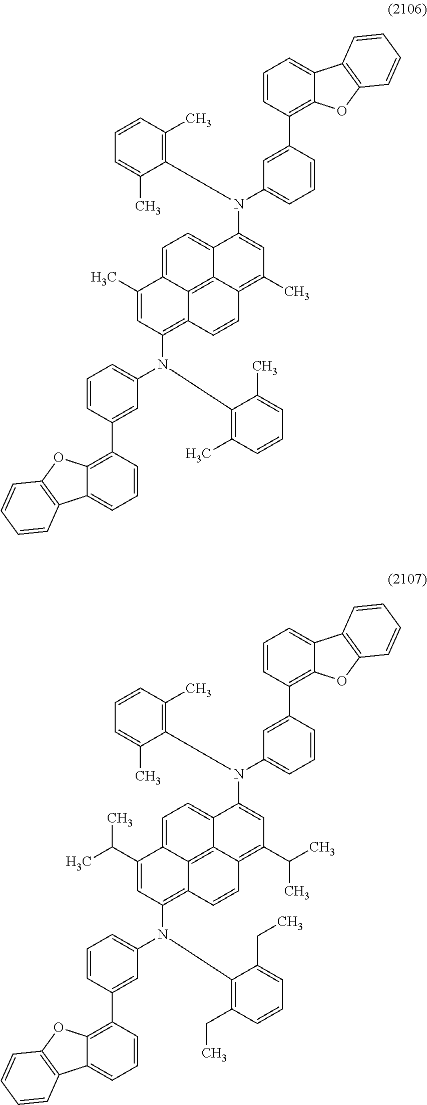 [SMILES: Cc1cccc(C)c1N(c1cccc(-c2cccc3c2oc2ccccc23)c1)c1cc(C)c2ccc3c(N(c4cccc(-c5cccc6c5oc5ccccc56)c4)c4c(C)cccc4C)cc(C)c4ccc1c2c43 ; CCc1cccc(CC)c1N(c1cccc(-c2cccc3c2oc2ccccc23)c1)c1cc(C(C)C)c2ccc3c(N(c4cccc(-c5cccc6c5oc5ccccc56)c4)c4c(C)cccc4C)cc(C(C)C)c4ccc1c2c43]

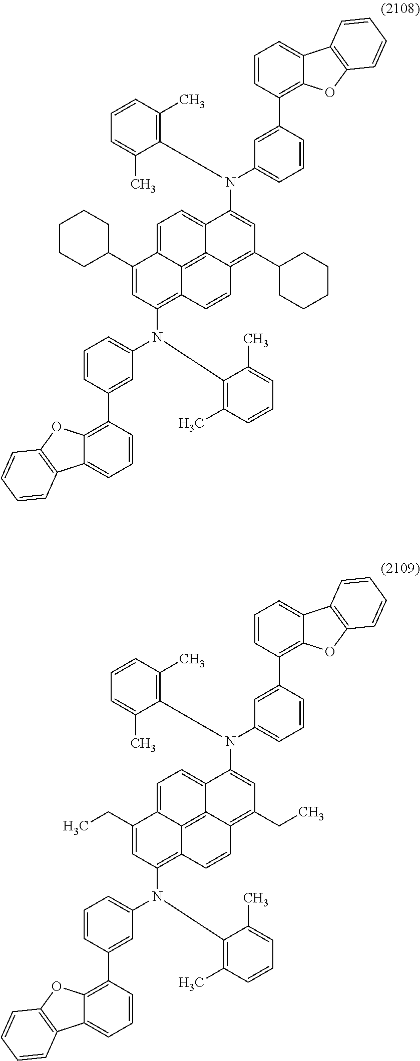 [SMILES: Cc1cccc(C)c1N(c1cccc(-c2cccc3c2oc2ccccc23)c1)c1cc(C2CCCCC2)c2ccc3c(N(c4cccc(-c5cccc6c5oc5ccccc56)c4)c4c(C)cccc4C)cc(C4CCCCC4)c4ccc1c2c43 ; CCc1cc(N(c2cccc(-c3cccc4c3oc3ccccc34)c2)c2c(C)cccc2C)c2ccc3c(CC)cc(N(c4cccc(-c5cccc6c5oc5ccccc56)c4)c4c(C)cccc4C)c4ccc1c2c34]

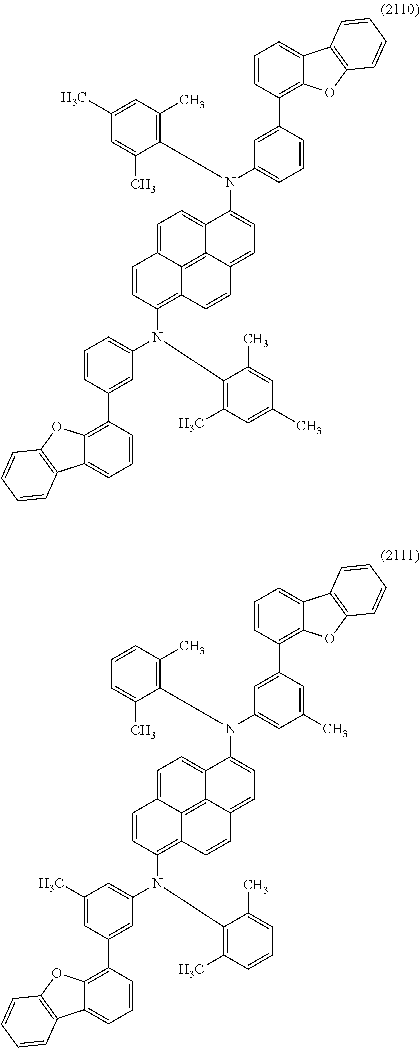 [SMILES: Cc1cc(C)c(N(c2cccc(-c3cccc4c3oc3ccccc34)c2)c2ccc3ccc4c(N(c5cccc(-c6cccc7c6oc6ccccc67)c5)c5c(C)cc(C)cc5C)ccc5ccc2c3c54)c(C)c1 ; Cc1cc(-c2cccc3c2oc2ccccc23)cc(N(c2c(C)cccc2C)c2ccc3ccc4c(N(c5cc(C)cc(-c6cccc7c6oc6ccccc67)c5)c5c(C)cccc5C)ccc5ccc2c3c54)c1]

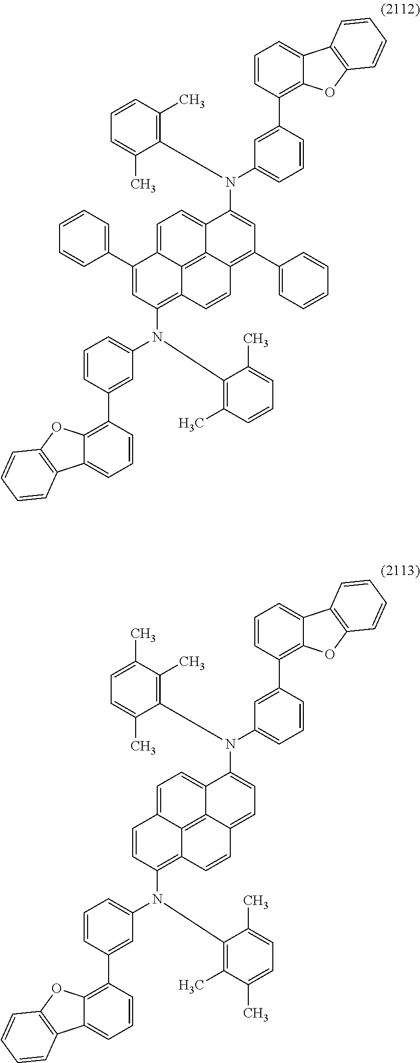 [SMILES: Cc1cccc(C)c1N(c1cccc(-c2cccc3c2oc2ccccc23)c1)c1cc(-c2ccccc2)c2ccc3c(N(c4cccc(-c5cccc6c5oc5ccccc56)c4)c4c(C)cccc4C)cc(-c4ccccc4)c4ccc1c2c43 ; Cc1ccc(C)c(N(c2cccc(-c3cccc4c3oc3ccccc34)c2)c2ccc3ccc4c(N(c5cccc(-c6cccc7c6oc6ccccc67)c5)c5c(C)ccc(C)c5C)ccc5ccc2c3c54)c1C]

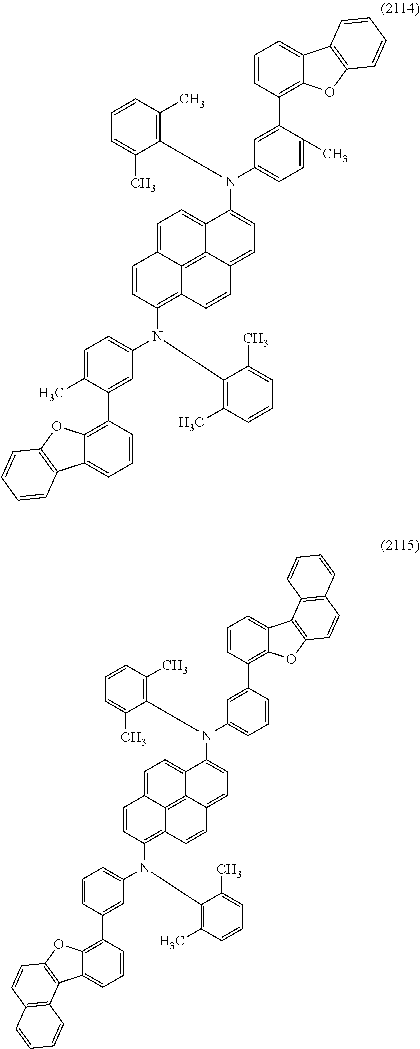 [SMILES: Cc1ccc(N(c2c(C)cccc2C)c2ccc3ccc4c(N(c5ccc(C)c(-c6cccc7c6oc6ccccc67)c5)c5c(C)cccc5C)ccc5ccc2c3c54)cc1-c1cccc2c1oc1ccccc12 ; Cc1cccc(C)c1N(c1cccc(-c2cccc3c2oc2ccc4ccccc4c23)c1)c1ccc2ccc3c(N(c4cccc(-c5cccc6c5oc5ccc7ccccc7c56)c4)c4c(C)cccc4C)ccc4ccc1c2c43]

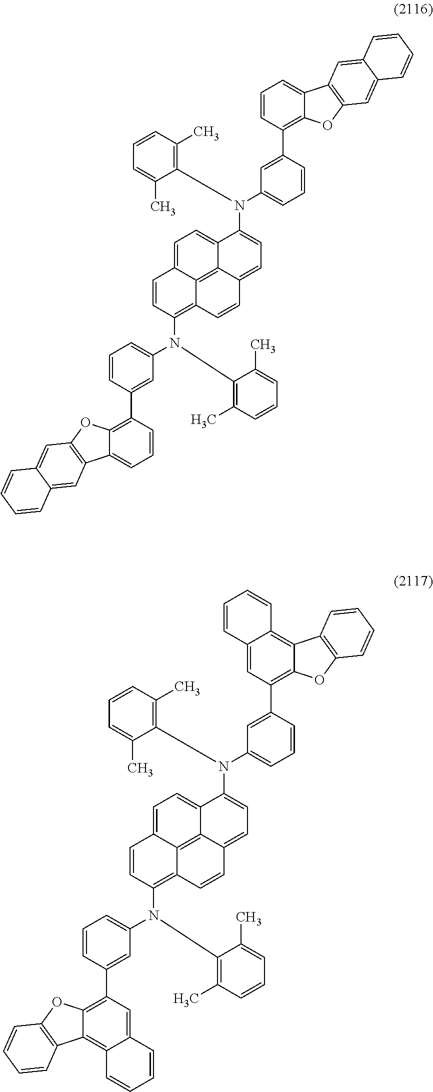 [SMILES: Cc1cccc(C)c1N(c1cccc(-c2cccc3c2oc2cc4ccccc4cc23)c1)c1ccc2ccc3c(N(c4cccc(-c5cccc6c5oc5cc7ccccc7cc56)c4)c4c(C)cccc4C)ccc4ccc1c2c43 ; Cc1cccc(C)c1N(c1cccc(-c2cc3ccccc3c3c2oc2ccccc23)c1)c1ccc2ccc3c(N(c4cccc(-c5cc6ccccc6c6c5oc5ccccc56)c4)c4c(C)cccc4C)ccc4ccc1c2c43]

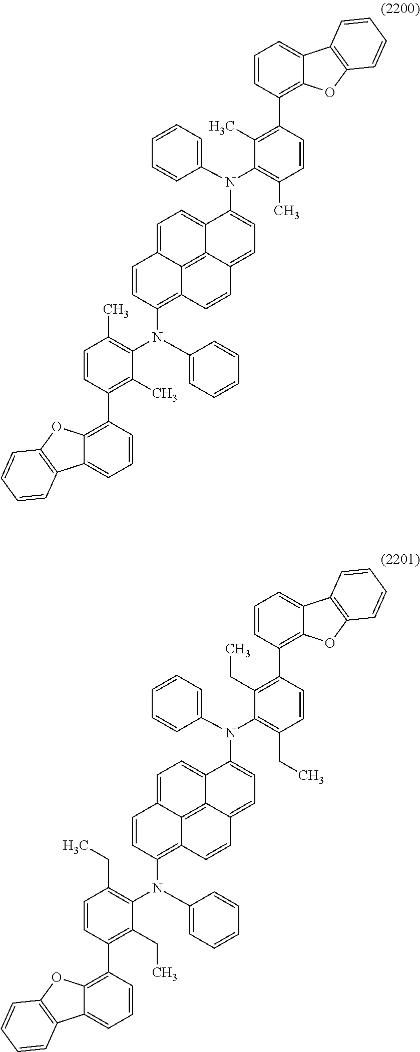 [SMILES: Cc1ccc(-c2cccc3c2oc2ccccc23)c(C)c1N(c1ccccc1)c1ccc2ccc3c(N(c4ccccc4)c4c(C)ccc(-c5cccc6c5oc5ccccc56)c4C)ccc4ccc1c2c43 ; CCc1ccc(-c2cccc3c2oc2ccccc23)c(CC)c1N(c1ccccc1)c1ccc2ccc3c(N(c4ccccc4)c4c(CC)ccc(-c5cccc6c5oc5ccccc56)c4CC)ccc4ccc1c2c43]

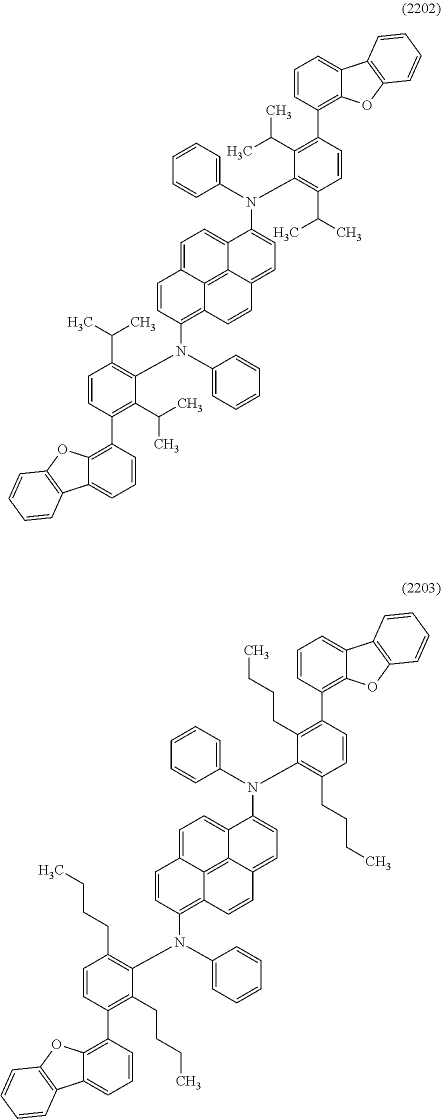 [SMILES: CC(C)c1ccc(-c2cccc3c2oc2ccccc23)c(C(C)C)c1N(c1ccccc1)c1ccc2ccc3c(N(c4ccccc4)c4c(C(C)C)ccc(-c5cccc6c5oc5ccccc56)c4C(C)C)ccc4ccc1c2c43 ; CCCCc1ccc(-c2cccc3c2oc2ccccc23)c(CCCC)c1N(c1ccccc1)c1ccc2ccc3c(N(c4ccccc4)c4c(CCCC)ccc(-c5cccc6c5oc5ccccc56)c4CCCC)ccc4ccc1c2c43]

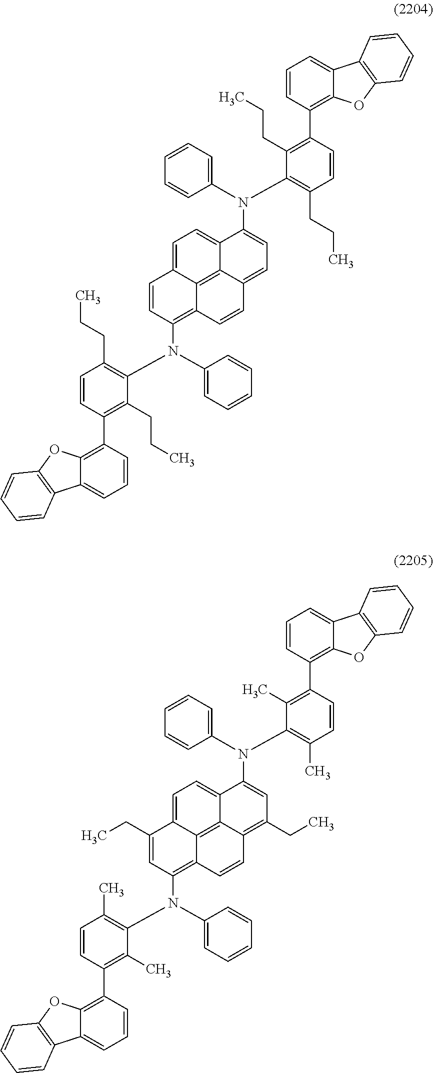 [SMILES: CCCc1ccc(-c2cccc3c2oc2ccccc23)c(CCC)c1N(c1ccccc1)c1ccc2ccc3c(N(c4ccccc4)c4c(CCC)ccc(-c5cccc6c5oc5ccccc56)c4CCC)ccc4ccc1c2c43 ; CCc1cc(N(c2ccccc2)c2c(C)ccc(-c3cccc4c3oc3ccccc34)c2C)c2ccc3c(CC)cc(N(c4ccccc4)c4c(C)ccc(-c5cccc6c5oc5ccccc56)c4C)c4ccc1c2c34]

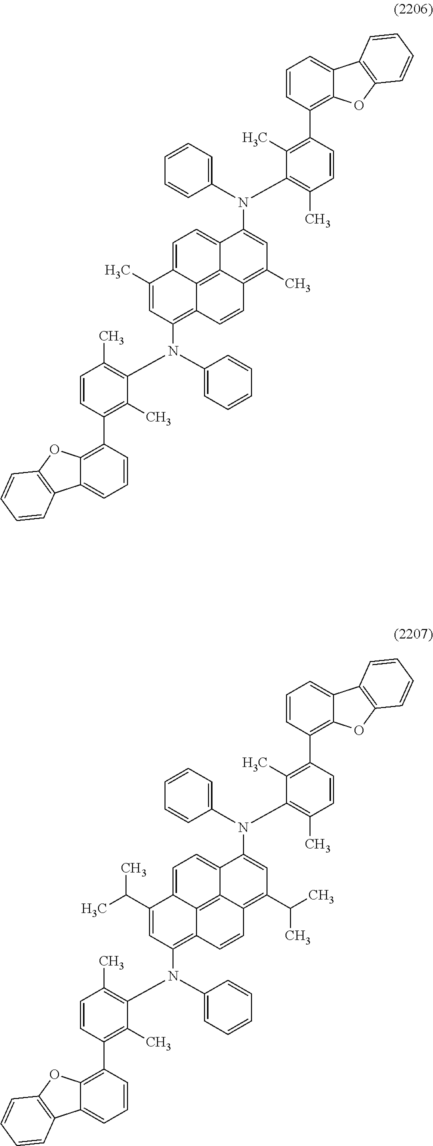 [SMILES: Cc1ccc(-c2cccc3c2oc2ccccc23)c(C)c1N(c1ccccc1)c1cc(C)c2ccc3c(N(c4ccccc4)c4c(C)ccc(-c5cccc6c5oc5ccccc56)c4C)cc(C)c4ccc1c2c43 ; Cc1ccc(-c2cccc3c2oc2ccccc23)c(C)c1N(c1ccccc1)c1cc(C(C)C)c2ccc3c(N(c4ccccc4)c4c(C)ccc(-c5cccc6c5oc5ccccc56)c4C)cc(C(C)C)c4ccc1c2c43]

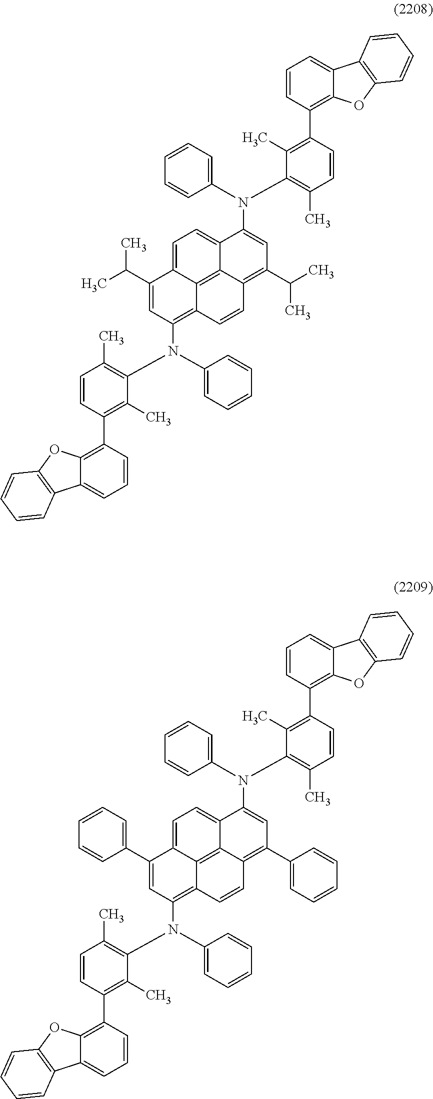 [SMILES: Cc1ccc(-c2cccc3c2oc2ccccc23)c(C)c1N(c1ccccc1)c1cc(C(C)C)c2ccc3c(N(c4ccccc4)c4c(C)ccc(-c5cccc6c5oc5ccccc56)c4C)cc(C(C)C)c4ccc1c2c43 ; Cc1ccc(-c2cccc3c2oc2ccccc23)c(C)c1N(c1ccccc1)c1cc(-c2ccccc2)c2ccc3c(N(c4ccccc4)c4c(C)ccc(-c5cccc6c5oc5ccccc56)c4C)cc(-c4ccccc4)c4ccc1c2c43]

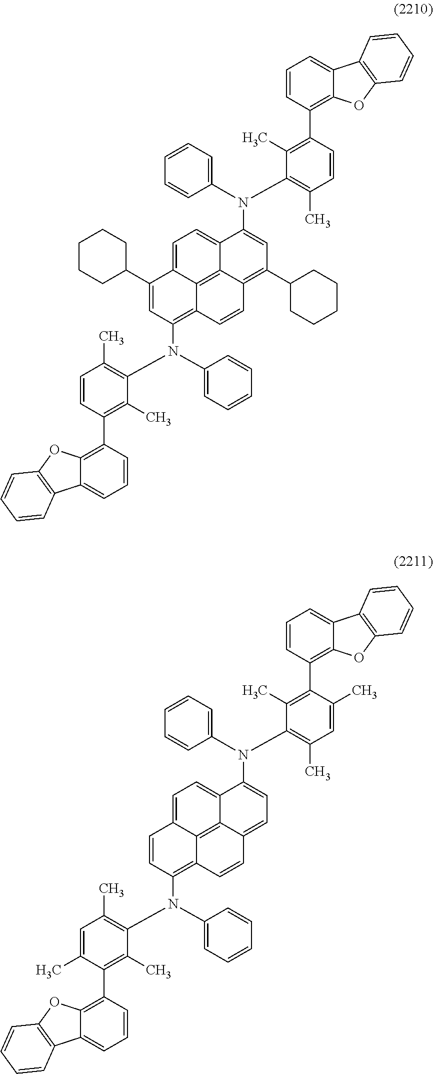 [SMILES: Cc1ccc(-c2cccc3c2oc2ccccc23)c(C)c1N(c1ccccc1)c1cc(C2CCCCC2)c2ccc3c(N(c4ccccc4)c4c(C)ccc(-c5cccc6c5oc5ccccc56)c4C)cc(C4CCCCC4)c4ccc1c2c43 ; Cc1cc(C)c(N(c2ccccc2)c2ccc3ccc4c(N(c5ccccc5)c5c(C)cc(C)c(-c6cccc7c6oc6ccccc67)c5C)ccc5ccc2c3c54)c(C)c1-c1cccc2c1oc1ccccc12]

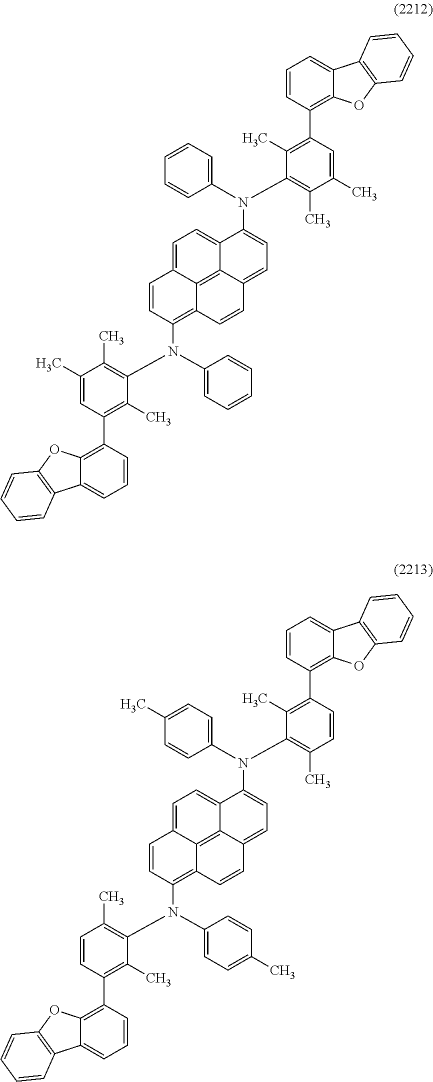 [SMILES: Cc1cc(-c2cccc3c2oc2ccccc23)c(C)c(N(c2ccccc2)c2ccc3ccc4c(N(c5ccccc5)c5c(C)c(C)cc(-c6cccc7c6oc6ccccc67)c5C)ccc5ccc2c3c54)c1C ; Cc1ccc(N(c2c(C)ccc(-c3cccc4c3oc3ccccc34)c2C)c2ccc3ccc4c(N(c5ccc(C)cc5)c5c(C)ccc(-c6cccc7c6oc6ccccc67)c5C)ccc5ccc2c3c54)cc1]

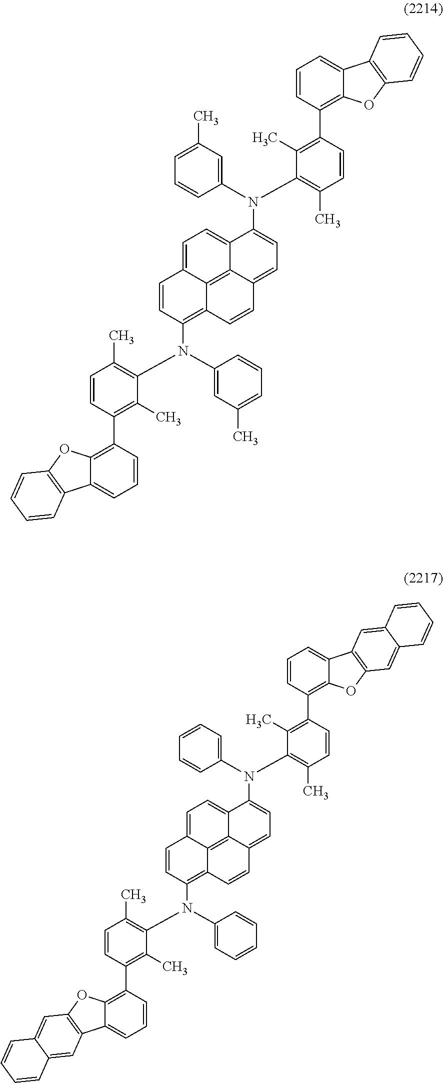 [SMILES: Cc1cccc(N(c2c(C)ccc(-c3cccc4c3oc3ccccc34)c2C)c2ccc3ccc4c(N(c5cccc(C)c5)c5c(C)ccc(-c6cccc7c6oc6ccccc67)c5C)ccc5ccc2c3c54)c1 ; Cc1ccc(-c2cccc3c2oc2cc4ccccc4cc23)c(C)c1N(c1ccccc1)c1ccc2ccc3c(N(c4ccccc4)c4c(C)ccc(-c5cccc6c5oc5cc7ccccc7cc56)c4C)ccc4ccc1c2c43]

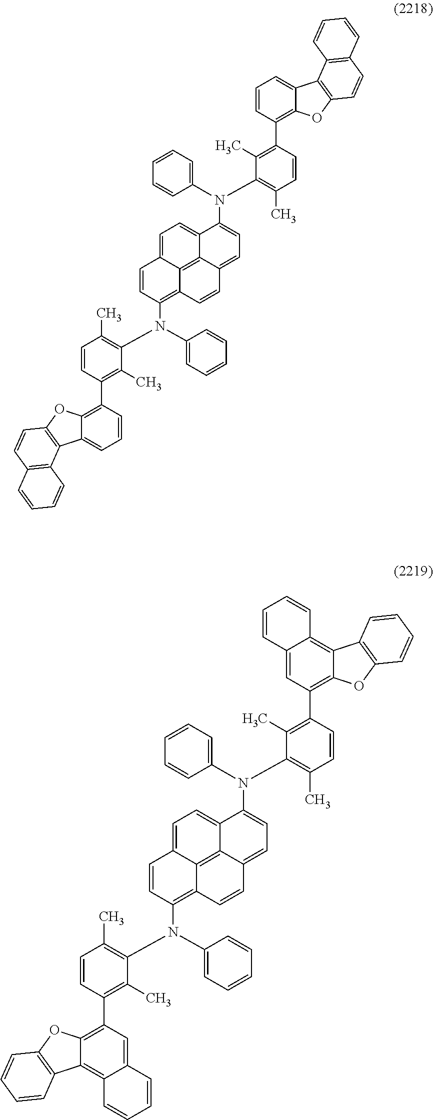 [SMILES: Cc1ccc(-c2cccc3c2oc2ccc4ccccc4c23)c(C)c1N(c1ccccc1)c1ccc2ccc3c(N(c4ccccc4)c4c(C)ccc(-c5cccc6c5oc5ccc7ccccc7c56)c4C)ccc4ccc1c2c43 ; Cc1ccc(-c2cc3ccccc3c3c2oc2ccccc23)c(C)c1N(c1ccccc1)c1ccc2ccc3c(N(c4ccccc4)c4c(C)ccc(-c5cc6ccccc6c6c5oc5ccccc56)c4C)ccc4ccc1c2c43]

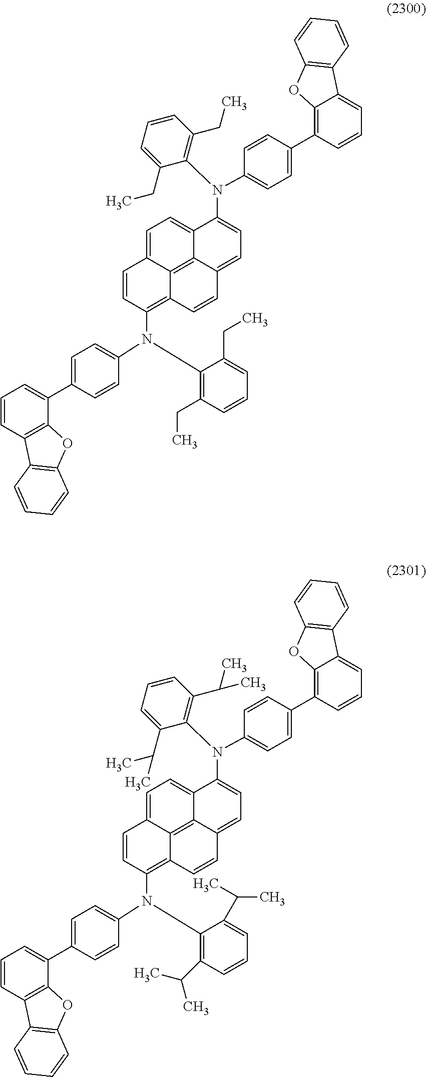 [SMILES: CCc1cccc(CC)c1N(c1ccc(-c2cccc3c2oc2ccccc23)cc1)c1ccc2ccc3c(N(c4ccc(-c5cccc6c5oc5ccccc56)cc4)c4c(CC)cccc4CC)ccc4ccc1c2c43 ; CC(C)c1cccc(C(C)C)c1N(c1ccc(-c2cccc3c2oc2ccccc23)cc1)c1ccc2ccc3c(N(c4ccc(-c5cccc6c5oc5ccccc56)cc4)c4c(C(C)C)cccc4C(C)C)ccc4ccc1c2c43]

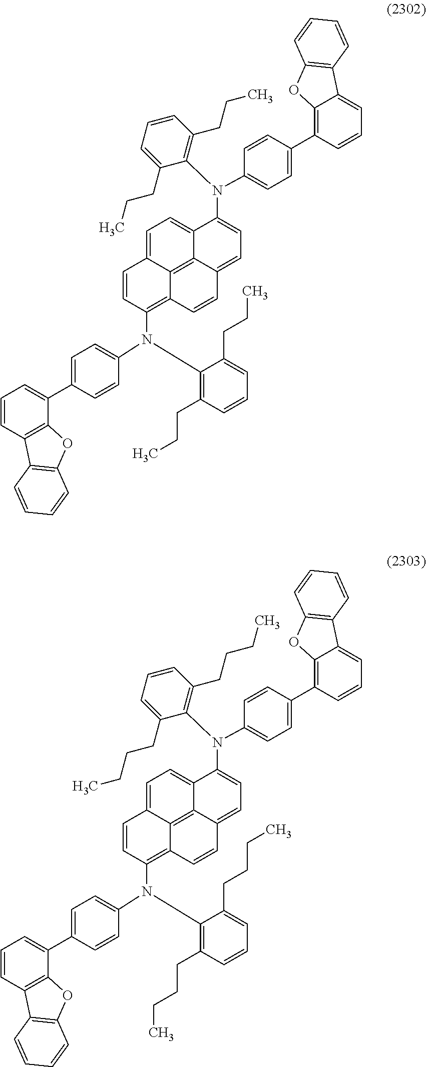 [SMILES: CCCc1cccc(CCC)c1N(c1ccc(-c2cccc3c2oc2ccccc23)cc1)c1ccc2ccc3c(N(c4ccc(-c5cccc6c5oc5ccccc56)cc4)c4c(CCC)cccc4CCC)ccc4ccc1c2c43 ; CCCCc1cccc(CCCC)c1N(c1ccc(-c2cccc3c2oc2ccccc23)cc1)c1ccc2ccc3c(N(c4ccc(-c5cccc6c5oc5ccccc56)cc4)c4c(CCCC)cccc4CCCC)ccc4ccc1c2c43]

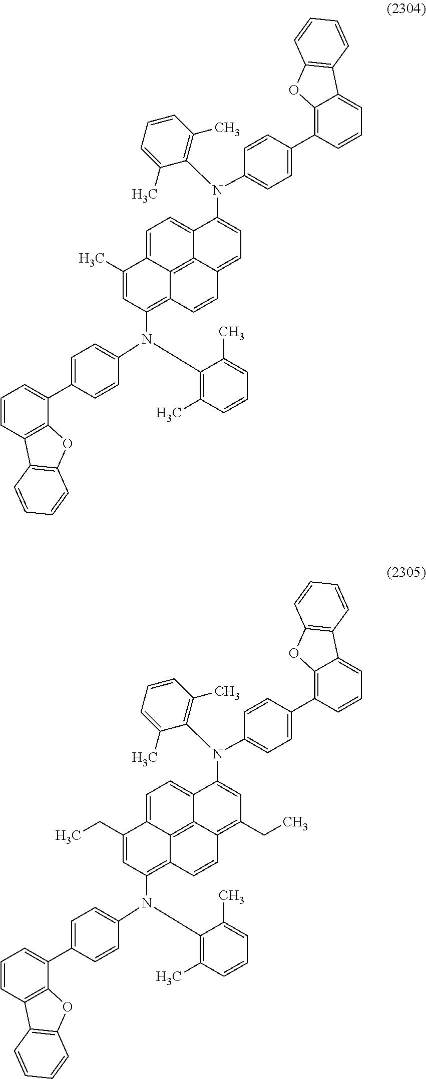 [SMILES: Cc1cccc(C)c1N(c1ccc(-c2cccc3c2oc2ccccc23)cc1)c1ccc2ccc3c(N(c4ccc(-c5cccc6c5oc5ccccc56)cc4)c4c(C)cccc4C)cc(C)c4ccc1c2c43 ; CCc1cc(N(c2ccc(-c3cccc4c3oc3ccccc34)cc2)c2c(C)cccc2C)c2ccc3c(CC)cc(N(c4ccc(-c5cccc6c5oc5ccccc56)cc4)c4c(C)cccc4C)c4ccc1c2c34]

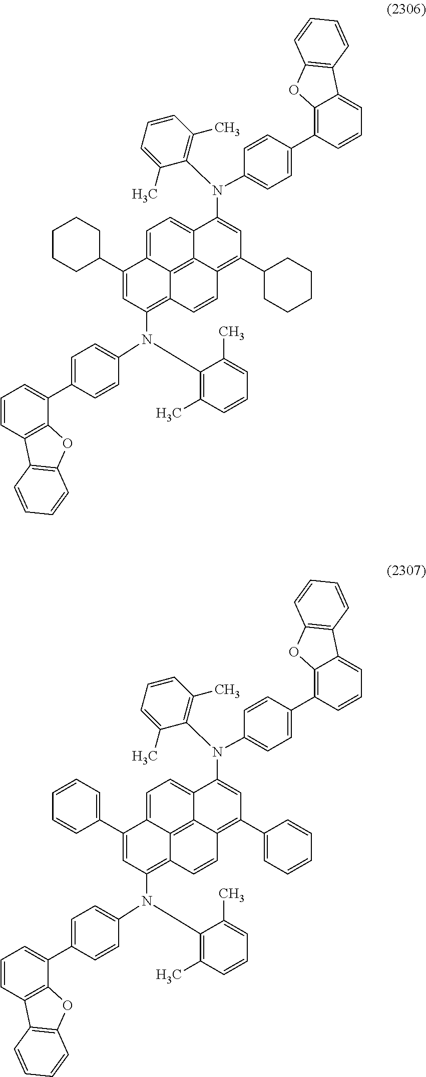 [SMILES: Cc1cccc(C)c1N(c1ccc(-c2cccc3c2oc2ccccc23)cc1)c1cc(C2CCCCC2)c2ccc3c(N(c4ccc(-c5cccc6c5oc5ccccc56)cc4)c4c(C)cccc4C)cc(C4CCCCC4)c4ccc1c2c43 ; Cc1cccc(C)c1N(c1ccc(-c2cccc3c2oc2ccccc23)cc1)c1cc(-c2ccccc2)c2ccc3c(N(c4ccc(-c5cccc6c5oc5ccccc56)cc4)c4c(C)cccc4C)cc(-c4ccccc4)c4ccc1c2c43]

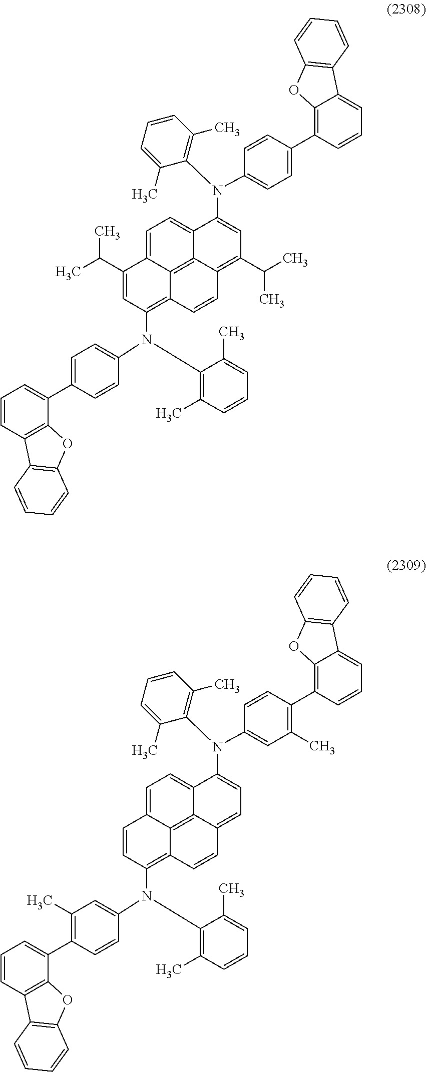 [SMILES: Cc1cccc(C)c1N(c1ccc(-c2cccc3c2oc2ccccc23)cc1)c1cc(C(C)C)c2ccc3c(N(c4ccc(-c5cccc6c5oc5ccccc56)cc4)c4c(C)cccc4C)cc(C(C)C)c4ccc1c2c43 ; Cc1cc(N(c2c(C)cccc2C)c2ccc3ccc4c(N(c5ccc(-c6cccc7c6oc6ccccc67)c(C)c5)c5c(C)cccc5C)ccc5ccc2c3c54)ccc1-c1cccc2c1oc1ccccc12]

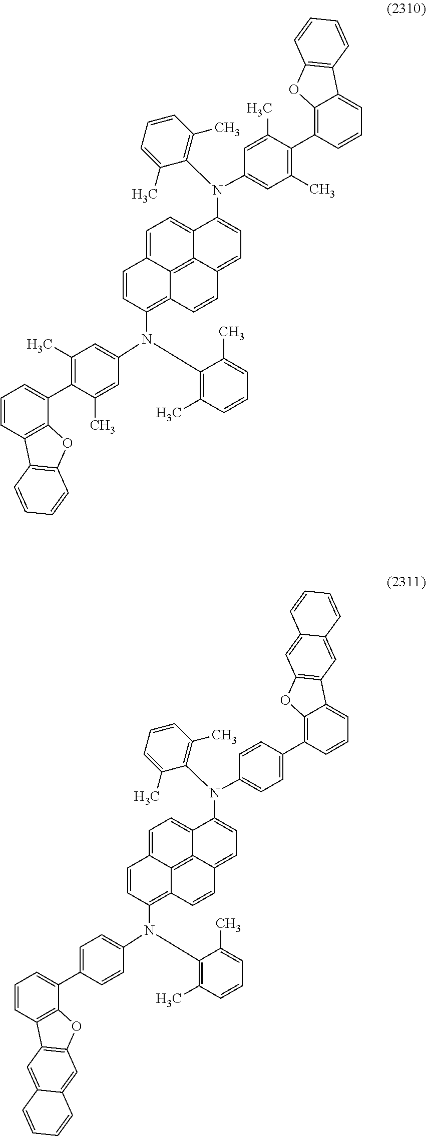 [SMILES: Cc1cc(N(c2c(C)cccc2C)c2ccc3ccc4c(N(c5cc(C)c(-c6cccc7c6oc6ccccc67)c(C)c5)c5c(C)cccc5C)ccc5ccc2c3c54)cc(C)c1-c1cccc2c1oc1ccccc12 ; Cc1cccc(C)c1N(c1ccc(-c2cccc3c2oc2cc4ccccc4cc23)cc1)c1ccc2ccc3c(N(c4ccc(-c5cccc6c5oc5cc7ccccc7cc56)cc4)c4c(C)cccc4C)ccc4ccc1c2c43]

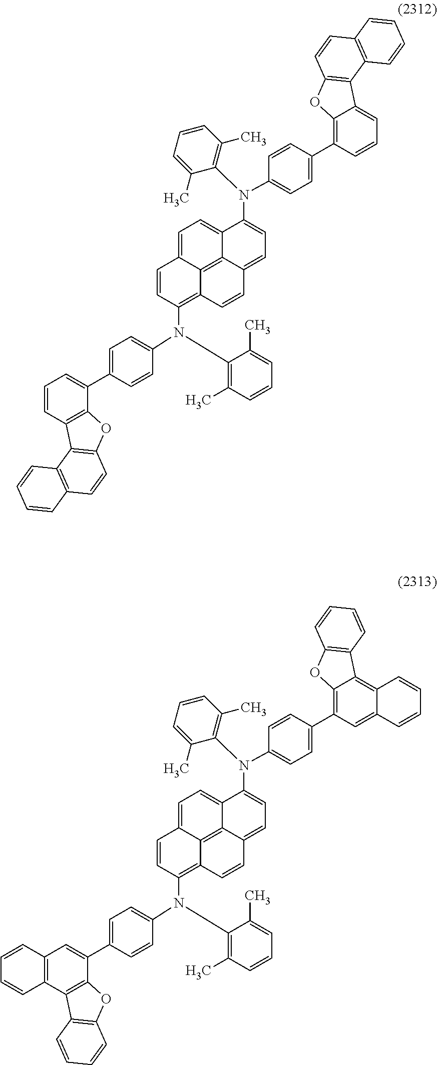 [SMILES: Cc1cccc(C)c1N(c1ccc(-c2cccc3c2oc2ccc4ccccc4c23)cc1)c1ccc2ccc3c(N(c4ccc(-c5cccc6c5oc5ccc7ccccc7c56)cc4)c4c(C)cccc4C)ccc4ccc1c2c43 ; Cc1cccc(C)c1N(c1ccc(-c2cc3ccccc3c3c2oc2ccccc23)cc1)c1ccc2ccc3c(N(c4ccc(-c5cc6ccccc6c6c5oc5ccccc56)cc4)c4c(C)cccc4C)ccc4ccc1c2c43]

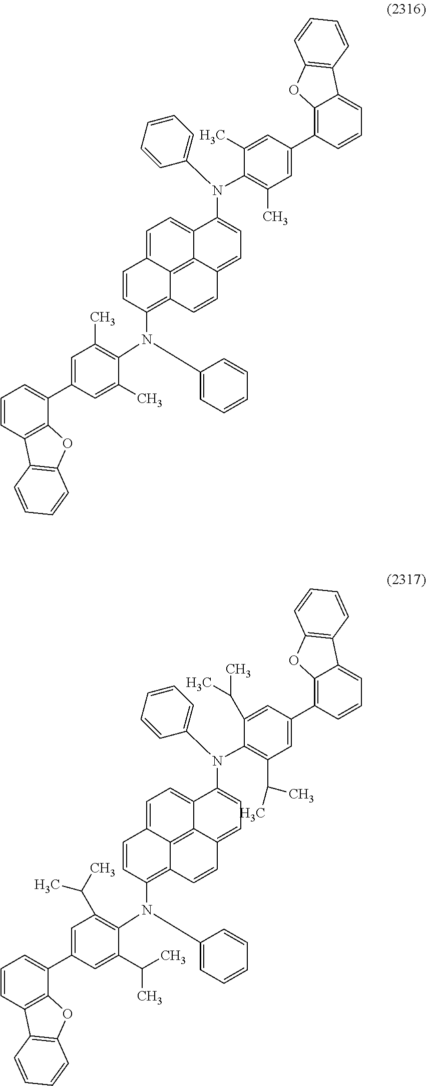 [SMILES: Cc1cc(-c2cccc3c2oc2ccccc23)cc(C)c1N(c1ccccc1)c1ccc2ccc3c(N(c4ccccc4)c4c(C)cc(-c5cccc6c5oc5ccccc56)cc4C)ccc4ccc1c2c43 ; CC(C)c1cc(-c2cccc3c2oc2ccccc23)cc(C(C)C)c1N(c1ccccc1)c1ccc2ccc3c(N(c4ccccc4)c4c(C(C)C)cc(-c5cccc6c5oc5ccccc56)cc4C(C)C)ccc4ccc1c2c43]

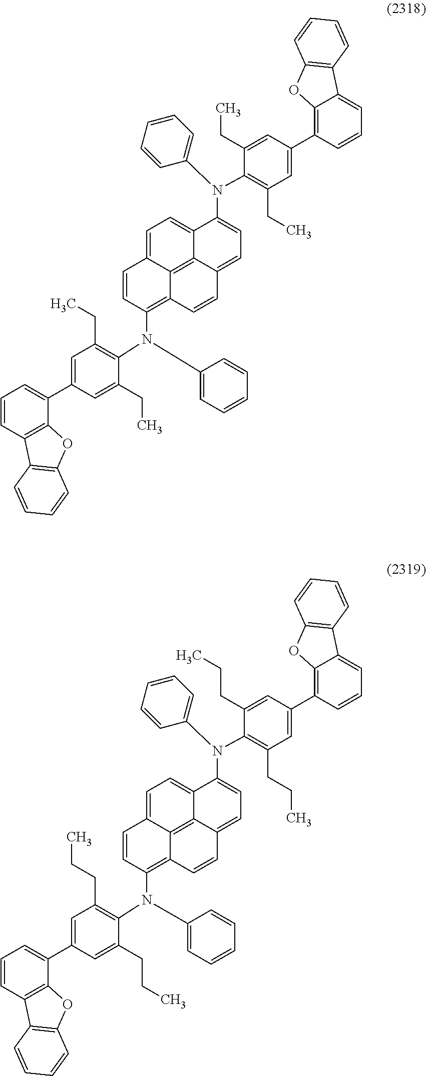 [SMILES: CCc1cc(-c2cccc3c2oc2ccccc23)cc(CC)c1N(c1ccccc1)c1ccc2ccc3c(N(c4ccccc4)c4c(CC)cc(-c5cccc6c5oc5ccccc56)cc4CC)ccc4ccc1c2c43 ; CCCc1cc(-c2cccc3c2oc2ccccc23)cc(CCC)c1N(c1ccccc1)c1ccc2ccc3c(N(c4ccccc4)c4c(CCC)cc(-c5cccc6c5oc5ccccc56)cc4CCC)ccc4ccc1c2c43]

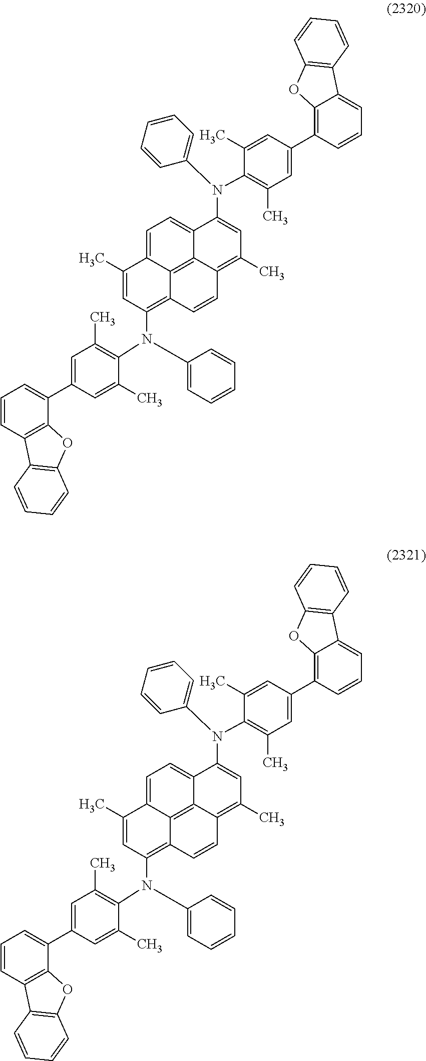 [SMILES: Cc1cc(-c2cccc3c2oc2ccccc23)cc(C)c1N(c1ccccc1)c1cc(C)c2ccc3c(N(c4ccccc4)c4c(C)cc(-c5cccc6c5oc5ccccc56)cc4C)cc(C)c4ccc1c2c43 ; Cc1cc(-c2cccc3c2oc2ccccc23)cc(C)c1N(c1ccccc1)c1cc(C)c2ccc3c(N(c4ccccc4)c4c(C)cc(-c5cccc6c5oc5ccccc56)cc4C)cc(C)c4ccc1c2c43]

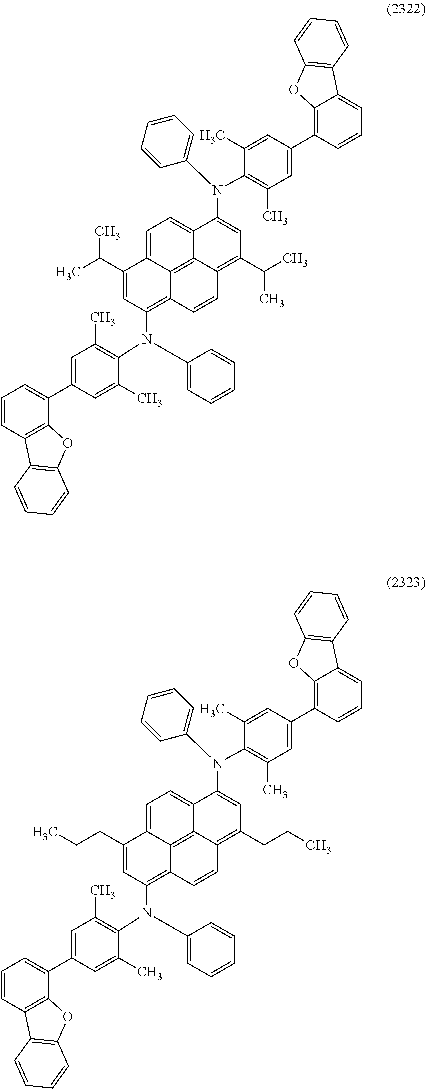 [SMILES: Cc1cc(-c2cccc3c2oc2ccccc23)cc(C)c1N(c1ccccc1)c1cc(C(C)C)c2ccc3c(N(c4ccccc4)c4c(C)cc(-c5cccc6c5oc5ccccc56)cc4C)cc(C(C)C)c4ccc1c2c43 ; CCCc1cc(N(c2ccccc2)c2c(C)cc(-c3cccc4c3oc3ccccc34)cc2C)c2ccc3c(CCC)cc(N(c4ccccc4)c4c(C)cc(-c5cccc6c5oc5ccccc56)cc4C)c4ccc1c2c34]

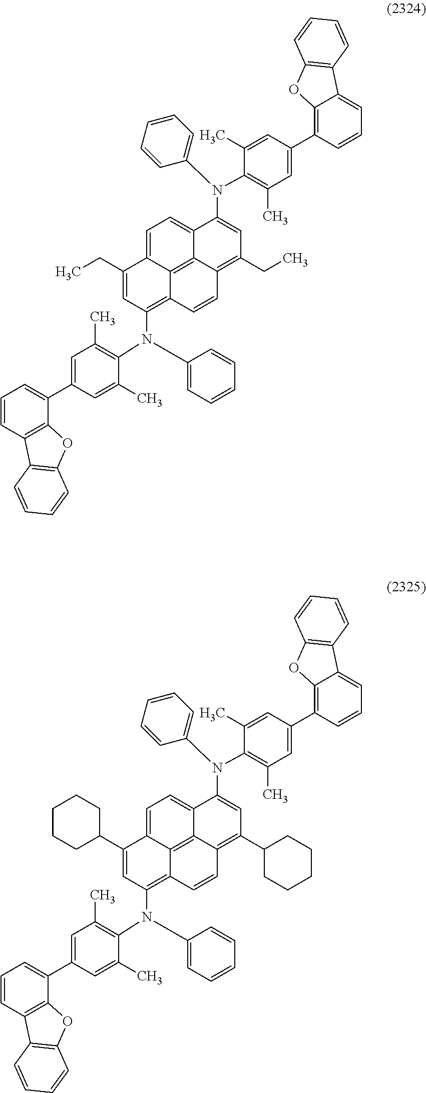 [SMILES: CCc1cc(N(c2ccccc2)c2c(C)cc(-c3cccc4c3oc3ccccc34)cc2C)c2ccc3c(CC)cc(N(c4ccccc4)c4c(C)cc(-c5cccc6c5oc5ccccc56)cc4C)c4ccc1c2c34 ; Cc1cc(-c2cccc3c2oc2ccccc23)cc(C)c1N(c1ccccc1)c1cc(C2CCCCC2)c2ccc3c(N(c4ccccc4)c4c(C)cc(-c5cccc6c5oc5ccccc56)cc4C)cc(C4CCCCC4)c4ccc1c2c43]

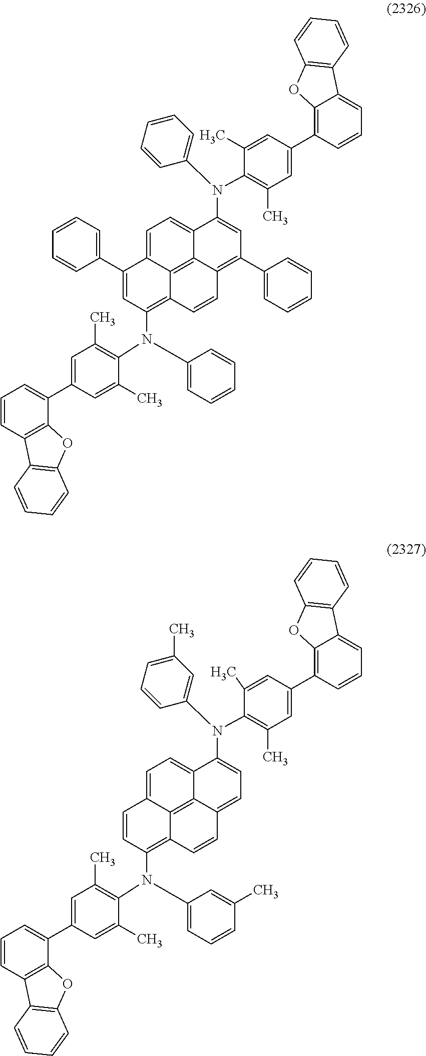 [SMILES: Cc1cc(-c2cccc3c2oc2ccccc23)cc(C)c1N(c1ccccc1)c1cc(-c2ccccc2)c2ccc3c(N(c4ccccc4)c4c(C)cc(-c5cccc6c5oc5ccccc56)cc4C)cc(-c4ccccc4)c4ccc1c2c43 ; Cc1cccc(N(c2c(C)cc(-c3cccc4c3oc3ccccc34)cc2C)c2ccc3ccc4c(N(c5cccc(C)c5)c5c(C)cc(-c6cccc7c6oc6ccccc67)cc5C)ccc5ccc2c3c54)c1]

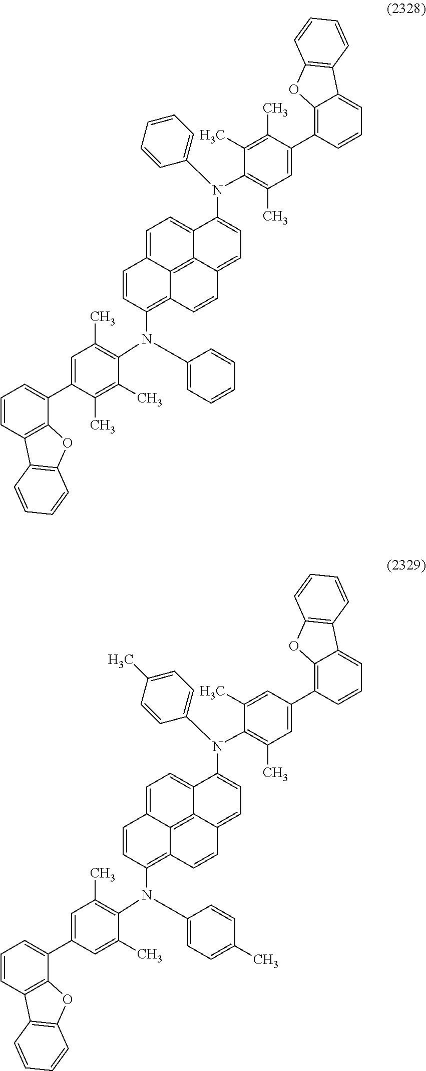 [SMILES: Cc1cc(-c2cccc3c2oc2ccccc23)c(C)c(C)c1N(c1ccccc1)c1ccc2ccc3c(N(c4ccccc4)c4c(C)cc(-c5cccc6c5oc5ccccc56)c(C)c4C)ccc4ccc1c2c43 ; Cc1ccc(N(c2c(C)cc(-c3cccc4c3oc3ccccc34)cc2C)c2ccc3ccc4c(N(c5ccc(C)cc5)c5c(C)cc(-c6cccc7c6oc6ccccc67)cc5C)ccc5ccc2c3c54)cc1]

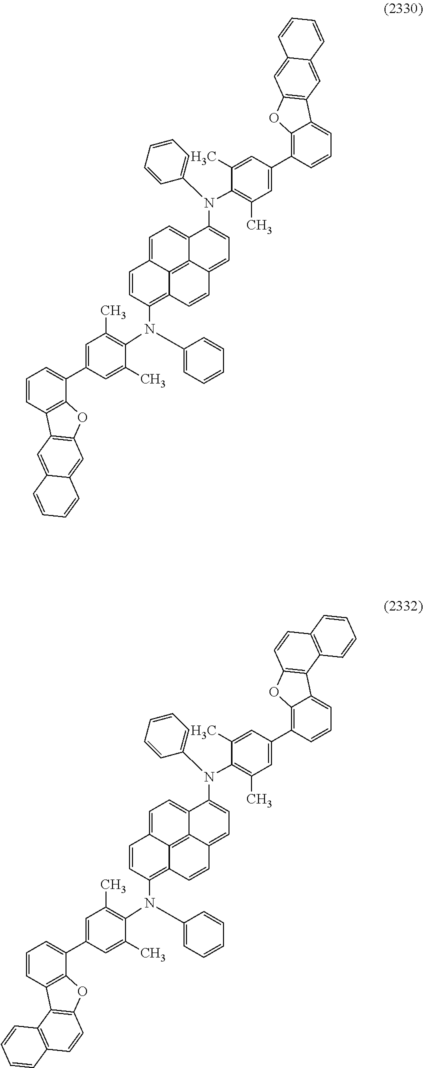 [SMILES: Cc1cc(-c2cccc3c2oc2cc4ccccc4cc23)cc(C)c1N(c1ccccc1)c1ccc2ccc3c(N(c4ccccc4)c4c(C)cc(-c5cccc6c5oc5cc7ccccc7cc56)cc4C)ccc4ccc1c2c43 ; Cc1cc(-c2cccc3c2oc2ccc4ccccc4c23)cc(C)c1N(c1ccccc1)c1ccc2ccc3c(N(c4ccccc4)c4c(C)cc(-c5cccc6c5oc5ccc7ccccc7c56)cc4C)ccc4ccc1c2c43]

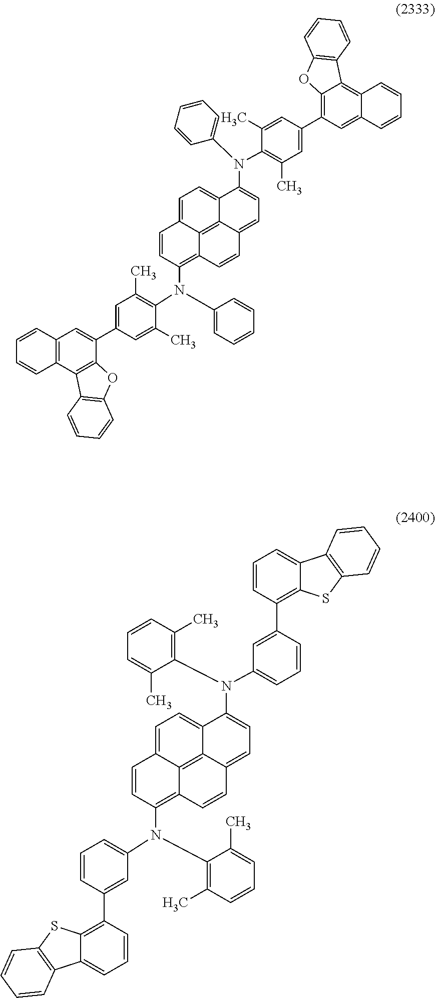 [SMILES: Cc1cc(-c2cc3ccccc3c3c2oc2ccccc23)cc(C)c1N(c1ccccc1)c1ccc2ccc3c(N(c4ccccc4)c4c(C)cc(-c5cc6ccccc6c6c5oc5ccccc56)cc4C)ccc4ccc1c2c43 ; Cc1cccc(C)c1N(c1cccc(-c2cccc3c2sc2ccccc23)c1)c1ccc2ccc3c(N(c4cccc(-c5cccc6c5sc5ccccc56)c4)c4c(C)cccc4C)ccc4ccc1c2c43]

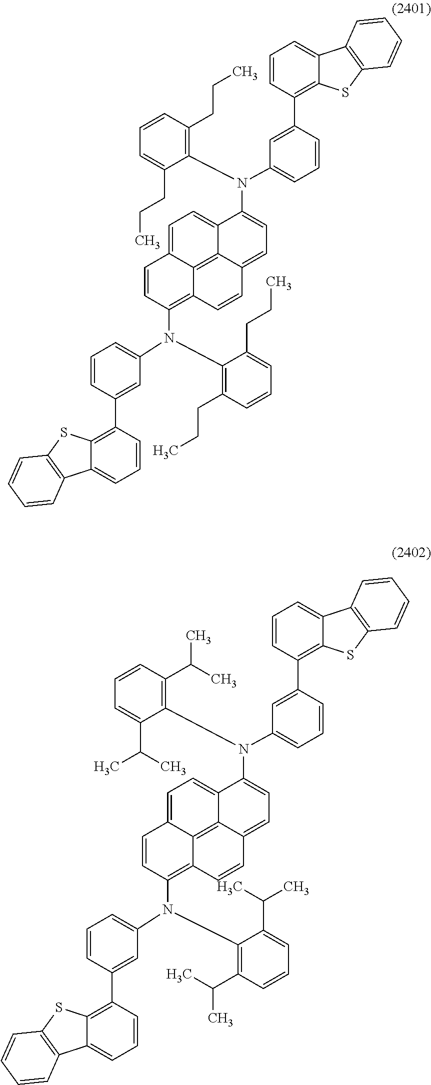 [SMILES: CCCc1cccc(CCC)c1N(c1cccc(-c2cccc3c2sc2ccccc23)c1)c1ccc2ccc3c(N(c4cccc(-c5cccc6c5sc5ccccc56)c4)c4c(CCC)cccc4CCC)ccc4ccc1c2c43 ; CC(C)c1cccc(C(C)C)c1N(c1cccc(-c2cccc3c2sc2ccccc23)c1)c1ccc2ccc3c(N(c4cccc(-c5cccc6c5sc5ccccc56)c4)c4c(C(C)C)cccc4C(C)C)ccc4ccc1c2c43]

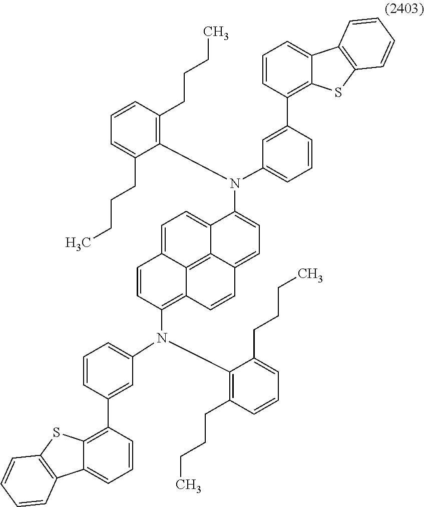 [SMILES: CCCCc1cccc(CCCC)c1N(c1cccc(-c2cccc3c2sc2ccccc23)c1)c1ccc2ccc3c(N(c4cccc(-c5cccc6c5sc5ccccc56)c4)c4c(CCCC)cccc4CCCC)ccc4ccc1c2c43]

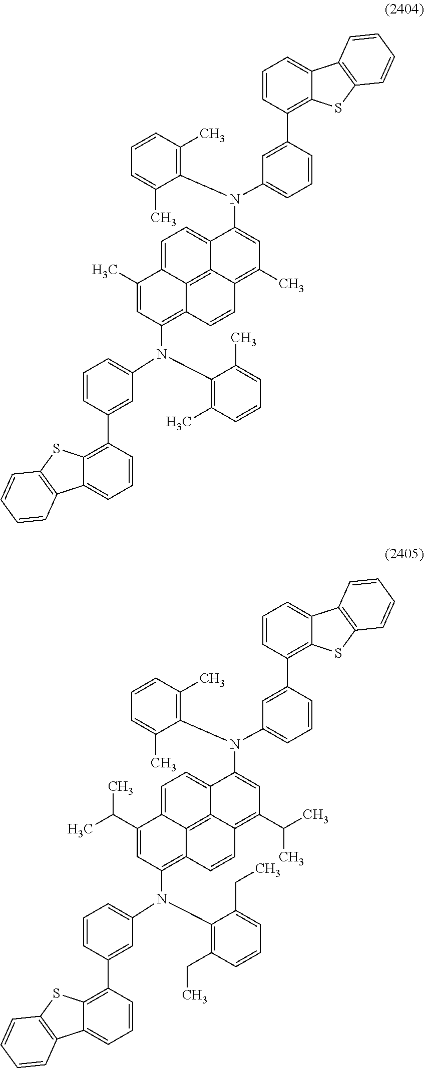 [SMILES: Cc1cccc(C)c1N(c1cccc(-c2cccc3c2sc2ccccc23)c1)c1cc(C)c2ccc3c(N(c4cccc(-c5cccc6c5sc5ccccc56)c4)c4c(C)cccc4C)cc(C)c4ccc1c2c43 ; CCc1cccc(CC)c1N(c1cccc(-c2cccc3c2sc2ccccc23)c1)c1cc(C(C)C)c2ccc3c(N(c4cccc(-c5cccc6c5sc5ccccc56)c4)c4c(C)cccc4C)cc(C(C)C)c4ccc1c2c43]

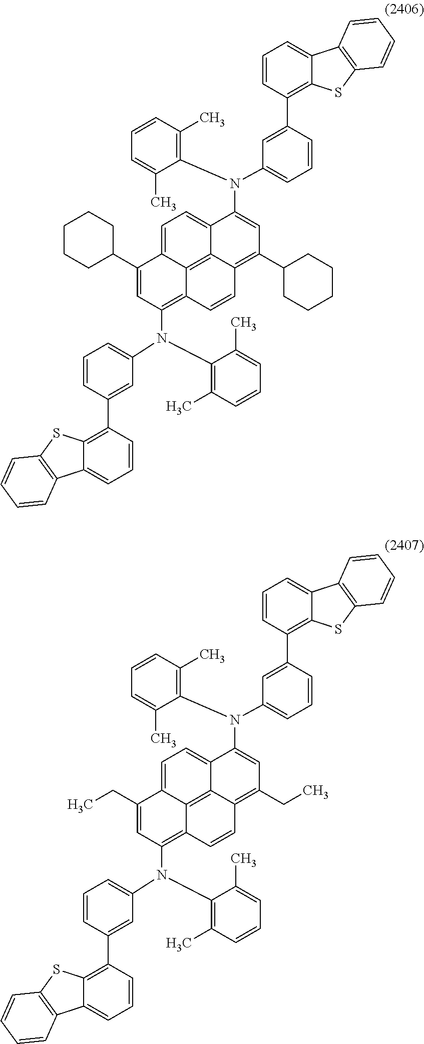 [SMILES: Cc1cccc(C)c1N(c1cccc(-c2cccc3c2sc2ccccc23)c1)c1cc(C2CCCCC2)c2ccc3c(N(c4cccc(-c5cccc6c5sc5ccccc56)c4)c4c(C)cccc4C)cc(C4CCCCC4)c4ccc1c2c43 ; CCc1cc(N(c2cccc(-c3cccc4c3sc3ccccc34)c2)c2c(C)cccc2C)c2ccc3c(CC)cc(N(c4cccc(-c5cccc6c5sc5ccccc56)c4)c4c(C)cccc4C)c4ccc1c2c34]

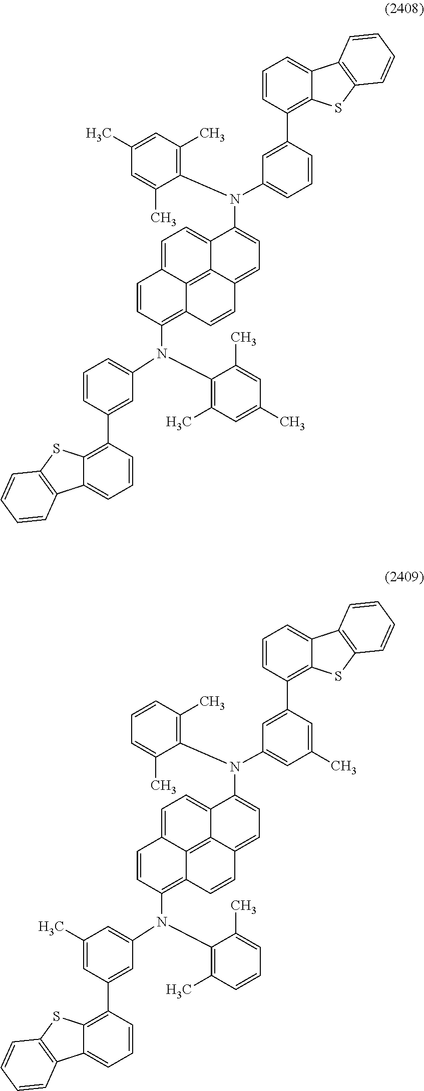 [SMILES: Cc1cc(C)c(N(c2cccc(-c3cccc4c3sc3ccccc34)c2)c2ccc3ccc4c(N(c5cccc(-c6cccc7c6sc6ccccc67)c5)c5c(C)cc(C)cc5C)ccc5ccc2c3c54)c(C)c1 ; Cc1cc(-c2cccc3c2sc2ccccc23)cc(N(c2c(C)cccc2C)c2ccc3ccc4c(N(c5cc(C)cc(-c6cccc7c6sc6ccccc67)c5)c5c(C)cccc5C)ccc5ccc2c3c54)c1]

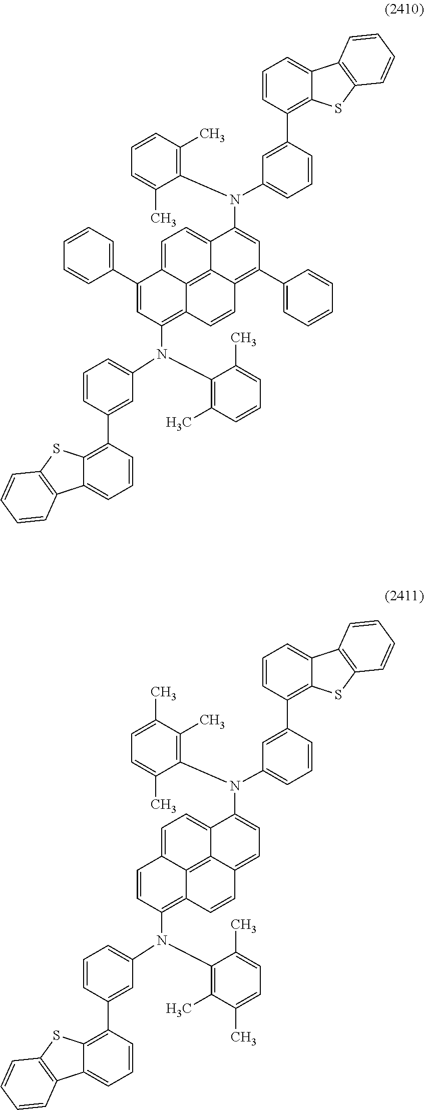 [SMILES: Cc1cccc(C)c1N(c1cccc(-c2cccc3c2sc2ccccc23)c1)c1cc(-c2ccccc2)c2ccc3c(N(c4cccc(-c5cccc6c5sc5ccccc56)c4)c4c(C)cccc4C)cc(-c4ccccc4)c4ccc1c2c43 ; Cc1ccc(C)c(N(c2cccc(-c3cccc4c3sc3ccccc34)c2)c2ccc3ccc4c(N(c5cccc(-c6cccc7c6sc6ccccc67)c5)c5c(C)ccc(C)c5C)ccc5ccc2c3c54)c1C]

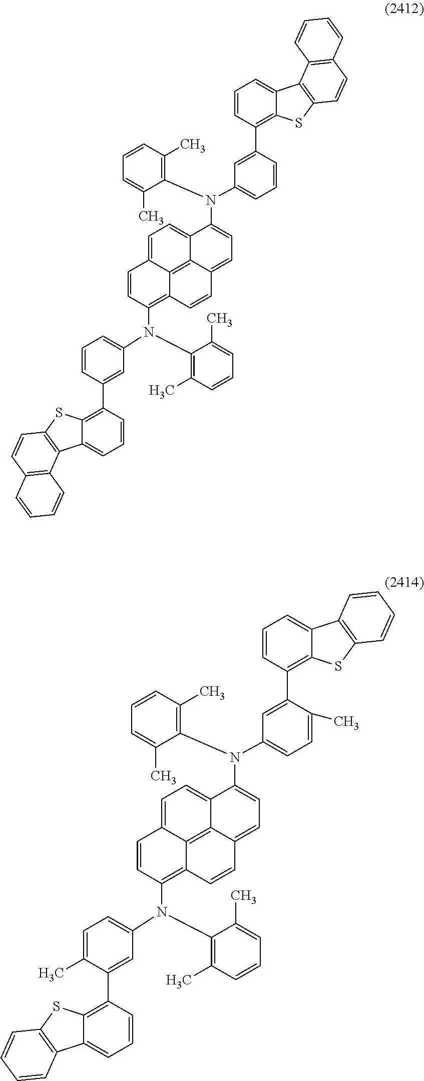 [SMILES: Cc1cccc(C)c1N(c1cccc(-c2cccc3c2sc2ccc4ccccc4c23)c1)c1ccc2ccc3c(N(c4cccc(-c5cccc6c5sc5ccc7ccccc7c56)c4)c4c(C)cccc4C)ccc4ccc1c2c43 ; Cc1ccc(N(c2c(C)cccc2C)c2ccc3ccc4c(N(c5ccc(C)c(-c6cccc7c6sc6ccccc67)c5)c5c(C)cccc5C)ccc5ccc2c3c54)cc1-c1cccc2c1sc1ccccc12]

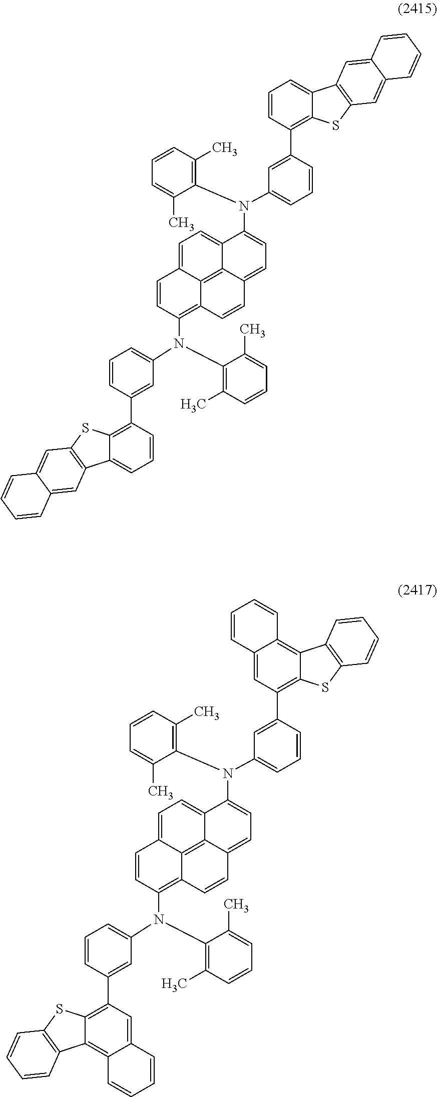 [SMILES: Cc1cccc(C)c1N(c1cccc(-c2cccc3c2sc2cc4ccccc4cc23)c1)c1ccc2ccc3c(N(c4cccc(-c5cccc6c5sc5cc7ccccc7cc56)c4)c4c(C)cccc4C)ccc4ccc1c2c43 ; Cc1cccc(C)c1N(c1cccc(-c2cc3ccccc3c3c2sc2ccccc23)c1)c1ccc2ccc3c(N(c4cccc(-c5cc6ccccc6c6c5sc5ccccc56)c4)c4c(C)cccc4C)ccc4ccc1c2c43]

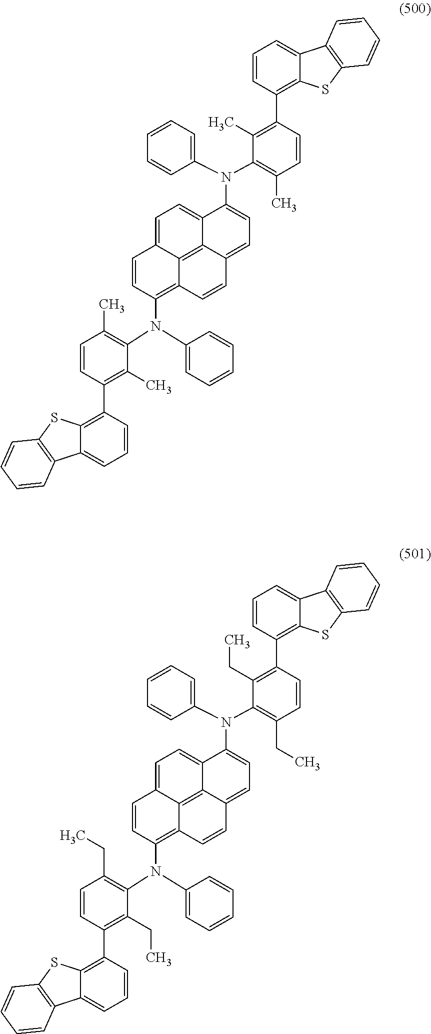 [SMILES: Cc1ccc(-c2cccc3c2sc2ccccc23)c(C)c1N(c1ccccc1)c1ccc2ccc3c(N(c4ccccc4)c4c(C)ccc(-c5cccc6c5sc5ccccc56)c4C)ccc4ccc1c2c43 ; CCc1ccc(-c2cccc3c2sc2ccccc23)c(CC)c1N(c1ccccc1)c1ccc2ccc3c(N(c4ccccc4)c4c(CC)ccc(-c5cccc6c5sc5ccccc56)c4CC)ccc4ccc1c2c43]

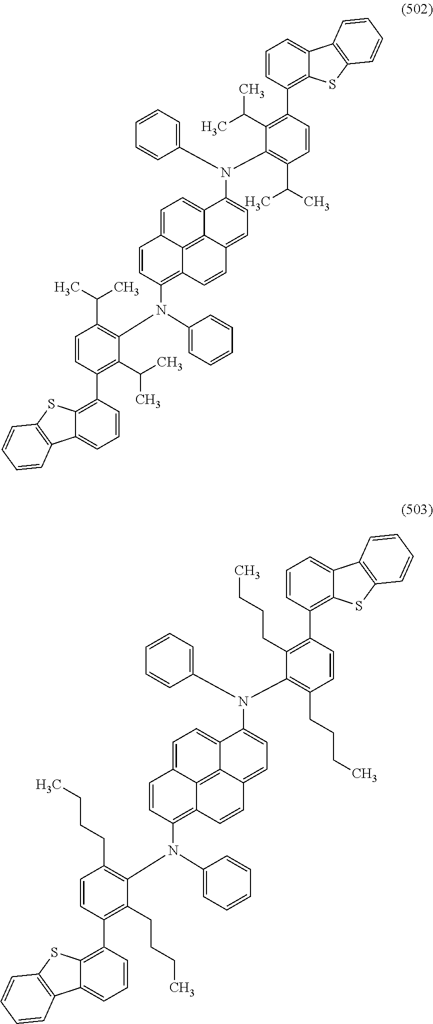 [SMILES: CC(C)c1ccc(-c2cccc3c2sc2ccccc23)c(C(C)C)c1N(c1ccccc1)c1ccc2ccc3c(N(c4ccccc4)c4c(C(C)C)ccc(-c5cccc6c5sc5ccccc56)c4C(C)C)ccc4ccc1c2c43 ; CCCCc1ccc(-c2cccc3c2sc2ccccc23)c(CCCC)c1N(c1ccccc1)c1ccc2ccc3c(N(c4ccccc4)c4c(CCCC)ccc(-c5cccc6c5sc5ccccc56)c4CCCC)ccc4ccc1c2c43]

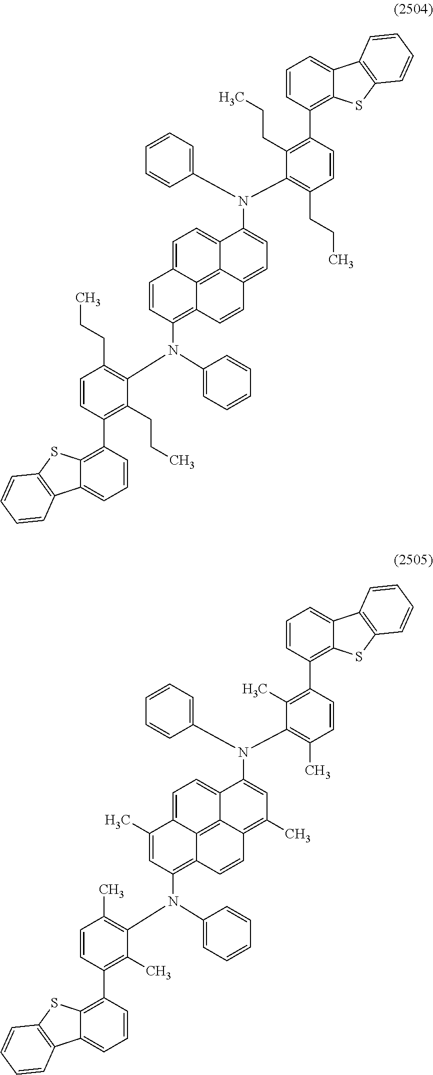 [SMILES: CCCc1ccc(-c2cccc3c2sc2ccccc23)c(CCC)c1N(c1ccccc1)c1ccc2ccc3c(N(c4ccccc4)c4c(CCC)ccc(-c5cccc6c5sc5ccccc56)c4CCC)ccc4ccc1c2c43 ; Cc1ccc(-c2cccc3c2sc2ccccc23)c(C)c1N(c1ccccc1)c1cc(C)c2ccc3c(N(c4ccccc4)c4c(C)ccc(-c5cccc6c5sc5ccccc56)c4C)cc(C)c4ccc1c2c43]

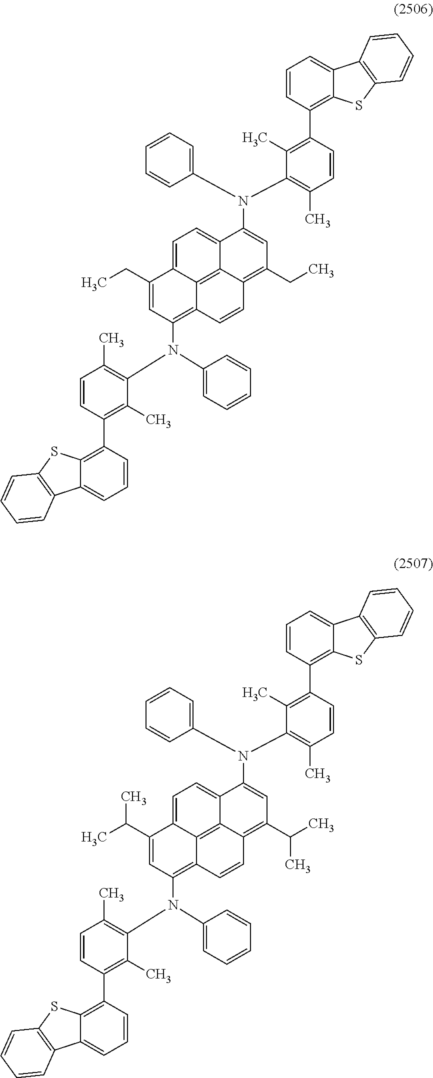 [SMILES: CCc1cc(N(c2ccccc2)c2c(C)ccc(-c3cccc4c3sc3ccccc34)c2C)c2ccc3c(CC)cc(N(c4ccccc4)c4c(C)ccc(-c5cccc6c5sc5ccccc56)c4C)c4ccc1c2c34 ; Cc1ccc(-c2cccc3c2sc2ccccc23)c(C)c1N(c1ccccc1)c1cc(C(C)C)c2ccc3c(N(c4ccccc4)c4c(C)ccc(-c5cccc6c5sc5ccccc56)c4C)cc(C(C)C)c4ccc1c2c43]

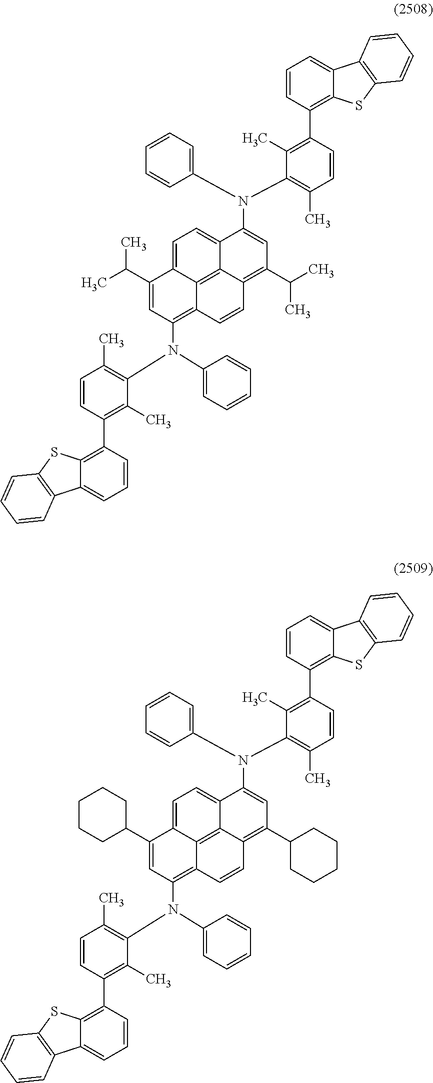 [SMILES: Cc1ccc(-c2cccc3c2sc2ccccc23)c(C)c1N(c1ccccc1)c1cc(C(C)C)c2ccc3c(N(c4ccccc4)c4c(C)ccc(-c5cccc6c5sc5ccccc56)c4C)cc(C(C)C)c4ccc1c2c43 ; Cc1ccc(-c2cccc3c2sc2ccccc23)c(C)c1N(c1ccccc1)c1cc(C2CCCCC2)c2ccc3c(N(c4ccccc4)c4c(C)ccc(-c5cccc6c5sc5ccccc56)c4C)cc(C4CCCCC4)c4ccc1c2c43]

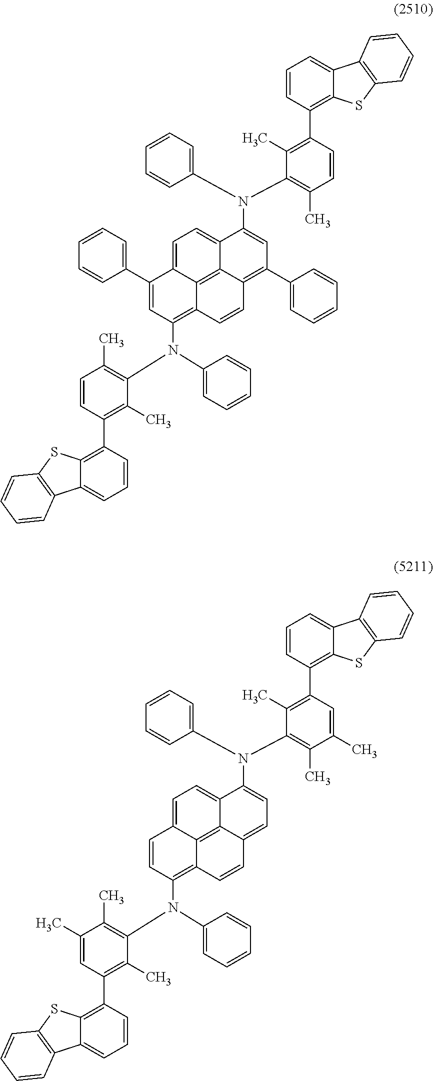 [SMILES: Cc1ccc(-c2cccc3c2sc2ccccc23)c(C)c1N(c1ccccc1)c1cc(-c2ccccc2)c2ccc3c(N(c4ccccc4)c4c(C)ccc(-c5cccc6c5sc5ccccc56)c4C)cc(-c4ccccc4)c4ccc1c2c43 ; Cc1cc(-c2cccc3c2sc2ccccc23)c(C)c(N(c2ccccc2)c2ccc3ccc4c(N(c5ccccc5)c5c(C)c(C)cc(-c6cccc7c6sc6ccccc67)c5C)ccc5ccc2c3c54)c1C]

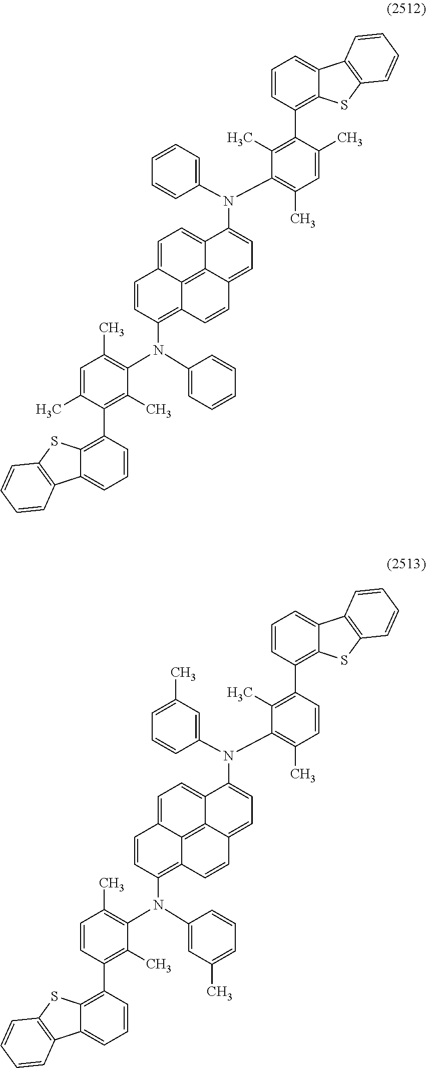 [SMILES: Cc1cc(C)c(N(c2ccccc2)c2ccc3ccc4c(N(c5ccccc5)c5c(C)cc(C)c(-c6cccc7c6sc6ccccc67)c5C)ccc5ccc2c3c54)c(C)c1-c1cccc2c1sc1ccccc12 ; Cc1cccc(N(c2c(C)ccc(-c3cccc4c3sc3ccccc34)c2C)c2ccc3ccc4c(N(c5cccc(C)c5)c5c(C)ccc(-c6cccc7c6sc6ccccc67)c5C)ccc5ccc2c3c54)c1]

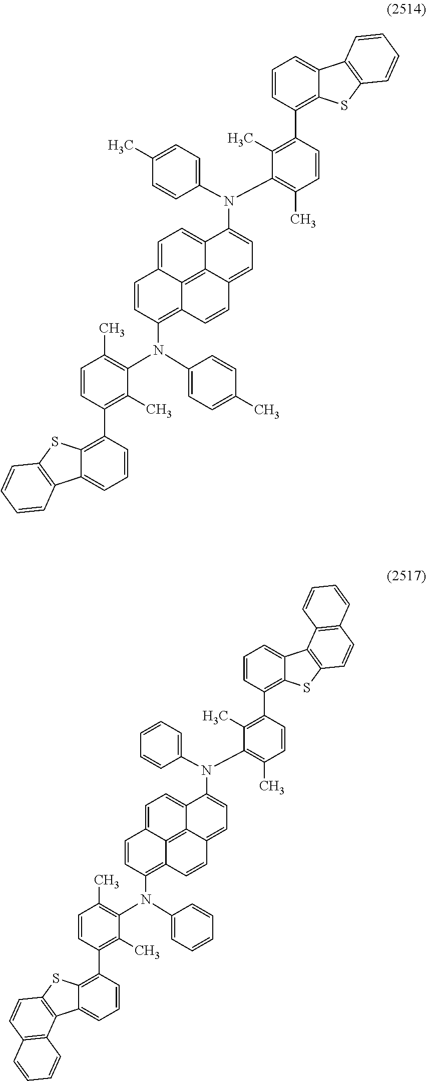 [SMILES: Cc1ccc(N(c2c(C)ccc(-c3cccc4c3sc3ccccc34)c2C)c2ccc3ccc4c(N(c5ccc(C)cc5)c5c(C)ccc(-c6cccc7c6sc6ccccc67)c5C)ccc5ccc2c3c54)cc1 ; Cc1ccc(-c2cccc3c2sc2ccc4ccccc4c23)c(C)c1N(c1ccccc1)c1ccc2ccc3c(N(c4ccccc4)c4c(C)ccc(-c5cccc6c5sc5ccc7ccccc7c56)c4C)ccc4ccc1c2c43]

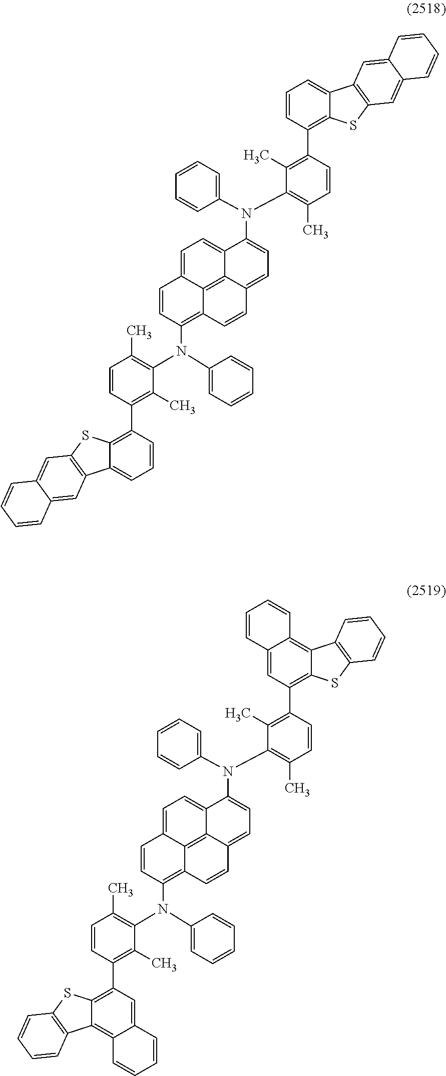 [SMILES: Cc1ccc(-c2cccc3c2sc2cc4ccccc4cc23)c(C)c1N(c1ccccc1)c1ccc2ccc3c(N(c4ccccc4)c4c(C)ccc(-c5cccc6c5sc5cc7ccccc7cc56)c4C)ccc4ccc1c2c43 ; Cc1ccc(-c2cc3ccccc3c3c2sc2ccccc23)c(C)c1N(c1ccccc1)c1ccc2ccc3c(N(c4ccccc4)c4c(C)ccc(-c5cc6ccccc6c6c5sc5ccccc56)c4C)ccc4ccc1c2c43]

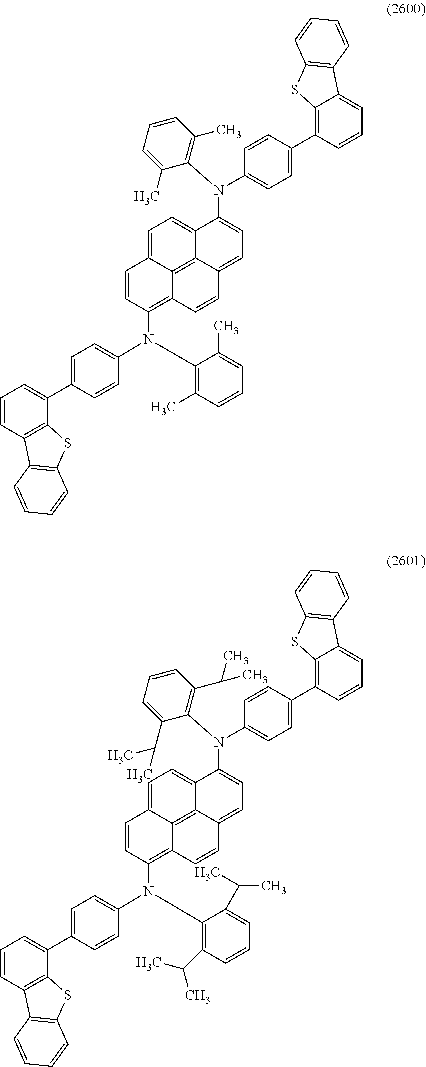 [SMILES: Cc1cccc(C)c1N(c1ccc(-c2cccc3c2sc2ccccc23)cc1)c1ccc2ccc3c(N(c4ccc(-c5cccc6c5sc5ccccc56)cc4)c4c(C)cccc4C)ccc4ccc1c2c43 ; CC(C)c1cccc(C(C)C)c1N(c1ccc(-c2cccc3c2sc2ccccc23)cc1)c1ccc2ccc3c(N(c4ccc(-c5cccc6c5sc5ccccc56)cc4)c4c(C(C)C)cccc4C(C)C)ccc4ccc1c2c43]

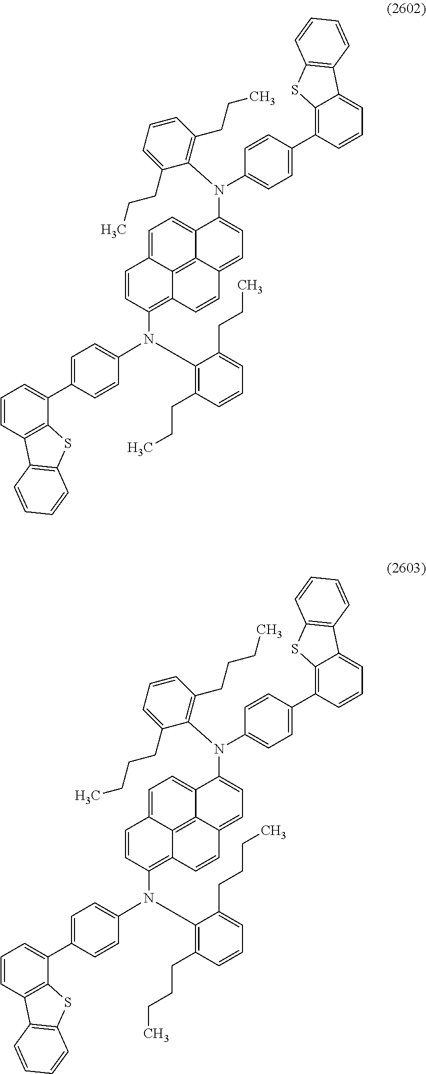 [SMILES: CCCc1cccc(CCC)c1N(c1ccc(-c2cccc3c2sc2ccccc23)cc1)c1ccc2ccc3c(N(c4ccc(-c5cccc6c5sc5ccccc56)cc4)c4c(CCC)cccc4CCC)ccc4ccc1c2c43 ; CCCCc1cccc(CCCC)c1N(c1ccc(-c2cccc3c2sc2ccccc23)cc1)c1ccc2ccc3c(N(c4ccc(-c5cccc6c5sc5ccccc56)cc4)c4c(CCCC)cccc4CCCC)ccc4ccc1c2c43]

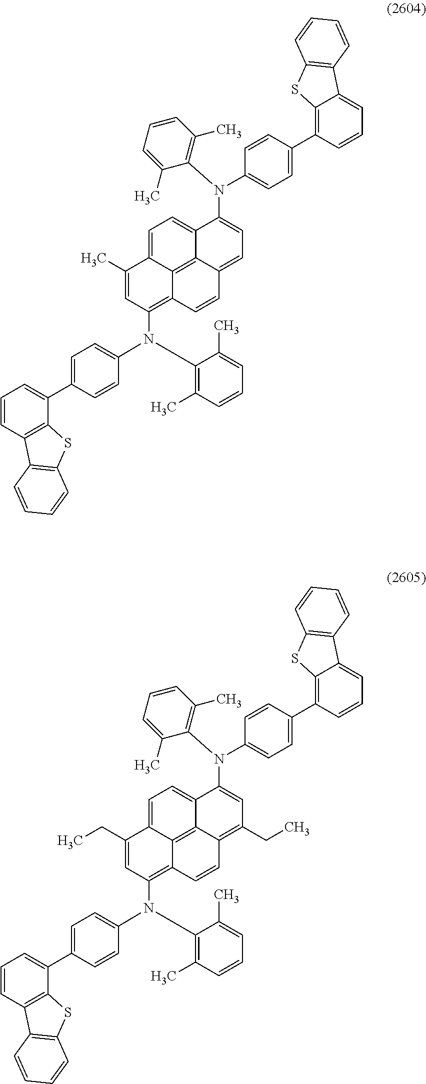 [SMILES: Cc1cccc(C)c1N(c1ccc(-c2cccc3c2sc2ccccc23)cc1)c1ccc2ccc3c(N(c4ccc(-c5cccc6c5sc5ccccc56)cc4)c4c(C)cccc4C)cc(C)c4ccc1c2c43 ; CCc1cc(N(c2ccc(-c3cccc4c3sc3ccccc34)cc2)c2c(C)cccc2C)c2ccc3c(CC)cc(N(c4ccc(-c5cccc6c5sc5ccccc56)cc4)c4c(C)cccc4C)c4ccc1c2c34]

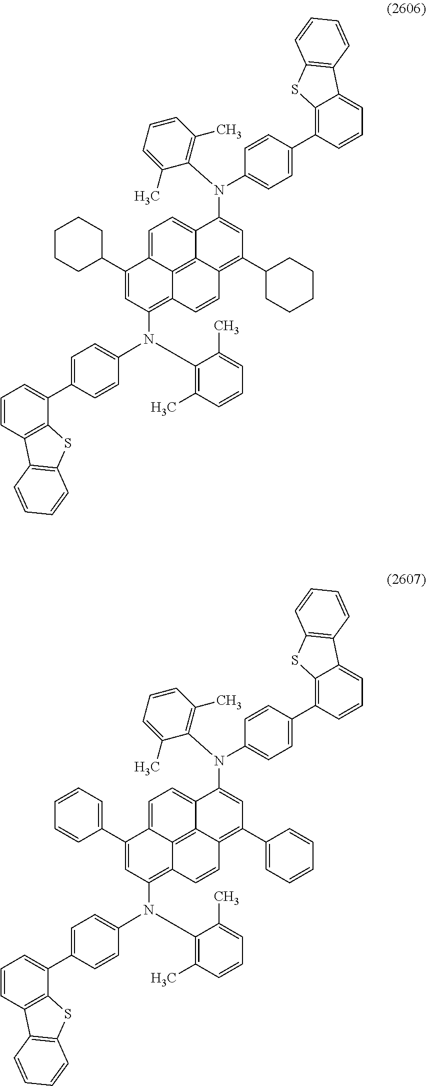 [SMILES: Cc1cccc(C)c1N(c1ccc(-c2cccc3c2sc2ccccc23)cc1)c1cc(C2CCCCC2)c2ccc3c(N(c4ccc(-c5cccc6c5sc5ccccc56)cc4)c4c(C)cccc4C)cc(C4CCCCC4)c4ccc1c2c43 ; Cc1cccc(C)c1N(c1ccc(-c2cccc3c2sc2ccccc23)cc1)c1cc(-c2ccccc2)c2ccc3c(N(c4ccc(-c5cccc6c5sc5ccccc56)cc4)c4c(C)cccc4C)cc(-c4ccccc4)c4ccc1c2c43]

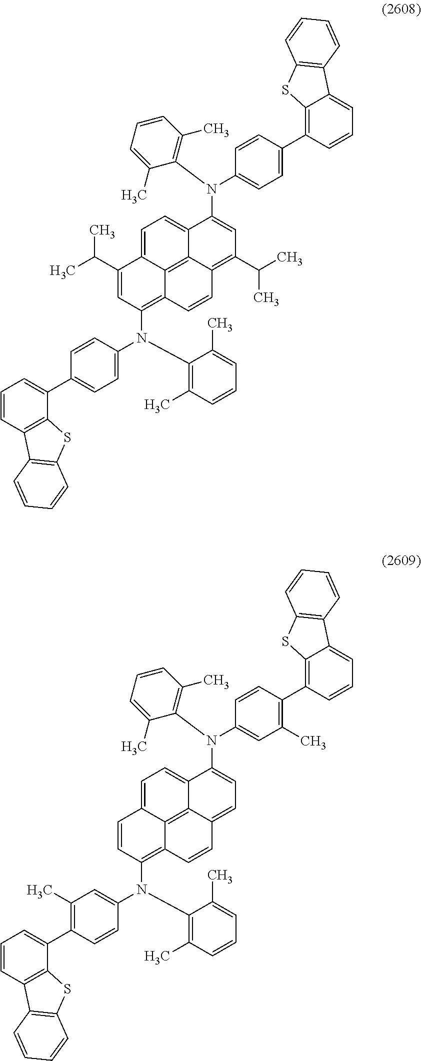 [SMILES: Cc1cccc(C)c1N(c1ccc(-c2cccc3c2sc2ccccc23)cc1)c1cc(C(C)C)c2ccc3c(N(c4ccc(-c5cccc6c5sc5ccccc56)cc4)c4c(C)cccc4C)cc(C(C)C)c4ccc1c2c43 ; Cc1cc(N(c2c(C)cccc2C)c2ccc3ccc4c(N(c5ccc(-c6cccc7c6sc6ccccc67)c(C)c5)c5c(C)cccc5C)ccc5ccc2c3c54)ccc1-c1cccc2c1sc1ccccc12]

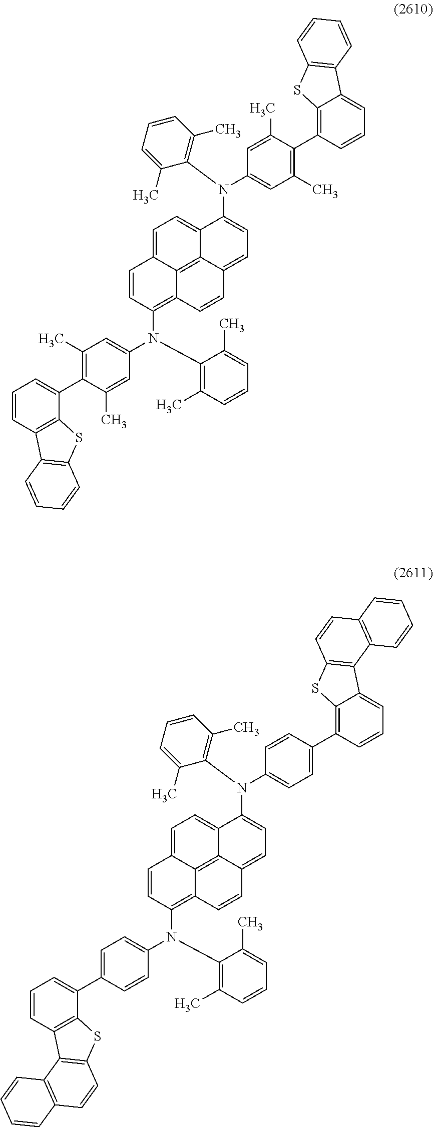 [SMILES: Cc1cc(N(c2c(C)cccc2C)c2ccc3ccc4c(N(c5cc(C)c(-c6cccc7c6sc6ccccc67)c(C)c5)c5c(C)cccc5C)ccc5ccc2c3c54)cc(C)c1-c1cccc2c1sc1ccccc12 ; Cc1cccc(C)c1N(c1ccc(-c2cccc3c2sc2ccc4ccccc4c23)cc1)c1ccc2ccc3c(N(c4ccc(-c5cccc6c5sc5ccc7ccccc7c56)cc4)c4c(C)cccc4C)ccc4ccc1c2c43]

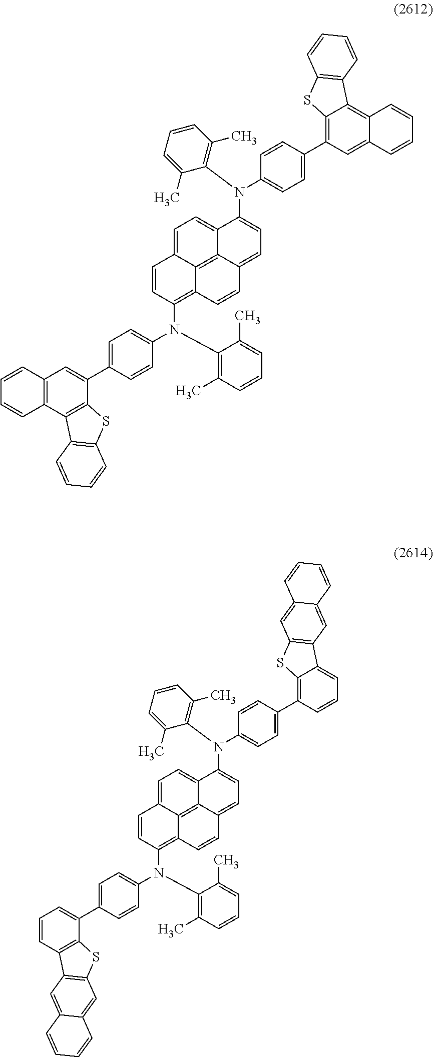 [SMILES: Cc1cccc(C)c1N(c1ccc(-c2cc3ccccc3c3c2sc2ccccc23)cc1)c1ccc2ccc3c(N(c4ccc(-c5cc6ccccc6c6c5sc5ccccc56)cc4)c4c(C)cccc4C)ccc4ccc1c2c43 ; Cc1cccc(C)c1N(c1ccc(-c2cccc3c2sc2cc4ccccc4cc23)cc1)c1ccc2ccc3c(N(c4ccc(-c5cccc6c5sc5cc7ccccc7cc56)cc4)c4c(C)cccc4C)ccc4ccc1c2c43]

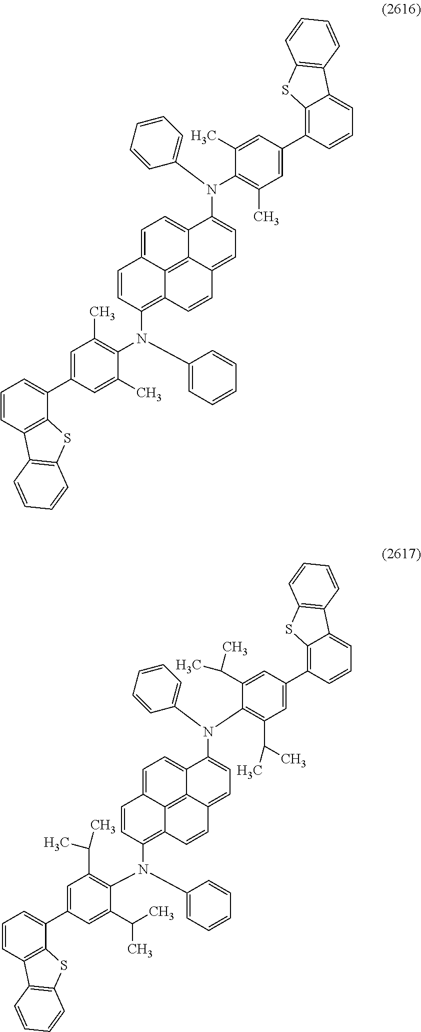 [SMILES: Cc1cc(-c2cccc3c2sc2ccccc23)cc(C)c1N(c1ccccc1)c1ccc2ccc3c(N(c4ccccc4)c4c(C)cc(-c5cccc6c5sc5ccccc56)cc4C)ccc4ccc1c2c43 ; CC(C)c1cc(-c2cccc3c2sc2ccccc23)cc(C(C)C)c1N(c1ccccc1)c1ccc2ccc3c(N(c4ccccc4)c4c(C(C)C)cc(-c5cccc6c5sc5ccccc56)cc4C(C)C)ccc4ccc1c2c43]

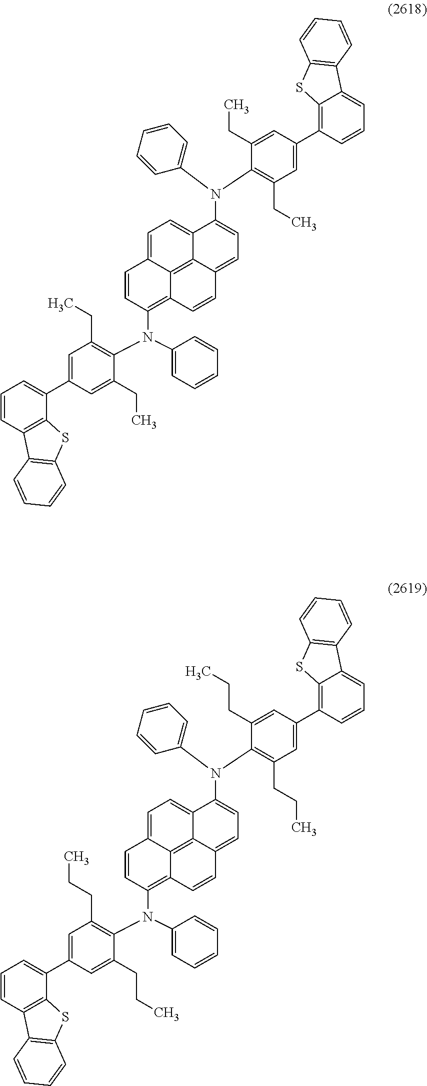 [SMILES: CCc1cc(-c2cccc3c2sc2ccccc23)cc(CC)c1N(c1ccccc1)c1ccc2ccc3c(N(c4ccccc4)c4c(CC)cc(-c5cccc6c5sc5ccccc56)cc4CC)ccc4ccc1c2c43 ; CCCc1cc(-c2cccc3c2sc2ccccc23)cc(CCC)c1N(c1ccccc1)c1ccc2ccc3c(N(c4ccccc4)c4c(CCC)cc(-c5cccc6c5sc5ccccc56)cc4CCC)ccc4ccc1c2c43]

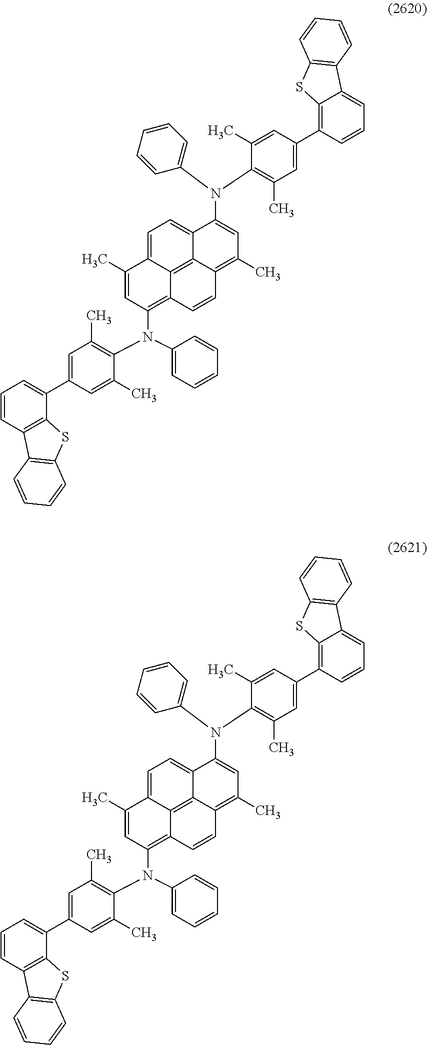 [SMILES: Cc1cc(-c2cccc3c2sc2ccccc23)cc(C)c1N(c1ccccc1)c1cc(C)c2ccc3c(N(c4ccccc4)c4c(C)cc(-c5cccc6c5sc5ccccc56)cc4C)cc(C)c4ccc1c2c43 ; Cc1cc(-c2cccc3c2sc2ccccc23)cc(C)c1N(c1ccccc1)c1cc(C)c2ccc3c(N(c4ccccc4)c4c(C)cc(-c5cccc6c5sc5ccccc56)cc4C)cc(C)c4ccc1c2c43]

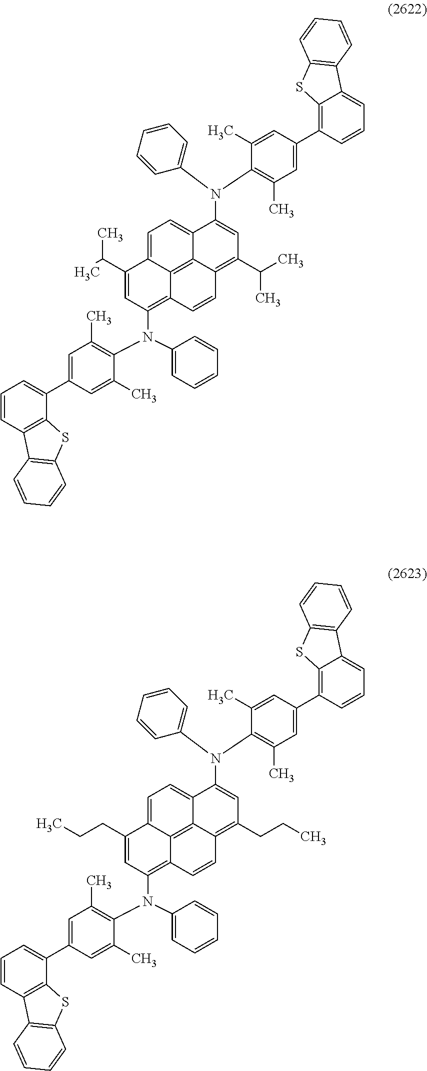 [SMILES: Cc1cc(-c2cccc3c2sc2ccccc23)cc(C)c1N(c1ccccc1)c1cc(C(C)C)c2ccc3c(N(c4ccccc4)c4c(C)cc(-c5cccc6c5sc5ccccc56)cc4C)cc(C(C)C)c4ccc1c2c43 ; CCCc1cc(N(c2ccccc2)c2c(C)cc(-c3cccc4c3sc3ccccc34)cc2C)c2ccc3c(CCC)cc(N(c4ccccc4)c4c(C)cc(-c5cccc6c5sc5ccccc56)cc4C)c4ccc1c2c34]

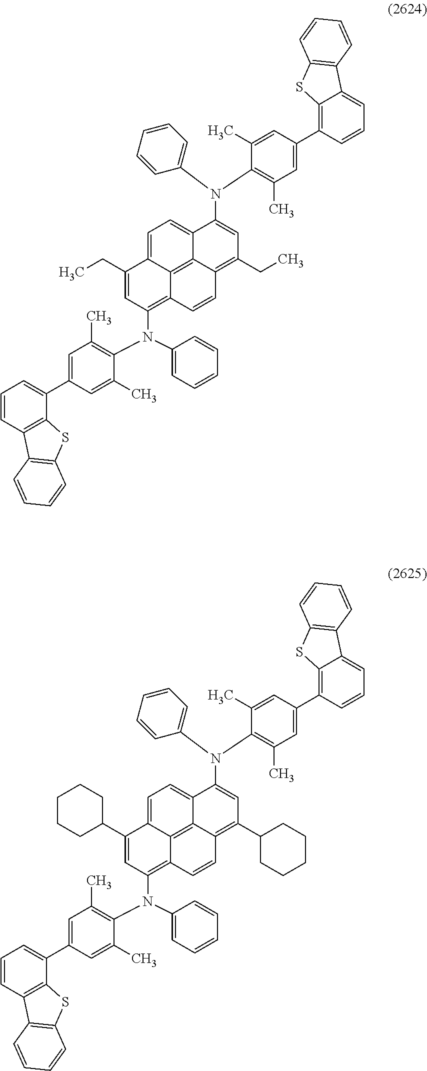 [SMILES: CCc1cc(N(c2ccccc2)c2c(C)cc(-c3cccc4c3sc3ccccc34)cc2C)c2ccc3c(CC)cc(N(c4ccccc4)c4c(C)cc(-c5cccc6c5sc5ccccc56)cc4C)c4ccc1c2c34 ; Cc1cc(-c2cccc3c2sc2ccccc23)cc(C)c1N(c1ccccc1)c1cc(C2CCCCC2)c2ccc3c(N(c4ccccc4)c4c(C)cc(-c5cccc6c5sc5ccccc56)cc4C)cc(C4CCCCC4)c4ccc1c2c43]

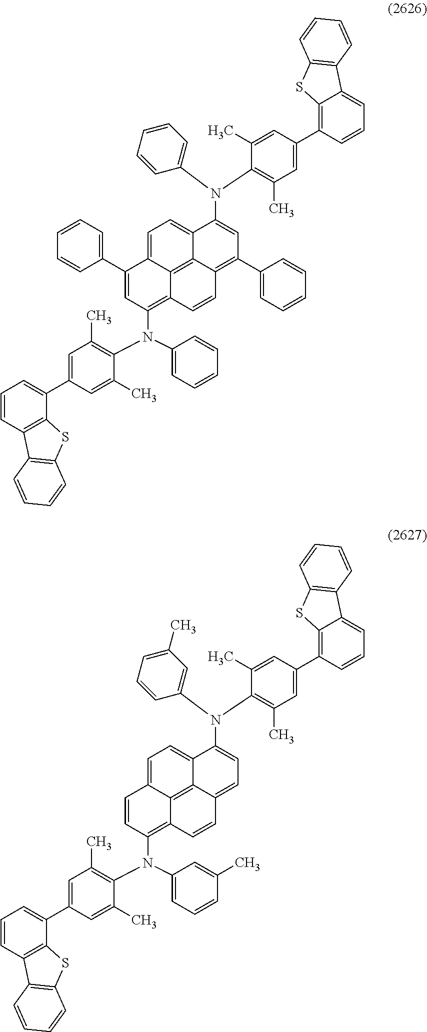 [SMILES: Cc1cc(-c2cccc3c2sc2ccccc23)cc(C)c1N(c1ccccc1)c1cc(-c2ccccc2)c2ccc3c(N(c4ccccc4)c4c(C)cc(-c5cccc6c5sc5ccccc56)cc4C)cc(-c4ccccc4)c4ccc1c2c43 ; Cc1cccc(N(c2c(C)cc(-c3cccc4c3sc3ccccc34)cc2C)c2ccc3ccc4c(N(c5cccc(C)c5)c5c(C)cc(-c6cccc7c6sc6ccccc67)cc5C)ccc5ccc2c3c54)c1]

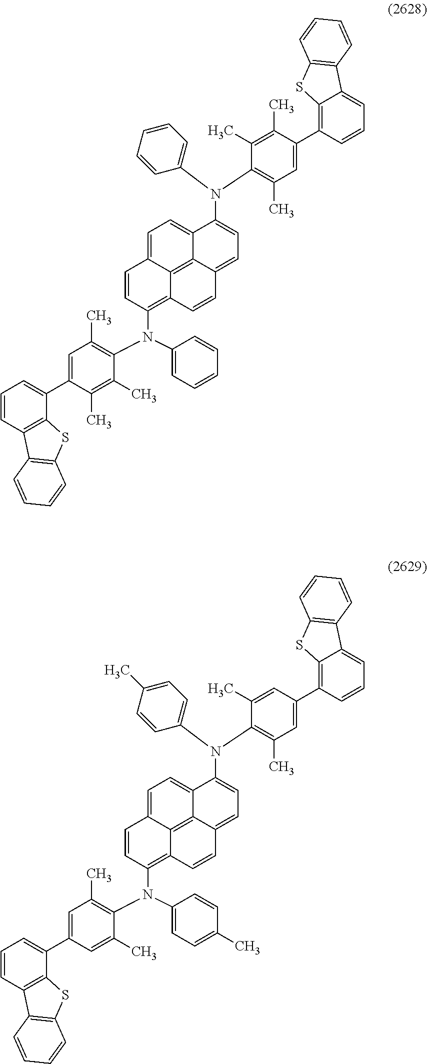 [SMILES: Cc1cc(-c2cccc3c2sc2ccccc23)c(C)c(C)c1N(c1ccccc1)c1ccc2ccc3c(N(c4ccccc4)c4c(C)cc(-c5cccc6c5sc5ccccc56)c(C)c4C)ccc4ccc1c2c43 ; Cc1ccc(N(c2c(C)cc(-c3cccc4c3sc3ccccc34)cc2C)c2ccc3ccc4c(N(c5ccc(C)cc5)c5c(C)cc(-c6cccc7c6sc6ccccc67)cc5C)ccc5ccc2c3c54)cc1]

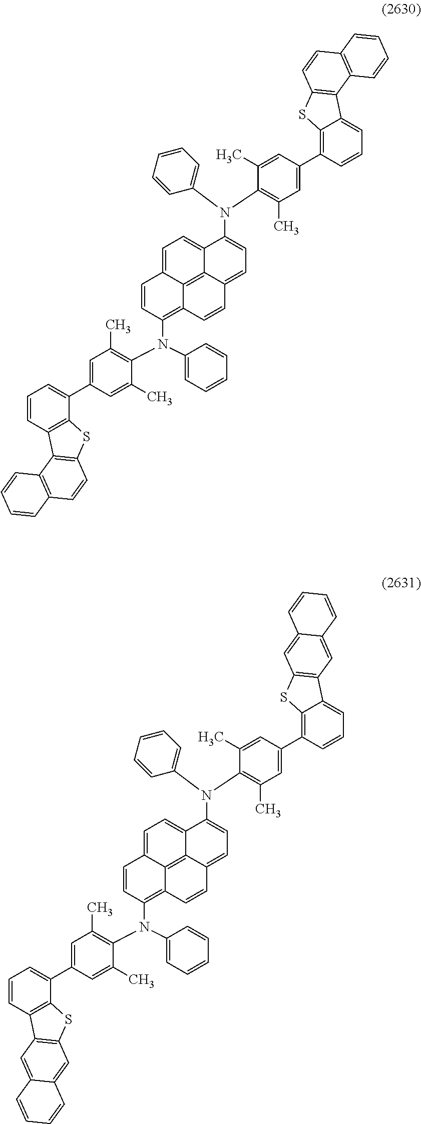 [SMILES: Cc1cc(-c2cccc3c2sc2ccc4ccccc4c23)cc(C)c1N(c1ccccc1)c1ccc2ccc3c(N(c4ccccc4)c4c(C)cc(-c5cccc6c5sc5ccc7ccccc7c56)cc4C)ccc4ccc1c2c43 ; Cc1cc(-c2cccc3c2sc2cc4ccccc4cc23)cc(C)c1N(c1ccccc1)c1ccc2ccc3c(N(c4ccccc4)c4c(C)cc(-c5cccc6c5sc5cc7ccccc7cc56)cc4C)ccc4ccc1c2c43]

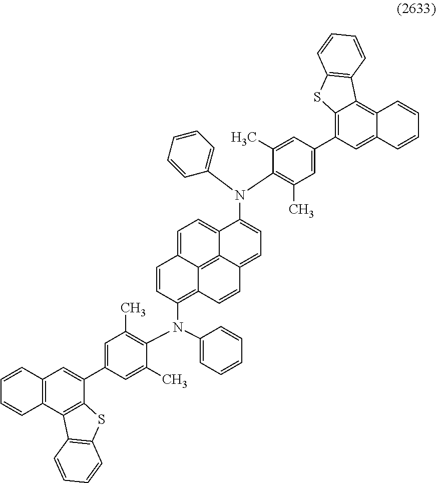 [SMILES: Cc1cc(-c2cc3ccccc3c3c2sc2ccccc23)cc(C)c1N(c1ccccc1)c1ccc2ccc3c(N(c4ccccc4)c4c(C)cc(-c5cc6ccccc6c6c5sc5ccccc56)cc4C)ccc4ccc1c2c43]

A variety of reactions can be employed as a method of synthesizing any of the organic compounds of embodiments of the present invention described above. The organic compounds represented by General Formula (G2-1) or General Formula (G2-2) can be synthesized through the following synthesis scheme, for example.

First, as shown in Synthesis Scheme (A-1), an aryl compound having a halogen group (a1) and an aryl compound having an amine (a2) are coupled, whereby an amine derivative (a3) can be obtained.

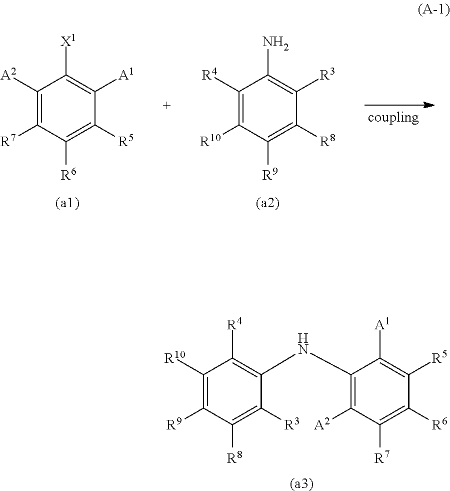

Note that X¹ in Synthesis Scheme (A-1) represents a halogen, preferably bromine or iodine, which has high reactivity, more preferably iodine.

In Synthesis Scheme (A-1), there are a variety of reaction conditions for the coupling reaction of an aryl compound having a halogen group and an aryl compound having amine (primary arylamine compound); for example, a synthesis method using a metal catalyst in the presence of a base can be applied.

The case where the Buchwald-Hartwig reaction is performed in Synthesis Scheme (A-1) is described. A palladium catalyst can be used as the metal catalyst, and a mixture of a palladium complex and a ligand thereof can be used as the palladium catalyst. Examples of the palladium complex include bis(dibenzylideneacetone)palladium(0), palladium(II) acetate, and tetrakis(triphenylphosphine)palladium(0). Examples of the ligand include tri(tert-butyl)phosphine, tri(n-hexyl)phosphine, tricyclohexylphosphine, 1,1′-bis(diphenylphosphino)ferrocene (abbreviation: DPPF), di(1-adamantyl)-n-butylphosphine, and tris(2,6-dimethoxyphenyl)phosphine. Examples of a substance that can be used as the base include organic bases such as sodium tert-butoxide, inorganic bases such as potassium carbonate, tripotassium phosphate, and cesium carbonate. In addition, this reaction is preferably performed in a solution, and examples of the solvent that can be used are toluene, xylene, benzene, and mesitylene. However, the catalyst, ligand, base, and solvent which can be used are not limited thereto. In addition, the reaction is preferably performed under an inert atmosphere of nitrogen, argon, or the like.

The case where an Ullmann reaction is used in Synthesis Scheme (A-1) is described. A copper catalyst can be used as the metal catalyst, and copper(I) iodide and copper(II) acetate are given as the copper catalyst. As an example of a substance which can be used for the base, an inorganic base such as potassium carbonate is given. The reaction is preferably performed in a solution, and examples of the solvent that can be used are 1,3-dimethyl-3,4,5,6-tetrahydro-2(1H)pyrimidinone (abbreviation: DMPU), toluene, xylene, benzene, mesitylene, and the like. However, the catalyst, base, and solvent which can be used are not limited to these examples. In addition, the reaction is preferably performed under an inert atmosphere of nitrogen, argon, or the like.

Note that a solvent having a high boiling point such as DMPU, xylene, or mesitylene is preferably used because, in an Ullmann reaction, a target substance can be obtained in a shorter time and at a higher yield when the reaction temperature is 100° C. or higher. A reaction temperature higher than 150° C. is further preferred and accordingly DMPU or mesitylene is more preferably used.

Next, as shown in Synthesis Scheme (A-2), the amine derivative (a3) and a halogenated arene derivative typified by halogenated pyrene derivative (a4) are coupled, so that an amine derivative represented by General Formula (G2-1) or (G2-2) can be obtained.

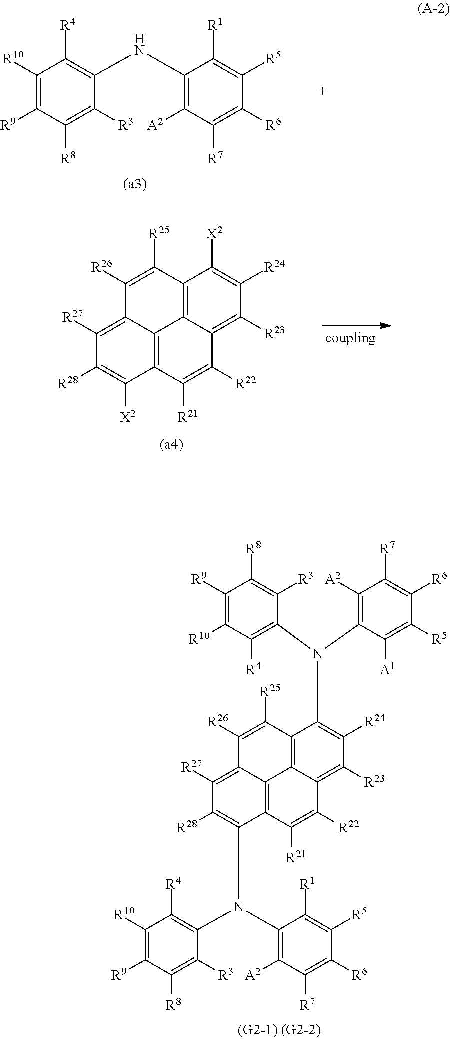

X² represents a halogen, preferably bromine or iodine, which has high reactivity, more preferably iodine. In that case, two equivalents of the amine derivative (a3) are reacted with the halogenated pyrene derivative (a4).

Note that in Synthesis Schemes (A-1) and (A-2), A¹ and A² each represent an alkyl group having 1 to 6 carbon atoms; at least one of R³ and R⁴ represents hydrogen and the other represents hydrogen or an alkyl group having 1 to 6 carbon atoms; and R⁵ to R¹⁰ and R²¹ to R²⁸ each independently represent hydrogen, an alkyl group having 1 to 6 carbon atoms, or an aryl group having 6 to 25 carbon atoms. Note that any one of R⁵ to R¹⁰ is a substituent represented by General Formula (g1-1) or (g1-2).

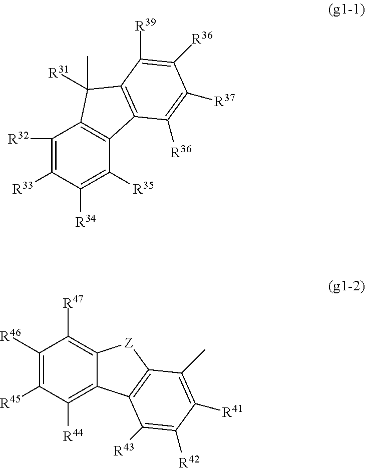

In General Formula (g1-1), R³¹ to R³⁹ each independently represent hydrogen, an alkyl group having 1 to 6 carbon atoms, or an aryl group having 6 to 25 carbon atoms.

In General Formula (g1-2), R⁴¹ to R⁴⁷ each independently represent hydrogen, an alkyl group having 1 to 6 carbon atoms, or an aryl group having 6 to 25 carbon atoms; and Z represents an oxygen atom or a sulfur atom.

In Synthesis Scheme (A-2), there are a variety of reaction conditions for the coupling reaction of an aryl compound having a halogen group and an aryl compound having amine (primary arylamine compound or a secondary arylamine compound); for example, a synthesis method using a metal catalyst in the presence of a base can be applied. Note that a Hartwig-Buchwald reaction or an Ullmann reaction can be employed in Synthesis Scheme (A-2) as in Synthesis Scheme (A-1).

To synthesize an organic compound of one embodiment of the present invention other than the organic compounds represented by General Formulae (G2-1) and (G2-2) in which substituents bonded to a pyrene skeleton are symmetric, different diphenylamine units may be bonded to the pyrene skeleton in Synthesis Scheme (A-2).

### <<Calculation Results and Consideration of Reduction in Spectrum Width>>

A 1,6-bis(diphenylamino)pyrene derivative has two diphenylamino groups, and each diphenylamino group has two phenyl groups. A 1,6-bis(diphenylamino)pyrene derivative in which an alkyl group is bonded to each of the two ortho positions of at least one of the two phenyl groups in each of the two diphenylamino groups has a narrower half width of an emission spectrum than a 1,6-bis(diphenylamino)pyrene derivative without the above structure. The reason is described below using the calculation results. Note that calculation was performed on N,N,N′,N′-tetraphenylpyrene-1,6-diamine in which alkyl groups are not bonded to the ortho positions (Calculation Model 1) and N,N-bis(2,6-dimethylphenyl)-N,N′-diphenylpyrene-1,6-diamine in which alkyl groups are bonded to the ortho positions (Calculation Model 2).

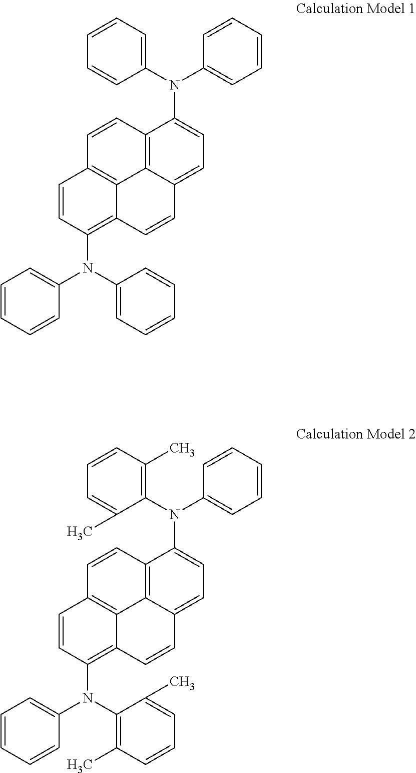 [SMILES: c1ccc(N(c2ccccc2)c2ccc3ccc4c(N(c5ccccc5)c5ccccc5)ccc5ccc2c3c54)cc1 ; Cc1cccc(C)c1N(c1ccccc1)c1ccc2ccc3c(N(c4ccccc4)c4c(C)cccc4C)ccc4ccc1c2c43]

First, structural optimization was performed on an excited state S1 and a ground state S0 of each of the above two models. A high performance computer (ICE X, manufactured by SGI Japan, Ltd.) was used for the calculation. Gaussian 09 was used as the quantum chemistry calculation program. The calculation method is as follows.

First, the most stable structure in the ground state S0 was calculated using the density functional theory. As a basis function, 6-311G (a basis function of a triple-split valence basis set using three contraction functions for each valence orbital) was applied to all the atoms. By the above basis function, for example, 1s to 3s orbitals are considered in the case of hydrogen atoms, while 1s to 4s and 2p to 4p orbitals are considered in the case of carbon atoms. To improve calculation accuracy, the p function and the d function as polarization basis sets were added respectively to hydrogen atoms and atoms other than hydrogen atoms. As a functional, B3LYP was used. The most stable structure in the excited state S1 is calculated by the time-dependent density functional theory on the basis of the most stable structure in the ground state S0. For the calculation, the same base function and functional used for the calculation of structure optimization in the ground state S0 are used.

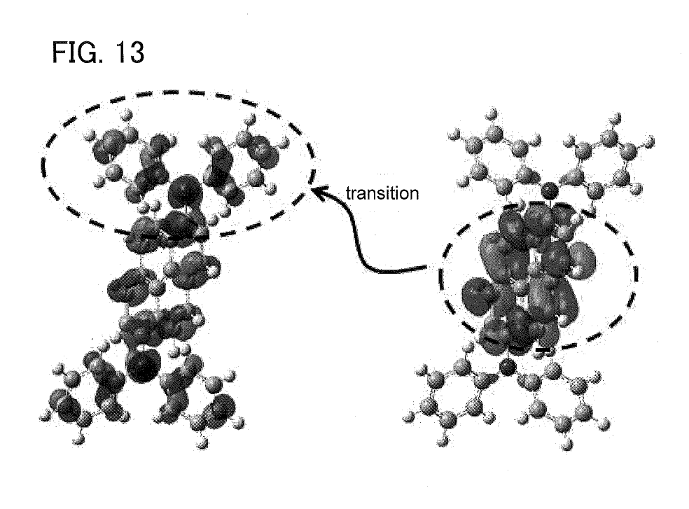

FIG. 13 shows main molecular orbitals that relates to the excited state S1 of Calculation Models 1 and 2 which are obtained by the calculations. FIG. 13 indicates that at the transition from the excited state S1 to the ground state S0 in Calculation Model 1, electrons transfer from a pyrene skeleton to a diphenylamine skeleton. This means that the structure of the diphenylamine skeleton changes as the structure of the pyrene skeleton changes. The structural change probably occurs in Calculation Model 2 as in Calculation Model 1.

In general, as a structural change by transition (Stokes shift) becomes larger, the transition number of vibrational levels is increased, so that the emission spectrum becomes broad. In terms of the relationship between a Stokes shift and a vibrational structure, the degree of structural change by transition between the ground state S0 and the excited state S1 in Calculation Model 1 and Calculation Model 2 is calculated.

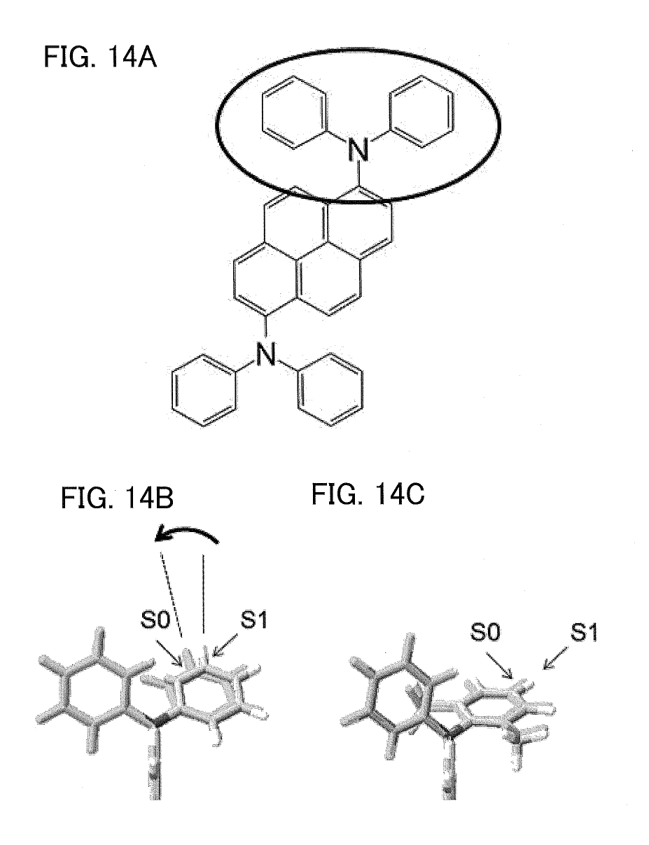

FIGS. 14A to 14C show the most stable structures, which are obtained by calculations, in the ground state S0 and the excited state S1 of Calculation Model 1 and Calculation Model 2. The most stable structures are overlapped with a pyrene skeleton.

FIGS. 14A to 14C indicate that in Calculation Model 1, a phenyl group of a diphenylamine skeleton broadly moves at the transition between the ground state S0 and the excited state S1. In contrast, in Calculation Model 2, movement of a phenyl group is suppressed by steric hindrance of a methyl group. In other words, the structural change by the transition in Calculation Model 2 is smaller (i.e., Stokes shift is smaller) than that in Calculation Model 1. This indicates that the emission spectrum of Calculation Model 2 is narrowed.

Next, to measure the degree of the structural change quantitatively, rearrangement energy λ(S0) and rearrangement energy λ(S1), which are released when structural relaxation is performed in the ground state S0 and the excited state S1, are obtained. Table 1 shows the calculation results.

TABLE 1

| | Calculation model 1 | Calculation model 2 |
|---|---|---|
| λ (S0) | 0.138 eV | 0.121 eV |
| λ (S1) | 0.142 eV | 0.126 eV |
| λ (S0) + λ (S1) | 0.281 eV | 0.247 eV |

Table 1 shows that the rearrangement energy of Calculation Model 2 is 10% smaller than that of Calculation Model 1. That is, structural change is suppressed in Calculation Model 2.

According to the above results, the 1,6-bis(diphenylamino)pyrene derivative (corresponds to Calculation Model 2) in which an alkyl group is bonded to each of the two ortho positions of at least one of the two phenyl groups in each of the two diphenylamino groups has a narrower half width of an emission spectrum than the 1,6-bis(diphenylamino)pyrene derivative without the above structure (corresponds to Calculation Model 1).

### <<Light-Emitting Element>>

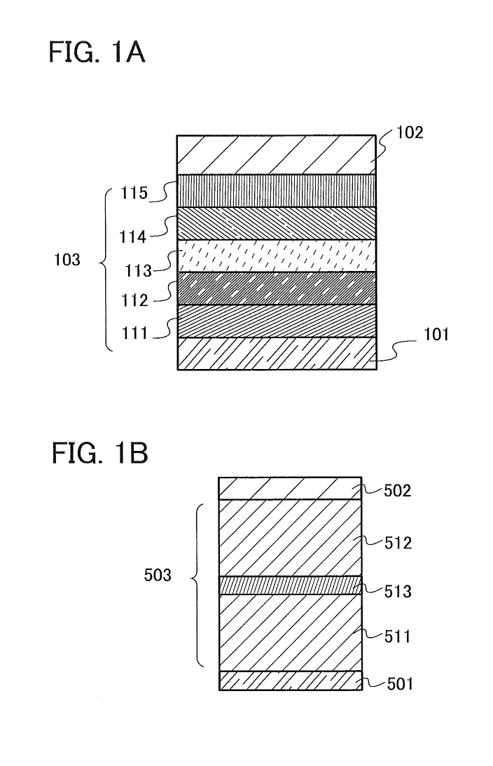

Next, an example of a light-emitting element which is one embodiment of the present invention is described in detail below with reference to FIG. 1A.

In this embodiment, the light-emitting element includes a pair of electrodes (a first electrode 101 and a second electrode 102), and an EL layer 103 provided between the first electrode 101 and the second electrode 102. Note that the first electrode 101 functions as an anode and that the second electrode 102 functions as a cathode.

Since the first electrode 101 functions as an anode, it is preferably formed using any of metals, alloys, electrically conductive compounds having a high work function (specifically, a work function of 4.0 eV or more), mixtures thereof, and the like. Specific examples include indium oxide-tin oxide (ITO: indium tin oxide), indium oxide-tin oxide containing silicon or silicon oxide, indium oxide-zinc oxide, and indium oxide containing tungsten oxide and zinc oxide (IWZO). Films of such electrically conductive metal oxides are usually formed by a sputtering method, but may be formed by application of a sol-gel method or the like. In an example of the formation method, indium oxide-zinc oxide is deposited by a sputtering method using a target obtained by adding 1 wt % to 20 wt % of zinc oxide to indium oxide. Further, a film of indium oxide containing tungsten oxide and zinc oxide (IWZO) can be formed by a sputtering method using a target in which tungsten oxide and zinc oxide are added to indium oxide at 0.5 wt % to 5 wt % and 0.1 wt % to 1 wt %, respectively. Another examples are gold (Au), platinum (Pt), nickel (Ni), tungsten (W), chromium (Cr), molybdenum (Mo), iron (Fe), cobalt (Co), copper (Cu), palladium (Pd), nitrides of metal materials (e.g., titanium nitride), and the like. Graphene can also be used. Note that when a composite material described later is used for a layer which is in contact with the first electrode 101 in the EL layer 103, an electrode material can be selected regardless of its work function.

It is preferable that the EL layer 103 be formed of stacked layers and the 1,6-bis(diphenylamino)pyrene derivative be contained in any of the stacked layers. Note that the 1,6-bis(diphenylamino)pyrene derivative is preferably used as an emission center substance in a light-emitting layer. The 1,6-bis(diphenylamino)pyrene derivative is preferably an organic compound represented by General Formula (G1-1), (G1-2), (G2-1), or (G2-2).

The stacked layer structure of the EL layer 103 can be formed by combining a hole-injection layer, a hole-transport layer, a light-emitting layer, an electron-transport layer, an electron-injection layer, a carrier-blocking layer, an intermediate layer, and the like as appropriate. In this embodiment, the EL layer 103 has a structure in which a hole-injection layer 111, a hole-transport layer 112, a light-emitting layer 113, an electron-transport layer 114, and an electron-injection layer 115 are stacked in this order over the first electrode 101. Specific examples of the materials forming the layers are given below.

The hole-injection layer 111 is a layer that contains a substance having a high hole-injection property. Molybdenum oxide, vanadium oxide, ruthenium oxide, tungsten oxide, manganese oxide, or the like can be used. Alternatively, the hole-injection layer 111 can be formed using a phthalocyanine-based compound such as phthalocyanine (abbreviation: H₂Pc) or copper phthalocyanine (abbreviation: CuPc), an aromatic amine compound such as 4,4′-bis[N-(4-diphenylaminophenyl)-N-phenylamino]biphenyl (abbreviation: DPAB) or N,N′-bis{4-[bis(3-methylphenyl)amino]phenyl}-N,N′-diphenyl-(1,1′-biphenyl)-4,4′-diamine (abbreviation: DNTPD), a high molecular compound such as poly(3,4-ethylenedioxythiophene)/poly(styrenesulfonic acid) (PEDOT/PSS), or the like.

Alternatively, a composite material in which a substance having a hole-transport property contains a substance having an acceptor property can be used for the hole-injection layer 111. Note that the use of such a substance having a hole-transport property which contains a substance having an acceptor property enables selection of a material used to form an electrode regardless of its work function. In other words, besides a material having a high work function, a material having a low work function can be used for the first electrode 101. As the substance having an acceptor property, 7,7,8,8-tetracyano-2,3,5,6-tetrafluoroquinodimethane (abbreviated to F₄-TCNQ), chloranil, and the like can be given. In addition, transition metal oxides can be given. Moreover, oxides of metals belonging to Groups 4 to 8 of the periodic table can be given. Specifically, it is preferable to use vanadium oxide, niobium oxide, tantalum oxide, chromium oxide, molybdenum oxide, tungsten oxide, manganese oxide, and rhenium oxide because of their high electron accepting properties. In particular, molybdenum oxide is more preferable because of its stability in the atmosphere, low hygroscopic property, and easiness of handling.

As the substance having a hole-transport property which is used for the composite material, any of a variety of organic compounds such as aromatic amine compounds, carbazole derivatives, aromatic hydrocarbons, and high molecular compounds (e.g., oligomers, dendrimers, or polymers) can be used. Note that the organic compound used for the composite material is preferably an organic compound having a high hole-transport property. Specifically, a substance having a hole mobility of 10⁻⁶ cm²/Vs or more is preferably used. Organic compounds that can be used as the substance having a hole-transport property in the composite material are specifically given below.

Examples of the aromatic amine compounds are N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), and the like.

Specific examples of the carbazole derivatives that can be used for the composite material are 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), and the like.

Other examples of the carbazole derivatives that can be used for the composite material are 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), 1,4-bis[4-(N-carbazolyl)phenyl]-2,3,5,6-tetraphenylbenzene, and the like.

Examples of the aromatic hydrocarbons that can be used for the composite material are 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, 2,5,8,11-tetra(tert-butyl)perylene, and the like. Besides, pentacene, coronene, or the like can also be used. The aromatic hydrocarbon which has a hole mobility of 1×10⁻⁶ cm²/Vs or more and which has 14 to 42 carbon atoms is particularly preferable.

Note that the aromatic hydrocarbons that can be used for the composite material may have a vinyl skeleton. Examples of the aromatic hydrocarbon having a vinyl group are 4,4′-bis(2,2-diphenylvinyl)biphenyl (abbreviation: DPVBi), 9,10-bis[4-(2,2-diphenylvinyl)phenyl]anthracene (abbreviation: DPVPA), and the like.

A high molecular compound such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), or poly[N,N′-bis(4-butylphenyl)-N,N′-bis(phenyl)benzidine] (abbreviation: poly-TPD) can also be used.

By providing the hole-injection layer 111, a high hole-injection property can be achieved to allow a light-emitting element to be driven at a low voltage.

The hole-transport layer 112 is a layer that contains a substance having a hole-transport property. Examples of the substance having a hole-transport property are aromatic amine compounds such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), and the like. The substances mentioned here have high hole-transport properties and are mainly ones that have a hole mobility of 10⁻⁶ cm²/Vs or more. An organic compound given as an example of the substance having a hole-transport property in the composite material described above can also be used for the hole-transport layer 112. A high molecular compound such as poly(N-vinylcarbazole) (abbreviation: PVK) or poly(4-vinyltriphenylamine) (abbreviation: PVTPA) can also be used. Note that the layer that contains a substance having a hole-transport property is not limited to a single layer, and may be a stack of two or more layers including any of the above substances.

The light-emitting layer 113 may be a layer that emits fluorescence, a layer that emits phosphorescence, or a layer emitting thermally activated delayed fluorescence (TADF).

Furthermore, the light-emitting layer 113 may be a single layer or include a plurality of layers containing different light-emitting substances.

A light-emitting material with a small Stokes shift is preferably used for the light-emitting layer 113. The use of the light-emitting material with a small Stokes shift brings many preferable effects described above.

The aforementioned 1,6-bis(diphenylamino)pyrene derivative is preferably used as a phosphorescent substance. The 1,6-bis(diphenylamino)pyrene derivative preferably has a structure in which an alkyl group is bonded to each of the two ortho positions of one phenyl group, and hydrogen is bonded to each of the two ortho positions of the other phenyl group. The 1,6-bis(diphenylamino)pyrene derivative with such a structure is easily synthesized. The 1,6-bis(diphenylamino)pyrene derivative is preferably an organic compound represented by General Formula (G1-1), (G1-2), (G2-1), or (G2-2).

A light-emitting element that contains the 1,6-bis(diphenylamino)pyrene derivative as a phosphorescent substance can emit excellent blue light. For example, the light-emitting element can emit blue light with a y-coordinate of the CIE chromaticity of 0.15 or smaller. The half width of light from the light-emitting element can be less than or equal to 40 nm, ideally less than or equal to 35 nm. The peak wavelength of light from the light-emitting element can be less than or equal to 465 nm.

In the case where the 1,6-bis(diphenylamino)pyrene derivative is not used as a phosphorescent substance, for example, materials given below can be used as the phosphorescent substance. Fluorescent substances other than the materials given below can also be used.

Examples of the fluorescent substance are 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9-phenyl-9H-fluoren-9-yl)phenyl]-N,N′-diphenylpyrene-1,6-diamine (abbreviation: 1,6FLPAPrn), N,N′-bis(3-methylphenyl)-N,N′-bis[3-(9-phenyl-9H-fluoren-9-yl)phenyl]-pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chhrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), {2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), {2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), and the like. Condensed aromatic diamine compounds typified by pyrenediamine compounds such as 1,6FLPAPrn and 1,6mMemFLPAPrn are preferable because of their high hole-trapping properties, high emission efficiency, and high reliability.

Examples of a material which can be used as a phosphorescent light-emitting substance in the light-emitting layer 113 are as follows.

The examples include organometallic iridium complexes having 4H-triazole skeletons, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); organometallic iridium complexes having 1H-triazole skeletons, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes having imidazole skeletons, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and organometallic iridium complexes in which a phenylpyridine derivative having an electron-withdrawing group is a ligand, such as bis[2-(4′,6′-difluorophenyl)pyridinato-N,C²′]iridium(III) tetrakis(1-pyrazolyl)borate (abbreviation: FIr6), bis[2-(4′,6′-difluorophenyl)pyridinato-N,C²′]iridium(III) picolinate (abbreviation: FIrpic), bis{2-[3′,5′-bis(trifluoromethyl)phenyl]pyridinato-N,C²′}iridium(III) picolinate (abbreviation: [Ir(CF₃ppy)₂(pic)]), and bis[2-(4′,6′-difluorophenyl)pyridinato-N,C²′]iridium(III) acetylacetonate (abbreviation: FIracac). These are compounds emitting blue phosphorescent light and have an emission peak at 440 nm to 520 nm.

Other examples include organometallic iridium complexes having pyrimidine skeletons, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having pyrazine skeletons, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having pyridine skeletons, such as tris(2-phenylpyridinato-N,C²′)iridium(III) (abbreviation: [Ir(ppy)₃]), bis(2-phenylpyridinato-N,C²′)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²′)iridium(III) (abbreviation: [Ir(pq)₃]), and bis(2-phenylquinolinato-N,C²′)iridium(III) acetylacetonate (abbreviation: [Ir(pq)₂(acac)]); and rare earth metal complexes such as tris(acetylacetonato)(monophenanthroline)terbium(III) (abbreviation: [Tb(acac)₃(Phen)]). These are mainly compounds emitting green phosphorescent light and have an emission peak at 500 nm to 600 nm. Note that organometallic iridium complexes having pyrimidine skeletons have distinctively high reliability and emission efficiency and thus are especially preferable.

Other examples include organometallic iridium complexes having pyrimidine skeletons, such as (diisobutyrylmethanato)bis[4,6-bis(3-methylphenyl)pyrimidinato]iridium(III) (abbreviation: [Ir(5mdppm)₂(dpm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having pyrazine skeletons, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having pyridine skeletons, such as tris(1-phenylisoquinolinato-N,C²′)iridium(III) (abbreviation: [Ir(piq)₃]) and bis(1-phenylisoquinolinato-N,C²′)iridium(III) acetylacetonate (abbreviation: [Ir(piq)₂(acac)]); platinum complexes such as 2,3,7,8,12,13,17,18-octaethyl-21H,23H-porphyrin platinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These are compounds emitting red phosphorescent light and have an emission peak at 600 nm to 700 mm. Further, organometallic iridium complexes having pyrazine skeletons can provide red light emission with favorable chromaticity.

As well as the above phosphorescent compounds, a variety of phosphorescent light-emitting substances may be selected and used.

Materials that can be used as a TADF material (a material emitting TADF), are given below.

As a material exhibiting TADF, materials given below can be used. A fullerene, a derivative thereof, an acridine derivative such as proflavine, and eosin can be given. Further, a metal-containing porphyrin, such as a porphyrin containing magnesium (Mg), zinc (Zn), cadmium (Cd), tin (Sn), platinum (Pt), indium (In), or palladium (Pd) can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂(OEP)), which are shown in the following structural formulae.

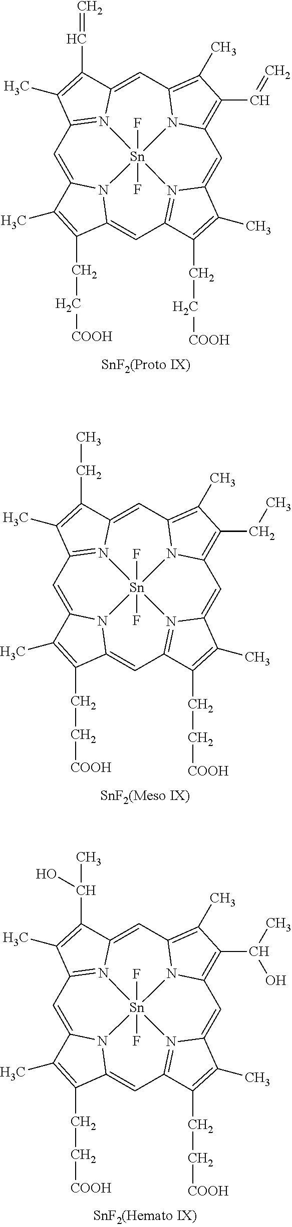

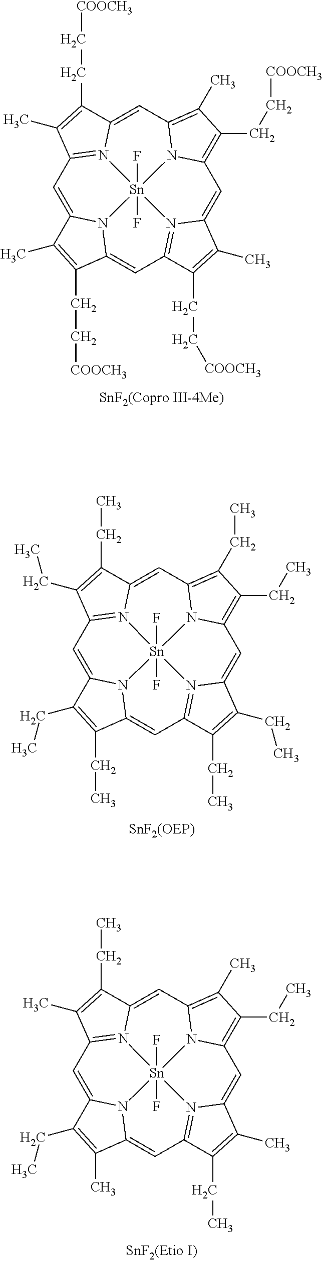

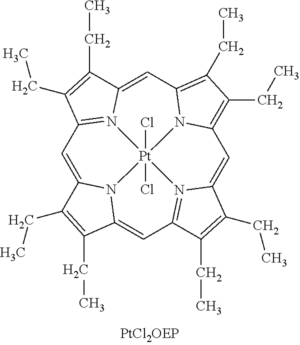

Alternatively, a heterocyclic compound having a π-electron rich heteroaromatic ring and a π-electron deficient heteroaromatic ring, such as 2-(biphenyl-4-yl)-4,6-bis(12-phenylindolo[2,3-a]carbazol-11-yl)-1,3,5-triazine (abbreviation: PIC-TRZ) shown in the following structural formula, can be used. The heterocyclic compound is preferably used because of the π-electron rich heteroaromatic ring and the π-electron deficient heteroaromatic ring, for which the electron-transport property and the hole-transport property are high. Note that a substance in which the π-electron rich heteroaromatic ring is directly bonded to the π-electron deficient heteroaromatic ring is particularly preferably used because the donor property of the π-electron rich heteroaromatic ring and the acceptor property of the π-electron deficient heteroaromatic ring are both high and the difference between the S₁ level and the T₁ level becomes small.

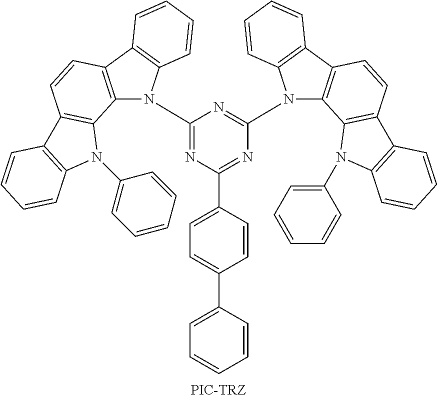 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

In the case of using the 1,6-bis(diphenylamino)pyrene derivative, materials that can be suitably used as the host material in the light-emitting layer are materials having an anthracene skeleton such as 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), and 9-phenyl-10-{4-(9-phenyl-9H-fluoren-9-yl)biphenyl-4′-yl}anthracene (abbreviation: FLPPA). The use of a substance having an anthracene skeleton as the host material makes it possible to obtain a light-emitting layer with high emission efficiency and high durability. In particular, CzPA, cgDBCzPA, 2mBnfPPA, and PCzPA are preferable because of their excellent characteristics.

In the case where a material other than the above-mentioned materials is used as a host material, various carrier-transport materials, such as a material having an electron-transport property or a material having a hole-transport property, can be used.

Examples of the material having an electron-transport property are a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); a heterocyclic compound having a polyazole skeleton such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), or 2-[3-(dibenzothiophen-4-yl)phenyl]-1-phenyl-1H-benzimidazole (abbreviation: mDBTBIm-II); a heterocyclic compound having a diazine skeleton such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Prn), or 4,6-bis[3-(4-dibenzothienyl)phenyl]pyrimidine (abbreviation: 4,6mDBTP2Prn-II); and a heterocyclic compound having a pyridine skeleton such as 3,5-bis[3-(9H-carbazol-9-yl)phenyl]pyridine (abbreviation: 35DCzPPy) or 1,3,5-tri[3-(3-pyridyl)-phenyl]benzene (abbreviation: TmPyPB). Among the above materials, a heterocyclic compound having a diazine skeleton and a heterocyclic compound having a pyridine skeleton have high reliability and are thus preferable. Specifically, a heterocyclic compound having a diazine (pyrimidine or pyrazine) skeleton has a high electron-transport property to contribute to a reduction in drive voltage.

Examples of the material having a hole-transport property include a compound having an aromatic amine skeleton such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]-fluoren-2-amine (abbreviation: PCBAF), or N-phenyl-N-[4-(9-phenyl-9H-carbazol-3-yl)phenyl]-spiro-9,9′-bifluoren-2-amine (abbreviation: PCBASF); a compound having a carbazole skeleton such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP); a compound having a thiophene skeleton such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); and a compound having a furan skeleton such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II). Among the above materials, a compound having an aromatic amine skeleton and a compound having a carbazole skeleton are preferable because these compounds are highly reliable and have high hole-transport properties to contribute to a reduction in drive voltage. Hole-transport materials can be selected from a variety of substances as well as from the hole-transport materials given above.

Note that the host material may be a mixture of a plurality of kinds of substances, and in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The ratio of the content of the material having a hole-transport property to the content of the material having an electron-transport property may be 1:9 to 9:1.

These mixed host materials may form an exciplex. When a combination of these materials is selected so as to form an exciplex that exhibits light emission whose wavelength overlaps the wavelength of a lowest-energy-side absorption band of the fluorescent substance, the phosphorescent substance, or the TADF material, energy is transferred smoothly and light emission can be obtained efficiently. Such a combination is preferable in that drive voltage can be reduced.

A more desirable structure of the light-emitting element is that an absorption spectrum peak on the longest wavelength side of the light-emitting material with a small Stokes shift overlaps with an emission spectrum of the host material. The absorption spectrum peak on the longest wavelength side of the light-emitting material shows absorption with the lowest energy in the light-emitting material. The light-emitting material with a small Stokes shift can be excited with low energy as compared with a normal light-emitting material. Therefore, with a host material whose emission spectrum overlaps with the absorption in the lowest energy region of the light-emitting material with a small Stokes shift, the most advantageous structure in energy efficiency can be obtained.

The light-emitting layer 113 having the above-described structure can be formed by co-evaporation by a vacuum evaporation method, or an inkjet method, a spin coating method, a dip coating method, or the like using a mixed solution.

The electron-transport layer 114 contains a material having an electron-transport property. For the electron-transport layer 114, the materials having an electron-transport property or having an anthracene skeleton, which are described above as materials for the host material, can be used.

Between the electron-transport layer and the light-emitting layer, a layer that controls transport of electron carriers may be provided. This is a layer formed by addition of a small amount of a substance having a high electron-trapping property to the aforementioned material having a high electron-transport property, and the layer is capable of adjusting carrier balance by retarding transport of electron carriers. Such a structure is very effective in preventing a problem (such as a reduction in element lifetime) caused when electrons pass through the light-emitting layer.

In addition, the electron-injection layer 115 may be provided in contact with the second electrode 102 between the electron-transport layer 114 and the second electrode 102. For the electron-injection layer 115, an alkali metal, an alkaline earth metal, or a compound thereof, such as lithium fluoride (LiF), cesium fluoride (CsF), or calcium fluoride (CaF₂), can be used. For example, a layer that is formed using a substance having an electron-transport property and contains an alkali metal, an alkaline earth metal, or a compound thereof can be used. In addition, an electride may be used for the electron-injection layer 115. Examples of the electride include a substance in which electrons are added at high concentration to calcium oxide-aluminum oxide. Note that a layer that is formed using a substance having an electron-transport property and contains an alkali metal or an alkaline earth metal is preferably used as the electron-injection layer 115, in which case electron injection from the second electrode 102 is efficiently performed.

For the second electrode 102, any of metals, alloys, electrically conductive compounds, and mixtures thereof which have a low work function (specifically, a work function of 3.8 eV or less) or the like can be used. Specific examples of such a cathode material are elements belonging to Groups 1 and 2 of the periodic table, such as alkali metals (e.g., lithium (Li) and cesium (Cs)), magnesium (Mg), calcium (Ca), and strontium (Sr), alloys thereof (e.g., MgAg and AlLi), rare earth metals such as europium (Eu) and ytterbium (Yb), alloys thereof, and the like. However, when the electron-injection layer is provided between the second electrode 102 and the electron-transport layer 114, for the second electrode 102, any of a variety of conductive materials such as Al, Ag, ITO, or indium oxide-tin oxide containing silicon or silicon oxide can be used regardless of the work function. Films of these electrically conductive materials can be formed by a sputtering method, an inkjet method, a spin coating method, or the like.

Any of a variety of methods can be used to form the EL layer 103 regardless whether it is a dry process or a wet process. For example, a vacuum evaporation method, an inkjet method, a spin coating method, or the like may be used. Different formation methods may be used for the electrodes or the layers.

In addition, the electrode may be formed by a wet method using a sol-gel method, or by a wet method using paste of a metal material. Alternatively, the electrode may be formed by a dry method such as a sputtering method or a vacuum evaporation method.

Light emission from the light-emitting element is extracted out through one or both of the first electrode 101 and the second electrode 102. Therefore, one or both of the first electrode 101 and the second electrode 102 is formed as a light-transmitting electrode.

Next, an embodiment of a light-emitting element with a structure in which a plurality of light-emitting units are stacked (hereinafter this type of light-emitting element is also referred to as a stacked element or a tandem element) is described with reference to FIG. 1B. This light-emitting element includes a plurality of light-emitting units between a pair of electrodes (a first electrode and a second electrode). One light-emitting unit has the same structure as the EL layer 103 illustrated in FIG. 1A. In other words, the light-emitting element illustrated in FIG. 1A includes a single light-emitting unit, and the light-emitting element illustrated in FIG. 1B includes a plurality of light-emitting units.

In FIG. 1B, an EL layer 503 including a stack of a first light-emitting unit 511, a charge generation layer 513, and a second light-emitting unit 512 is provided between a first electrode 501 and a second electrode 502. The first electrode 501 and the second electrode 502 correspond, respectively, to the first electrode 101 and the second electrode 102 illustrated in FIG. 1A, and can be formed using the materials given in the description for FIG. 1A. Furthermore, the first light-emitting unit 511 and the second light-emitting unit 512 may have the same structure or different structures.

The charge generation layer 513 contains a composite material of an organic compound and a metal oxide. As this composite material of an organic compound and a metal oxide, the composite material that can be used for the hole-injection layer 111 illustrated in FIG. 1A can be used. Since the composite material of an organic compound and a metal oxide is superior in carrier-injection property and carrier-transport property, low-voltage driving or low-current driving can be realized. Note that when a surface of a light-emitting unit on the anode side is in contact with the charge generation layer, the charge generation layer can also serve as a hole-injection layer of the light-emitting unit; thus, a hole-injection layer does not need to be formed in the light-emitting unit.

Note that the charge-generation layer 513 may be formed by stacking a layer containing the above composite material and a layer containing another material. For example, a layer containing the above composite material and a layer containing a compound with a high electron-transport property and a compound selected from the compounds with an electron donating property may be stacked. Alternatively, a layer containing a composite material of an organic compound and a metal oxide and a transparent conductive film may be stacked.

An electron-injection buffer layer may be provided between the charge-generation layer 513 and the light-emitting unit on the anode side of the charge-generation layer. The electron-injection buffer layer is a stack of a very thin alkali metal layer and an electron-relay layer containing a substance having an electron-transport property. The very thin alkali metal layer corresponds to the electron-injection layer 115 and has a function of lowering an electron injection barrier. The electron-relay layer has a function of preventing an interaction between the alkali metal layer and the charge-generation layer and smoothly transferring electrons. The LUMO level of the substance having an electron-transport property which is contained in the electron-relay layer is set to be between the LUMO level of an substance having an acceptor property in the charge-generation layer 513 and the LUMO level of a substance contained in a layer in contact with the electron-injection buffer layer in the light-emitting unit on the anode side. As a specific value of the energy level, the LUMO level of the substance having an electron-transport property which is contained in the electron-relay layer is preferably greater than or equal to −5.0 eV, more preferably greater than or equal to −5.0 eV and less than or equal to −3.0 eV. Note that as the substance having an electron-transport property which is contained in the electron-relay layer, a metal complex having a metal-oxygen bond and an aromatic ligand or a phthalocyanine-based material is preferably used. In that case, the alkali metal layer of the electron-injection buffer layer serves as the electron-injection layer in the light-emitting unit on the anode side; thus, the electron-injection layer does not need to be faulted over the light-emitting unit.

The charge-generation layer 513 provided between the first light-emitting unit 511 and the second light-emitting unit 512 may have any structure as far as electrons can be injected to a light-emitting unit on one side and holes can be injected to a light-emitting unit on the other side when a voltage is applied between the first electrode 501 and the second electrode 502. For example, in FIG. 1B, any layer can be used as the charge generation layer 513 as long as the layer injects electrons into the first light-emitting unit 511 and holes into the second light-emitting unit 512 when a voltage is applied such that the potential of the first electrode is higher than that of the second electrode.

The light-emitting element having two light-emitting units is described with reference to FIG. 1B; however, the present invention can be similarly applied to a light-emitting element in which three or more light-emitting units are stacked. With a plurality of light-emitting units partitioned by the charge-generation layer between a pair of electrodes, it is possible to provide an element which can emit light with high luminance with the current density kept low and has a long lifetime. A light-emitting device that can be driven at a low voltage and has low power consumption can be realized.

Furthermore, when emission colors of the light-emitting units are made different, light emission having a desired color can be obtained from the light-emitting element as a whole. For example, it is easy to enable a light-emitting element having two light-emitting units to emit white light as the whole element when the emission colors of the first light-emitting unit are red and green and the emission color of the second light-emitting unit is blue.

### <<Micro Optical Resonator (Microcavity) Structure>>

A light-emitting element with a microcavity structure is formed with the use of a reflective electrode and a semi-transmissive and semi-reflective electrode as the pair of electrodes. The reflective electrode and the semi-transmissive and semi-reflective electrode correspond to the first electrode and the second electrode. The light-emitting element with a microcavity structure includes at least an EL layer between the reflective electrode and the semi-transmissive and semi-reflective electrode. The EL layer includes at least a light-emitting layer serving as a light-emitting region.

Light emitted in all directions from the light-emitting layer included in the EL layer is reflected and resonated by the reflective electrode and the semi-transmissive and semi-reflective electrode. Note that the reflective electrode is formed using a conductive material having reflectivity, and a film whose visible light reflectivity is 40% to 100%, preferably 70% to 100%, and whose resistivity is 1×10⁻² Ωcm or lower is used. In addition, the semi-transmissive and semi-reflective electrode is formed using a conductive material having reflectivity and a light-transmitting property, and a film whose visible light reflectivity is 20% to 80%, preferably 40% to 70%, and whose resistivity is 1×10⁻² Ωcm or lower is used.

In the light-emitting element, by changing thicknesses of the transparent conductive film, the composite material, the carrier-transport material, and the like, the optical path length between the reflective electrode and the semi-transmissive and semi-reflective electrode can be changed. Thus, light with a wavelength that is resonated between the reflective electrode and the semi-transmissive and semi-reflective electrode can be intensified while light with a wavelength that is not resonated therebetween can be attenuated.

Note that light that is reflected back by the reflective electrode (first reflected light) considerably interferes with light that directly enters the semi-transmissive and semi-reflective electrode from the light-emitting layer (first incident light). For this reason, the optical path length between the reflective electrode and the light-emitting layer is preferably adjusted to (2n−1)λ/4 (n is a natural number of 1 or larger and λ is a wavelength of light to be amplified). By adjusting the optical path length, the phases of the first reflected light and the first incident light can be aligned with each other and the light emitted from the light-emitting layer can be further amplified.

Note that in the above structure, the EL layer may be formed of light-emitting layers or may be a single light-emitting layer. The tandem light-emitting element described above may be combined with the EL layers; for example, a light-emitting element may have a structure in which a plurality of EL layers is provided, a charge-generation layer is provided between the EL layers, and each EL layer is formed of light-emitting layers or a single light-emitting layer.

With the microcavity structure, emission intensity with a specific wavelength in the front direction can be increased, whereby power consumption can be reduced. In particular, a light-emitting element that uses the 1,6-bis(diphenylamino)pyrene derivative, which has a narrow half width of an emission spectrum and a sharp spectrum, as an emission center substance can have excellent emission efficiency because the microcavity structure brings a significant light emission amplification effect.

### <<Light-Emitting Device>>

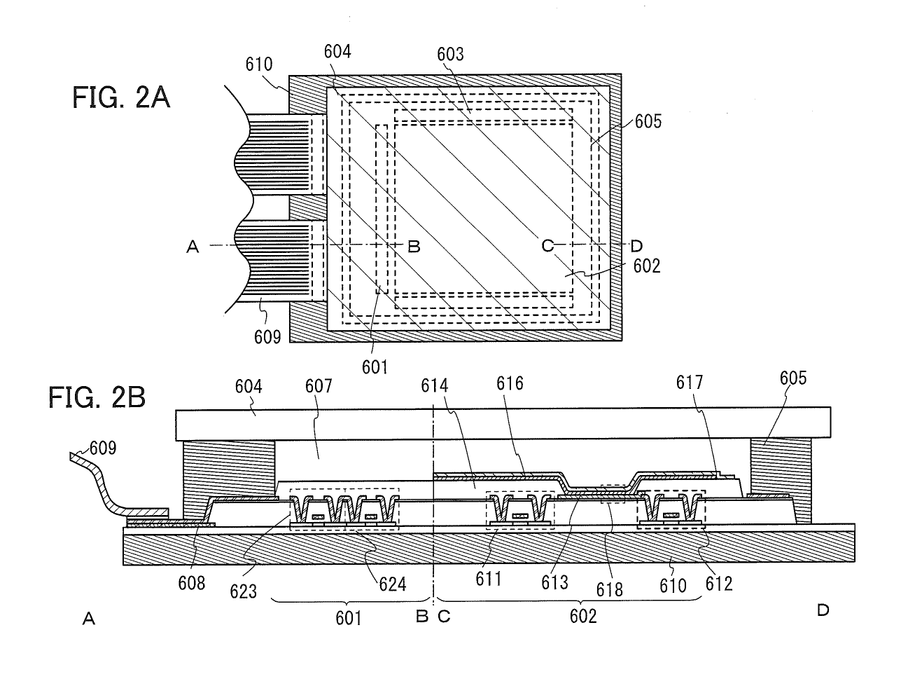

A light-emitting device of one embodiment of the present invention is described using FIGS. 2A and 2B. Note that FIG. 2A is a top view illustrating the light-emitting device and FIG. 2B is a cross-sectional view of FIG. 2A taken along lines A-B and C-D. This light-emitting device includes a driver circuit portion (source line driver circuit) 601, a pixel portion 602, and a driver circuit portion (gate line driver circuit) 603, which can control light emission of a light-emitting element and illustrated with dotted lines. A reference numeral 604 denotes a sealing substrate; 605, a sealing material; and a portion surrounded by the sealing material 605 is a space 607.

Reference numeral 608 denotes a wiring for transmitting signals to be input to the source line driver circuit 601 and the gate line driver circuit 603 and receiving signals such as a video signal, a clock signal, a start signal, and a reset signal from an flexible printed circuit (FPC) 609 serving as an external input terminal. Although only the FPC is illustrated here, a printed wiring board (PWB) may be attached to the FPC. The light-emitting device in the present specification includes, in its category, not only the light-emitting device itself but also the light-emitting device provided with the FPC or the PWB.

Next, a cross-sectional structure will be described with reference to FIG. 2B. The driver circuit portion and the pixel portion are Ruined over an element substrate 610; the source line driver circuit 601, which is a driver circuit portion, and one of the pixels in the pixel portion 602 are illustrated here.

As the source line driver circuit 601, a CMOS circuit in which an n-channel FET 623 and a p-channel FET 624 are combined is formed. In addition, the driver circuit may be formed with any of a variety of circuits such as a CMOS circuit, a PMOS circuit, or an NMOS circuit. Although a driver integrated type in which the driver circuit is formed over the substrate is illustrated in this embodiment, the driver circuit is not necessarily formed over the substrate, and the driver circuit can be formed outside, not over the substrate.

The pixel portion 602 includes a plurality of pixels including a switching FET 611, a current controlling FET 612, and a first electrode 613 electrically connected to a drain of the current controlling FET 612. One embodiment of the present invention is not limited to the structure. The pixel portion 602 may include three or more FETs and a capacitor in combination.

The kind and crystallinity of a semiconductor used for the FETs is not particularly limited; an amorphous semiconductor or a crystalline semiconductor may be used. Examples of the semiconductor used for the FETs include Group 14 semiconductors (e.g., silicon), Group 13 semiconductors (e.g., gallium), compound semiconductors (including oxide semiconductors), and organic semiconductors. Oxide semiconductors are particularly preferable. Examples of the oxide semiconductor include an In—Ga oxide and an In-M-Zn oxide (M is Al, Ga, Y, Zr, La, Ce, or Nd). Note that an oxide semiconductor that has an energy gap of 2 eV or more, preferably 2.5 eV or more, further preferably 3 eV or more is preferably used, in which case the off-state current of the transistors can be reduced.

Note that to cover an end portion of the first electrode 613, an insulator 614 is formed. The insulator 614 can be formed using a positive photosensitive acrylic resin film here.

The insulator 614 is formed to have a curved surface with curvature at its upper or lower end portion in order to obtain favorable coverage. For example, in the case where positive photosensitive acrylic is used for a material of the insulator 614, only the upper end portion of the insulator 614 preferably has a curved surface with a curvature radius (0.2 μm to 3 μm). As the insulator 614, either a negative photosensitive resin or a positive photosensitive resin can be used.

An EL layer 616 and a second electrode 617 are formed over the first electrode 613. The first electrode 613, the EL layer 616, and the second electrode 617 correspond, respectively, to the first electrode 101, the EL layer 103, and the second electrode 102 in FIG. 1A or to the first electrode 501, the EL layer 503, and the second electrode 502 in FIG. 1B.

The EL layer 616 preferably contains the 1,6-bis(diphenylamino)pyrene derivative in which an alkyl group is bonded to each of the two ortho positions of at least one of the two phenyl groups in each of the two diphenylamino groups. The 1,6-bis(diphenylamino)pyrene derivative is preferably an organic compound represented by General Formula (G1-1), (G1-2), (G2-1), or (G2-2). The 1,6-bis(diphenylamino)pyrene derivative is preferably used as an emission center substance in a light-emitting layer.

The sealing substrate 604 is attached to the element substrate 610 with the sealing material 605, so that a light-emitting element 618 is provided in the space 607 surrounded by the element substrate 610, the sealing substrate 604, and the sealing material 605. The space 607 may be filled with filler such as an inert gas (such as nitrogen or argon), or the sealing material 605. It is preferable that the sealing substrate 604 be provided with a recessed portion and a drying agent 625 be provided in the recessed portion, in which case deterioration due to influence of moisture can be suppressed.

An epoxy-based resin or glass frit is preferably used for the sealing material 605. It is preferable that such a material do not transmit moisture or oxygen as much as possible. As the element substrate 610 and the sealing substrate 604, a glass substrate, a quartz substrate, or a plastic substrate framed of fiber reinforced plastic (FRP), poly(vinyl fluoride) (PVF), polyester, or acrylic can be used.

Note that in this specification and the like, a transistor or a light-emitting element can be formed using any of a variety of substrates, for example. The type of a substrate is not limited to a certain type. As the substrate, a semiconductor substrate (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, a base material film, or the like can be used, for example. As an example of a glass substrate, a barium borosilicate glass substrate, an aluminoborosilicate glass substrate, a soda lime glass substrate, or the like can be given. Examples of the flexible substrate, the attachment film, the base film, and the like are substrates of plastics typified by polyethylene terephthalate (PET), polyethylene naphthalate (PEN), and polyether sulfone (PES). Another example is a synthetic resin such as acrylic. Alternatively, polypropylene, polyester, polyvinyl fluoride, polyvinyl chloride, or the like can be used. Alternatively, polyamide, polyimide, aramid, epoxy, an inorganic vapor deposition film, paper, or the like can be used. Specifically, the use of semiconductor substrates, single crystal substrates, SOI substrates, or the like enables the manufacture of small-sized transistors with a small variation in characteristics, size, shape, or the like and with high current capability. A circuit using such transistors achieves lower power consumption of the circuit or higher integration of the circuit.

Alternatively, a flexible substrate may be used as the substrate, and the transistor or the light-emitting element may be provided directly on the flexible substrate. Still alternatively, a separation layer may be provided between the substrate and the transistor or the substrate and the light-emitting element. The separation layer can be used when part or the whole of a semiconductor device formed over the separation layer is separated from the substrate and transferred onto another substrate. In such a case, the transistor can be transferred to a substrate having low heat resistance or a flexible substrate. For the separation layer, a stack including inorganic films, which are a tungsten film and a silicon oxide film, or an organic resin film of polyimide or the like formed over a substrate can be used, for example.

In other words, a transistor or a light-emitting element may be formed using one substrate, and then transferred to another substrate. Examples of a substrate to which a transistor or a light-emitting element is transferred include, in addition to the above-described substrates over which transistors can be formed, a paper substrate, a cellophane substrate, an aramid film substrate, a polyimide film substrate, a stone substrate, a wood substrate, a cloth substrate (including a natural fiber (e.g., silk, cotton, or hemp), a synthetic fiber (e.g., nylon, polyurethane, or polyester), a regenerated fiber (e.g., acetate, cupra, rayon, or regenerated polyester), or the like), a leather substrate, and a rubber substrate. When such a substrate is used, a transistor with excellent characteristics or a transistor with low power consumption can be formed, a device with high durability or high heat resistance can be provided, or reduction in weight or thickness can be achieved.

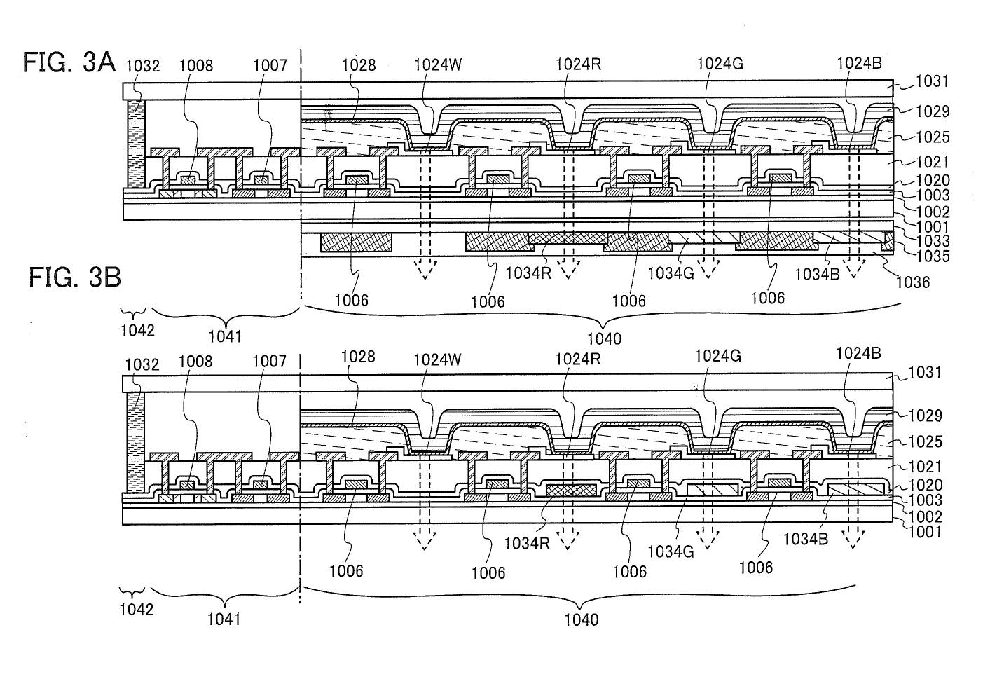

FIGS. 3A and 3B each illustrate an example of a light-emitting device in which full color display is achieved by formation of a light-emitting element exhibiting white light emission and with the use of coloring layers (color filters) and the like. In FIG. 3A, a substrate 1001, a base insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, first electrodes 1024W, 1024R, 1024G, and 1024B of light-emitting elements, a partition 1025, an EL layer 1028, a second electrode 1029 of the light-emitting elements, a sealing substrate 1031, a sealing material 1032, and the like are illustrated.

In FIG. 3A, coloring layers (a red coloring layer 1034R, a green coloring layer 1034G, and a blue coloring layer 1034B) are provided on a transparent base material 1033. A black layer (a black matrix) 1035 may be additionally provided. The transparent base material 1033 provided with the coloring layers and the black layer is positioned and fixed to the substrate 1001. Note that the coloring layers and the black layer are covered with an overcoat layer 1036. In FIG. 3A, light emitted from part of the light-emitting layer does not pass through the coloring layers, while light emitted from the other part of the light-emitting layer passes through the coloring layers. Since light which does not pass through the coloring layers is white and light which passes through any one of the coloring layers is red, blue, or green, an image can be displayed using pixels of the four colors.

A light-emitting element that uses the 1,6-bis(diphenylamino)pyrene derivative as one of emission center substances can emit blue light with high efficiency without light loss caused by a color filter because the derivative has a narrow half width of an emission spectrum and a sharp spectrum.

FIG. 3B illustrates an example in which the coloring layers (the red coloring layer 1034R, the green coloring layer 1034G, and the blue coloring layer 1034B) are provided between the gate insulating film 1003 and the first interlayer insulating film 1020. As in the structure, the coloring layers may be provided between the substrate 1001 and the sealing substrate 1031.

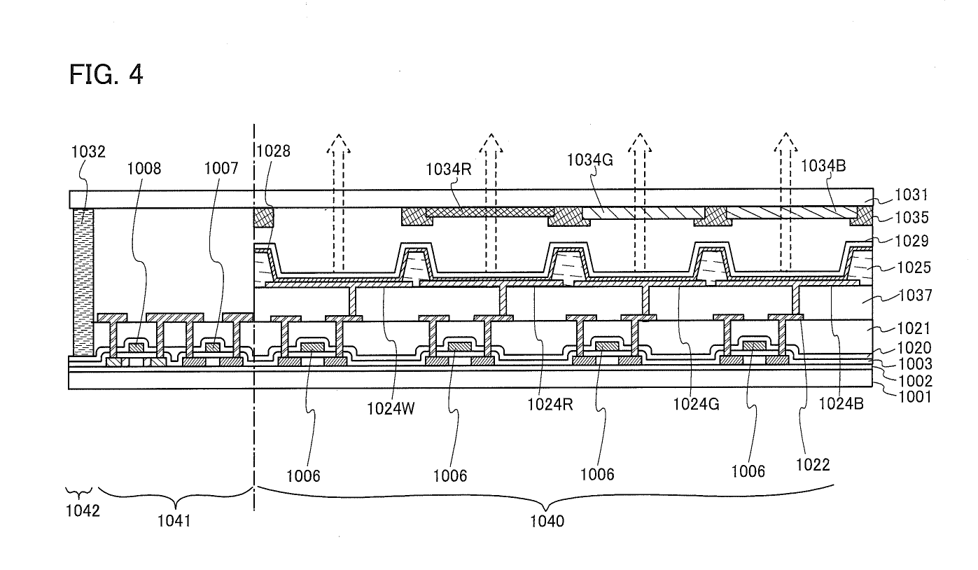

The above-described light-emitting device is a light-emitting device having a structure in which light is extracted from the substrate 1001 side where the FETs are formed (a bottom emission structure), but may be a light-emitting device having a structure in which light is extracted from the sealing substrate 1031 side (a top emission structure). FIG. 4 is a cross-sectional view of a light-emitting device having a top emission structure. In this case, a substrate which does not transmit light can be used as the substrate 1001. The process up to the step of forming a connection electrode which connects the FET and the anode of the light-emitting element is performed in a manner similar to that of the light-emitting device having a bottom emission structure. Then, a third interlayer insulating film 1037 is formed to cover an electrode 1022. This insulating film may have a planarization function. The third interlayer insulating film 1037 can be formed using a material similar to that of the second interlayer insulating film 1021, and can alternatively be formed using any of other various materials.

The first electrodes 1024W, 1024R, 1024G, and 1024B of the light-emitting elements each serve as an anode here, but may serve as a cathode. Further, in the case of a light-emitting device having a top emission structure as illustrated in FIG. 4, the first electrodes are preferably reflective electrodes. The EL layer 1028 is formed to have a structure similar to the structure of the EL layer 103 in FIG. 1A or the EL layer 503 in FIG. 1B, with which white light emission can be obtained.

In the case of a top emission structure as illustrated in FIG. 4, sealing can be performed with the sealing substrate 1031 on which the coloring layers (the red coloring layer 1034R, the green coloring layer 1034G, and the blue coloring layer 1034B) are provided. The sealing substrate 1031 may be provided with the black layer (black matrix) 1035 which is positioned between pixels. The coloring layers (the red coloring layer 1034R, the green coloring layer 1034G, and the blue coloring layer 1034B) and the black layer may be covered with the overcoat layer 1036. Note that a light-transmitting substrate is used as the sealing substrate 1031.

Although an example in which full color display is performed using four colors of red, green, blue, and white is shown here, there is no particular limitation and full color display using three colors of red, green, and blue or four colors of red, green, blue, and yellow may be performed.

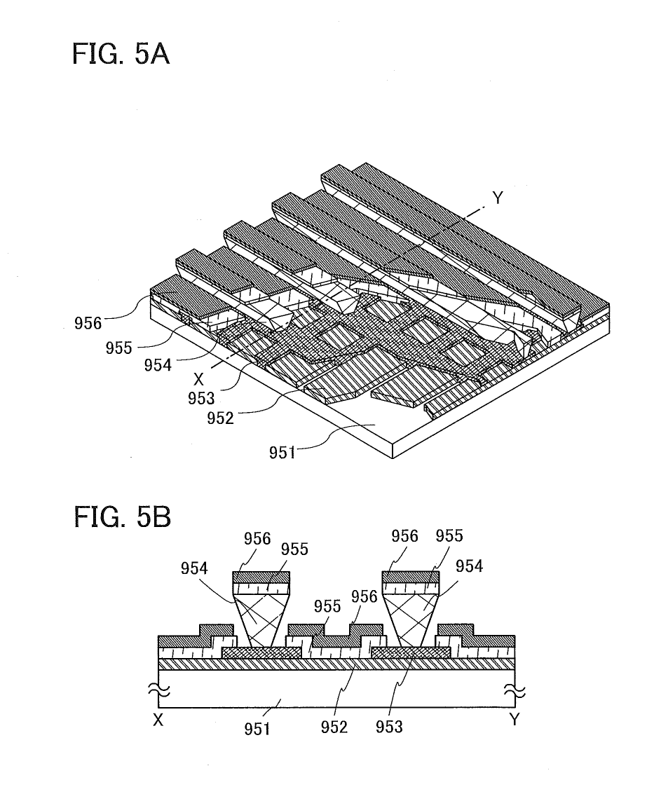

FIGS. 5A and 5B illustrate a passive matrix light-emitting device which is one embodiment of the present invention. FIG. 5A is a perspective view of the light-emitting device, and FIG. 5B is a cross-sectional view of FIG. 5A taken along line X-Y. In FIGS. 5A and 5B, an EL layer 955 is provided between an electrode 952 and an electrode 956 over a substrate 951. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. In other words, a cross section taken along the direction of the short side of the partition layer 954 is trapezoidal, and the lower side (a side in contact with the insulating layer 953, which is one of a pair of parallel sides of the trapezoidal cross section) is shorter than the upper side (a side not in contact with the insulating layer 953, which is the other one of the pair of parallel sides). The partition layer 954 thus provided can prevent defects in the light-emitting element due to static electricity or others.

Since many minute light-emitting elements arranged in a matrix can each be controlled with the FETs formed in the pixel portion, the above-described light-emitting device can be suitably used as a display device for displaying images.

### <<Lighting Device>>

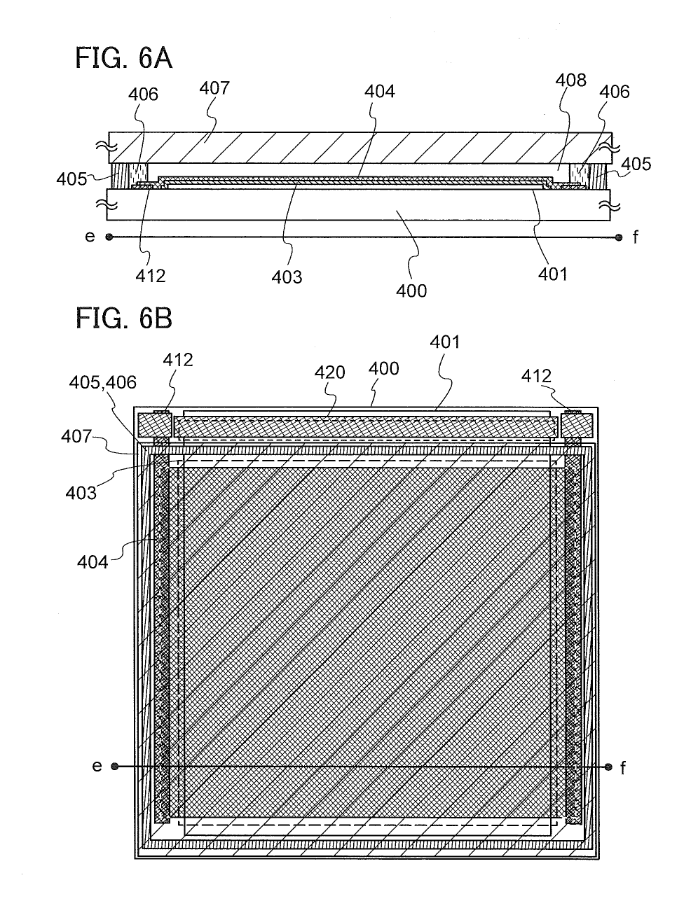

A lighting device which is one embodiment of the present invention is described with reference to FIGS. 6A and 6B. FIG. 6B is a top view of the lighting device, and FIG. 6A is a cross-sectional view of FIG. 6B taken along line e-f.

In the lighting device, a first electrode 401 is formed over a substrate 400 which is a support and has a light-transmitting property. The first electrode 401 corresponds to the first electrode 101 in FIG. 1A. When light is extracted through the first electrode 401 side, the first electrode 401 is formed using a material having a light-transmitting property.

A pad 412 for applying a voltage to a second electrode 404 is provided over the substrate 400.

An EL layer 403 is formed over the first electrode 401. The EL layer 403 corresponds to, for example, the EL layer 103 in FIG. 1A or the EL layer 503 in FIG. 1B. Refer to the descriptions for the structure.

The second electrode 404 is formed to cover the EL layer 403. The second electrode 404 corresponds to the second electrode 102 in FIG. 1A. The second electrode 404 is formed using a material having high reflectance when light is extracted through the first electrode 401 side. The second electrode 404 is connected to the pad 412, whereby a voltage is applied.

A light-emitting element is formed with the first electrode 401, the EL layer 403, and the second electrode 404. The substrate 400 provided with the light-emitting element is fixed to a sealing substrate 407 with sealing materials 405 and 406 and sealing is performed, whereby the lighting device is completed. It is possible to use only either the sealing material 405 or the sealing material 406. In addition, the inner sealing material 406 (not shown in FIG. 6B) can be mixed with a desiccant, whereby moisture is adsorbed and the reliability is increased.

When parts of the pad 412 and the first electrode 401 are extended to the outside of the sealing materials 405 and 406, the extended parts can serve as external input terminals. An IC chip 420 mounted with a converter or the like may be provided over the external input terminals.

### <<Electronic Device>>

Examples of an electronic device which is one embodiment of the present invention are described. Examples of the electronic device are television devices (also referred to as TV or television receivers), monitors for computers and the like, cameras such as digital cameras and digital video cameras, digital photo frames, mobile phones (also referred to as cell phones or mobile phone devices), portable game machines, portable information terminals, audio playback devices, and large game machines such as pachinko machines. Specific examples of these electronic devices are given below.

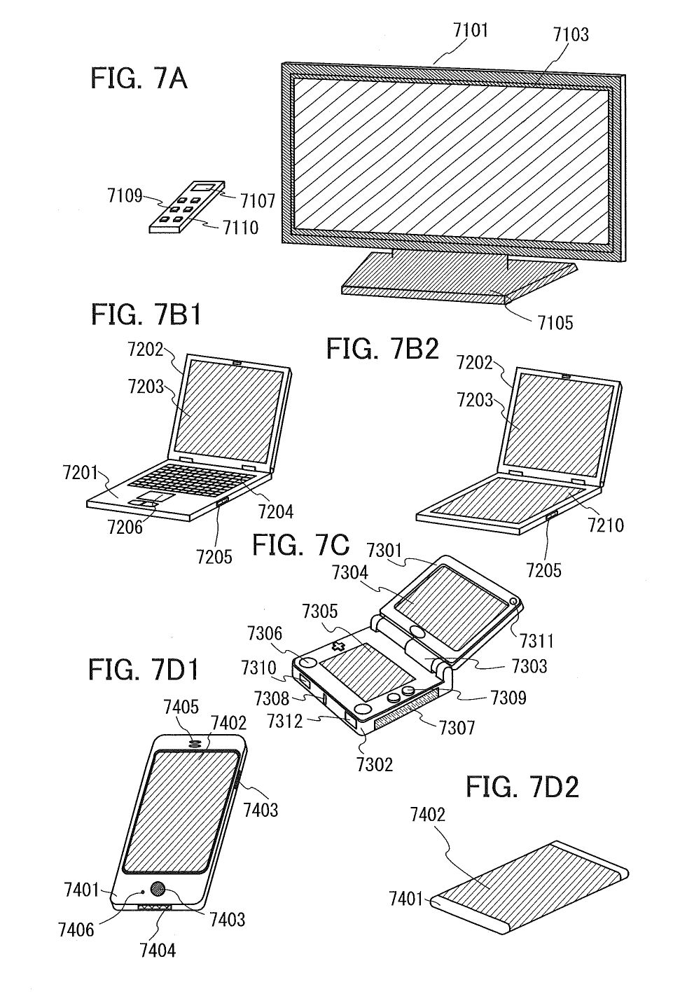

FIG. 7A illustrates an example of a television device. In the television device, a display portion 7103 is incorporated in a housing 7101. In addition, here, the housing 7101 is supported by a stand 7105. Images can be displayed on the display portion 7103, and in the display portion 7103, light-emitting elements are arranged in a matrix.

The television device can be operated with an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television device is provided with a receiver, a modem, and the like. With the use of the receiver, general television broadcasting can be received. Moreover, when the television device is connected to a communication network with or without wires via the modem, one-way (from a sender to a receiver) or two-way (between a sender and a receiver or between receivers) information communication can be performed.

FIG. 7B1 illustrates a computer, which includes a main body 7201, a housing 7202, a display portion 7203, a keyboard 7204, an external connection port 7205, a pointing device 7206, and the like. Note that this computer is manufactured by using light-emitting elements arranged in a matrix in the display portion 7203. The computer illustrated in FIG. 7B1 may have a structure illustrated in FIG. 7B2. A computer illustrated in FIG. 7B2 is provided with a second display portion 7210 instead of the keyboard 7204 and the pointing device 7206. The second display portion 7210 is a touch screen, and input can be performed by operation of display for input on the second display portion 7210 with a finger or a dedicated pen. The second display portion 7210 can also display images other than the display for input. The display portion 7203 may be also a touch screen. Connecting the two screens with a hinge can prevent troubles; for example, the screens can be prevented from being cracked or broken while the computer is being stored or carried.

FIG. 7C illustrates a portable game machine, which includes two housings, a housing 7301 and a housing 7302, which are connected with a joint portion 7303 so that the portable game machine can be opened or folded. The housing 7301 incorporates a display portion 7304 including light-emitting elements arranged in a matrix, and the housing 7302 incorporates a display portion 7305. In addition, the portable game machine illustrated in FIG. 7C includes a speaker portion 7306, a storage medium insertion portion 7307, an LED lamp 7308, an input means (an operation key 7309, a connection terminal 7310, a sensor 7311 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), or a microphone 7312), and the like. The structure of the portable game machine is not limited to the above structure as long as the light-emitting device may be used for at least both of the display portion 7304 and the display portion 7305. The portable game machine illustrated in FIG. 7C has a function of reading out a program or data stored in a storage medium to display it on the display portion, and a function of sharing information with another portable game machine by wireless communication. The portable game machine illustrated in FIG. 7C can have a variety of functions without limitation to the above.

FIGS. 7D1 and 7D2 illustrate an example of a portable information terminal. The portable information terminal is provided with a display portion 7402 incorporated in a housing 7401, operation buttons 7403, an external connection port 7404, a speaker 7405, a microphone 7406, and the like. Note that the portable information terminal has the display portion 7402 including light-emitting elements arranged in a matrix.

Information can be input to the portable information terminal illustrated in FIGS. 7D1 and 7D2 by touching the display portion 7402 with a finger or the like. In this case, operations such as making a call and creating an e-mail can be performed by touching the display portion 7402 with a finger or the like.

There are mainly three screen modes of the display portion 7402. The first mode is a display mode mainly for displaying an image. The second mode is an input mode mainly for inputting information such as characters. The third mode is a display-and-input mode in which two modes of the display mode and the input mode are combined.

For example, in the case of making a call or creating an e-mail, a text input mode mainly for inputting text is selected for the display portion 7402 so that text displayed on a screen can be inputted. In this case, it is preferable to display a keyboard or number buttons on almost the entire screen of the display portion 7402.

When a detection device including a sensor such as a gyroscope or an acceleration sensor for detecting inclination is provided inside the mobile phone, screen display of the display portion 7402 can be automatically changed by determining the orientation of the mobile phone (whether the mobile phone is placed horizontally or vertically).

The screen modes are switched by touch on the display portion 7402 or operation with the operation buttons 7403 of the housing 7401. The screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, when input by touching the display portion 7402 is not performed for a certain period while a signal detected by an optical sensor in the display portion 7402 is detected, the screen mode may be controlled so as to be switched from the input mode to the display mode.

The display portion 7402 may function as an image sensor. For example, an image of a palm print, a fingerprint, or the like is taken by the display portion 7402 while in touch with the palm or the finger, whereby personal authentication can be performed. Further, by providing a backlight or a sensing light source which emits near-infrared light in the display portion, an image of a finger vein, a palm vein, or the like can be taken.

Note that in the above electronic devices, any of the structures described in this specification can be combined as appropriate.

The display portion preferably includes a light-emitting element including an organic compound of one embodiment of the present invention. Since the light-emitting element can be a light-emitting element with high emission efficiency, the electronic device can have low power consumption. In addition, the light-emitting element can have high heat resistance.

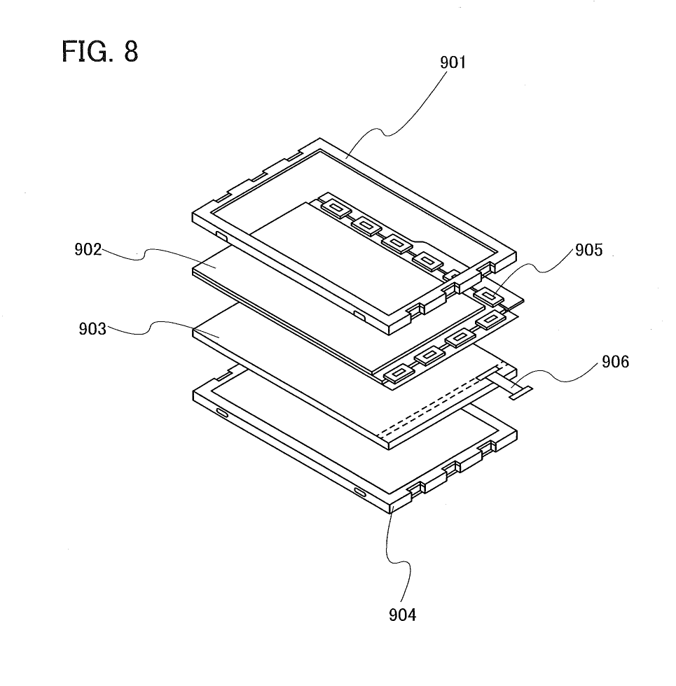

FIG. 8 illustrates an example of a liquid crystal display device including the light-emitting element. The liquid crystal display device illustrated in FIG. 8 includes a housing 901, a liquid crystal layer 902, a backlight unit 903, and a housing 904. The liquid crystal layer 902 is connected to a driver IC 905. The light-emitting element is used for the backlight unit 903, to which current is supplied through a terminal 906.

As the light-emitting element, a light-emitting element including the organic compound of one embodiment of the present invention is preferably used. By including the light-emitting element, the backlight of the liquid crystal display device can have low power consumption. In addition, the backlight can have high heat resistance.

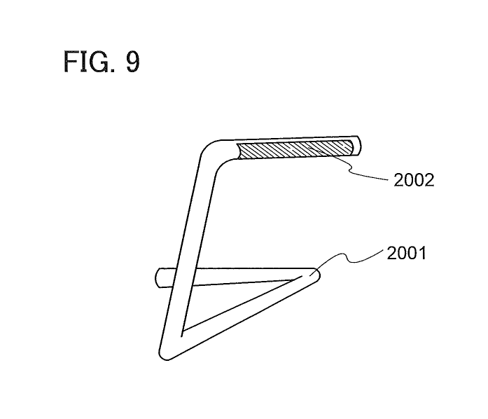

FIG. 9 illustrates an example of a desk lamp which is one embodiment of the present invention. The desk lamp illustrated in FIG. 9 includes a housing 2001 and a light source 2002, and a lighting device including a light-emitting element is used as the light source 2002.

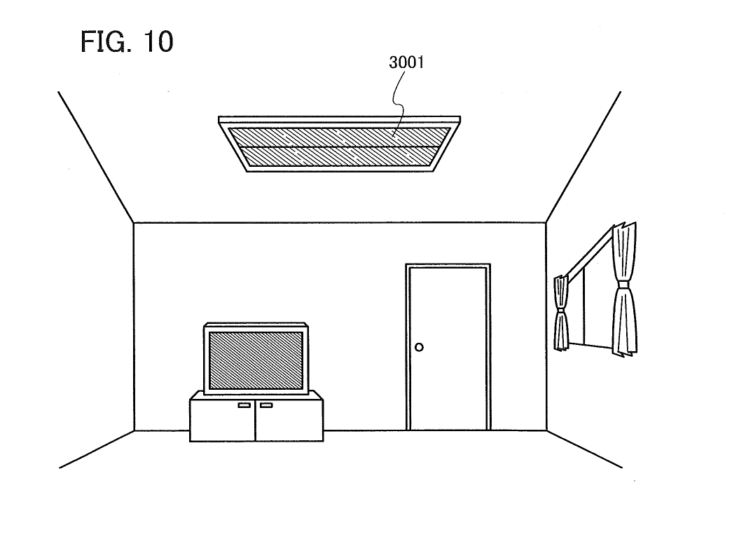

FIG. 10 illustrates an example of an indoor lighting device 3001. A light-emitting element including the organic compound of one embodiment of the present invention is preferably used in the lighting device 3001.

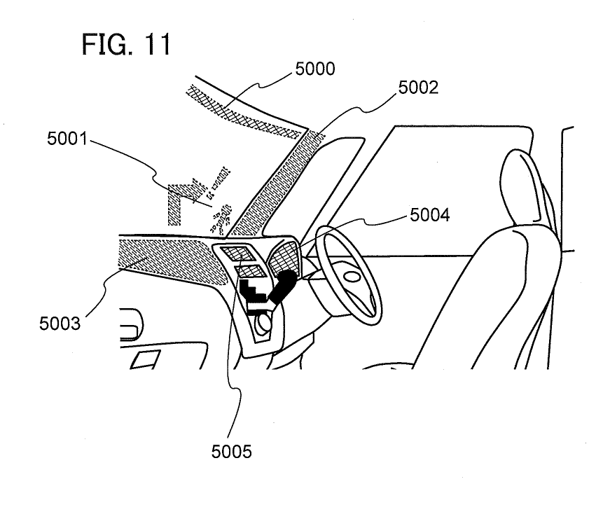

An automobile which is one embodiment of the present invention is illustrated in FIG. 11. In the automobile, light-emitting elements are used for a windshield and a dashboard. Display regions 5000 to 5005 are provided by using the light-emitting elements. The light-emitting elements preferably include the organic compound of one embodiment of the present invention, and can have low power consumption. This also suppresses power consumption of the display regions 5000 to 5005, showing suitability for use in an automobile.

The display regions 5000 and 5001 are provided in the automobile windshield including the light-emitting elements. When a first electrode and a second electrode are formed of electrodes having light-transmitting properties in these light-emitting elements, what is called a see-through display device, through which the opposite side can be seen, can be obtained. Such a see-through display device can be provided even in the automobile windshield, without hindering the vision. Note that in the case where a transistor for driving or the like is provided, a transistor having a light-transmitting property, such as an organic transistor using an organic semiconductor material or a transistor using an oxide semiconductor, is preferably used.

The display region 5002 is provided in a pillar portion using a light-emitting element. The display region 5002 can compensate for the view hindered by the pillar portion by showing an image taken by an imaging unit provided in the car body. Similarly, a display region 5003 provided in the dashboard can compensate for the view hindered by the car body by showing an image taken by an imaging unit provided in the outside of the car body, which leads to elimination of blind areas and enhancement of safety. Showing an image so as to compensate for the area which a driver cannot see makes it possible for the driver to confirm safety easily and comfortably.

The display region 5004 and the display region 5005 can provide a variety of kinds of information such as navigation information, a speedometer, a tachometer, a mileage, a fuel meter, a gearshift indicator, and air-condition setting. The content or layout of the display can be changed freely by a user as appropriate. Note that such information can also be shown by the display regions 5000 to 5003. The display regions 5000 to 5005 can also be used as lighting devices.

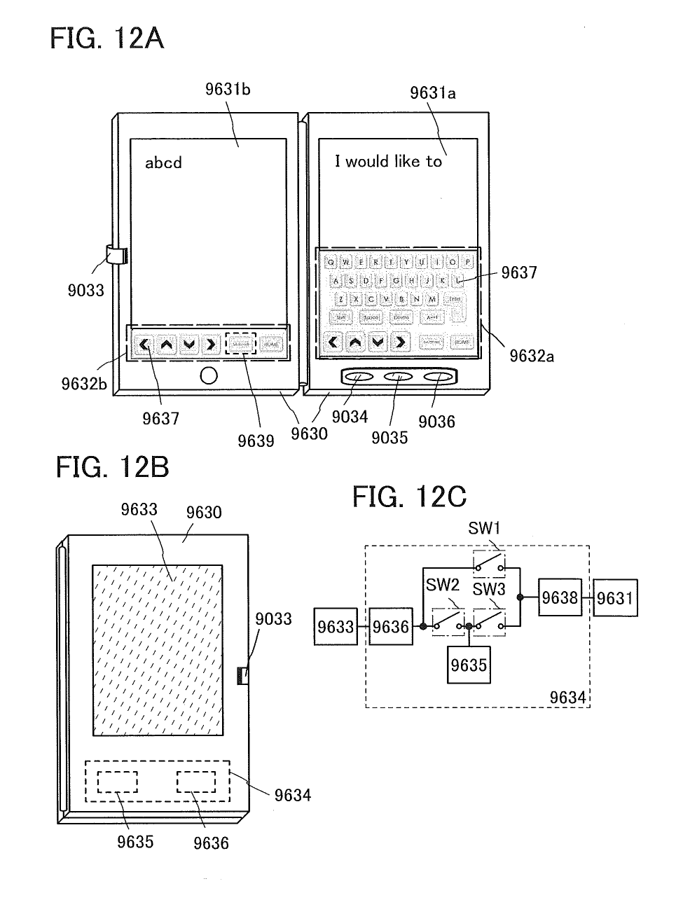

FIGS. 12A and 12B illustrate an example of a foldable tablet terminal. FIG. 12A illustrates the tablet terminal which is unfolded. The tablet terminal includes a housing 9630, a display portion 9631a, a display portion 9631b, a display mode switch 9034, a power switch 9035, a power-saving mode switch 9036, and a clasp 9033. Note that in the tablet terminal, one or both of the display portion 9631a and the display portion 9631b is/are formed using a light-emitting device which includes the light-emitting element of one embodiment of the present invention.

Part of the display portion 9631a can be a touchscreen region 9632a and data can be input when a displayed operation key 9637 is touched. Although half of the display portion 9631a has only a display function and the other half has a touchscreen function, one embodiment of the present invention is not limited to the structure. The whole display portion 9631a may have a touchscreen function. For example, a keyboard can be displayed on the entire region of the display portion 9631a so that the display portion 9631a is used as a touchscreen, and the display portion 9631b can be used as a display screen.

Like the display portion 9631a, part of the display portion 9631b can be a touchscreen region 9632b. When a switching button 9639 for showing/hiding a keyboard on the touchscreen is touched with a finger, a stylus, or the like, the keyboard can be displayed on the display portion 9631b.

Touch input can be performed in the touchscreen region 9632a and the touchscreen region 9632b at the same time.

The display mode switch 9034 can switch the display between portrait mode, landscape mode, and the like, and between monochrome display and color display, for example. The power-saving mode switch 9036 can control display luminance in accordance with the amount of external light in use of the tablet terminal sensed by an optical sensor incorporated in the tablet terminal. Another sensing device including a sensor such as a gyroscope or an acceleration sensor for sensing inclination may be incorporated in the tablet terminal, in addition to the optical sensor.

Although FIG. 12A illustrates an example in which the display portion 9631a and the display portion 9631b have the same display area, one embodiment of the present invention is not limited to the example. The display portion 9631a and the display portion 9631b may have different display areas and different display quality. For example, higher definition images may be displayed on one of the display portions 9631a and 9631b.

FIG. 12B illustrates the tablet terminal which is folded. The tablet terminal in this embodiment includes the housing 9630, a solar cell 9633, a charge and discharge control circuit 9634, a battery 9635, and a DCDC converter 9636. In FIG. 12B, a structure including the battery 9635 and the DCDC converter 9636 is illustrated as an example of the charge and discharge control circuit 9634.

Since the tablet terminal is foldable, the housing 9630 can be closed when the tablet terminal is not in use. As a result, the display portion 9631a and the display portion 9631b can be protected, thereby providing a tablet terminal with high endurance and high reliability for long-term use.

The tablet terminal illustrated in FIGS. 12A and 12B can have other functions such as a function of displaying various kinds of data (e.g., a still image, a moving image, and a text image), a function of displaying a calendar, a date, the time, or the like on the display portion, a touch-input function of operating or editing the data displayed on the display portion by touch input, and a function of controlling processing by various kinds of software (programs).

The solar cell 9633 provided on a surface of the tablet terminal can supply power to the touchscreen, the display portion, a video signal processing portion, or the like. Note that a structure in which the solar cell 9633 is provided on one or both surfaces of the housing 9630 is preferable because the battery 9635 can be charged efficiently.

The structure and operation of the charge and discharge control circuit 9634 illustrated in FIG. 12B are described with reference to a block diagram of FIG. 12C. FIG. 12C illustrates the solar cell 9633, the battery 9635, the DCDC converter 9636, a converter 9638, switches SW1 to SW3, and a display portion 9631. The battery 9635, the DCDC converter 9636, the converter 9638, and the switches SW1 to SW3 correspond to the charge and discharge control circuit 9634 illustrated in FIG. 12B.

First, description is made on an example of the operation in the case where power is generated by the solar cell 9633 with the use of external light. The voltage of the power generated by the solar cell is raised or lowered by the DCDC converter 9636 so as to be voltage for charging the battery 9635. Then, when power from the solar cell 9633 is used for the operation of the display portion 9631, the switch SW1 is turned on and the voltage of the power is raised or lowered by the converter 9638 so as to be voltage needed for the display portion 9631. When images are not displayed on the display portion 9631, the switch SW1 is turned off and the switch SW2 is turned on so that the battery 9635 is charged.

Although the solar cell 9633 is described as an example of a power generation means, the power generation means is not particularly limited, and the battery 9635 may be charged by another power generation means such as a piezoelectric element or a thermoelectric conversion element (Peltier element). The battery 9635 may be charged by a non-contact power transmission module capable of performing charging by transmitting and receiving power wirelessly (without contact), or any of the other charge means used in combination, and the power generation means is not necessarily provided.

Note that the organic compound of one embodiment of the present invention can be used for an organic thin-film solar cell. Specifically, the organic compound can be used in a carrier-transport layer since the organic compound has a carrier-transport property. The organic compound can be photoexcited and hence can be used in a power generation layer.

One embodiment of the present invention is not limited to the tablet terminal having the shape illustrated in FIGS. 12A to 12C as long as the display portion 9631 is included.

### Example 1

In this example, a method of synthesizing N,N′-bis(2,6-dimethylphenyl)-N,N′-bis[3-(9-phenyl-9H-fluoren-9-yl)phenyl]pyrene-1,6-diamine (abbreviation: 1,6oDMemFLPAPrn), which is an organic compound of one embodiment of the present invention, is described. A structural formula of 1,6oDMemFLPAPrn is shown below.

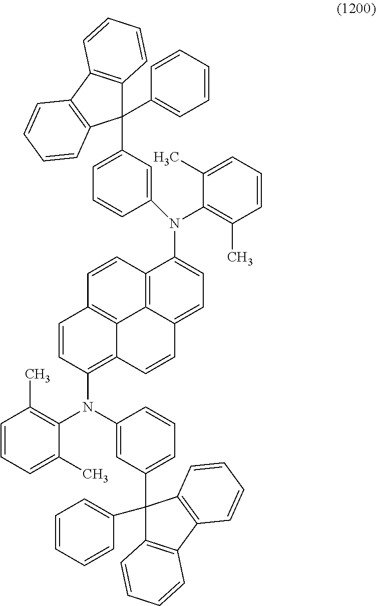 [SMILES: Cc1cccc(C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C)cccc4C)ccc4ccc1c2c43]

### Step 1: Synthesis of N-(2,6-dimethylphenyl)-N-[3-(9-phenyl-9H-fluoren-9-yl)phenyl]amine (abbreviation: oDMemFLPA)

Into a 200-mL three-neck flask were put 2.7 g (6.9 mol) of 9-(3-bromophenyl)-9-phenyl fluorine and 2.0 g (21.0 mol) of sodium tert-butoxide, and the air in the flask was replaced with nitrogen. Then, 35.0 mL of toluene, 0.9 mL (6.9 mol) of 2,6-dimethylaniline, 0.5 mL of a 10% hexane solution of tri(tert-butyl)phosphine, and 42 mg (0.1 mmol) of bis(dibenzylideneacetone)palladium(0) were added thereto, the temperature of the mixture was set to 90° C. and the mixture was stirred for 13.0 hours. After the stirring, suction filtration through Florisil (produced by Wako Pure Chemical Industries, Ltd., Catalog No. 540-00135), Celite (produced by Wako Pure Chemical Industries, Ltd., Catalog No. 531-16855), and alumina was carried out to obtain a filtrate. The filtrate was concentrated to give a solid, which was then purified by silica gel column chromatography (the developing solvent has a 3:1 ratio of hexane to toluene), so that 3.0 g of the target compound was obtained in a yield of 99%. This compound was identified as N-(2,6-dimethylphenyl)-N-[3-(9-phenyl-9H-fluoren-9-yl)phenyl]amine, which was the target substance, by nuclear magnetic resonance (¹H NMR). A synthesis scheme of Step 1 is shown below.

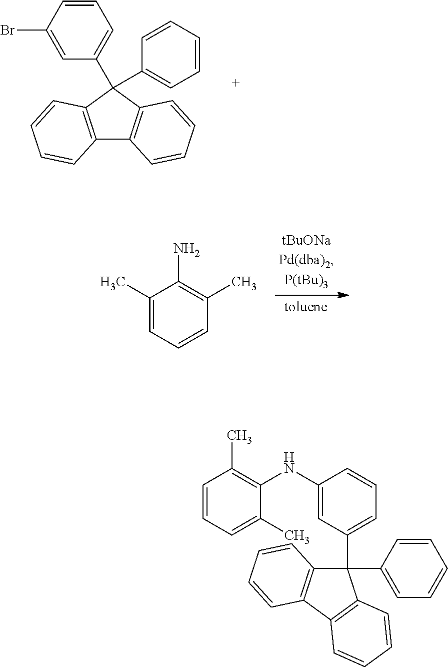

¹H NMR data of the obtained substance are as follows: ¹H NMR (CDCl₃, 500 MHz): δ=2.13 (s, 6H), 5.04 (s, 1H), 7.18 (dd, J=8.0, 2.0 Hz, 1H), 6.44 (t, J=2.0 Hz, 1H), 6.57 (d, J=8.5 Hz, 1H), 6.94-7.05 (m, 4H), 7.17-7.19 (m, 5H), 7.24-7.27 (m, 2H), 7.34 (t, J=7.5 Hz, 2H), 7.40 (d, J=7.5 Hz, 2H), 7.74 (d, J=7.5 Hz, 2H).

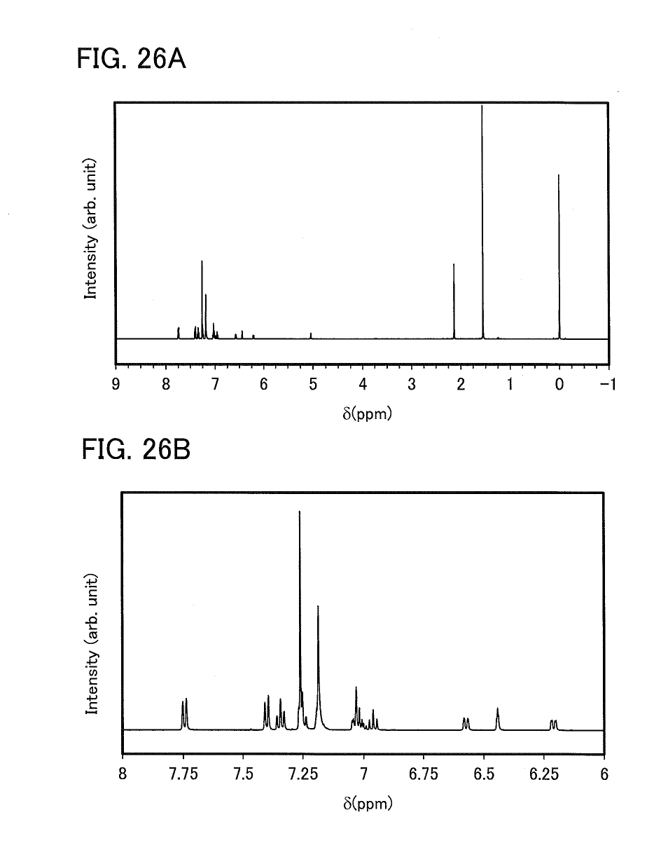

FIGS. 26A and 26B are ¹H-NMR charts. Note that FIG. 26B is a chart showing an enlarged part of FIG. 26A in the range of 6.00 ppm to 8.00 ppm. This indicates that oDMemFLPA was obtained.

### Step 2: Synthesis of 1,6oDMemFLPAPrn

Into a 100-mL three-neck flask were put 0.7 g (1.8 mmol) of 1,6-dibromopyrene, 1.8 g (4.1 mmol) of N-(2,6-dimethylphenyl)-N-[3-(9-phenyl-9H-fluoren-9-yl)phenyl]amine, and 0.6 g (5.9 mmol) of sodium tert-butoxide, and the air in the flask was replaced with nitrogen. To this mixture were added 19.0 mL of xylene and 0.5 mL of a 10% hexane solution of tri(tert-butyl)phosphine. The temperature of this mixture was set to 80° C., 37.5 mg (0.1 mmol) of bis(dibenzylideneacetone)palladium(0) was added, and the mixture was stirred and refluxed for 6.8 hours. After the stirring, 32.9 mg (0.1 mmol) of bis(dibenzylideneacetone)palladium(0) was added, and the mixture was stirred at 80° C. for 1.0 hour and refluxed for 1.2 hours. Then, the mixture was suction filtered, the obtained residue was dissolved in toluene, and this mixture was suction filtered through Florisil (produced by Wako Pure Chemical Industries, Ltd., Catalog No. 540-00135), Celite (produced by Wako Pure Chemical Industries, Ltd., Catalog No. 531-16855), and alumina to give a filtrate. The obtained filtrate was concentrated to obtain a solid. To the solid was added 200 mL of toluene, and the mixture was refluxed and left overnight. The mixture was suction-filtered to give 0.93 g of the target compound in a yield of 47%. A synthesis scheme of Step 2 is shown below.

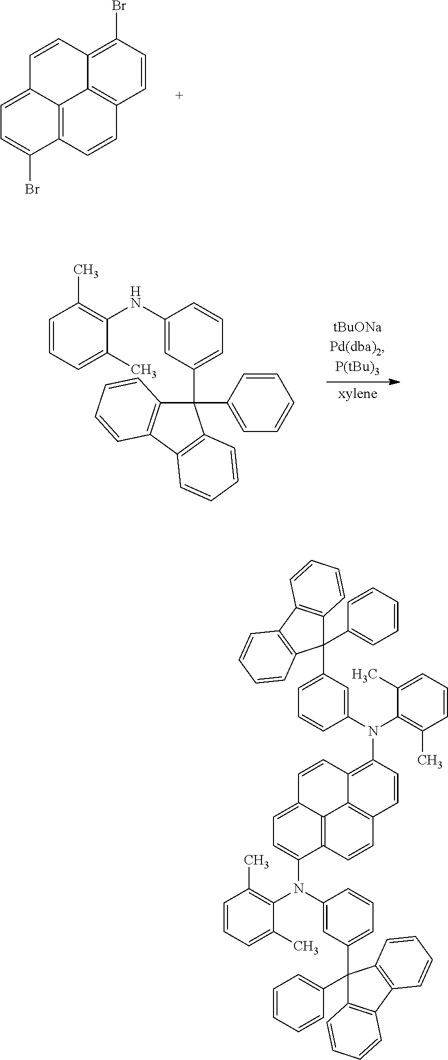 [SMILES: Brc1ccc2ccc3c(Br)ccc4ccc1c2c43 ; Cc1cccc(C)c1Nc1cccc(C2(c3ccccc3)c3ccccc3-c3ccccc32)c1 ; Cc1cccc(C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C)cccc4C)ccc4ccc1c2c43]

By a train sublimation method, 0.9 g of the obtained solid was purified. In the purification by sublimation, the solid was heated at 336° C. for 2.5 hours at a pressure of 1.2×10⁻² Pa without an argon gas stream. After the purification by sublimation, 0.8 g of the target yellow solid was obtained at a collection rate of 87%.

The obtained substance was analyzed by ¹H NMR. The measurement results are as follows. ¹H NMR (CDCl₃, 500 MHz): δ=2.05-2.13 (m, 12H), 6.46-7.18 (m, 36H), 7.36-7.46 (m, 3H), 7.52-7.60 (m, 3H), 7.66-7.72 (m, 2H), 7.86-7.92 (m, 4H).

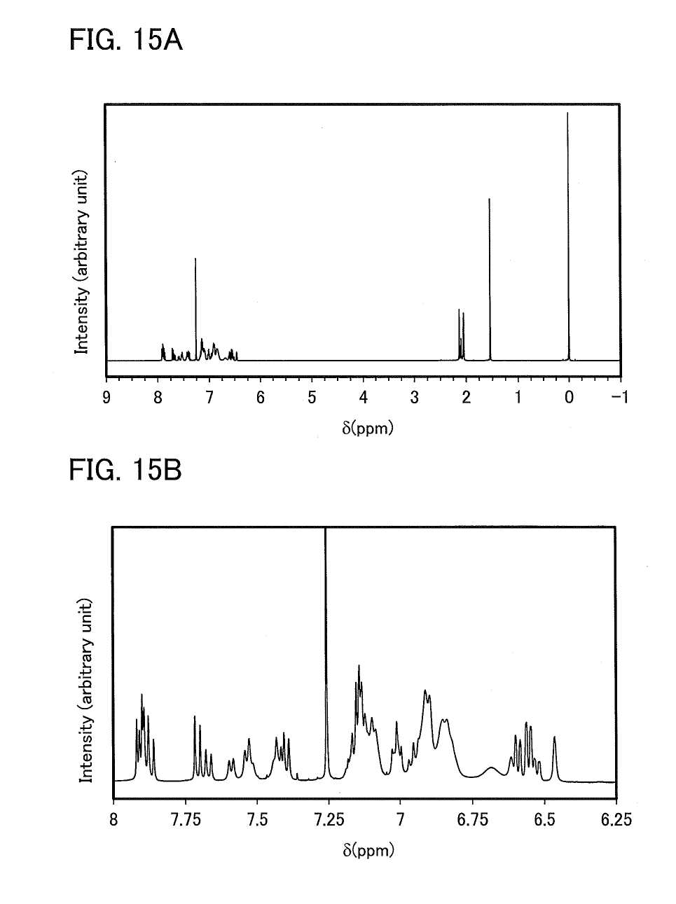

The ¹H NMR chart is shown in FIGS. 15A and 15B. Note that FIG. 15B is a chart showing an enlarged part of FIG. 15A in the range of 6.25 ppm to 8.00 ppm. The charts reveal that 1,6oDMemFLPAPrn represented by the above Structural formula (1200), which is an organic compound of one embodiment of the present invention, was obtained.

Thermogravimetry-differential thermal analysis (TG-DTA) of obtained 1,6oDMemFLPAPrn was performed. A high vacuum differential type differential thermal balance (TG/DTA 2410SA, manufactured by Bruker AXS K.K.) was used for the measurement. The measurement was carried out under a nitrogen stream (a flow rate of 200 mL/min) and a normal pressure at a temperature rising rate of 10° C./min. From the relationship between weight and temperature (thermogravimetry), it was understood that the 5% weight loss temperature was higher than or equal to 500° C., which is indicative of high heat resistance.

Next, 1,6oDMemFLPAPrn was analyzed by liquid chromatography mass spectrometry (LC/MS). The analysis by LC/MS was carried out with Acquity UPLC (manufactured by Waters Corporation) and Xevo G2 Tof MS (manufactured by Waters Corporation).

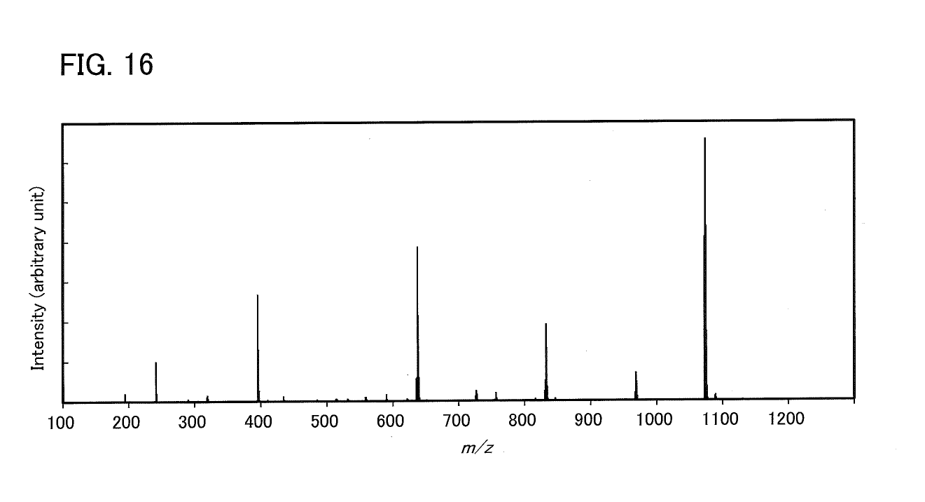

In the MS analysis, ionization was carried out by an electrospray ionization (abbreviation: ESI) method. At this time, the capillary voltage and the sample cone voltage were set to 3.0 kV and 30 V, respectively, and detection was performed in a positive mode. A component which underwent the ionization under the above-mentioned conditions was collided with an argon gas in a collision cell to dissociate into product ions. Energy (collision energy) for the collision with argon was 70 eV. The mass range for the measurement was m/z=100 to 1300. FIG. 16 shows the measurement results.

Next, ultraviolet-visible absorption spectra (hereinafter, simply referred to as “absorption spectra”) and emission spectra of 1,6oDMemFLPAPrn in a toluene solution and in a solid thin film were measured. The solid thin film was formed over a quartz substrate by a vacuum evaporation method. The absorption spectra were measured with an ultraviolet-visible light spectrophotometer (V550 type manufactured by JASCO Corporation). The emission spectra were measured with a fluorescence spectrophotometer (FS920 manufactured by Hamamatsu Photonics K.K.).

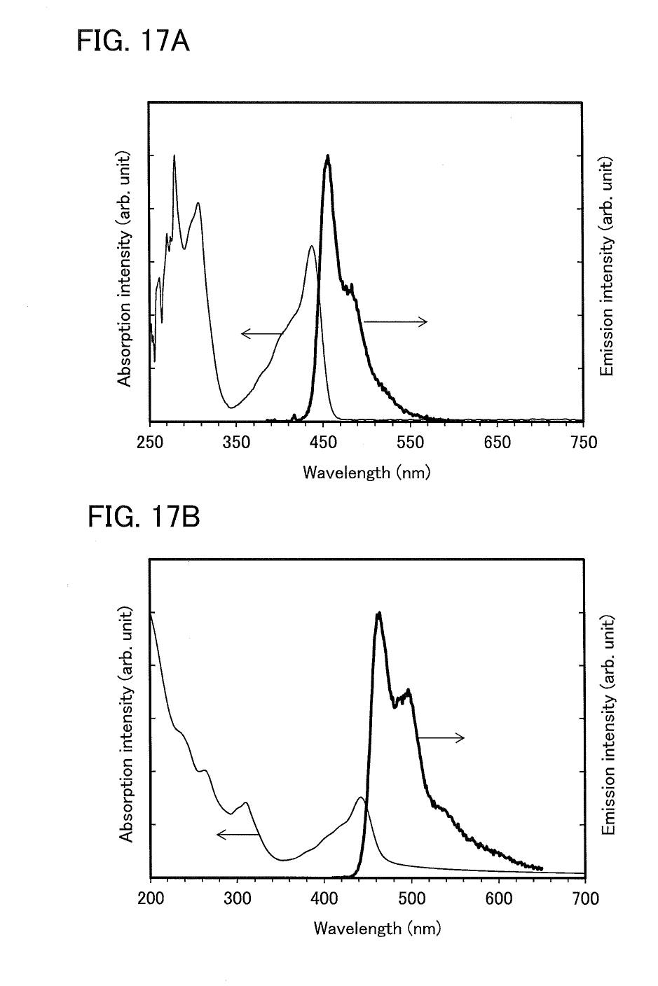

FIGS. 17A and 17B show measurement results. As seen in FIGS. 17A and 17B, an absorption peak of 1,6oDMemFLPAPrn in the toluene solution was observed at around 438 nm, and absorption peaks of 1,6oDMemFLPAPrn in a thin film were observed at around 443 nm, 421 nm, 400 nm, 382 nm, 310 nm, 301 nm, 263 nm, and 246 nm. An emission wavelength peak of 1,6oDMemFLPAPrn in the toluene solution was observed at around 457 nm, and emission wavelength peaks of 1,6oDMemFLPAPrn in the thin film were observed at around 530 nm, 497 nm, and 464 nm.

The ionization potential of 1,6oDMemFLPAPrn in a thin film state was measured by a photoelectron spectrometer (AC-3, manufactured by Riken Keiki, Co., Ltd.) in the air. The obtained value of the ionization potential was converted into a negative value, so that the HOMO level of 1,6oDMemFLPAPrn was −5.61 eV. From the data of the absorption spectrum of the thin film, the absorption edge of 1,6oDMemFLPAPrn, which was obtained from Tauc plot with an assumption of direct transition, was 2.69 eV. Therefore, the optical energy gap of 1,6oDMemFLPAPrn in a solid state is estimated to 2.69 eV According to the values of the HOMO level obtained above and this energy gap, the LUMO level of 1,6oDMemFLPAPrn can be estimated to −2.92 V.

### Example 2

In this example, a light-emitting element of one embodiment of the present invention (Light-emitting element 1) and a Comparative light-emitting element 1 are described. Structure formulae of organic compounds used for Light-emitting element 1 and Comparative light-emitting element 1 are shown below.

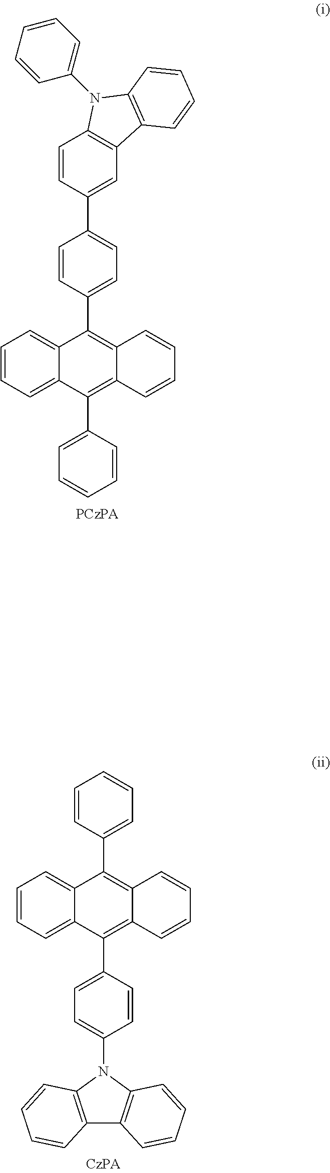 [SMILES: c1ccc(-c2c3ccccc3c(-c3ccc(-c4ccc5c(c4)c4ccccc4n5-c4ccccc4)cc3)c3ccccc23)cc1 ; c1ccc(-c2c3ccccc3c(-c3ccc(-n4c5ccccc5c5ccccc54)cc3)c3ccccc23)cc1]

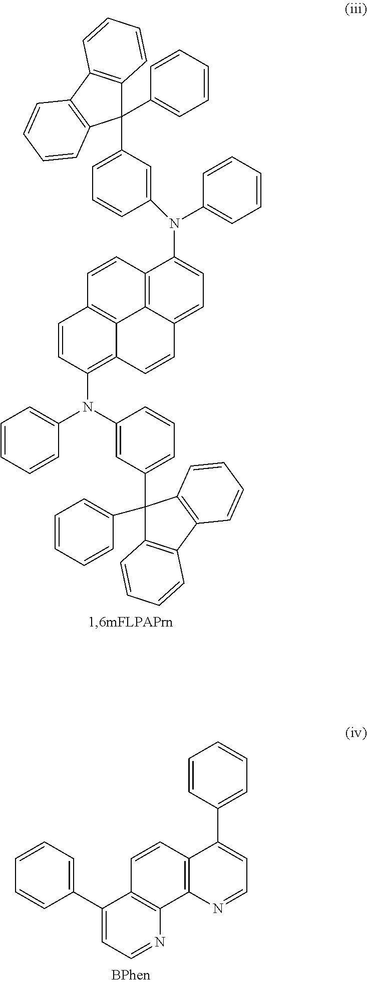 [SMILES: c1ccc(N(c2cccc(C3(c4ccccc4)c4ccccc4-c4ccccc43)c2)c2ccc3ccc4c(N(c5ccccc5)c5cccc(C6(c7ccccc7)c7ccccc7-c7ccccc76)c5)ccc5ccc2c3c54)cc1 ; c1ccc(-c2ccnc3c2ccc2c(-c4ccccc4)ccnc23)cc1]

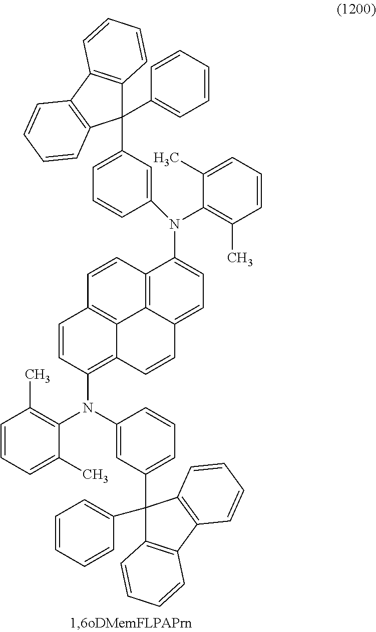 [SMILES: Cc1cccc(C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C)cccc4C)ccc4ccc1c2c43]

### (Method of Manufacturing Light-Emitting Element 1)

First, a film of indium tin oxide containing silicon oxide (ITSO) was formed over a glass substrate by a sputtering method, so that the first electrode 101 was formed. The thickness of the first electrode 101 was set to 110 nm and the area of the electrode was set to 2 mm×2 mm. Here, the first electrode 101 is an electrode that functions as an anode of a light-emitting element.

Next, in pretreatment for forming the light-emitting element over the substrate, a surface of the substrate was washed with water and baked at 200° C. for an hour, and then UV ozone treatment was performed for 370 seconds.

Then, the substrate was transferred into a vacuum evaporation apparatus whose pressure was reduced to approximately 10⁻⁴ Pa, vacuum baking at 170° C. for 30 minutes was performed in a heating chamber of the vacuum evaporation apparatus, and then the substrate was cooled down for approximately 30 minutes.

Then, the substrate over which the first electrode 101 was formed was fixed to a substrate holder provided in the vacuum evaporation apparatus so that the surface on which the first electrode 101 was formed faced downward. The pressure in the vacuum evaporation apparatus was reduced to about 10⁻⁴ Pa. After that, over the first electrode 101, 9-phenyl-3-[4-(10-phenyl-9-anthryl)phenyl]-9H-carbazole (abbreviation: PCzPA) represented by the above Structural formula (i) and molybdenum(VI) oxide were deposited by co-evaporation by an evaporation method using resistance heating, so that the hole-injection layer 111 was formed. The thickness of the hole-injection layer 111 was set to 50 nm, and the weight ratio of PCzPA to molybdenum oxide was adjusted to 4:2 (=PCzPA:molybdenum oxide). Note that the co-evaporation method refers to an evaporation method in which evaporation is carried out from a plurality of evaporation sources at the same time in one treatment chamber.

Next, a film of PCzPA was formed to a thickness of 10 nm over the hole-injection layer 111 to form the hole-transport layer 112.

Furthermore, over the hole-transport layer 112, the light-emitting layer 113 was formed by co-evaporation of 9-[4-(10-phenyl-9-anthryl)phenyl]-9H-carbazole (abbreviation: CzPA) represented by Structural formula (ii) and N,N′-bis(2,6-dimethylphenyl)-N,N′-bis[3-(9-phenyl-9H-fluoren-9-yl)phenyl]pyrene-1,6-diamine (abbreviation: 1,6oDMemFLPAPrn) represented by Structural formula (1200) with a weight ratio of 1:0.01 (=CzPA: 1,6oDMemFLPAPrn) to a thickness of 25 nm.

Then, the electron-transport layer 114 was formed over the light-emitting layer 113 in such a way that a 10-nm-thick film of CzPA was formed and a 15-nm-thick film of bathophenanthroline (abbreviation: BPhen) represented by Structural formula (iv) was formed.

After the formation of the electron-transport layer 114, lithium fluoride (LiF) was deposited by evaporation to a thickness of 1 nm to form the electron-injection layer 115. Finally, aluminum was deposited by evaporation to a thickness of 200 nm to form the second electrode 102 functioning as a cathode. Through the above-described steps, Light-emitting element 1 of this example was fabricated.

### (Method of Fabricating Light-Emitting Element 2)

Light-emitting element 2 was fabricated in the same manner as Light-emitting element 1 except that the light-emitting layer 113 was formed to a thickness of 25 nm by co-evaporation such that the weight ratio of CzPA to 1,6oDMemFLPAPrn in the light-emitting layer 113 was 1:0.03 (=CzPA:1,6oDMemFLPAPrn).

### (Method of Fabricating Comparative Light-Emitting Element 1)

Comparative light-emitting element 1 was fabricated in the same manner as Light-emitting element 1 except that 1,6oDMemFLPAPrn in the light-emitting layer 113 of Light-emitting element 1 was replaced with N,N′-bis[3-(9-phenyl-9H-fluoren-9-yl)phenyl]-N,N′-diphenylpyrene-1,6-diamine (abbreviation: 1,6mFLPAPrn) represented by Structural formula (iii).

The element structures of Light-emitting element 1, Light-emitting element 2, and Comparative light-emitting element 1 are listed in Table 2.

TABLE 2

| | Hole- | Hole- | | | | |
|---|---|---|---|---|---|---|
| | injection | transport | | Electron- | | Electron- |
| | layer | layer | Light-emitting layer | transport layer | | injection |
| | 50 nm | 10 nm | 25 nm | 10 nm | 15 nm | layer |
| Light-emitting | PCzPA:MoOx | PCzPA | CzPA:1, | CzPA | BPhen | LiF |
| element 1 | 4:2 | | 6oDMemFLPAPrn | | | |
| | | | 1:0.01 | | | |
| Light-emitting | | | CzPA:1, | | | |
| element 2 | | | 6oDMemFLPAPrn | | | |
| | | | 1:0.03 | | | |
| Comparative | | | CzPA:1, | | | |
| light-emitting | | | 6mFLPAPrn | | | |
| element 1 | | | 1:0.03 | | | |

Light-emitting elements 1 and 2 and Comparative light-emitting element 1 were each sealed using a glass substrate in a glove box containing a nitrogen atmosphere so as not to be exposed to the air (specifically, a sealing material was applied onto an outer edge of the element and UV treatment and heat treatment at 80° C. for an hour were performed at the time of sealing). Then, reliability of these light-emitting elements was measured. Note that the measurements were performed at room temperature (in an atmosphere kept at 25° C.).

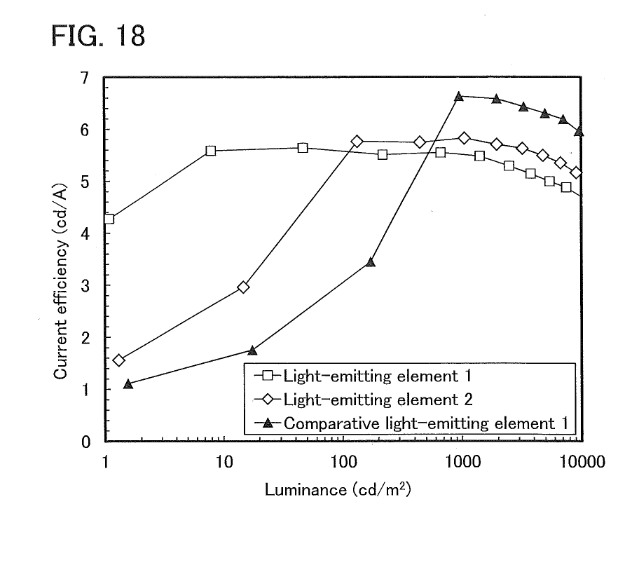

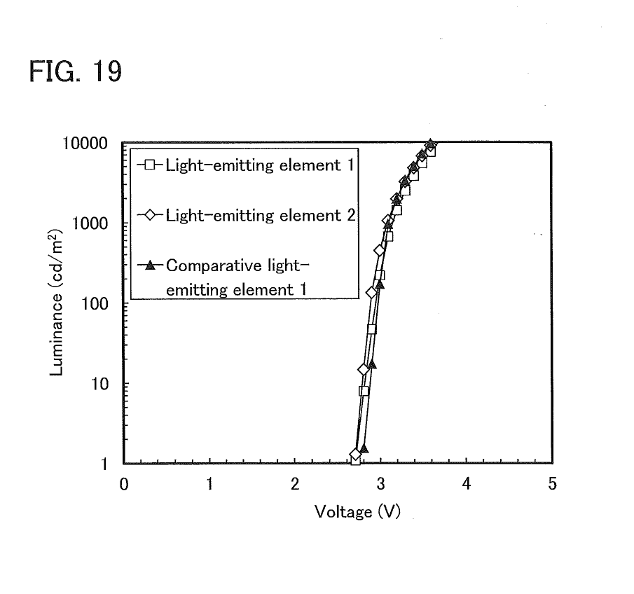

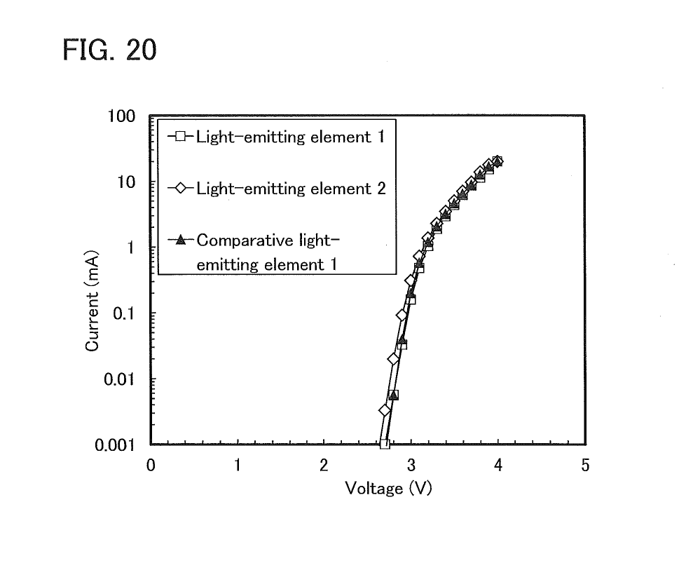

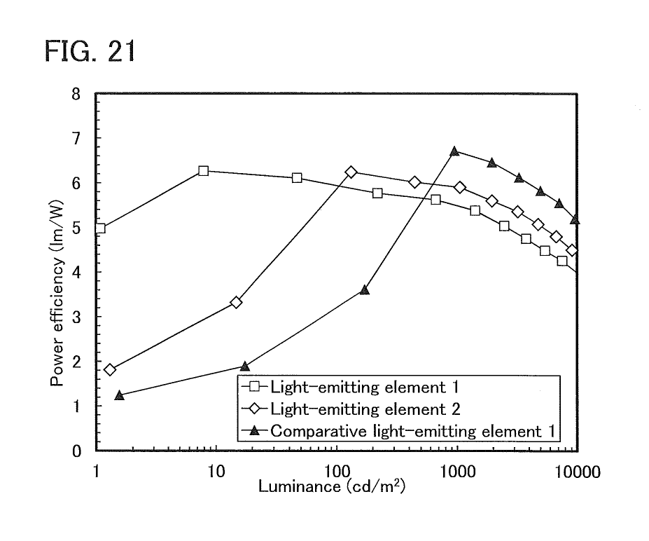

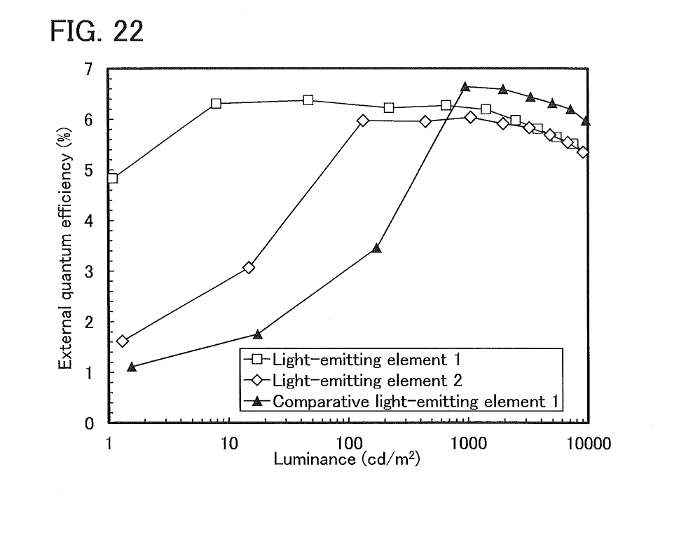

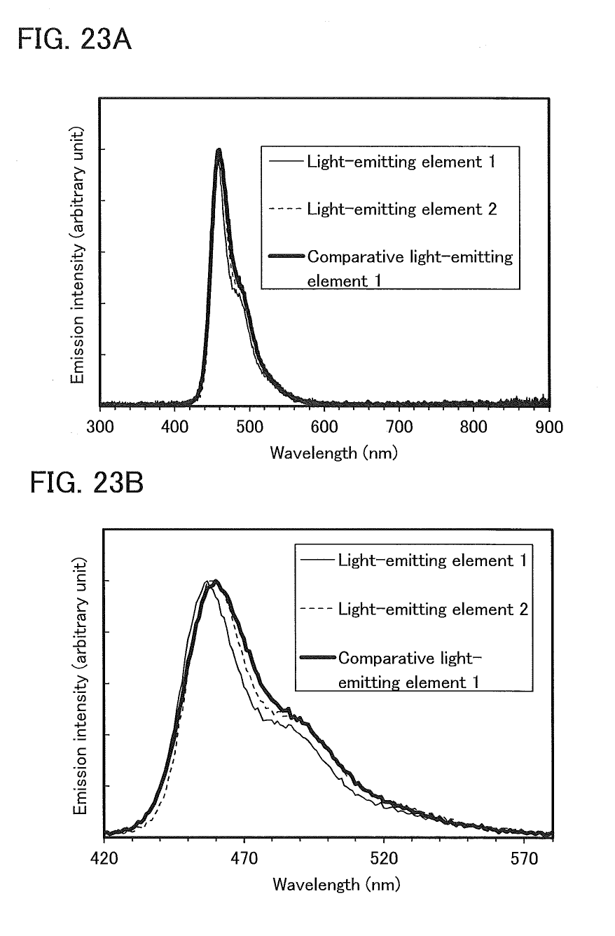

FIG. 18 shows luminance-current efficiency characteristics of Light-emitting elements 1 and 2 and Comparative light-emitting element 1. FIG. 19 shows voltage-luminance characteristics of thereof. FIG. 20 shows voltage-current characteristics thereof. FIG. 21 shows luminance-power efficiency characteristics thereof. FIG. 22 shows luminance-external quantum efficiency characteristics thereof. FIGS. 23A and 23B show emission spectra thereof.

The results show that Light-emitting element 1 and Comparative light-emitting element 1 both have favorable characteristics. FIG. 23B is an enlarged view of the spectrum ranging from 400 nm to 600 nm in FIG. 23A. As can be seen from FIG. 23B, each of Light-emitting element 1 and Light-emitting element 2 has a narrower spectrum than Comparative light-emitting element 1, and has a smaller peak wavelength than Comparative light-emitting element 1.

The external quantum efficiency of each of Light-emitting element 1 and Light-emitting element 2 is similar to that of Comparative light-emitting element 1. Although the maximum values of emission spectra shown in FIGS. 23A and 23B are normalized to 1, the maximum value of an emission intensity of each of Light-emitting element 1 and Light-emitting element 2, which have substantially the same quantum efficiency and each have a small half width of an emission spectrum, is larger than the maximum value of an emission intensity of Comparative light-emitting element 1. In view of a small amount of light decayed by the cavity effect or a small amount of light intercepted with a color filter, with the use of 1,6oDMemFLPAPrn, which is a 1,6-bis(diphenylamino)pyrene derivative of one embodiment of the present invention, a light-emitting element with extremely high emission efficiency or a light-emitting element with extremely low power consumption can be obtained.

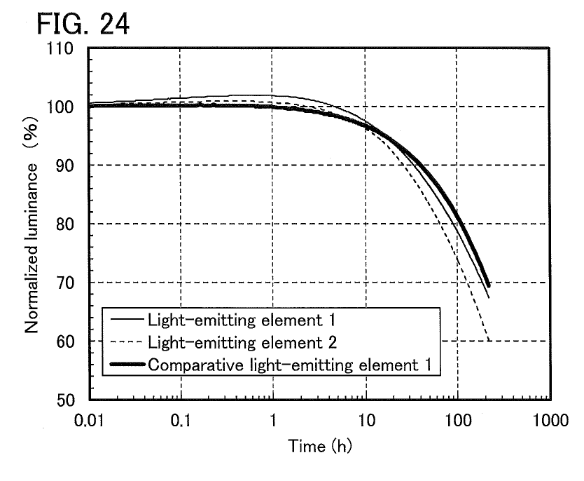

Light-emitting element 1 and Comparative light-emitting element 1 were driven at a constant current of 2.81 mA. A luminance change with driving time was measured on the assumption that the initial luminance is 100. FIG. 24 shows the measurement results. FIG. 24 indicates that Light-emitting element 1, Light-emitting element 2, and Comparative light-emitting element 1 have favorable characteristics.

Light-emitting element 1 including the 1,6-bis(diphenylamino)pyrene derivative in which an alkyl group is bonded to each of the two ortho positions of at least one of the two phenyl groups in each of the two diphenylamino groups (1,6oDMemFLPAPrn is used in this example) as a phosphorescent substance has characteristics similar to those of Comparative light-emitting element 1 including a 1,6-bis(diphenylamino)pyrene derivative without the above structure (1,6mFLPAPrn). In addition, Light-emitting element 1 has a narrower half width of an emission spectrum than Comparative light-emitting element 1.

### Example 3

In this example, a method of synthesizing N,N′-bis[3-(dibenzofuran-4-yl)-2,6-dimethylphenyl]-N,N′-diphenylpyrene-1,6-diamine (abbreviation: 1,6mFrBAPrn-04), which is an organic compound of one embodiment of the present invention, is described. A structural formula of 1,6mFrBAPrn-04 is shown below.

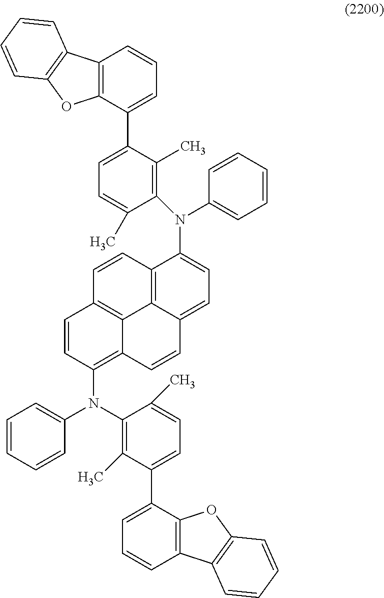 [SMILES: Cc1ccc(-c2cccc3c2oc2ccccc23)c(C)c1N(c1ccccc1)c1ccc2ccc3c(N(c4ccccc4)c4c(C)ccc(-c5cccc6c5oc5ccccc56)c4C)ccc4ccc1c2c43]

### Step 1: Synthesis of N-[3-(dibenzofuran-4-yl)-2,6-dimethylphenyl]-N-phenylamine (abbreviation: mFrBA-04)

Into a 200-mL three-neck flask was put 5.2 g (26.1 mmol) of 3-bromo-2,6-dimethylaniline, and the air in the flask was replaced with nitrogen. To this flask were added 98.0 mL of toluene, 32.0 mL of ethanol, 6.6 g (31.3 mmol) of 4-dibenzofuran boronic acid, 0.4 g (1.3 mmol) of tris(2-methylphenyl)phosphine, and 25.8 mL of a potassium carbonate solution (2 mol/L). The mixture was degassed, and 0.09 g (0.4 mmol) of palladium(II) acetate was added thereto. The mixture was stirred at 90° C. for 15.5 hours. After the stirring, toluene and water were added to the mixture, an organic layer and an aqueous layer were separated, and the aqueous layer was extracted twice with toluene and extracted twice with ethyl acetate. The extracted solution was combined with the organic layer and dried with magnesium sulfate. The obtained mixture was gravity filtered to remove magnesium sulfate, and the obtained filtrate was concentrated to give a solid.

Next, 4.0 g (42.0 mmol) of sodium tert-butoxide was added to a 200-mL three-neck flask, and the air in the flask was replaced with nitrogen. To the flask were added 30.0 mL of toluene, 4.0 g of the obtained solid dissolved in 40.0 mL of toluene, 1.5 mL (13.9 mmol) of 2-bromobenzene, and 0.5 mL of a 10% hexane solution of tri(tert-butyl)phosphine. To this mixture was added 33.1 mg (0.1 mmol) of bis(dibenzylideneacetone)palladium(0), and stirring was performed at 80° C. for 10.2 hours. After the stirring, the mixture was filtered through Florisil, Celite, and alumina to obtain a filtrate. The obtained filtrate was concentrated to give a solid. The solid was purified by silica gel column chromatography (developing solvent:a mixed solvent of hexane:toluene=3:1). Accordingly, 3.3 g of the target compound was obtained in a yield of 89%. A synthesis scheme of Step 1 is shown below.

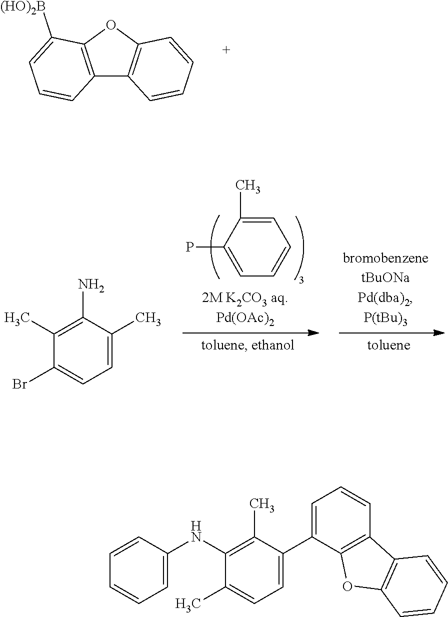

¹H NMR data of the obtained substance are as follows: ¹H NMR (CDCl₃, 500 MHz): δ=2.09 (s, 3H), 2.31 (s, 3H), 5.34 (s, 1H), 6.63 (d, J=8.0 Hz, 2H), 6.78 (t, J=7.5 Hz, 1H), 7.19-7.27 (m, 4H), 7.33-7.45 (m, 4H), 7.52 (d, J=8.0 Hz, 1H), 7.95-7.99 (m, 2H).

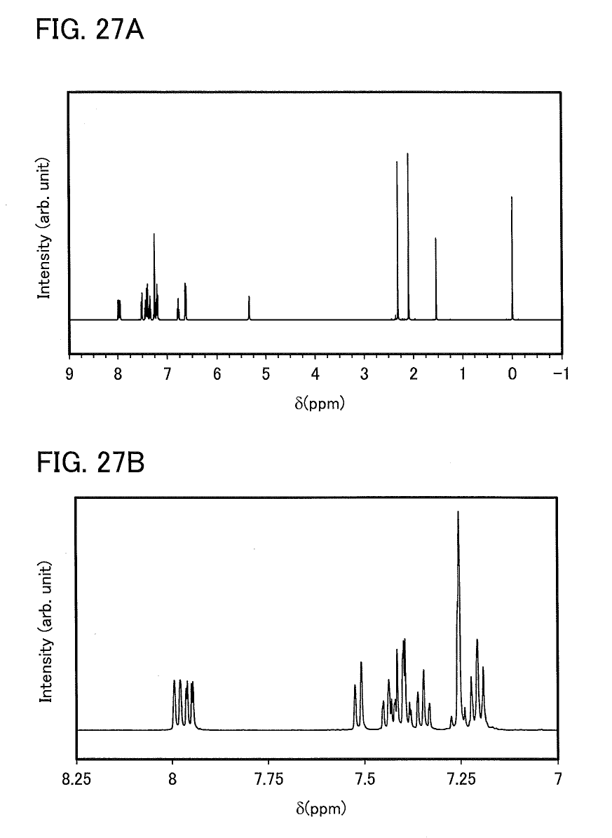

FIGS. 27A and 27B are ¹H-NMR charts. Note that FIG. 27B is a chart showing an enlarged part of FIG. 27A in the range of 7.00 ppm to 8.25 ppm. This indicates that mFrBA-04 was obtained.

### Step 2: Synthesis of [3-(dibenzofuran-4-yl)-2,6-dimethylphenyl]-N,N-diphenylpyrene-1,6-diamine (1,6mFrBAPrn-04)

Into a 100-mL three-neck flask were put 0.8 g (2.2 mmol) of 1,6-dibromopyrene, 1.6 g (4.4 mmol) of N-[3-(dibenzofuran-4-yl)-2,6-dimethylphenyl]-N-phenylamine, and 0.6 g (6.6 mmol) of sodium tert-butoxide, and the air in the flask was replaced with nitrogen. To this mixture were added 22.0 mL of xylene and 0.5 mL of a 10% hexane solution of tri(tert-butyl)phosphine. The temperature of this mixture was set to 80° C., 37.2 mg (0.1 mmol) of bis(dibenzylideneacetone)palladium(0) was added, and the mixture was refluxed while being stirred for 7.3 hours. After the stirring, 40.2 mg (0.1 mmol) of bis(dibenzylideneacetone)palladium(0) was added, the mixture was stirred for 8.3 hours, 41.0 mg (0.1 mmol) of bis(dibenzylideneacetone)palladium(0) was added, and the mixture was stirred for 8.5 hours. Then, the mixture was suction filtered, the obtained residue was dissolved in toluene, and this mixture was suction filtered through Florisil (produced by Wako Pure Chemical Industries, Ltd., Catalog No. 540-00135), Celite (produced by Wako Pure Chemical Industries, Ltd., Catalog No. 531-16855), and alumina to give a filtrate. The obtained filtrate was concentrated to give a solid. The obtained solid was purified by silica gel column chromatography (developing solvent:a mixed solvent of hexane:toluene=3:1) to give a solid. Recrystallization of the obtained solid from a mixed solvent of toluene and hexane was performed to give 0.8 g of the target yellow solid in a yield of 42%. A synthesis scheme of Step 2 is shown below.

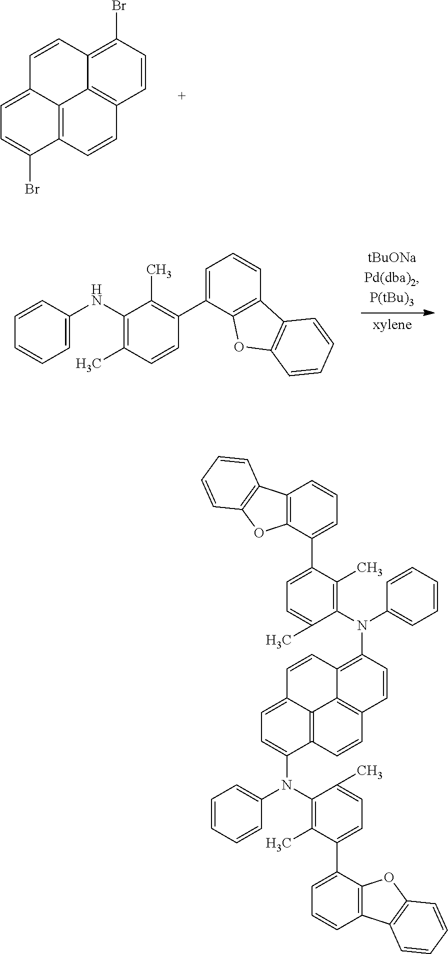 [SMILES: Brc1ccc2ccc3c(Br)ccc4ccc1c2c43 ; Cc1ccc(-c2cccc3c2oc2ccccc23)c(C)c1Nc1ccccc1 ; Cc1ccc(-c2cccc3c2oc2ccccc23)c(C)c1N(c1ccccc1)c1ccc2ccc3c(N(c4ccccc4)c4c(C)ccc(-c5cccc6c5oc5ccccc56)c4C)ccc4ccc1c2c43]

By a train sublimation method, 0.8 g of the obtained yellow solid was purified. In the purification by sublimation, the yellow solid was heated at 352° C. at a pressure of 4.1×10⁻² Pa without an argon gas stream. After the purification by sublimation, 0.7 g of the target yellow solid was obtained at a collection rate of 78%.

The obtained substance was analyzed by ¹H NMR, and the measurement data are as follows: ¹H NMR (CDCl₃, 500 MHz): δ=2.00 (d, J=5.0 Hz, 6H), 2.19 (d, J=5.5 Hz, 6H), 6.73-6.89 (m, 6H), 7.14-7.46 (m, 18H), 7.61 (d, J=8.5 Hz, 2H), 7.81 (d, =9.0 Hz, 2H), 7.92-8.02 (m, 8H).

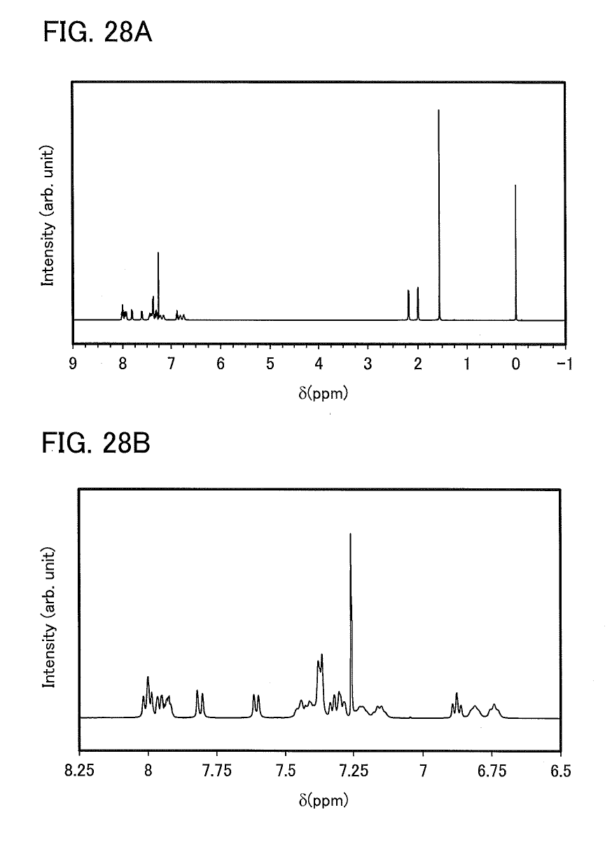

The ¹H NMR chart is shown in FIGS. 28A and 28B. Note that FIG. 28B is a chart showing an enlarged part of FIG. 28A in the range of 6.50 ppm to 8.25 ppm. The charts reveal that 1,6mFrBAPrn-04 represented by the above Structural formula (2200), which is an organic compound of one embodiment of the present invention, was obtained.

Thermogravimetry-differential thermal analysis (TG-DTA) of obtained 1,6mFrBAPrn-04 was performed. A high vacuum differential type differential thermal balance (TG/DTA 2410SA, manufactured by Bruker AXS K.K.) was used for the measurement. The measurement was carried out under a nitrogen stream (a flow rate of 200 mL/min) and a normal pressure at a temperature rising rate of 10° C./min. From the relationship between weight and temperature (thermogravimetry), it was understood that the 5% weight loss temperature was 487° C., which is indicative of high heat resistance.

Next, 1,6mFrBAPrn-04 was analyzed by liquid chromatography mass spectrometry (LC/MS). The analysis by LC/MS was carried out with Acquity UPLC (manufactured by Waters Corporation) and Xevo G2 Tof MS (manufactured by Waters Corporation).

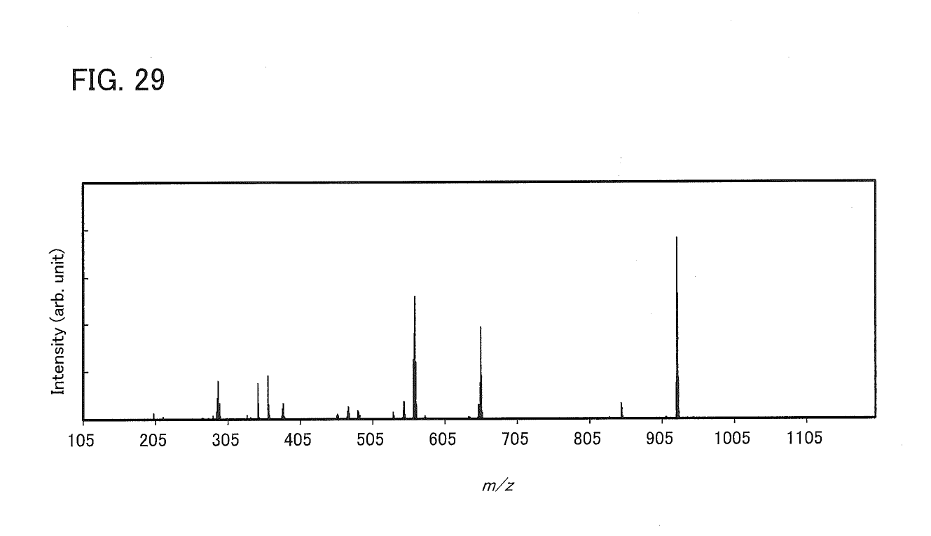

In the MS analysis, ionization was carried out by an electrospray ionization (abbreviation: ESI) method. At this time, the capillary voltage and the sample cone voltage were set to 3.0 kV and 30 V, respectively, and detection was performed in a positive mode. A component which underwent the ionization under the above-mentioned conditions was collided with an argon gas in a collision cell to dissociate into product ions. Energy (collision energy) for the collision with argon was 70 eV. The mass range for the measurement was m/z=100 to 1200. FIG. 29 shows the measurement results.

Next, ultraviolet-visible absorption spectra (hereinafter, simply referred to as “absorption spectra”) and emission spectra of 1,6mFrBAPrn-04 in a toluene solution and in a solid thin film were measured. The solid thin film was formed over a quartz substrate by a vacuum evaporation method. The absorption spectra were measured with an ultraviolet-visible light spectrophotometer (V550 type manufactured by JASCO Corporation). The emission spectra were measured with a fluorescence spectrophotometer (FS920 manufactured by Hamamatsu Photonics K.K.).

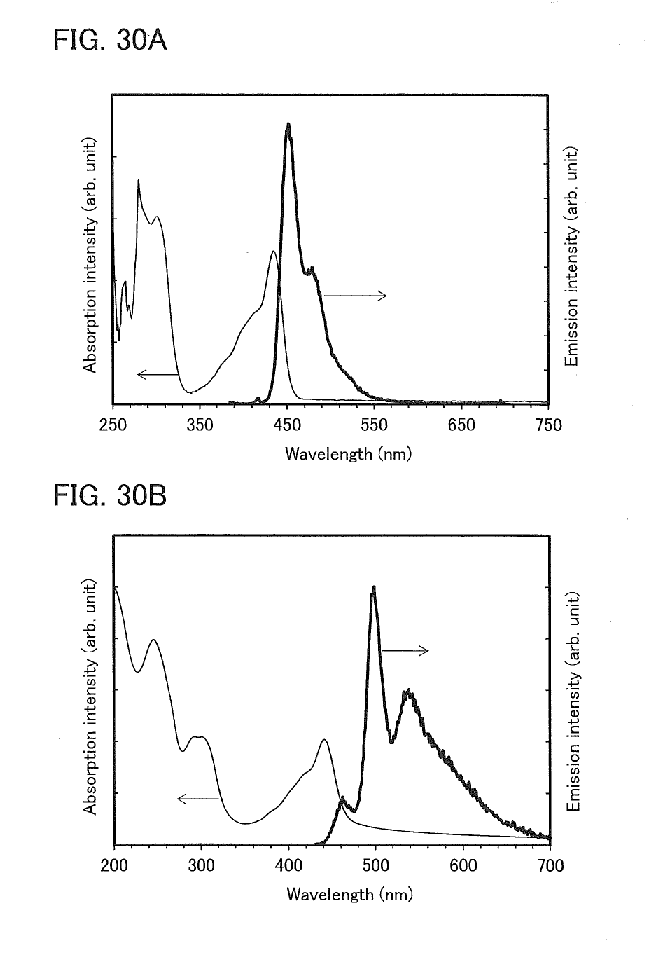

FIGS. 30A and 30B show measurement results. As seen in FIGS. 30A and 30B, an absorption peak of 1,6mFrBAPrn-04 in the toluene solution was observed at around 435 nm, and absorption peaks of 1,6mFrBAPrn-04 in a thin film were observed at around 441 nm, 420 nm, 383 nm, 302 nm, 292 nm, and 246 nm. An emission wavelength peak of 1,6mFrBAPrn-04 in the toluene solution was observed at around 452 nm, and emission wavelength peaks of 1,6mFrBAPrn-04 in the thin film were observed at around 538 nm, 498 nm, and 462 nm.

The ionization potential of 1,6mFrBAPrn-04 in a thin film state was measured by a photoelectron spectrometer (AC-3, manufactured by Riken Keiki, Co., Ltd.) in the air. The obtained value of the ionization potential was converted into a negative value, so that the HOMO level of 1,6mFrBAPrn-04 was −5.66 eV. From the data of the absorption spectrum of the thin film, the absorption edge of 1,6mFrBAPrn-04, which was obtained from Tauc plot with an assumption of direct transition, was 2.69 eV. Therefore, the optical energy gap of 1,6mFrBAPrn-04 in a solid state is estimated to 2.69 eV. According to the values of the HOMO level obtained above and this energy gap, the LUMO level of 1,6mFrBAPrn-04 can be estimated to −2.97 V.

### Example 4

In this example, a method of synthesizing N,N′-bis[3-(dibenzofuran-4-yl)phenyl]-N,N′-bis(2,6-dimethylphenyl)pyrene-1,6-diamine (abbreviation: 1,6oDMemFrBAPrn), which is an organic compound of one embodiment of the present invention, is described. A structural formula of 1,6oDMemFrBAPrn is shown below.

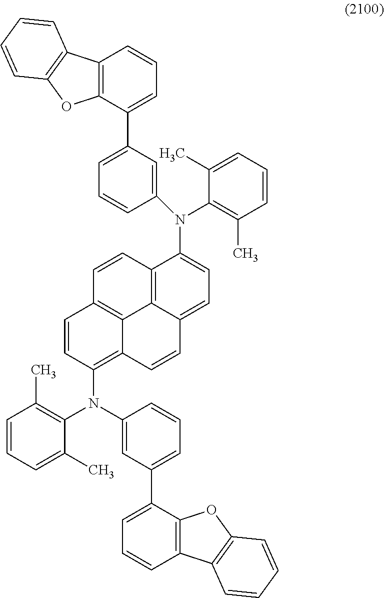 [SMILES: Cc1cccc(C)c1N(c1cccc(-c2cccc3c2oc2ccccc23)c1)c1ccc2ccc3c(N(c4cccc(-c5cccc6c5oc5ccccc56)c4)c4c(C)cccc4C)ccc4ccc1c2c43]

### Step 1: Synthesis of N-[3-(dibenzofuran-4-yl)phenyl]-N-(2,6-dimethylphenyl)amine (abbreviation: oDMemFrBA)

Into a 200-mL three-neck flask were put 4.4 g (13.8 mmol) of 4-(3-bromophenyl)dibenzofuran and 4.0 g (41.3 mmol) of sodium tert-butoxide, and the air in the flask was replaced with nitrogen. Then, 2.6 mL (21.0 mmol) of 2,6-dimethylaniline, 65.0 mL of toluene, 0.5 mL of a 10% hexane solution of tri(tert-butyl)phosphine, and 57.4 mg (0.1 mmol) of bis(dibenzylideneacetone)palladium(0) were added thereto, the mixture of the mixture was set to 90° C., and the mixture was stirred for 6.5 hours. After the stirring, toluene and water were added to the mixture, an organic layer and an aqueous layer were separated, and the aqueous layer was extracted three times with toluene. The extracted solution was combined with the organic layer and dried with magnesium sulfate. The obtained mixture was gravity filtered to remove magnesium sulfate, and a filtrate was obtained. The filtrate was concentrated to give a solid. The solid was purified by silica gel column chromatography (the developing solvent has a 5:1 ratio of hexane to toluene). Accordingly, 4.9 g of the target compound was obtained in a yield of 98%. A synthesis scheme of Step 1 is shown below.

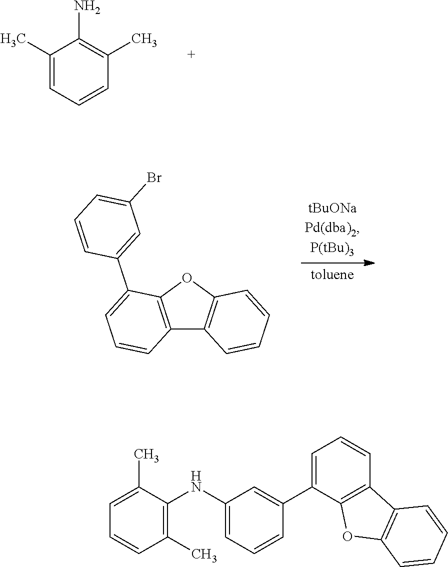

¹H NMR data of the obtained substance are as follows: ¹H NMR (CDCl₃, 500 MHz): δ=2.32 (s, 6H), 5.35 (s, 1H), 6.60-6.62 (m, 1H), 7.06-7.10 (m, 2H), 7.15 (d, J=7.5 Hz, 2H), 7.28-7.39 (m, 4H), 7.46 (t, J=8.0 Hz, 1H), 7.55-7.57 (m, 2H), 7.89 (d, J=7.5 Hz, 1H), 7.96 (d, J=8.0 Hz, 1H).

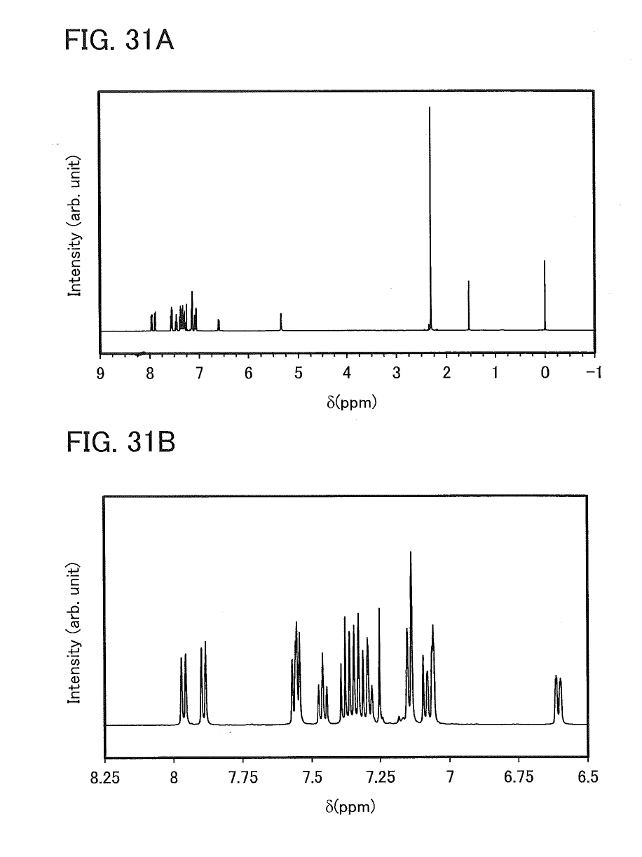

FIGS. 31A and 31B are ¹H-NMR charts. Note that FIG. 31B is a chart showing an enlarged part of FIG. 31A in the range of 6.5 ppm to 8.25 ppm. This indicates that oDMemFrBA was obtained.

### Step 2: Synthesis of N,N′-bis[3-(dibenzofuran-4-yl)phenyl]-N,N′-bis(2,6-dimethylphenyl)pyrene-1,6-diamine (abbreviation: 1,6oDMemFrBAPrn)

Into a 100-mL three-neck flask were put 0.8 g (2.2 mmol) of 1,6-dibromopyrene, 1.6 g (4.3 mmol) of N-[3-(dibenzofuran-4-yl)phenyl]-N-(2,6-dimethylphenyl)amine, and 0.6 g (6.6 mmol) of sodium tert-butoxide, and the air in the flask was replaced with nitrogen. To this mixture were added 21.0 mL of xylene and 0.8 mL of a 10% hexane solution of tri(tert-butyl)phosphine. The temperature of this mixture was set to 80° C., 31.2 mg (0.1 mmol) of bis(dibenzylideneacetone)palladium(0) was added, and the mixture was stirred for 3.0 hours. After the stirring, 27.2 mg (0.05 mmol) of bis(dibenzylideneacetone)palladium (0) was added, the temperature of this mixture was set to 120° C., and stirring was performed for 1.5 hours. Then, the mixture was suction filtered, and the obtained residue was dissolved in toluene. This mixture was suction filtered through Florisil (produced by Wako Pure Chemical Industries, Ltd., Catalog No. 540-00135), Celite (produced by Wako Pure Chemical Industries, Ltd., Catalog No. 531-16855), and alumina to give a filtrate. The obtained filtrate was concentrated to give a solid. The obtained solid was washed with toluene, so that 0.6 g of the target solid was obtained in a yield of 30%. A synthesis scheme of Step 2 is shown below.

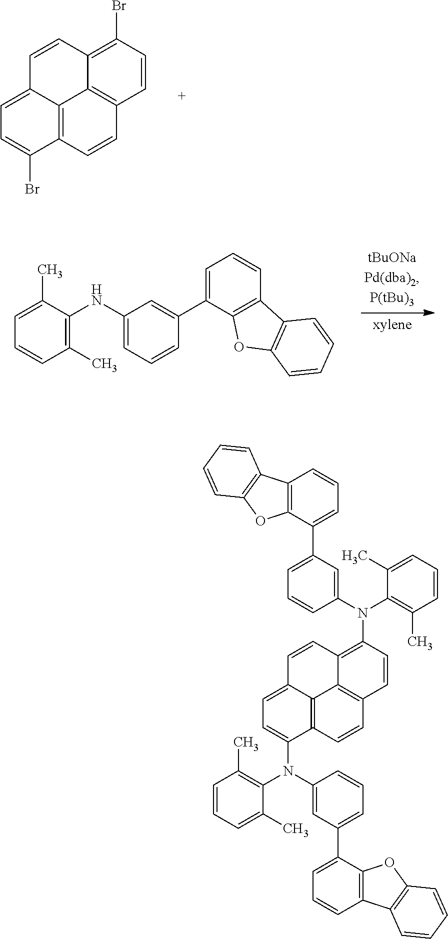 [SMILES: Brc1ccc2ccc3c(Br)ccc4ccc1c2c43 ; Cc1cccc(C)c1Nc1cccc(-c2cccc3c2oc2ccccc23)c1 ; Cc1cccc(C)c1N(c1cccc(-c2cccc3c2oc2ccccc23)c1)c1ccc2ccc3c(N(c4cccc(-c5cccc6c5oc5ccccc56)c4)c4c(C)cccc4C)ccc4ccc1c2c43]

By a train sublimation method, 0.6 g of the obtained solid was purified. In the purification by sublimation, the solid was heated at 330° C. for 2.0 hours and at 338° C. for 2.5 hours at a pressure of 1.3×10⁻² Pa without an argon gas stream. After the purification by sublimation, 0.4 g of the solid of 1,6oDMemFrBAPrn was obtained at a collection rate of 69%.

Thermogravimetry-differential thermal analysis (TG-DTA) of obtained 1,6oDMemFrBAPrn was performed. A high vacuum differential type differential thermal balance (TG/DTA 2410SA, manufactured by Bruker AXS K.K.) was used for the measurement. The measurement was carried out under a nitrogen stream (a flow rate of 200 mL/min) and a normal pressure at a temperature rising rate of 10° C./min From the relationship between weight and temperature (thermogravimetry), it was understood that the 5% weight loss temperature was 476° C., which is indicative of high heat resistance.

Next, 1,6oDMemFrBAPrn was analyzed by liquid chromatography mass spectrometry (LC/MS). The analysis by LC/MS was carried out with Acquity UPLC (manufactured by Waters Corporation) and Xevo G2 Tof MS (manufactured by Waters Corporation).

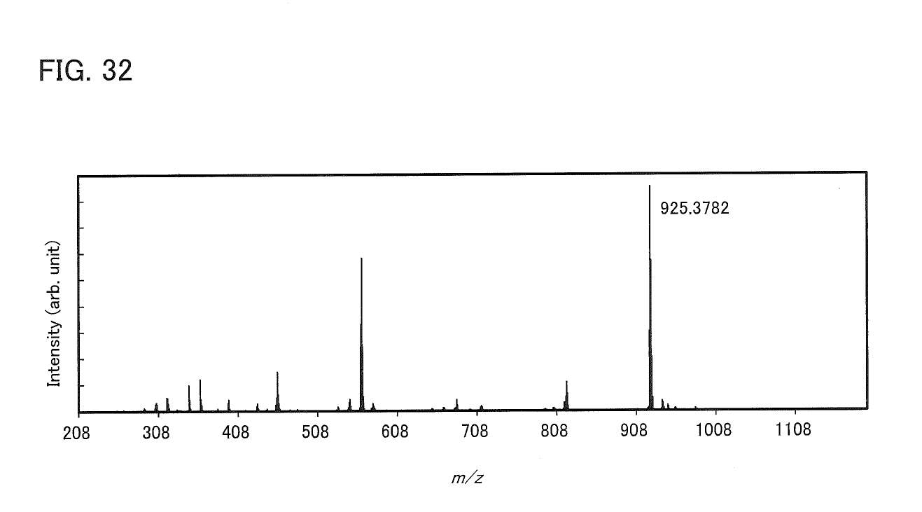

In the MS analysis, ionization was carried out by an electrospray ionization (abbreviation: ESI) method. At this time, the capillary voltage and the sample cone voltage were set to 3.0 kV and 30 V, respectively, and detection was performed in a positive mode. A component which underwent the ionization under the above-mentioned conditions was collided with an argon gas in a collision cell to dissociate into product ions. Energy (collision energy) for the collision with argon was 70 eV. The mass range for the measurement was m/z=100 to 1200. FIG. 32 shows the measurement results.

Next, ultraviolet-visible absorption spectra (hereinafter, simply referred to as “absorption spectra”) and emission spectra of 1,6oDMemFrBAPrn in a toluene solution and in a solid thin film were measured. The solid thin film was formed over a quartz substrate by a vacuum evaporation method. The absorption spectra were measured with an ultraviolet-visible light spectrophotometer (V550 type manufactured by JASCO Corporation). The emission spectra were measured with a fluorescence spectrophotometer (FS920 manufactured by Hamamatsu Photonics K.K.).

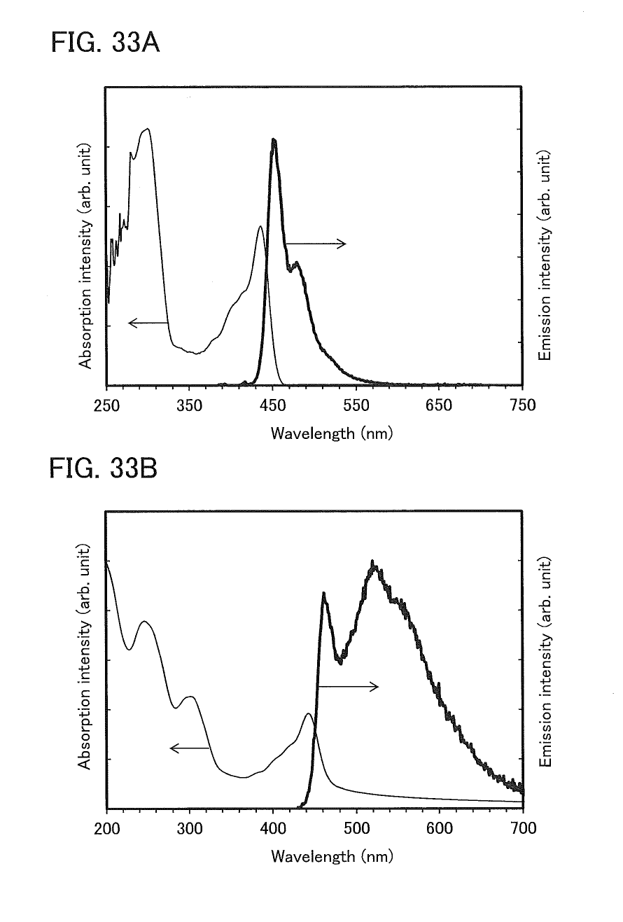

FIGS. 33A and 33B show measurement results. As seen in FIGS. 33A and 33B, an absorption peak of 1,6oDMemFrBAPrn in the toluene solution was observed at around 436 nm, and absorption peaks of 1,6oDMemFrBAPrn in a thin film were observed at around 443 nm, 419 nm, 403 nm, 381 nm, 302 nm, and 246 nm. An emission wavelength peak of 1,6oDMemFrBAPrn in the toluene solution was observed at around 453 nm, and emission wavelength peaks of 1,6oDMemFrBAPrn in the thin film were observed at around 550 nm, 532 nm, 462 nm, and 442 nm.

The ionization potential of 1,6oDMemFrBAPrn in a thin film state was measured by a photoelectron spectrometer (AC-3, manufactured by Riken Keiki, Co., Ltd.) in the air. The obtained value of the ionization potential was converted into a negative value, so that the HOMO level of 1,6oDMemFrBAPrn was −5.67 eV. From the data of the absorption spectrum of the thin film, the absorption edge of 1,6oDMemFrBAPrn, which was obtained from Tauc plot with an assumption of direct transition, was 2.68 eV. Therefore, the optical energy gap of 1,6oDMemFrBAPrn in a solid state is estimated to 2.68 eV. According to the values of the HOMO level obtained above and this energy gap, the LUMO level of 1,6oDMemFrBAPrn can be estimated to −2.99 V.

### Example 5

In this example, a light-emitting element of one embodiment of the present invention (Light-emitting element 3) and a Comparative light-emitting element 2 are described. Structure formulae of organic compounds used for Light-emitting element 3 and Comparative light-emitting element 2 are shown below.

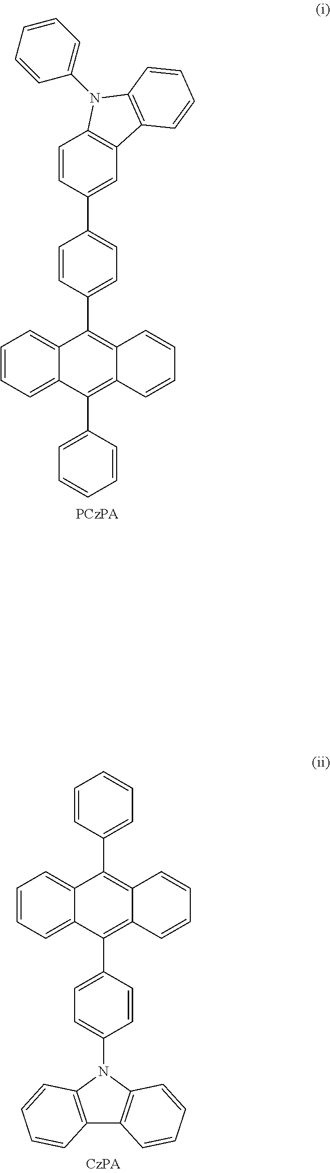 [SMILES: c1ccc(-c2c3ccccc3c(-c3ccc(-c4ccc5c(c4)c4ccccc4n5-c4ccccc4)cc3)c3ccccc23)cc1 ; c1ccc(-c2c3ccccc3c(-c3ccc(-n4c5ccccc5c5ccccc54)cc3)c3ccccc23)cc1]

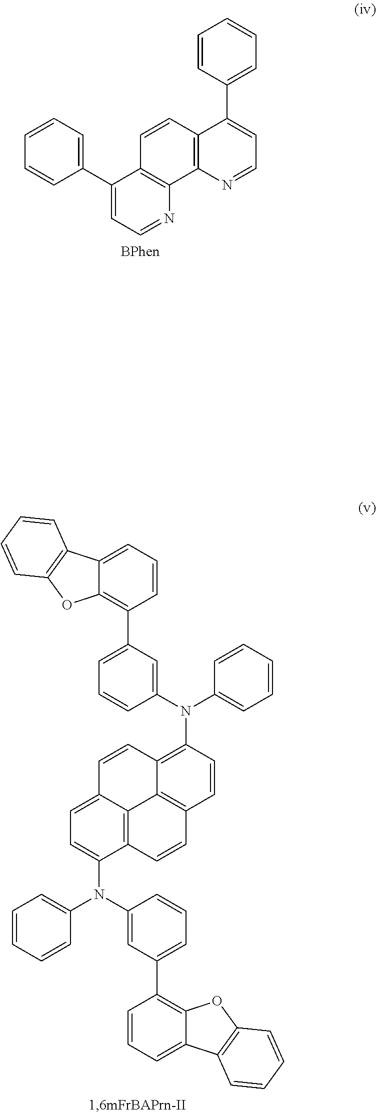 [SMILES: c1ccc(-c2ccnc3c2ccc2c(-c4ccccc4)ccnc23)cc1 ; c1ccc(N(c2cccc(-c3cccc4c3oc3ccccc34)c2)c2ccc3ccc4c(N(c5ccccc5)c5cccc(-c6cccc7c6oc6ccccc67)c5)ccc5ccc2c3c54)cc1]

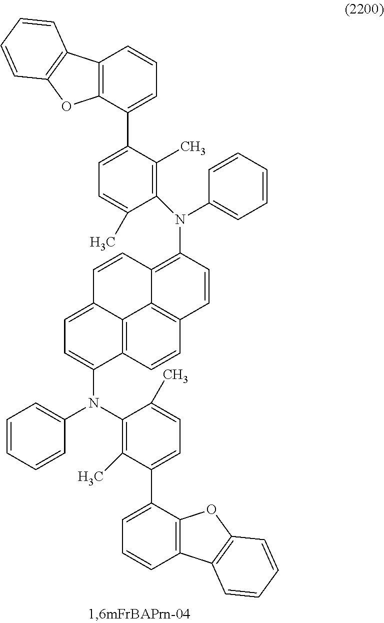 [SMILES: Cc1ccc(-c2cccc3c2oc2ccccc23)c(C)c1N(c1ccccc1)c1ccc2ccc3c(N(c4ccccc4)c4c(C)ccc(-c5cccc6c5oc5ccccc56)c4C)ccc4ccc1c2c43]

### (Method of Manufacturing Light-Emitting Element 3)

First, a film of indium tin oxide containing silicon oxide (ITSO) was formed over a glass substrate by a sputtering method, so that the first electrode 101 was formed. The thickness of the first electrode 101 was set to 110 nm and the area of the electrode was set to 2 mm×2 mm. Here, the first electrode 101 is an electrode that functions as an anode of a light-emitting element.

Next, in pretreatment for forming the light-emitting element over the substrate, a surface of the substrate was washed with water and baked at 200° C. for an hour, and then UV ozone treatment was performed for 370 seconds.

Then, the substrate was transferred into a vacuum evaporation apparatus whose pressure was reduced to approximately 10⁻⁴ Pa, vacuum baking at 170° C. for 30 minutes was performed in a heating chamber of the vacuum evaporation apparatus, and then the substrate was cooled down for approximately 30 minutes.

Then, the substrate over which the first electrode 101 was formed was fixed to a substrate holder provided in the vacuum evaporation apparatus so that the surface on which the first electrode 101 was formed faced downward. The pressure in the vacuum evaporation apparatus was reduced to about 10⁻⁴ Pa. After that, over the first electrode 101, 9-phenyl-3-[4-(10-phenyl-9-anthryl)phenyl]-9H-carbazole (abbreviation: PCzPA) represented by the above Structural formula (i) and molybdenum(VI) oxide were deposited by co-evaporation by an evaporation method using resistance heating, so that the hole-injection layer 111 was formed. The thickness of the hole-injection layer 111 was set to 50 nm, and the weight ratio of PCzPA to molybdenum oxide was adjusted to 4:2 (=PCzPA:molybdenum oxide). Note that the co-evaporation method refers to an evaporation method in which evaporation is carried out from a plurality of evaporation sources at the same time in one treatment chamber.

Next, a film of PCzPA was formed to a thickness of 10 nm over the hole-injection layer 111 to form the hole-transport layer 112.

Furthermore, over the hole-transport layer 112, the light-emitting layer 113 was formed by co-evaporation of 9-[4-(10-phenyl-9-anthryl)phenyl]-9H-carbazole (abbreviation: CzPA) represented by Structural formula (ii) and N,N′-bis[3-(dibenzofuran-4-yl)-2,6-dimethylphenyl]-N,N′-diphenylpyrene-1,6-diamine (abbreviation: 1,6mFrBAPrn-04) represented by Structural formula (2200) with a weight ratio of 1:0.03 (=CzPA: 1,6mFrBAPrn-04) to a thickness of 25 nm.

Then, the electron-transport layer 114 was formed over the light-emitting layer 113 in such a way that a 10-nm-thick film of CzPA was formed and a 15-nm-thick film of bathophenanthroline (abbreviation: BPhen) represented by Structural formula (iv) was formed.

After the formation of the electron-transport layer 114, lithium fluoride (LiF) was deposited by evaporation to a thickness of 1 nm to form the electron-injection layer 115. Finally, aluminum was deposited by evaporation to a thickness of 200 nm to form the second electrode 102 functioning as a cathode. Through the above-described steps, Light-emitting element 3 of this example was fabricated.

### (Method of Fabricating Comparative Light-Emitting Element 2)

Comparative light-emitting element 2 was fabricated in the same manner as Light-emitting element 3 except that 1,6mFrBAPrn-04 in the light-emitting layer 113 of Light-emitting element 3 was replaced with N,N′-bis[3-(dibenzofuran-4-yl)phenyl]-N,N′-diphenylpyrene-1,6-diamine (abbreviation: 1,6mFrBAPrn-II) represented by Structural formula (v).

The element structures of Light-emitting element 3 and Comparative light-emitting element 2 are listed in Table 3.

TABLE 3

| | | Hole- | | Electron- | | |
|---|---|---|---|---|---|---|
| | Hole- | transport | | transport | | Electron- |
| | injection layer | layer | Light-emitting layer | layer | | injection |
| | 50 nm | 10 nm | 25 nm | 10 nm | 15 nm | layer |
| Light-emitting | PCzPA:MoOx | PCzPA | CzPA:1,6mFrBAPrn-04 | CzPA | BPhen | LiF |
| element 3 | 4:2 | | 1:0.03 | | | |
| Comparative | | | CzPA:1,6mFrBAPrn-II | | | |
| light-emitting | | | 1:0.03 | | | |
| element 2 | | | | | | |

Light-emitting element 3 and Comparative light-emitting element 2 were each sealed using a glass substrate in a glove box containing a nitrogen atmosphere so as not to be exposed to the air (specifically, a sealing material was applied onto an outer edge of the element and UV treatment and heat treatment at 80° C. for an hour were performed at the time of sealing). Then, reliability of these light-emitting elements was measured. Note that the measurements were performed at room temperature (in an atmosphere kept at 25° C.).

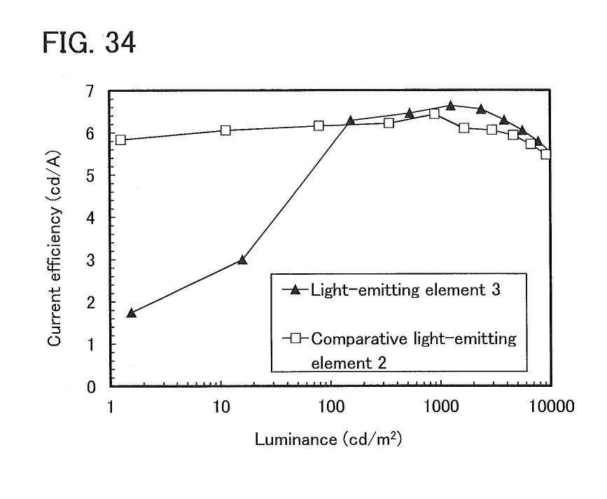

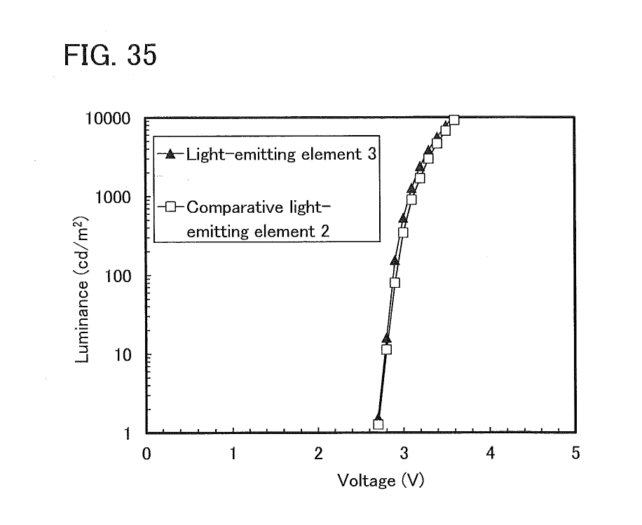

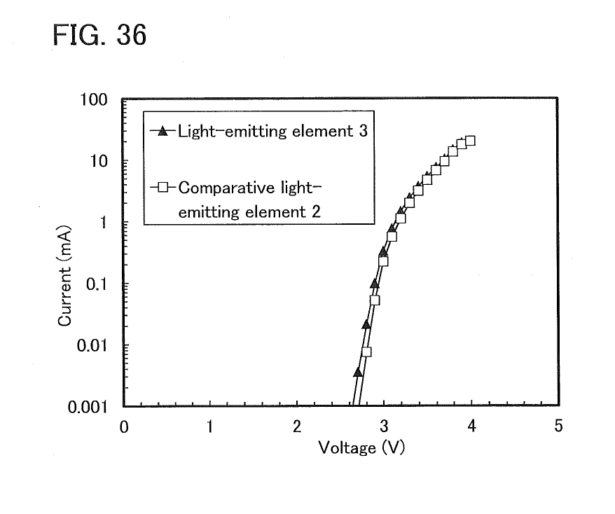

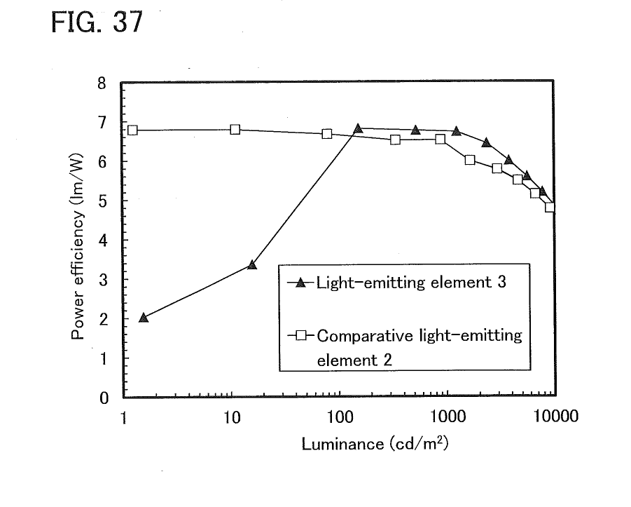

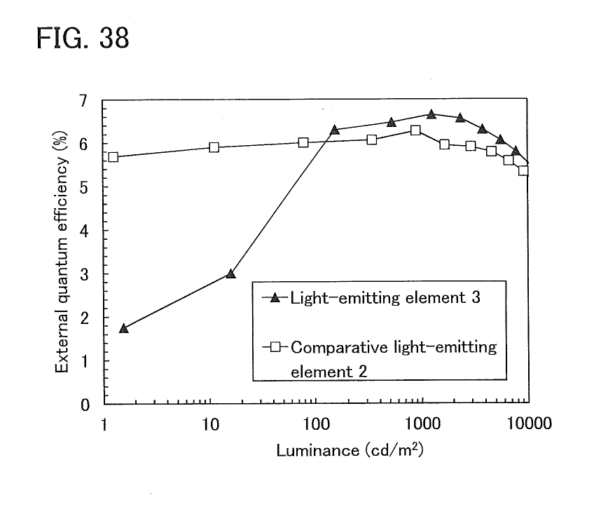

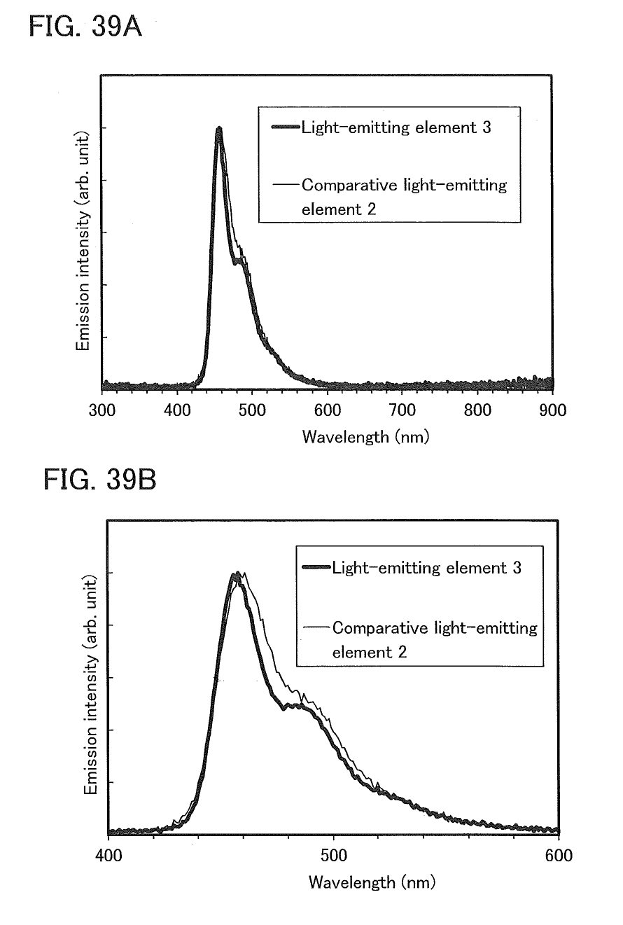

FIG. 34 shows luminance-current efficiency characteristics of Light-emitting element 3 and Comparative light-emitting element 2. FIG. 35 shows voltage-luminance characteristics of thereof. FIG. 36 shows voltage-current characteristics thereof. FIG. 37 shows luminance-power efficiency characteristics thereof. FIG. 38 shows luminance-external quantum efficiency characteristics thereof. FIGS. 39A and 39B show emission spectra thereof.

The results show that Light-emitting element 3 and Comparative light-emitting element 2 both have favorable characteristics. Particularly in a luminance region with a practical luminance of 100 cd/m² or higher, Light-emitting element 3 exhibits better characteristics than Comparative light-emitting element 2. FIG. 39B is an enlarged view of the spectrum ranging from 400 nm to 600 nm in FIG. 39A. As can be seen from FIG. 39B, Light-emitting element 3 has a narrower spectrum than Comparative light-emitting element 2, and has a smaller peak wavelength than Comparative light-emitting element 2.

The external quantum efficiency of Light-emitting element 3 in a luminance region with the practical luminance is better than that of Comparative light-emitting element 2. Although the maximum values of emission spectra shown in FIGS. 39A and 39B are normalized to 1, the maximum value of an emission intensity of Light-emitting element 3, which has high quantum efficiency and a small half width of an emission spectrum, is larger than the maximum value of an emission intensity of Comparative light-emitting element 2. In view of a small amount of light decayed by the cavity effect or a small amount of light intercepted with a color filter, with the use of 1,6mFrBAPrn-04, which is a 1,6-bis(diphenylamino)pyrene derivative of one embodiment of the present invention, a light-emitting element with extremely high emission efficiency or a light-emitting element with extremely low power consumption can be obtained.

Light-emitting element 3 was driven at a constant current of 2.5 mA, and after 260 hours, 66% of the luminance was maintained. In 1,6mFrBAPrn-04, two methyl groups are bonded to a phenyl group having a dibenzofuranyl group among two phenyl groups of diphenylamine. 1,6mFrBAPrn-04 is an organic compound that enables fabrication of a light-emitting element with high reliability.

Light-emitting element 3 including the 1,6-bis(diphenylamino)pyrene derivative in which an alkyl group is bonded to each of the two ortho positions of at least one of the two phenyl groups in each of the two diphenylamino groups (1,6mFrBAPrn-04 is used in this example) as a phosphorescent substance has characteristics higher than or similar to those of Comparative light-emitting element 2 including a 1,6-bis(diphenylamino)pyrene derivative without the above structure (1,6mFrBAPrn-II). In addition, Light-emitting element 3 has a narrower half width of an emission spectrum than Comparative light-emitting element 2.

### Example 6

In this example, a light-emitting element (Light-emitting element 4) of one embodiment of the present invention is described. Structural formulae of organic compounds used in Light-emitting element 4 are shown below.

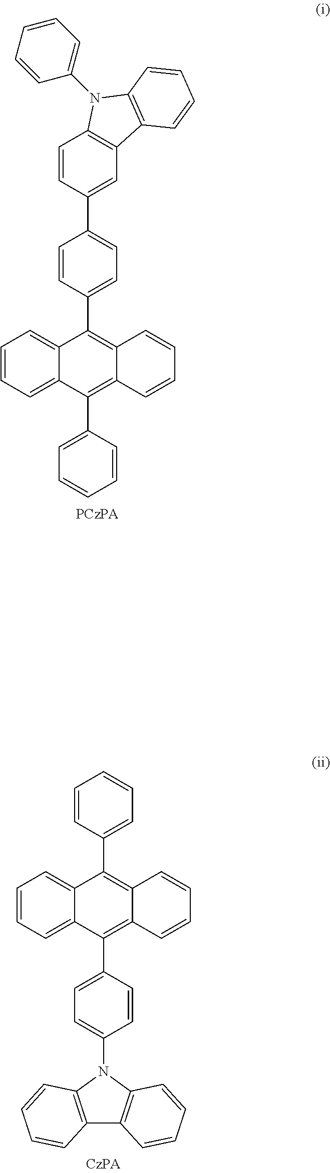 [SMILES: c1ccc(-c2c3ccccc3c(-c3ccc(-c4ccc5c(c4)c4ccccc4n5-c4ccccc4)cc3)c3ccccc23)cc1 ; c1ccc(-c2c3ccccc3c(-c3ccc(-n4c5ccccc5c5ccccc54)cc3)c3ccccc23)cc1]

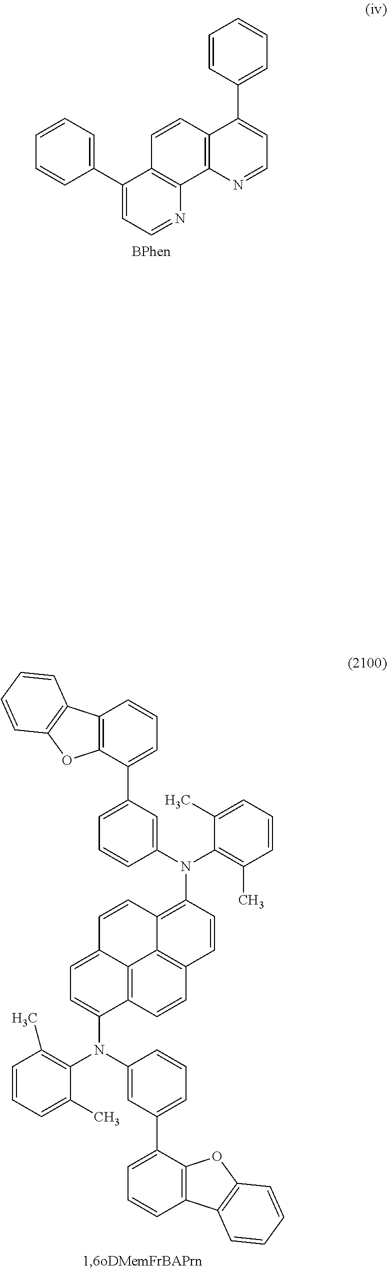 [SMILES: c1ccc(-c2ccnc3c2ccc2c(-c4ccccc4)ccnc23)cc1 ; Cc1cccc(C)c1N(c1cccc(-c2cccc3c2oc2ccccc23)c1)c1ccc2ccc3c(N(c4cccc(-c5cccc6c5oc5ccccc56)c4)c4c(C)cccc4C)ccc4ccc1c2c43]

### (Method of Manufacturing Light-Emitting Element 4)

First, a film of indium tin oxide containing silicon oxide (ITSO) was formed over a glass substrate by a sputtering method, so that the first electrode 101 was formed. The thickness of the first electrode 101 was set to 110 nm and the area of the electrode was set to 2 mm×2 mm. Here, the first electrode 101 is an electrode that functions as an anode of a light-emitting element.

Next, in pretreatment for forming the light-emitting element over the substrate, a surface of the substrate was washed with water and baked at 200° C. for an hour, and then UV ozone treatment was performed for 370 seconds.

Then, the substrate was transferred into a vacuum evaporation apparatus whose pressure was reduced to approximately 10⁻⁴ Pa, vacuum baking at 170° C. for 30 minutes was performed in a heating chamber of the vacuum evaporation apparatus, and then the substrate was cooled down for approximately 30 minutes.

Then, the substrate over which the first electrode 101 was formed was fixed to a substrate holder provided in the vacuum evaporation apparatus so that the surface on which the first electrode 101 was formed faced downward. The pressure in the vacuum evaporation apparatus was reduced to about 10⁻⁴ Pa. After that, over the first electrode 101, 9-phenyl-3-[4-(10-phenyl-9-anthryl)phenyl]-9H-carbazole (abbreviation: PCzPA) represented by the above Structural formula (i) and molybdenum(VI) oxide were deposited by co-evaporation by an evaporation method using resistance heating, so that the hole-injection layer 111 was formed. The thickness of the hole-injection layer 111 was set to 50 nm, and the weight ratio of PCzPA to molybdenum oxide was adjusted to 4:2 (=PCzPA:molybdenum oxide). Note that the co-evaporation method refers to an evaporation method in which evaporation is carried out from a plurality of evaporation sources at the same time in one treatment chamber.

Next, a film of PCzPA was formed to a thickness of 10 nm over the hole-injection layer 111 to form the hole-transport layer 112.

Furthermore, over the hole-transport layer 112, the light-emitting layer 113 was formed by co-evaporation of 9-[4-(10-phenyl-9-anthryl)phenyl]-9H-carbazole (abbreviation: CzPA) represented by Structural formula (ii) and N,N′-bis[3-(dibenzofuran-4-yl)phenyl]-N,N′-bis(2,6-dimethylphenyl)pyrene-1,6-diamine (abbreviation: 1,6oDMemFrBAPrn) represented by Structural formula (2100) with a weight ratio of 1:0.03 (=CzPA:1,6oDMemFrBAPrn) to a thickness of 25 nm.

Then, the electron-transport layer 114 was formed over the light-emitting layer 113 in such a way that a 10-nm-thick film of CzPA was formed and a 15-nm-thick film of bathophenanthroline (abbreviation: BPhen) represented by Structural formula (iv) was formed.

After the formation of the electron-transport layer 114, lithium fluoride (LiF) was deposited by evaporation to a thickness of 1 nm to form the electron-injection layer 115. Finally, aluminum was deposited by evaporation to a thickness of 200 nm to form the second electrode 102 functioning as a cathode. Through the above-described steps, Light-emitting element 4 of this example was fabricated.

The element structure of Light-emitting element 4 is listed in Table 4.

TABLE 4

| | | Hole- | | | | |
|---|---|---|---|---|---|---|
| | Hole- | transport | | Electron- | | Electron- |
| | injection layer | layer | Light-emitting layer | transport layer | | injection |
| | 50 nm | 10 nm | 25 nm | 10 nm | 15 nm | layer |
| Light- | PCzPA:MoOx | PCzPA | CzPA:1, | CzPA | BPhen | LiF |
| emitting | 4:2 | | 6oDMemFrBAPrn | | | |
| element 4 | | | 1:0.03 | | | |

Light-emitting element 4 was sealed using a glass substrate in a glove box containing a nitrogen atmosphere so as not to be exposed to the air (specifically, a sealing material was applied onto an outer edge of the element and UV treatment and heat treatment at 80° C. for an hour were performed at the time of sealing). Then, reliability of these light-emitting elements was measured. Note that the measurements were performed at room temperature (in an atmosphere kept at 25° C.).

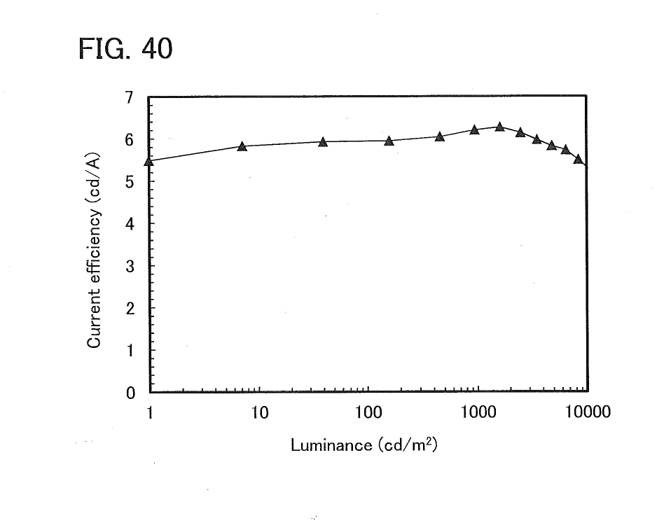

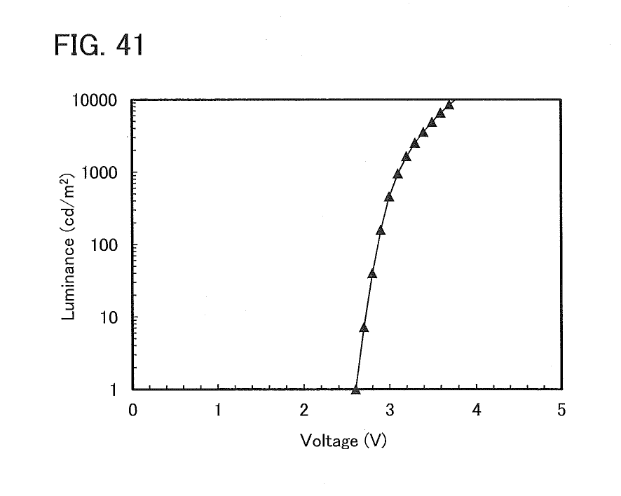

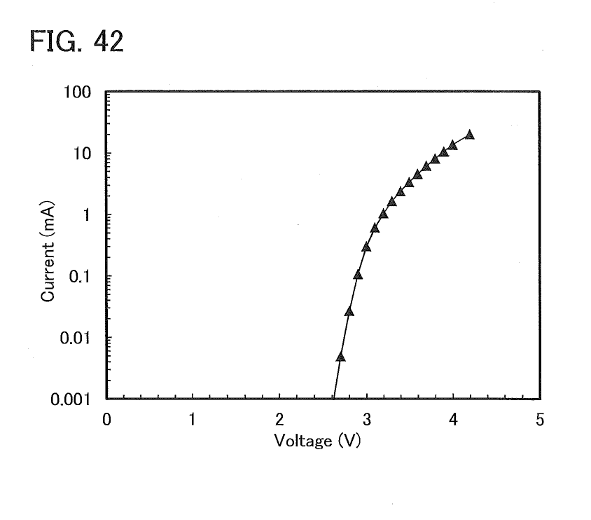

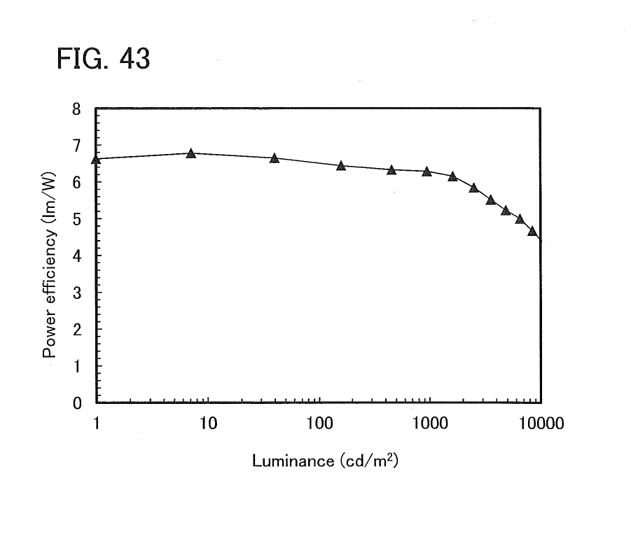

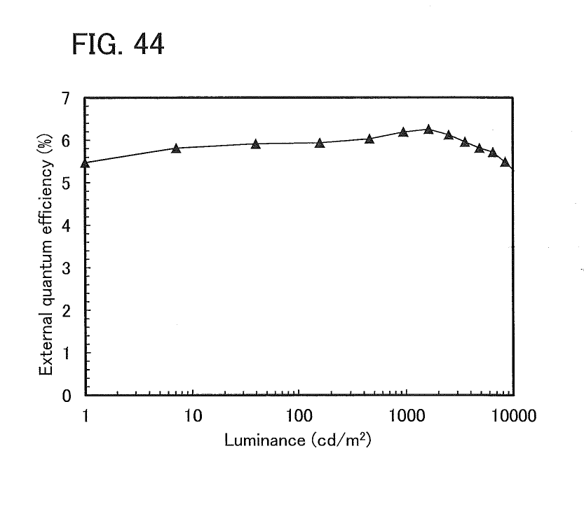

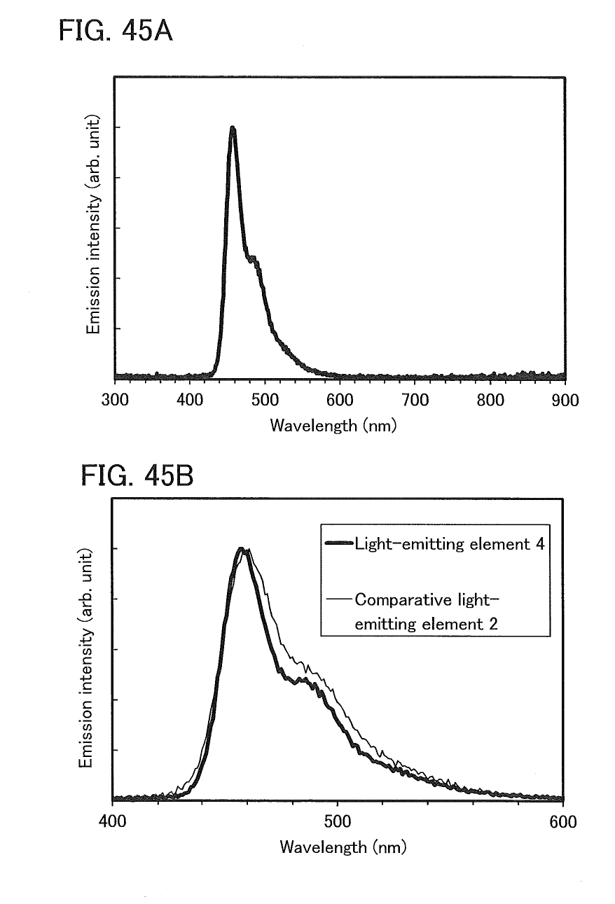

FIG. 40 shows luminance-current efficiency characteristics of Light-emitting element 4. FIG. 41 shows voltage-luminance characteristics of thereof. FIG. 42 shows voltage-current characteristics thereof. FIG. 43 shows luminance-power efficiency characteristics thereof. FIG. 44 shows luminance-external quantum efficiency characteristics thereof. FIGS. 45A and 45B show emission spectra thereof.

The results show that Light-emitting element 4 has favorable characteristics. FIG. 45B is an enlarged view of the spectrum ranging from 400 nm to 600 nm in FIG. 45A, and overlapped with the emission spectrum of Comparative light-emitting element 2 fabricated in Example 5 for reference. As can be seen from FIG. 45B, Light-emitting element 4 has a narrower spectrum than Comparative light-emitting element 2, and has a smaller peak wavelength than Comparative light-emitting element 2.

The external quantum efficiency of Light-emitting element 4 in a luminance region with the practical luminance is similar to that of Comparative light-emitting element 2 of Example 5. Although the maximum values of emission spectra shown in FIGS. 45A and 45B are normalized to 1, the maximum value of an emission intensity of Light-emitting element 4, which has substantially the same quantum efficiency and has a small half width of an emission spectrum, is larger than the maximum value of an emission intensity of Comparative light-emitting element 2. In view of a small amount of light decayed by the cavity effect or a small amount of light intercepted with a color filter, with the use of 1,6oDMemFrBAPrn, which is a 1,6-bis(diphenylamino)pyrene derivative of one embodiment of the present invention, a light-emitting element with extremely high emission efficiency or a light-emitting element with extremely low power consumption can be obtained.

Light-emitting element 4 was driven at a constant current of 2.5 mA, and after 89 hours, 65% of the luminance was maintained.

Light-emitting element 4 including the 1,6-bis(diphenylamino)pyrene derivative in which an alkyl group is bonded to each of the two ortho positions of at least one of the two phenyl groups in each of the two diphenylamino groups (1,6oDMemFrBAPrn is used in this example) as a phosphorescent substance has a narrower half width of an emission spectrum than Comparative light-emitting element 2 including a 1,6-bis(diphenylamino)pyrene derivative without the above structure (1,6mFrBAPrn-II).

### Example 7

In this example, an emission spectrum of the 1,6-bis(diphenylamino)pyrene derivative of one embodiment of the present invention in which an alkyl group is bonded to each of the two ortho positions of at least one of the two phenyl groups in each of the two diphenylamino groups is compared with an emission spectrum of a 1,6-bis(diphenylamino)pyrene derivative without the above structure, and the comparison results are shown.

Structural formulae of organic compounds used in this example are shown below.

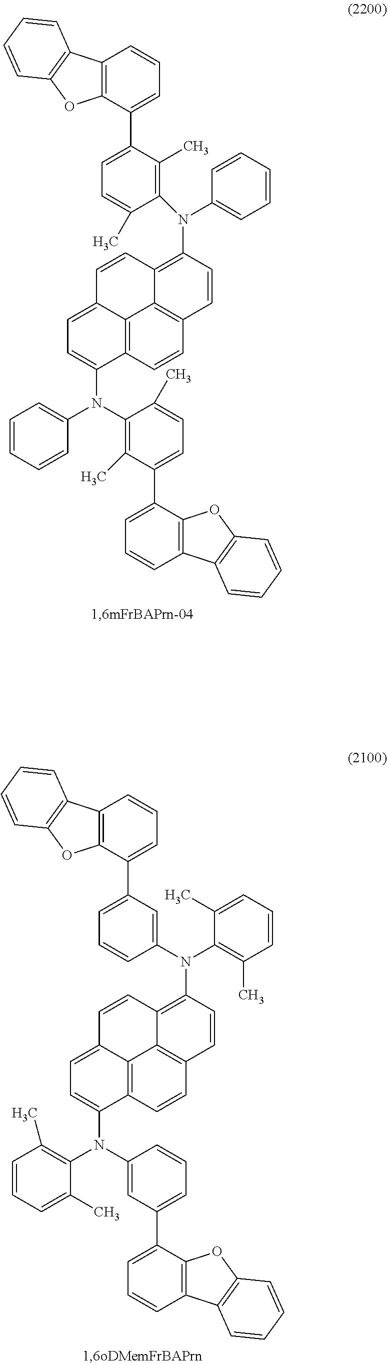 [SMILES: Cc1ccc(-c2cccc3c2oc2ccccc23)c(C)c1N(c1ccccc1)c1ccc2ccc3c(N(c4ccccc4)c4c(C)ccc(-c5cccc6c5oc5ccccc56)c4C)ccc4ccc1c2c43 ; Cc1cccc(C)c1N(c1cccc(-c2cccc3c2oc2ccccc23)c1)c1ccc2ccc3c(N(c4cccc(-c5cccc6c5oc5ccccc56)c4)c4c(C)cccc4C)ccc4ccc1c2c43]

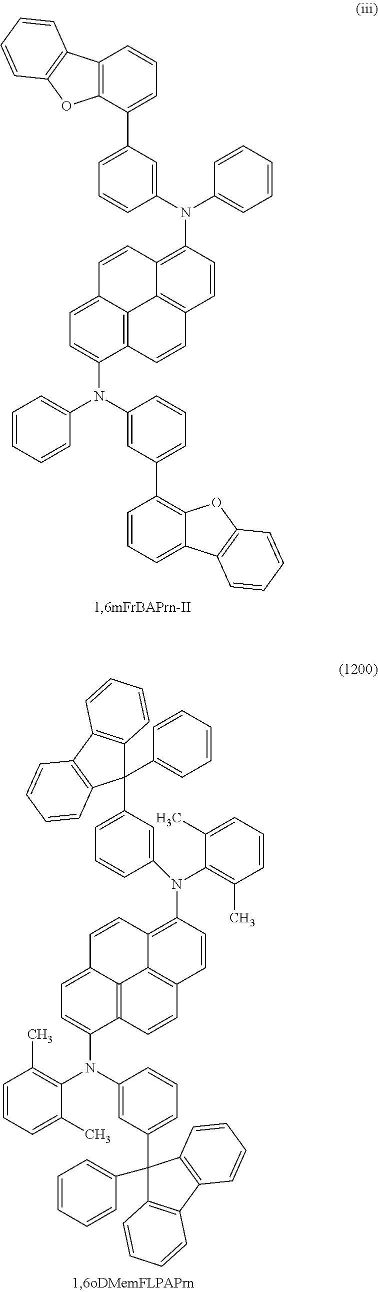 [SMILES: c1ccc(N(c2cccc(-c3cccc4c3oc3ccccc34)c2)c2ccc3ccc4c(N(c5ccccc5)c5cccc(-c6cccc7c6oc6ccccc67)c5)ccc5ccc2c3c54)cc1 ; Cc1cccc(C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C)cccc4C)ccc4ccc1c2c43]

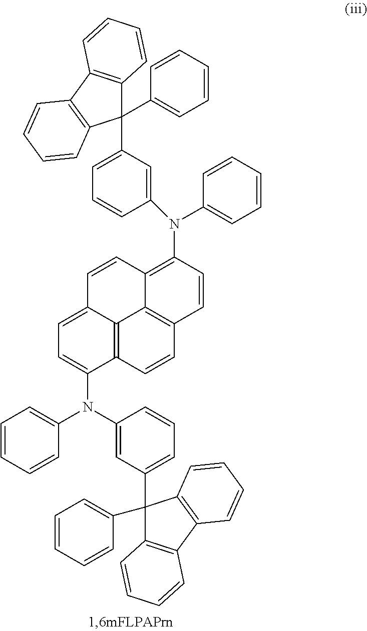 [SMILES: c1ccc(N(c2cccc(C3(c4ccccc4)c4ccccc4-c4ccccc43)c2)c2ccc3ccc4c(N(c5ccccc5)c5cccc(C6(c7ccccc7)c7ccccc7-c7ccccc76)c5)ccc5ccc2c3c54)cc1]

Among the organic compounds shown above, 1,6mFrBAPrn-04, 1,6oDMemFrBAPrn, and 1,6oDMemFLPAPrn are each the 1,6-bis(diphenylamino)pyrene derivative of one embodiment of the present invention. In each of two diphenylamino groups in the derivative, an alkyl group is bonded to each of the two ortho positions of at least one the two phenyl groups in each of the two diphenylamino groups. Meanwhile, 1,6mFrBAPrn-II and 1,6mFLPAPrn are each a 1,6-bis(diphenylamino)pyrene derivative without the above structure, and used as comparative examples.

As shown in the above structural formulae, a structural difference between the 1,6-bis(diphenylamino)pyrene derivative of one embodiment of the present invention and the 1,6-bis(diphenylamino)pyrene derivative of a comparative example is only whether two methyl groups are bonded to the ortho positions (with respect to the pyrene skeleton) of a phenyl or phenylene group bonded to a diphenylamine.

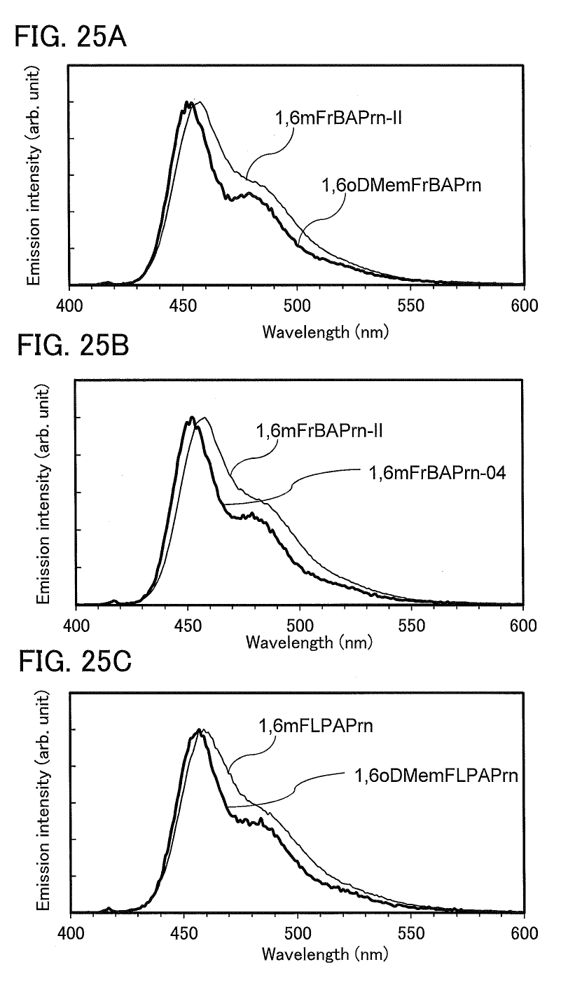

FIGS. 25A to 25C show emission spectra of the compounds in a toluene solution. FIG. 25A shows emission spectra of 1,6oDMemFrBAPrn and 1,6mFrBAPrn-II, FIG. 25B shows emission spectra of 1,6mFrBAPrn-04 and 1,6mFrBAPrn-II, and FIG. 25C shows emission spectra of 1,6oDMemFLPAPrn and 1,6mFLPAPrn.

In each graph, the 1,6-bis(diphenylamino)pyrene derivative of one embodiment of the present invention has a narrower half width of an emission spectrum, a narrower spectrum, and a peak wavelength on a shorter wavelength side.

Table 5 lists an absorption wavelength (energy), an emission wavelength (energy), and a difference between the absorption wavelength and the emission wavelength of each organic compound. The difference corresponds to a Stokes shift of an organic compound.

TABLE 5

| | Absorption | | Emission | | | |
|---|---|---|---|---|---|---|
| | wavelength | | wavelength | | Difference | |
| | (nm) | (eV) | (nm) | (eV) | (nm) | (eV) |
| 1,6mFrBAPrn-04 | 435 | 2.851 | 452 | 2.743 | 17 | 0.107 |
| 1,6oDMemFrBAPrn | 436 | 2.844 | 453 | 2.737 | 17 | 0.107 |
| 1,6oDMemFLPAPrn | 438 | 2.831 | 457 | 2.713 | 19 | 0.118 |
| 1,6mFrBAPrn-II | 428 | 2.897 | 458 | 2.707 | 30 | 0.190 |
| 1,6mFLPAPrn | 430 | 2.884 | 459 | 2.702 | 29 | 0.182 |

Table 5 shows that 1,6mFrBAPrn-04, 1,6oDMemFrBAPrn, and 1,6oDMemFLPAPrn, each of which is a 1,6-bis(diphenylamino)pyrene derivative of one embodiment of the present invention, have a Stokes shift of 0.18 eV or smaller. This value is smaller than the Stokes shift of each of 1,6mFrBAPrn-II and 1,6mFLPAPrn, which are comparative examples. Note that the Stokes shift is preferably 0.15 eV or smaller, more preferably 0.12 eV or smaller.

Accordingly, the 1,6-bis(diphenylamino)pyrene derivative of one embodiment of the present invention has a smaller Stokes shift than a 1,6-bis(diphenylamino)pyrene derivative without the structure of one embodiment of the present invention. Therefore, the 1,6-bis(diphenylamino)pyrene derivative of one embodiment of the present invention emits light with a narrow half width of an emission spectrum and thus can provide blue light with excellent color purity. A light-emitting element including the 1,6-bis(diphenylamino)pyrene derivative can reduce energy loss with use of a microcavity structure or an color filter; therefore, the light-emitting element can have high efficiency and emit excellent blue light easily as compared with a conventional light-emitting element. Moreover, the light-emitting element can emit excellent blue light with low excitation energy, which means the light-emitting element consumes less power.

This application is based on Japanese Patent Application serial no. 2014-032002 filed with Japan Patent Office on Feb. 21, 2014 and Japanese Patent Application serial no. 2014-031853 filed with Japan Patent Office on Feb. 21, 2014, the entire contents of which are hereby incorporated by reference.

What is claimed is:

1. A light-emitting element comprising:
a pair of electrodes; and
an EL layer between the pair of electrodes;
wherein the EL layer comprises at least a light-emitting material,
wherein the light-emitting material is a 1,6-bis(diphenylamino)pyrene derivative, and
wherein a structural change between an excited state and a ground state in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

2. The light-emitting element according to claim 1,
wherein the 1,6-bis(diphenylamino)pyrene derivative comprises two diphenylamino groups,
wherein each of the two diphenylamino groups comprises two phenyl groups,
wherein an alkyl group is bonded to each of two ortho positions of one of the two phenyl groups, and
wherein hydrogen is bonded to each of two ortho positions of the other of the two phenyl groups.

3. The light-emitting element according to claim 1, wherein a Stokes shift of the light-emitting material is less than or equal to 0.18 eV.

4. The light-emitting element according to claim 1,
wherein the EL layer further comprises a host material,
wherein the light-emitting material is dispersed in the host material, and
wherein an absorption spectrum peak of the light-emitting material on the longest wavelength side overlaps with an emission spectrum of the host material.

5. A light-emitting element comprising:
a pair of electrodes; and
an EL layer between the pair of electrodes;
wherein the EL layer comprises at least a light-emitting material,
wherein the light-emitting material is a 1,6-bis(diphenylamino)pyrene derivative,
wherein the 1,6-bis(diphenylamino)pyrene derivative comprises two diphenylamino groups,
wherein each of the two diphenylamino groups comprises two phenyl groups, and
wherein an alkyl group is bonded to each of two ortho positions of at least one of the two phenyl groups.

6. The light-emitting element according to claim 5,
wherein a structural change between an excited state and a ground state in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

7. A light-emitting element according to claim 5,
wherein a Stokes shift of the 1,6-bis(diphenylamino)pyrene derivative is smaller than that of a 1,6-bis(diphenylamino)pyrene derivative in which hydrogen is bonded to ortho positions of two phenyl groups of each of two diphenylamino groups.

8. A light-emitting element according to claim 5,
wherein a half width of an emission spectrum of the 1,6-bis(diphenylamino)pyrene derivative is narrower than that of a 1,6-bis(diphenylamino)pyrene derivative in which hydrogen is bonded to ortho positions of two phenyl groups of each of two diphenylamino groups.

9. The light-emitting element according to claim 5, wherein a Stokes shift of the light-emitting material is less than or equal to 0.18 eV.

10. The light-emitting element according to claim 5,
wherein the EL layer further comprises a host material,
wherein the light-emitting material is dispersed in the host material, and
wherein an absorption spectrum peak of the light-emitting material on the longest wavelength side overlaps with an emission spectrum of the host material.

11. A 1,6-bis(diphenylamino)pyrene derivative comprising two diphenylamino groups,
wherein each of the two diphenylamino groups comprises two phenyl groups,
wherein an alkyl group is bonded to each of two ortho positions of at least one of the two phenyl groups, and
wherein a structural change between an excited state and a ground state in the 1,6-bis(diphenylamino)pyrene derivative is smaller than that in a 1,6-bis(diphenylamino)pyrene derivative in which hydrogen is bonded to ortho positions of two phenyl groups of each of two diphenylamino groups.

12. The 1,6-bis(diphenylamino)pyrene derivative according to claim 11, wherein in each of the two diphenylamino groups, an alkyl group is bonded to two ortho positions of one phenyl group, and hydrogen is bonded to two ortho positions of the other phenyl group.

13. The 1,6-bis(diphenylamino)pyrene derivative according to claim 11, wherein a Stokes shift of the 1,6-bis(diphenylamino)pyrene derivative is less than or equal to 0.18 eV.

14. The 1,6-bis(diphenylamino)pyrene derivative according to claim 11, wherein a half width of an emission spectrum of the 1,6-bis(diphenylamino)pyrene derivative is less than or equal to 40 nm.

15. The 1,6-bis(diphenylamino)pyrene derivative according to claim 11, wherein an emission peak wavelength of the 1,6-bis(diphenylamino)pyrene derivative is less than or equal to 465 nm.

16. An organic compound represented by General Formula (G1-2): 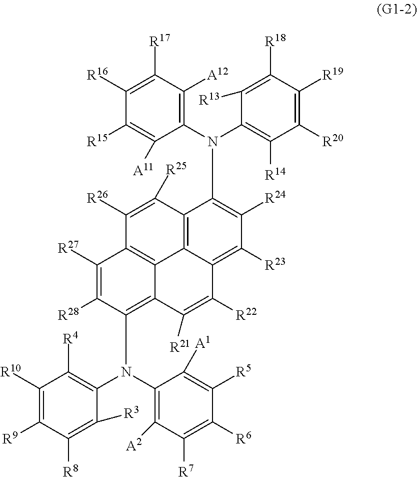
wherein:
A¹, A², A¹¹, and A¹² each represent an alkyl group having 1 to 6 carbon atoms;
at least one of R³ and R⁴ represents hydrogen and the other represents hydrogen or an alkyl group having 1 to 6 carbon atoms;
at least one of R¹³ and R¹⁴ represents hydrogen and the other represents hydrogen or an alkyl group having 1 to 6 carbon atoms;
R⁵ to R¹⁰, R¹⁵ to R²⁰, and R²¹ to R²⁸ each independently represent hydrogen, an alkyl group having 1 to 6 carbon atoms, or an aryl group having 6 to 25 carbon atoms; and
any one of R⁵ to R¹⁰ and any one of R¹⁵ to R²⁰ are substituents represented by General Formula (g1-2): 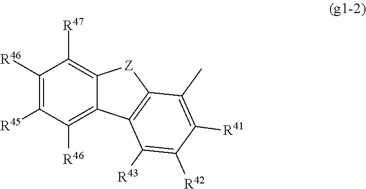
wherein:
R⁴¹ to R⁴⁷ each independently represent hydrogen, an alkyl group having 1 to 6 carbon atoms, or an aryl group having 6 to 25 carbon atoms; and
Z represents an oxygen atom and a sulfur atom.

17. The organic compound according to claim 16, wherein the organic compound is represented by Structural formula (2100):

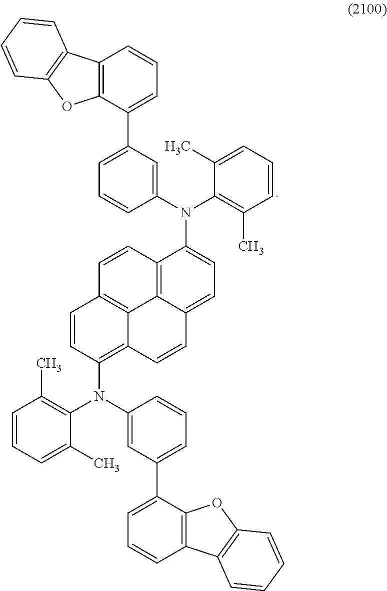 [SMILES: Cc1cccc(C)c1N(c1cccc(-c2cccc3c2oc2ccccc23)c1)c1ccc2ccc3c(N(c4cccc(-c5cccc6c5oc5ccccc56)c4)c4c(C)cccc4C)ccc4ccc1c2c43]

18. The organic compound according to claim 16, wherein the organic compound is represented by Structural formula (2200):

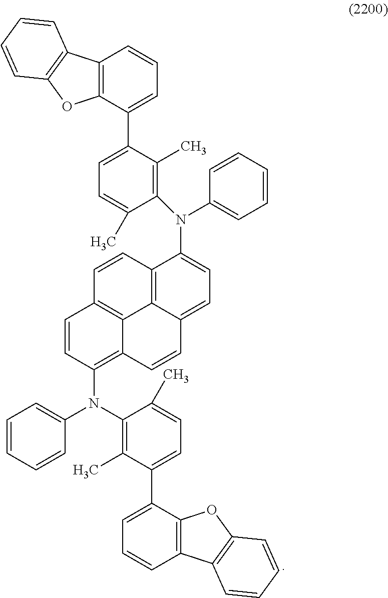 [SMILES: Cc1ccc(-c2cccc3c2oc2ccccc23)c(C)c1N(c1ccccc1)c1ccc2ccc3c(N(c4ccccc4)c4c(C)ccc(-c5cccc6c5oc5ccccc56)c4C)ccc4ccc1c2c43]

19. An organic compound represented by General Formula (G1-1): 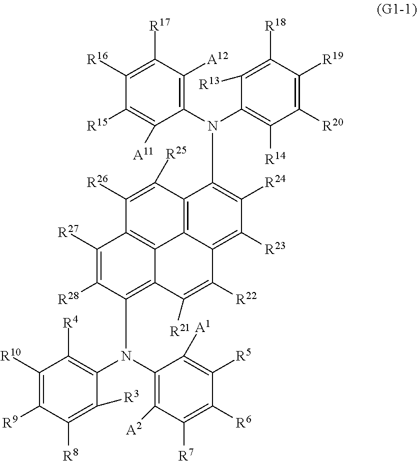
wherein:
A¹, A², A¹¹, and A¹² each represent an alkyl group having 1 to 6 carbon atoms;
at least one of R³ and R⁴ represents hydrogen and the other represents hydrogen or an alkyl group having 1 to 6 carbon atoms;
at least one of R¹³ and R¹⁴ represents hydrogen and the other represents hydrogen or an alkyl group having 1 to 6 carbon atoms;
R⁵ to R¹⁰, R¹⁵ to R²⁰, and R²¹ to R²⁸ each independently represent hydrogen, an alkyl group having 1 to 6 carbon atoms, or an aryl group having 6 to 25 carbon atoms; and
any one of R⁵ to R¹⁰ and any one of R¹⁵ to R²⁰ are substituents represented by General Formula (g1-1): 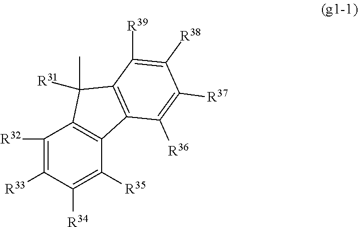
wherein R³¹ to R³⁹ each independently represent hydrogen, an alkyl group having 1 to 6 carbon atoms, or an aryl group having 6 to 25 carbon atoms.

20. The organic compound according to claim 19, wherein the organic compound is represented by Structural formula (1200): 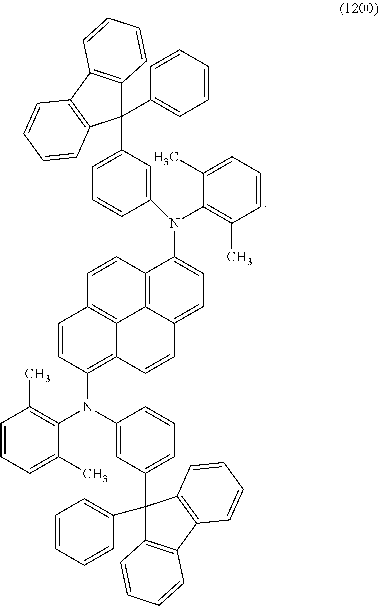 [SMILES: Cc1cccc(C)c1N(c1cccc(C2(c3ccccc3)c3ccccc3-c3ccccc32)c1)c1ccc2ccc3c(N(c4cccc(C5(c6ccccc6)c6ccccc6-c6ccccc65)c4)c4c(C)cccc4C)ccc4ccc1c2c43]
